(19) United States
(12) Patent Application Publication
(10) Pub. No.: US 2020/0062696 A1
(43) Pub. Date: Feb. 27, 2020

(54) COMPOSITIONS AND METHODS FOR INHIBITING RETICULON 4

(71) Applicant: THE REGENTS OF THE UNIVERSITY OF CALIFORNIA, Oakland, CA (US)

(72) Inventors: Daniel K. Nomura, Berkeley, CA (US); James A. Olzmann, Oakland, CA (US); Leslie A. Bateman, Boston, MA (US); Truc B. Nguyen, Albany, CA (US); David K. Miyamoto, Cambridge, MA (US); Tucker R. Huffman, El Dorado Hills, CA (US); Allison M. Roberts, Kensington, CA (US)

(21) Appl. No.: 16/482,947

(22) PCT Filed: Feb. 2, 2018
(86) PCT No.: PCT/US2018/016650

§ 371 Date: Aug. 1, 2019

Related U.S. Application Data
(60) Provisional application No. 62/454,681, filed on Feb. 3, 2017.
(60) Provisional application No. 62/471,865, filed on Mar. 15, 2017.

(51) Int. Cl. C07C 233/27; A61P 35/00; C07D 295/033; C07C 233/09; C07D 295/104

(52) U.S. Cl. CPC C07C 233/27; A61P 35/00; A61K 45/06; C07C 233/09; C07D 295/104; C07D 295/033

(57) ABSTRACT

Disclosed herein, inter alia, are compositions and methods useful for inhibiting reticulon 4 (RTN4).

### CROSS-REFERENCES TO RELATED APPLICATIONS

This application claims the benefit of U.S. Provisional Application No. 62/454,681, filed Feb. 3, 2017, and U.S. Provisional Application No. 62/471,865, filed Mar. 15, 2017, which are incorporated herein by reference in their entirety and for all purposes.

### STATEMENT AS TO RIGHTS TO INVENTIONS MADE UNDER FEDERALLY SPONSORED RESEARCH AND DEVELOPMENT

This invention was made with government support under CA172667 and GM112948 awarded by the National Institutes of Health and under W81XWH-15-1-0050 awarded by ARMY/MRMC. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 052103-503001WO Sequence Listing_ST25.txt, created Jan. 11, 2018, 166,113 bytes, machine format IBM-PC, MS Windows operating system, is hereby incorporated by reference.

### BACKGROUND

In the United States, it is estimated that over 134,000 people will be diagnosed with colorectal cancer and over 49,000 patients will die from colorectal cancer¹. Current therapeutic strategies for colorectal cancer include resection and non-specific therapies such as radiation or chemotherapy². Unfortunately, these treatment strategies are insufficient for aggressive and metastatic colorectal cancers, and thus better strategies are needed to discover both novel anti-cancer agents and targets for combatting colorectal cancer. Towards this goal, identifying new anti-cancer targets, druggable nodes, and lead small-molecules are critical for combatting colorectal cancer. Disclosed herein, inter alia, are solutions to these and other problems in the art.

### BRIEF SUMMARY

Herein are provided, inter alia, compounds capable of modulating the level of activity of reticulon 4 and methods of using the same.

In an aspect is provided a compound having the formula:

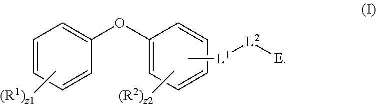

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl. The symbol z1 is an integer from 0 to 5. R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}—C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Two adjacent R² substituents may optionally be joined to form a substituted or unsubstituted cycloalkyl, substituted or unsubstituted heterocycloalkyl, substituted or unsubstituted aryl, or substituted or unsubstituted heteroaryl. The symbol z2 is an integer from 0 to 4. L¹ is a bond, —S(O)₂—, —NR⁴—, —O—, —S—, —C(O)—, —C(O)NR⁴—, —NR^{4C}(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}—, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. E is an electrophilic moiety. Each R^{1A}, R^{1B}, R^{1C}, R^{1D}, R^{2A}, R^{2B}, R^{2C}, R^{2D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{1A} and R^{1B} substituents bonded to the same nitrogen atom may optionally be joined to form a substituted or unsubstituted heterocycloalkyl or substituted or unsubstituted heteroaryl. R^{2A} and R^{2B} substituents bonded to the same nitrogen atom may optionally be joined to form a substituted or unsubstituted heterocycloalkyl or substituted or unsubstituted heteroaryl. R^{4A} and R^{4B} substituents bonded to the same nitrogen atom may optionally be joined to form a substituted or unsubstituted heterocycloalkyl or substituted or unsubstituted heteroaryl. R^{5A} and R^{5B} substituents bonded to the same nitrogen atom may optionally be joined to form a substituted or unsubstituted heterocycloalkyl or substituted or unsubstituted heteroaryl. Each X, X¹, X², X⁴, and X⁵ is independently —F, —Cl, —Br, or —I. The symbols n1, n2, n4, and n5 are independently an integer from 0 to 4. The symbols m1, m2, m4, m5, v1, v2, v4, and v5 are independently an integer from 1 to 2.

In an aspect is provided a pharmaceutical composition including a Reticulon 4 inhibitor and a pharmaceutically acceptable excipient.

In an aspect is provided a pharmaceutical composition including a compound described herein, or pharmaceutically acceptable salt thereof, and a pharmaceutically acceptable excipient.

In an aspect is provided a method of treating cancer, the method including administering to a subject in need thereof an effective amount of a Reticulon 4 inhibitor.

In an aspect is provided a method of treating cancer including administering to a subject in need thereof an effective amount of a compound described herein.

In an aspect is provided a method of treating a disease associated with reticulon 4 activity including administering to a subject in need thereof an effective amount of a Reticulon 4 inhibitor.

In an aspect is provided a method of inhibiting reticulon 4 activity including contacting the reticulon 4 with a Reticulon 4 inhibitor.

In an aspect is provided a method of inhibiting reticulon 4 activity including contacting the reticulon 4 with a compound described herein.

In an aspect is provided a reticulon 4 protein covalently bonded to a Reticulon 4 inhibitor.

In an aspect is provided a reticulon 4 protein covalently bonded to a compound described herein.

### BRIEF DESCRIPTION OF THE DRAWINGS

FIG. 1A-1E. Coupling Screening of Cysteine-Reactive Covalent Ligands with isoTOP-ABPP to Identify Anti-Cancer Compounds, Targets, and Druggable Hotspots for Colorectal Cancer. (FIG. 1A) We screened a library of cysteine-reactive fragment-based covalent ligands in colorectal cancer cells to identify compounds that impair colorectal cancer pathogenicity and used isoTOP-ABPP platforms to identify the targets and druggable hotspots within these targets. (FIG. 1B) The compounds tested, from left to right in the top chart of FIG. 1B (i.e. survival), are DKM 3-30, DKM 3-16, DKM 2-40, DKM 2-91, DKM 2-101, DKM 3-10, DKM 2-94, DKM 2-76, DKM 2-80, TRH 1-55, TRH 1-12, DKM 3-7, DKM 2-95, DKM 3-43, DKM 2-98, DKM 3-36, TRH 1-32, DKM 3-41, DKM 3-70, DKM 2-37, TRH 1-50, DKM 3-5, DKM 2-108, DKM 3-31, DKM 2-83, DKM 2-59, TRH 1-53, DKM 3-32, DKM 2-93, DKM 2-84, DKM 2-113, DKM 3-9, DKM 2-114, DKM 3-13, DKM 2-34, DKM 2-47, DKM 3-29, DKM 2-49, DKM 2-71, DKM 2-43, DKM 2-017, DKM 2-67, DKM 2-50, DKM 2-31, DKM 2-48, DKM 2-32, DKM 2-33, DKM 2-52, DKM 2-39, TRH 1-13, DKM 2-72, DKM 2-58, TRH 1-19, DKM 2-120, DKM 3-42, DKM 2-42, DKM 2-97, DKM 2-60, DKM 2-86, DKM 2-110, TRH 1-20, DKM 2-62, DKM 3-11, DKM 3-4, DKM 2-116, DKM 2-102, DKM 3-12, DKM 2-111, DKM 2-103, DKM 2-100, DKM 2-109, TRH 1-27, DKM 2-106, DKM 3-8, and TRH 1-54. The compounds tested, from left to right in the bottom chart of FIG. 1B (i.e. proliferation), are DKM 2-94, DKM 2-71, DKM 2-98, DKM 2-83, DKM 2-80, DKM 2-76, DKM 3-70, DKM 2-52, TRH 1-55, DKM 3-30, DKM 2-93, DKM 2-91, DKM 3-16, TRH 1-53, DKM 2-67, DKM 2-37, DKM 2-59, TRH 1-50, DKM 3-10, DKM 3-5, DKM 2-84, DKM 2-48, DKM 2-95, TRH 1-12, DKM 2-116, DKM 3-41, DKM 3-13, DKM 3-43, DKM 3-32, DKM 2-62, DKM 2-110, DKM 2-108, DKM 2-120, DKM 2-109, DKM 2-97, DKM 2-101, DKM 3-36, DKM 2-40, DKM 2-107, DKM 3-31, DKM 2-100, DKM 3-7, TRH 1-32, DKM 2-72, DKM 3-9, DKM 2-106, DKM 2-60, DKM 2-86, DKM 3-8, DKM 2-34, DKM 2-111, DKM 3-12, DKM 2-49, DKM 2-39, DKM 2-114, DKM 2-47, DKM 2-103, DKM 3-42, DKM 2-32, DKM 2-33, DKM 2-58, DKM 2-31, DKM 3-11, TRH 1-19, DKM 3-4, DKM 3-29, TRH 1-20, TRH 1-27, DKM 2-43, TRH 1-13, DKM 2-50, DKM 2-102, DKM 2-42, TRH 1-54, and DKM 2-113. Cysteine-reactive covalent ligand screening in SW620 colorectal cancer cells: we screened a cysteine-reactive fragment library consisting of acrylamides and chloroacetamides in SW620 colorectal cancer cells (50 μM) to identify any leads that significantly impaired SW620 serum-free cell survival and proliferation. Survival and proliferation were assessed after 48 h by Hoescht staining. (FIG. 1C, 1D) Shown is the structure of the lead covalent ligand DKM 3-30 (FIG. 1C) that significantly (p<0.05) impaired SW620 cell survival and proliferation (FIG. 1D). (FIG. 1E) SW620 tumor xenograft growth in immune-deficient SCID mice. Mice were subcutaneously injected with SW620 cells to initiate the tumor xenograft study and treatments of mice were initiated with vehicle or DKM 3-30 (50 mg/kg ip, once per day) ten days initiation of the xenograft study. Data in (FIGS. 1B, 1D, 1E) are presented as mean±sem, n=3-8/group. Significance expressed as *p<0.05 compared to vehicle-treated controls. Raw data for screen can be found in Table 1.

FIG. 2A-2D. DKM 3-30 Targets C1101 on RTN4. (FIG. 2A) IsoTOP-ABPP analysis of DKM 3-30 in SW620 colorectal cancer cells. SW620 proteomes were pre-treated with DMSO or DKM 3-30 (50 μM) prior to labeling proteomes with IAyne and appending a biotin-azide handle bearing a TEV protease recognition site and an isotopically light (for DMSO-treated) and heavy (for DKM 3-30-treated) tag. DMSO and treated proteomes were then mixed in a 1:1 ratio and subsequently avidin-enriched, tryptically digested, and then probe-modified tryptic peptides were released by TEV protease and analyzed using quantitative proteomic approaches. IsoTOP-ABPP data represents mean light to heavy ratios for those probe-modified peptides identified in at least 2 out of 3 biological replicates. A light to heavy ratio of 1 indicates that the probe-labeled cysteine-bearing peptide was not bound by the covalent ligands, whereas a ratio >3 indicates bound sites. Also shown on the right are competition studies of DKM 3-30 against IAyne labeling of pure human RTN4 protein. Pure proteins were pre-incubated with the designated concentrations of ligand, followed by labeling with IAyne and visualization of labeling by subsequent click-chemistry mediated appendage of rhodamine-azide, SDS/PAGE, and in-gel fluorescence detection. (FIG. 2B) IsoTOP-ABPP analysis of cysteine-reactivity in pooled primary human colorectal tumors. Nine primary human colorectal tumors were pooled together and labeled with 100 or 10 μM of IAyne followed by subsequent isoTOP-ABPP analysis. Shown are ratios of heavy (100 μM) to light (10 μM) peptides. (FIGS. 2C, 2D) Serum-free cell survival and proliferation (48 h) and tumor xenograft growth in immune-deficient SCID mice from transient siRNA or stable shRNA knockdown of RTN4 in SW620 cells. Expression was determined by qPCR. All data shown represents n=3-6/group. Data in (FIGS. 2C, 2D) are presented as mean±sem. Significance is expressed as *p<0.05 compared to vehicle-treated or si or shControls. Raw data for (FIGS. 2A, 2B) can be found in Table 2.

FIG. 3A-3F. DKM 3-30 disrupts the ER tubular network. (FIG. 3A) A schematic illustration depicts the proposed topology of Rtn4 and the position of C1101 modified by DKM 3-30 (indicated by an arrow). A homology model of human Rtn4 illustrates the membrane-associated portion (lower), the cytosolically accessible portion (upper), and the position of C1101 (central dark gray). (FIG. 3B) U2OS cells expressing GFP-tagged Sec6113, an ER marker, were treated with DKM 3-30 (50 μM) for 16 hr and the ER (light gray/white) and nucleus (dark gray) of fixed cells visualized by fluorescence microscopy. Scale bar=10 μm. (FIGS. 3C, 3D) U2OS cells expressing GFP-tagged Sec61β were treated with vehicle (DMSO) (FIG. 3C) or DKM 3-30 (50 μM) (FIG. 3D) and ER morphology visualized by time-lapse fluorescence microscopy. Time (min) is indicated on each panel. Bottom panels indicate boxed region. (FIG. 3E) U2OS cells were transiently transfected with control or RTN4 siRNA and expression determined by qPCR. Data are presented as mean±sem, n=3. Significance is expressed as *p<0.05. (FIG. 3F) U2OS cells expressing GFP-tagged Sec61β were transfected with siRNAs as in panel (FIG. 3D) and the ER (light gray/white) and nucleus (dark gray) of fixed cells visualized by fluorescence microscopy. Scale bar=10 μm.

FIGS. 4A-4C. DKM 3-30 disrupts nuclear envelope morphology during mitosis. (FIGS. 4A-4C) U2OS cells expressing GFP-tagged Sec61β were treated with vehicle (DMSO) or DKM 3-30 (50 μM) and the ER morphology of mitotic cells visualized by time-lapse fluorescence microscopy. Time (min) is indicated on each panel. Panels (FIGS. 4A, 4B) provide examples of mitotic cells. Enlarged images following mitosis show the nuclear envelope. White arrowheads indicate a GFP-Sec61β structure bisecting the nucleus of a cell incubated with DKM 3-30. Panel (FIG. 4C) shows alterations in the nuclear envelope structure, followed by cell death at the 800 min time point. Bottom panels indicate boxed region.

FIG. 5. Body weight of mice treated with DKM 3-30 in tumor xenograft studies. Mice from tumor xenograft studies shown in FIG. 1E were weighed at the end of the study. The mice treated with DKM 3-30 did not show any significant changes in body weight compared to vehicle-treated control mice. Data are presented as mean±sem, n=8 mice/group.

FIG. 6. Sequence alignment of human and Xenopus laevis RTN4. The position of the shared cysteine in human RTN4 (C1101) and Xenopus laevis RTN4 (C952) is indicated by the red arrow. The shaded amino acids indicate shared sequence identity (black) or similarity (gray). The human RTN4 presenting in FIG. 6 corresponds to UniProt ID Q9NQC3, having the full sequence described herein as SEQ ID NO: 331. The Xenopus laevis RTN4 presenting in FIG. 6 corresponds to UniProt ID Q6JRV0, having the full sequence described herein as SEQ ID NO:332.

FIG. 7. Sequence alignment of the reticulon homology domain from human reticulon proteins. The reticulon homology domain consists of the tandem hydrophobic regions and the intervening linker region. C1101 of RTN4 is indicated by the arrow. The shaded amino acids indicate shared sequence identity (black) or similarity (gray). The sequences in FIG. 7 include, from top to bottom, UniProt O75298 (RTN2a) SEQ ID NO:333, UniProt O75298-2 (RTN2b) SEQ ID NO:334, UniProt O95197 (RTN3a) SEQ ID NO:335, UniProt O95197-2 (RTN3b) SEQ ID NO:336, UniProt O95197-3 (RTN3c) SEQ ID NO:337, UniProt Q16799 (RTN1a) SEQ ID NO:338, UniProt Q16799-2 (RTN1b) SEQ ID NO:339, UniProt Q16799-3 (RTN1c) SEQ ID NO:340, UniProt Q9NQC3 (RTN4a) SEQ ID NO:331, UniProt Q9NQC3-2 (RTN4b) SEQ ID NO:341, and UniProt Q9NQC3-3 (RTN4c) SEQ ID NO:342.

FIG. 8. ER Morphology in SW620 Colorectal Cancer Cells. SW620 cells expressing GFP-tagged Sec61β were treated with DKM 3-30 (50 μM) for the indicated times and the ER (light gray/white) and nuclear (dark gray) morphology visualized by fluorescence microscopy.

FIGS. 9A-9B. DKM 3-30 alters ER morphology. (FIGS. 9A, 9B) U2OS cells expressing GFP-tagged Sec61β were treated with DKM 3-30 (50 μM) and ER morphology visualized by time-lapse fluorescence microscopy. Time (min) is indicated on each panel. Bottom panels indicate boxed region.

FIG. 10. DKM 3-30 modifies C1101 of Rtn4. C1101 projects into the cytoplasm and laterally towards a prominent groove in the surface of Rtn4. Covalent modification of C1101 with DKM 3-30 may interact with, or modify the location of, surrounding residues that line the groove, including E1105, E1078, S1079, A1082, I1083, K1090, Y1091, S1094, G1097, and H1098. These interactions or modifications could result in local or global structural derangements that could influence Rtn4 functions, interactions with lipids, and/or interactions with protein binding partners.

FIGS. 11A-11C. DKM 3-30 and analogs. (FIG. 11A) Structures of DKM 3-30 and analogs. (FIG. 11B) Gel-based ABPP analysis showing competition side-by-side competition studies of DKM 3-30, YP 1-46, and AMR 1-125 against IA-rhodamine labelling of pure human RTN4. Shown are the 50% inhibitory concentration (IC50) values for each compound. (FIG. 11C) Serum-free cell survival of U2OS (48 h) or SW620 (24 h) cells treated with DMSO vehicle or each compound (50 μM). Data in (C) are presented as mean±sem. Significance is expressed as *p<0.001 compared to vehicle-treated controls.

FIG. 12. Effect of DKM 3-30 in Mouse Embryonic Fibroblast (MEF) cells expressing human RTN4. C1101 in human RTN4 is instead a serine in mouse RTN4. DKM 3-30 does not induce apoptosis in GFP-expressing MEF cells, but induces apoptosis in MEF cells expressing human RTN4-GFP. GFP or RTN4-GFP expressing MEF cells were treated with DKM 3-30 (50 μM) for 0, 8, or 16 h and apoptotic cells (propidium iodine positive and Annexin-V positive) were assessed by flow cytometry. Data are presented as mean±sem. Significance is expressed as *p<0.05 compared to 0 h time-point.

FIG. 13. AMR 1-125, but not YP 146, alters ER morphology. U2OS cells expressing GFP-tagged Sec61β were incubated with control, 1 μM AMR 1-125, or 50 μM YP 146 and the ER morphology was visualized by time-lapse fluorescence microscopy. Time (min) is indicated on each panel. Bottom panels indicate boxed region.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, or S), and wherein the nitrogen and sulfur atoms may optionally be oxidized, and the nitrogen heteroatom may optionally be quaternized. The heteroatom(s) (e.g., O, N, P, S, B, As, or Si) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

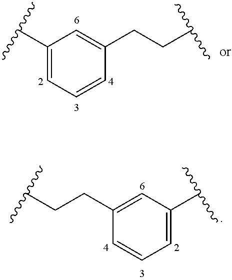 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkyl aryl ene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′), —X′—(C″R″R′″)_{d}—, where s and d are independently integers of from 0 to 3, and X′ is —O—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:

(A) oxo,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
(i) oxo,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl, heteroalkyl, cycloalkyl, heterocycloalkyl, aryl, heteroaryl, substituted with at least one substituent selected from:
(a) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCH Br₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(b) alkyl, heteroalkyl, cycloalkyl, heterocycloalkyl, aryl, heteroaryl, substituted with at least one substituent selected from: oxo,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)— or (S)— or, as (D)- or (L)- for amino acids, and individual isomers are encompassed within the scope of the present invention. The compounds of the present invention do not include those that are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R³ and optionally differently.

A “covalent cysteine modifier moiety” as used herein refers to a substituent that is capable of reacting with the sulfhydryl functional group of a cysteine amino acid (e.g. cysteine corresponding to C1101 of the human reticulon 4) to form a covalent bond. Thus, the covalent cysteine modifier moiety is typically electrophilic.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

A “Reticulon 4 inhibitor” and “RTN 4 inhibitor” is a substance (e.g., oligonucleotide, protein, composition, or compound) that negatively affects (e.g. decreases) the activity or function of reticulon 4 relative to the activity or function of reticulon 4 in the absence of the inhibitor (e.g., wherein the reticulon 4 inhibitor binds reticulon 4). A “reticulon 4 inhibitor compound” or “RTN 4 inhibitor compound” or “RTN 4 inhibitor compound” refers to a compound (e.g. a compound described herein) that reduces the activity of reticulon 4 when compared to a control, such as absence of the compound or a compound with known inactivity. In embodiments, a Reticulon 4 inhibitor is a compound described herein.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A polypeptide, or a cell is “recombinant” when it is artificial or engineered, or derived from or contains an artificial or engineered protein or nucleic acid (e.g. non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

An amino acid residue in a protein “corresponds” to a given residue when it occupies the same essential structural and/or spatial position within the protein as the given residue in a reference sequence. For example, a selected residue in a selected protein corresponds to Cys1101 when the selected residue occupies the same essential structural and/or spatial position as Cys1101 in SEQ ID NO:331. In some embodiments, where a selected protein is aligned for maximum homology with the human reticulon 4 protein, the position in the aligned selected protein aligning with Cys1101 is said to correspond to Cys1101. Instead of a primary sequence alignment, a three dimensional structural alignment can also be used, e.g., where the three dimensional structure of the selected protein is aligned for maximum correspondence with the human reticulon 4 protein (reference sequence) and the overall structures compared. In this case, the amino acid that occupies the same essential structural position as Cys1101 in the structural model relative to the reference sequence is said to correspond to the Cys1101 residue.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease.

As defined herein, the term “inhibition”, “inhibitor”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “reticulon 4” and “RTN 4” and “RTN4” refer to a protein (including homologs, isoforms, and functional fragments thereof) with reticulon 4 activity. The term includes any recombinant or naturally-occurring form of reticulon 4 or variants thereof that maintain reticulon 4 activity (e.g. within at least 30%, 40%, 50%, 60%, 70%, 80%, 90%, 95%, or 100% activity compared to wildtype reticulon 4). In embodiments, the reticulon 4 protein encoded by the RTN 4 gene has the amino acid sequence set forth in or corresponding to Entrez 57142, UniProt Q9NQC3, or RefSeq (protein) NP_065393. In embodiments, the reticulon 4 gene has the nucleic acid sequence set forth in RefSeq (mRNA) NM_020532. In embodiments, the amino acid sequence or nucleic acid sequence is the sequence known at the time of filing of the present application. In embodiments, the sequence corresponds to NP_065393.1. In embodiments, the sequence corresponds to NM_020532.4. In embodiments, the reticulon 4 is a human reticulon 4, such as a human cancer causing reticulon 4.

In embodiments, the RTN4 sequence corresponds to UniProt ID Q9NQC3, and has the sequence:

(SEQ ID NO: 331)
MEDLDQSPLVSSSDSPPRPQPAFKYQFVREPEDEEEEEEEEEEDEDEDLE
ELEVLERKPAAGLSAAPVPTAPAAGAPLMDFGNDFVPPAPRGPLPAAPPV
APERQPSWDPSPVSSTVPAPSPLSAAAVSPSKLPEDDEPPARPPPPPPAS
VSPQAEPVWTPPAPAPAAPPSTPAAPKRRGSSGSVDETLFALPAASEPVI
RSSAENMDLKEQPGNTISAGQEDFPSVLLETAASLPSLSPLSAASFKEHE
YLGNLSTVLPTEGTLQENVSEASKEVSEKAKTLLIDRDLTEFSELEYSEM
GSSFSVSPKAESAVIVANPREEIIVKNKDEEEKLVSNNILHNQQELPTAL
TKLVKEDEVVSSEKAKDSFNEKRVAVEAPMREEYADFKPFERVWEVKDSK
EDSDMLAAGGKIESNLESKVDKKCFADSLEQTNHEKDSESSNDDTSFPST
PEGIKDRSGAYITCAPFNPAATESIATNIFPLLGDPTSENKTDEKKIEEK
KAQIVTEKNTSTKTSNPFLVAAQDSETDYVTTDNLTKVTEEVVANMPEGL
TPDLVQEACESELNEVTGTKIAYETKMDLVQTSEVMQESLYPAAQLCPSF
EESEATPSPVLPDIVMEAPLNSAVPSAGASVIQPSSSPLEASSVNYESIK
HEPENPPPYEEAMSVSLKKVSGIKEEIKEPENINAALQETEAPYISIACD
LIKETKLSAEPAPDFSDYSEMAKVEQPVPDHSELVEDSSPDSEPVDLFSD
DSIPDVPQKQDETVMLVKESLTETSFESMIEYENKEKLSALPPEGGKPYL
ESFKLSLDNTKDTLLPDEVSTLSKKEKIPLQMEELSTAVYSNDDLFISKE
AQIRETETFSDSSPIETIDEFPTLISSKTDSFSKLAREYTDLEVSHKSEI
ANAPDGAGSLPCTELPHDLSLKNIQPKVEEKISFSDDFSKNGSATSKVLL
LPPDVSALATQAEIESIVKPKVLVKEAEKKLPSDTEKEDRSPSAIFSAEL
SKTSVVDLLYWRDIKKTGVVFGASLELLLSLTVESIVSVTAYIALALLSV
TISFRIYKGVIQAIQKSDEGHPFRAYLESEVAISEELVQKYSNSALGHVN
CTIKELRRLFLVDDLVDSLKFAVLMWVFTYVGALFNGLTLLILALISLFS
VPVIYERHQAQIDHYLGLANKNVKDAMAKIQAKIPGLKRKAE

In embodiments, the RTN4 sequence corresponds to UniProt ID Q6JRV0, and has the following sequence:

(SEQ ID NO: 332)
MDEQSPDISSSHSGDERREPAQPGERKPWDDLDDVLDLTGGAGQFSQPFS
GSHPARDIEEEEEDEEEERGAWKDSLEPSPVEEEPGSIDSISPVSPHSPA
VPSAPMEEPERPPAPCTAPSGSVDENLFTLPAASAHLMHASADKIMEPYS
TVSTGQEEFASVLLQSTASLSSLPSLSTDSSKEHAETVAFPTGLAATEAL
QEPTDNMYSVSRITSHLPLSDNLESKALDQVKEEVIFSEKGYVVDHPTSQ
QETISEEHAKLYSQSAKEMFSGMLQSVAPPHEEFTDIKEVYDPYVDFKPF
MSSKSGDVGYEVSDVAEKFQVDVGRLNLESAVKHEEKSSEEMEIDSISDD
ISPLTPELLPDSTDYDMFATVEQNIPFSFGGGHVAGNKTDEKKIEDIEAQ
KTSVGFGLKVATVNPFYNESAQESEYVTTHVATHVSTKPEGPTPDIVQEA
YESEAYDTGIPKQKYESNIDLVQTAANSVQEKVSPTAQAPARLEETDSVS
SPVLPDIVMEAPLASALETVALKPDISPVGIKPPARVEKTKAEPEKPPSY
EEAVTEVLQNQDLAAALGGSKQGAVVEETETPYISIACDLIKGTESVASG
FTEFSKLKQNEFESQFMEPSDESSPDSECSEPSYKQWDSEVVQKEAFSIK
TESVNAQSIIIPEQKQVFDQKSEESSPSKSYLDSFQPEICVSKATSDLFA
KGLTTLLQEKPLQMEELDEGLSLEKIPCTKYSPVSESPEPRPSPVPEDLS
SKLGDIQKEVLIAKQPEDKVQKNRSNLDFVPENTEFTPAVQKPDDSGKAV
SDTFGGLDTTTKGGSAVHEVKVDKPKPPSKEDDGSKLPKKESKASTVSSS
DFMNSVVDLIYWRDIKRSGVVFGASLFLLLSLSVFSIVSVLAYIALALLS
VTISLRIYKGILQAIQKSEEGHPFRSILESNLAVPEDLVQKYCNVALNHV
NCTVKELRHLFLVEDLVDSLKFAVLMWVFTYIGALFNGLTLLIVALISLF
SIPVIYERHQTQVDHYLALVNKNLKSTSDLILSKVPGLKRKAE.

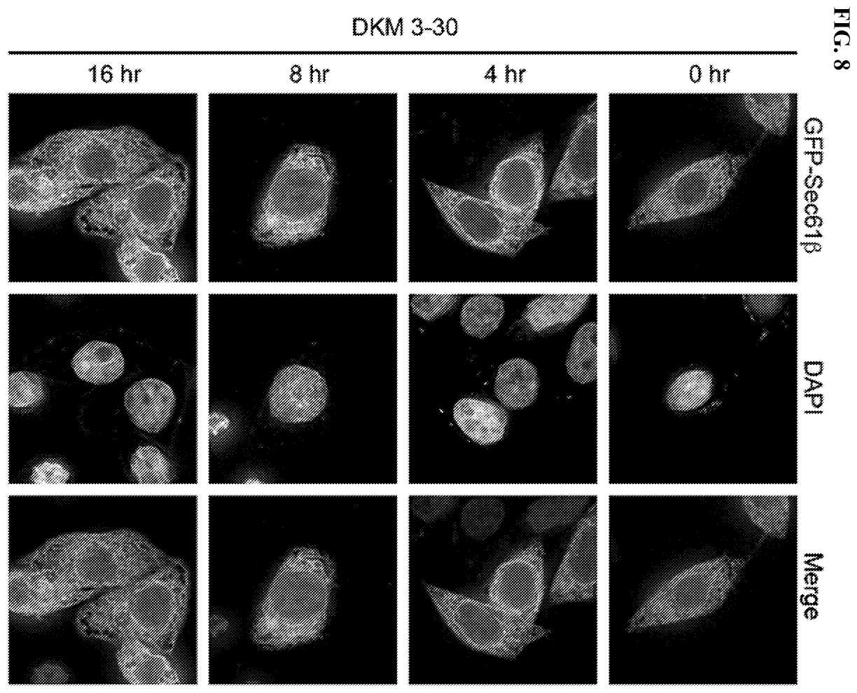

As observed in FIG. 7, UniProt O75298 (RTN2a) has the following sequence:

(SEQ ID NO: 333)
MGQVLPVFAHCKEAPSTASSTPDSTEGGNDDSDFRELHTAREFSEEDEET
TSQDWGTPRELTFSYIAFDGVVGSGGRRDSTARRPRPQGRSVSEPRDQHP
QPSLGDSLESIPSLSQSPEPGRRGDPDTAPPSERPLEDLRLRLDHLGWVA
RGTGSGEDSSTSSSTPLEDEEPQEPNRLETGEAGEELDLRLRLAQPSSPE
VLTPQLSPGSGTPQAGTPSPSRSRDSNSGPEEPLLEEEEKQWGPLEREPV
RGQCLDSTDQLEFTVEPRLLGTAMEWLKTSLLLAVYKTVPILELSPPLWT
AIGWVQRGPTPPTPVLRVLLKWAKSPRSSGVPSLSLGADMGSKVADLLYW
KDTRTSGVVFTGLMVSLLCLLHFSIVSVAAHLALLLLCGTISLRVYRKVL
QAVHRGDGANPFQAYLDVDLTLTREQTERLSHQITSRVVSAATQLRHFFL
VEDLVDSLKLALLFYILTFVGAIFNGLTLLILGVIGLFTIPLLYRQHQAQ
IDQYVGLVTNQLSHIKAKIRAKIPGTGALASAAAAVSGSKAKAE

As observed in FIG. 7, UniProt O75298-2 (RTN2b) has the following sequence:

(SEQ ID NO: 334)
MGQVLPVFAHCKEAPSTASSTPDSTEGGNDDSDFRELHTAREFSEEDEEE
TTSQDWGTPRELTESYTAFDGVVGSGGRRDSTARRPRPQGRSVSEPRDQH
PQPSLGDSLESIPSLSQSPEPGRRGDPDTAPPSERPLEDLRLRLDHLGWV
ARGTGSGEDSSTSSSTPLEDEEPQEPNRLETGEAGEELDLRLRLAQPSSP
EVLTPQLSPGSGTPQAGTPSPSRSRDSNSGPEEPLLEEEEKQWGPLEREP
VRGQCLDSTDQLEFTVEPRLLVADLLYWKDTRTSGVVETGLMVSLLCLLH
FSIVSVAAHLALLLLCGTISLRVYRKVLQAVHRGDGANPFQAYLDVDLTL
TREQTERLSHQITSRVVSAATQLRHFELVEDLVDSLKLALLFYILTFVGA
IENGLTLLILGVIGLETIPLLYRQHQAQIDQYVGLVTNQLSHIKAKIRAK
IPGTGALASAAAAVSGSKAKAE

As observed in FIG. 7, UniProt O95197 (RTN3a) has the following sequence:

(SEQ ID NO: 335)
MAEPSAATQSHSISSSSFGAEPSAPGGGGSPGACPALGTKSCSSSCADSF
VSSSSSQPVSLFSTSQEGLSSLCSDEPSSEIMTSSFLSSSEIHNTGLTIL
HGEKSHVLGSQPILAKEGKDHLDLLDMKKMEKPQGTSNNVSDSSVSLAAG
VHCDRPSIPASFPEHPAFLSKKIGQVEEQIDKETKNPNGVSSREAKTALD
ADDRFTLLTAQKPPTEYSKVEGIYTYSLSPSKVSGDDVIEKDSPESPFEV
IIDKAAFDKEFKDSYKESTDDFGSWSVHTDKESSEDISETNDKLFPLRNK
EAGRYPMSALLSRQFSHTNAALEEVSRCVNDMHNFTNEILTWDLVPQVKQ
QTDKSSDCITKTTGLDMSEYNSEIPVVNLKTSTHQKTPVCSIDGSTPITK
STGDWAEASLQQENAITGKPVPDSLNSTKEESIKGVQGNMQKQDDTLAEL
PGSPPEKCDSLGSGVATVKVVLPDDHLKDEMDWQSSALGEITEADSSGES
DDTVIEDITADTSFENNKIQAEKPVSIPSAVVKTGEREIKEIPSCEREEK
TSKNFEELVSDSELHQDQPDILGRSPASEAACSKVPDTNVSLEDVSEVAP
EKPITTENPKLPSTVSPNVFNETEFSLNVTTSAYLESLHGKNVKHIDDSS
PEDLIAAFTETRDKGIVDSERNAFKAISEKMTDFKTTPPVEVLHENESGG
SEIKDIGSKYSEQSKETNGSEPLGVFPTQGTPVASLDLEQEQLTIKALKE
LGERQVEKSTSAQRDAELPSEEVLKQTFTFAPESWPQRSYDILERNVKNG
SDLGISQKPITIRETTRVDAVSSLSKTELVKKHVLARLLTDFSVHDLIFW
RDVKKTGFVFGTTLIMLLSLAAFSVISVVSYLILALLSVTISFRIYKSVI
QAVQKSEEGHPFKAYLDVDITLSSEAFHNYMNAAMVHINRALKLIIRLFL
VEDLVDSLKLAVFMWLMTYVGAVFNGITLLILAELLIFSVPIVYEKYKTQ
IDHYVGIARDQTKSIVEKIQAKLPGIAKKKAE

As observed in FIG. 7, UniProt O95197-2 (RTN3b) has the following sequence:

(SEQ ID NO: 336)
MAEPSAATQSHSISSSSFGAEPSAPGGGGSPGACPALGTKSCSSSCAEGL
SSLCSDEPSSEIMTSSELSSSEIHNTGLTILHGEKSHVLGSQPILAKEGK
DHLDLLDMKKMEKPQGTSNNVSDSSVSLAAGVHCDRPSIPASEPEHPAEL
SKKIGQVEEQIDKETKNPNGVSSREAKTALDADDRFTLLTAQKPPTEYSK
VEGIYTYSLSPSKVSGDDVIEKDSPESPFEVIIDKAAFDKEFKDSYKEST
DDEGSWSVHTDKESSEDISETNDKLEPLRNKEAGRYPMSALLSRQFSHTN
AALEEVSRCVNDMHNFTNEILTWDLVPQVKQQTDKSSDCITKTTGLDMSE
YNSEIPVVNLKTSTHQKTPVCSIDGSTPITKSTGDWAEASLQQENAITGK
PVPDSLNSTKEFSIKGVQGNMQKQDDTLAELPGSPPEKCDSLGSGVATVK
VVLPDDHLKDEMDWQSSALGEITEADSSGESDDTVIEDITADTSFENNKI
QAEKPVSIPSAVVKTGEREIKEIPSCEREEKTSKNFEELVSDSELHQDQP
DILGRSPASEAACSKVPDTNVSLEDVSEVAPEKPITTENPKLPSTVSPNV
FNETEFSLNVITSAYLESLHGKNVKHIDDSSPEDLIAAFTETRDKGIVDS
ERNAFKAISEKMTDEKTIPPVEVLHENESGGSEIKDIGSKYSEQSKETNG
SEPLGVEPTQGTPVASLDLEQEQLTIKALKELGERQVEKSTSAQRDAELP
SEEVLKQTFTFAPESWPQRSYDILERNVKNGSDLGISQKPITIRETTRVD
AVSSLSKTELVKKHVLARLLTDFSVHDLIFWRDVKKTGFVFGTTLIMLLS
LAAFSVISVVSYLILALLSVTISFRIYKSVIQAVQKSEEGHPFKAYLDVD
ITLSSEAFHNYMNAAMVHINRALKLIIRLFLVEDLVDSLKLAVFMWLMTY
VGAVENGITLLILAELLIFSVPIVYEKYKTQIDHYVGIARDQTKSIVEKI
QAKLPGIAKKKAE

As observed in FIG. 7, UniProt O95197-3 (RTN3c) has the following sequence:

(SEQ ID NO: 337)
MAEPSAATQSHSISSSSFGAEPSAPGGGGSPGACPALGTKSCSSSCAVHD
LIFWRDVKKTGFVFGTTLIMLLSLAAFSVISVVSYLILALLSVTISFRIY
KSVIQAVQKSEEGHPFKAYLDVDITLSSEAFHNYMNAAMVHINRALKLII
RLFLVEDLVDSLKLAVFMWLMTYVGAVFNGITLLILAELLIFSVPIVYEK
YKTQIDHYVGIARDQTKSIVEKIQAKLPGIAKKKAE

As observed in FIG. 7, UniProt Q16799 (RTN1a) has the following sequence:

(SEQ ID NO: 338)
MAAPGDPQDELLPLAGPGSQWLRHRGEGENEAVTPKGATPAPQAGEPSPG
LGARAREAASREAGSGPARQSPVAMETASTGVAGVSSAMDHTFSTTSKDG
EGSCYTSLISDICYPPQEDSTYFTGILQKENGHVTISESPEELGTPGPSL
PDVPGIESRGLFSSDSGIEMTPAESTEVNKILADPLDQMKAEAYKYIDIT
RPEEVKHQEQHHPELEDKDLDFKNKDTDISIKPEGVREPDKPAPVEGKII
KDHLLEESTFAPYIDDLSEEQRRAPQITTPVKITLTEIEPSVETTTQEKT
PEKQDICLKPSPDTVPTVTVSEPEDDSPGSITPPSSGTEPSAAESQGKGS
ISEDELITAIKEAKGLSYETAENPRPVGQLADRPEVKARSGPPTIPSPLD
HEASSAESGDSEIELVSEDPMAAEDALPSGYVSFGHVGGPPPSPASPSIQ
YSILREEREAELDSELIIESCDASSASEESPKREQDSPPMKPSALDAIRE
ETGVRAEERAPSRRGLAEPGSFLDYPSTEPQPGPELPPGDGALEPETPML
PRKPEEDSSSNQSPAATKGPGPLGPGAPPPLLFLNKQKAIDLLYWRDIKQ
TGIVEGSFLLLLFSLTQFSVVSVVAYLALAALSATISFRIYKSVLQAVQK
TDEGHPFKAYLELEITLSQEQIQKYTDCLQFYVNSTLKELRRLFLVQDLV
DSLKFAVLMWLLTYVGALFNGLTLLLMAVVSMFTLPVVYVKHQAQIDQYL
GLVRTHINAVVAKIQAKIPGAKRHAE

As observed in FIG. 7, UniProt Q16799-2 (RTN1b) has the following sequence:

(SEQ ID NO: 339)
MAAEDALPSGYVSFGHVGGPPPSPASPSIQYSILREEREAELDSELIIES
CDASSASEESPKREQDSPPMKPSALDAIREETGVRAEERAPSRRGLAEPG
SFLDYPSTEPQPGPELPPGDGALEPETPMLPRKPEEDSSSNQSPAATKGP
GPLGPGAPPPLLFLNKQKAIDLLYWRDIKQTGIVEGSFLLLLFSLTQFSV
VSVVAYLALAALSATISFRIYKSVLQAVQKTDEGHPFKAYLELEITLSQE
QIQKYTDCLQFYVNSTLKELRRLFLVQDLVDSLKFAVLMWLLTYVGALFN
GLTLLLMAVVSMFTLPVVYVKHQAQIDQYLGLVRTHINAVVAKIQAKIPG
AKRHAE

As observed in FIG. 7, UniProt Q16799-3 (RTN1c) has the following sequence:

(SEQ ID NO: 340)
MQATADSTKMDCVWSNWKSQATDLLYWRDIKQTGIVFGSFLLLLFSLTQF
SVVSVVAYLALAALSATISFRIYKSVLQAVQKTDEGHPFKAYLELEITLS
QEQIQKYTDCLQFYVNSTLKELRRLFLVQDLVDSLKFAVLMWLLTYVGAL
FNGLTLLLMAVVSMFTLPVVYVKHQAQIDQYLGLVRTHINAVVAKIQAKI
PGAKRHAE

As observed in FIG. 7, UniProt Q9NQC3 (RTN4a) has the following sequence:

(SEQ ID NO: 341)
MEDLDQSPLVSSSDSPPRPQPAFKYQFVREPEDEEEEEEEEEEDEDEDLE
ELEVLERKPAAGLSAAPVPTAPAAGAPLMDFGNDFVPPAPRGPLPAAPPV
APERQPSWDPSPVSSTVPAPSPLSAAAVSPSKLPEDDEPPARPPPPPPAS
VSPQAEPVWTPPAPAPAAPPSTPAAPKRRGSSGSVDETLFALPAASEPVI
RSSAENMDLKEQPGNTISAGQEDFPSVLLETAASLPSLSPLSAASEKEHE
YLGNLSTVLPTEGTLQENVSEASKEVSEKAKTLLIDRDLTEFSELEYSEM
GSSFSVSPKAESAVIVANPREEIIVKNKDEEEKLVSNNILHNQQELPTAL
TKLVKEDEVVSSEKAKDSFNEKRVAVEAPMREEYADFKPFERVWEVKDSK
EDSDMLAAGGKIESNLESKVDKKCFADSLEQTNHEKDSESSNDDTSFPST
PEGIKDRSGAYITCAPFNPAATESIATNIFPLLGDPTSENKTDEKKIEEK
KAQIVTEKNTSTKTSNPFLVAAQDSETDYVTTDNLTKVTEEVVANMPEGL
TPDLVQEACESELNEVTGTKIAYETKMDLVQTSEVMQESLYPAAQLCPSF
EESEATPSPVLPDIVMEAPLNSAVPSAGASVIQPSSSPLEASSVNYESIK
HEPENPPPYEEAMSVSLKKVSGIKEEIKEPENINAALQETEAPYISIACD
LIKETKLSAEPAPDFSDYSEMAKVEQPVPDHSELVEDSSPDSEPVDLFSD
DSIPDVPQKQDETVMLVKESLTETSFESMIEYENKEKLSALPPEGGKPYL
ESFKLSLDNTKDTLLPDEVSTLSKKEKIPLQMEELSTAVYSNDDLFISKE
AQIRETETESDSSPIEIIDEFPTLISSKTDSFSKLAREYTDLEVSHKSEI
ANAPDGAGSLPCTELPHDLSLKNIQPKVEEKISFSDDFSKNGSATSKVLL
LPPDVSALATQAEIESIVKPKVLVKEAEKKLPSDTEKEDRSPSAIFSAEL
SKTSVVDLLYWRDIKKTGVVFGASLELLLSLTVESIVSVTAYIALALLSV
TISFRIYKGVIQATQKSDEGHPFRAYLESEVAISEELVQKYSNSALGHVN
CTIKELRRLFLVDDLVDSLKFAVLMWVETYVGALENGLTLLILALISLFS
VPVIYERHQAQIDHYLGLANKNVKDAMAKIQAKIPGLKRKAE

As observed in FIG. 7, UniProt Q9NQC3-2 (RTN4b) has the following sequence:

(SEQ ID NO: 342)
MEDLDQSPLVSSSDSPPRPQPAFKYQFVREPEDEEEEEEEEEEDEDEDLE
ELEVLERKPAAGLSAAPVPTAPAAGAPLMDFGNDFVPPAPRGPLPAAPPV
APERQPSWDPSPVSSTVPAPSPLSAAAVSPSKLPEDDEPPARPPPPPPAS
VSPQAEPVWTPPAPAPAAPPSTPAAPKRRGSSGSVVVDLLYWRDIKKTGV
VFGASLFLLLSLIVESIVSVTAYIALALLSVTISFRIYKGVIQAIQKSDE
GHPFRAYLESEVAISEELVQKYSNSALGHVNCTIKELRRLFLVDDLVDSL
KFAVLMWVFTYVGALFNGLTLLILALISLFSVPVIYERHQAQIDHYLGLA
NKNVKDAMAKIQAKIPGLKRKAE

As observed in FIG. 7, UniProt Q9NQC3-3 (RTN4c) has the following sequence:

(SEQ ID NO: 343)
MDGQKKNWKDKVVDLLYWRDIKKTGVVFGASLFLLLSLTVFSIVSVTAYI
ALALLSVTISFRIYKGVIQAIQKSDEGHPFRAYLESEVAISEELVQKYSN
SALGHVNCTIKELRRLFLVDDLVDSLKFAVLMWVFTYVGALFNGLTLLIL
ALISLFSVPVIYERHQAQIDHYLGLANKNVKDAMAKIQAKIPGLKRKAE

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The terms “disease” or “condition” refer to a state of being or health status of a patient or subject capable of being treated with the compounds or methods provided herein. The disease may be a cancer. The disease may be stroke. The disease may be an inflammatory disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemia, carcinomas and sarcomas. Exemplary cancers that may be treated with a compound or method provided herein include brain cancer, glioma, glioblastoma, neuroblastoma, prostate cancer, colorectal cancer, pancreatic cancer, cervical cancer, gastric cancer, ovarian cancer, lung cancer, and cancer of the head. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the term “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Strussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff s disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing. In embodiments, the treating or treatment is no prophylactic treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “modulator” refers to a composition that increases or decreases the level of a target molecule or the function of a target molecule or the physical state of the target of the molecule. In some embodiments, a reticulon 4 associated disease modulator is a compound that reduces the severity of one or more symptoms of a disease associated with reticulon 4 (e.g. cancer). A reticulon 4 modulator is a compound that increases or decreases the activity or function or level of activity or level of function of reticulon 4.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer associated with reticulon 4 activity, reticulon 4 associated cancer, reticulon 4 associated disease) means that the disease (e.g. cancer) is caused by (in whole or in part), or a symptom of the disease is caused by (in whole or inpart) the substance or substance activity or function. For example, a cancer associated with reticulon 4 activity or function may be a cancer that results (entirely or partially) from aberrant reticulon 4 function (e.g. enzyme activity, protein-protein interaction, signaling pathway) or a cancer wherein a particular symptom of the disease is caused (entirely or partially) by aberrant reticulon 4 activity or function. As used herein, what is described as being associated with a disease, if a causative agent, could be a target for treatment of the disease. For example, a cancer associated with reticulon 4 activity or function or a reticulon 4 associated cancer, may be treated with a reticulon 4 modulator or reticulon 4 inhibitor, in the instance where reticulon 4 activity or function (e.g. signaling pathway activity) causes the cancer.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propogated to other signaling pathway components. For example, binding of a reticulon 4 protein with a compound as described herein may reduce the interactions between the reticulon 4 protein and downstream effectors or signaling pathway components, resulting in changes in cell growth, proliferation, or survival.

The term “electrophilic chemical moiety” is used in accordance with its plain ordinary chemical meaning and refers to a chemical group (e.g., monovalent chemical group) that is electrophilic.

The term “nucleophilic chemical moiety” is used in accordance with its plain ordinary chemical meaning and refers to a chemical group (e.g., monovalent chemical group) that is nucleophilic.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

An “antisense nucleic acid” as referred to herein is a nucleic acid (e.g., DNA or RNA molecule) that is complementary to at least a portion of a specific target nucleic acid (e.g., a nucleic acid coding for one or more amino acids corresponding to E1105, C1101, E1078, S1079, A1082, I1083, K1090, Y1091, S1094, G1097, and H1098 of SEQ ID NO:331) and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid (e.g., a nucleic acid coding for one or more amino acids corresponding to E1105, C1101, E1078, S1079, A1082, I1083, K1090, Y1091, S1094, G1097, and H1098 of SEQ ID NO:331). In embodiments, the antisense nucleic acid hybridizes to the target nucleic acid (e.g. a nucleic acid coding for one or more amino acids corresponding to E1105, C1101, E1078, S1079, A1082, I1083, K1090, Y1091, S1094, G1097, and H1098 of SEQ ID NO:331) in vitro. In embodiments, the antisense nucleic acid hybridizes to the target nucleic acid (e.g. a nucleic acid coding for one or more amino acids corresponding to E1105, C1101, E1078, S1079, A1082, I1083, K1090, Y1091, S1094, G1097, and H1098 of SEQ ID NO:331) in a cell. In embodiments, the antisense nucleic acid hybridizes to the target nucleic acid (e.g. a nucleic acid coding for one or more amino acids corresponding to E1105, C1101, E1078, S1079, A1082, I1083, K1090, Y1091, S1094, G1097, and H1098 of SEQ ID NO:331) in an organism. In embodiments, the antisense nucleic acid hybridizes to the target nucleic acid (e.g. a nucleic acid coding for one or more amino acids corresponding to E1105, C1101, E1078, S1079, A1082, I1083, K1090, Y1091, S1094, G1097, and H1098 of SEQ ID NO:331) under physiological conditions. Antisense nucleic acids may comprise naturally occurring nucleotides or modified nucleotides such as, e.g., phosphorothioate, methylphosphonate, and -anomeric sugar-phosphate, backbonemodified nucleotides.

In the cell, the antisense nucleic acids hybridize to the corresponding RNA (e.g., a nucleic acid coding for one or more amino acids corresponding to E1105, C1101, E1078, S1079, A1082, I1083, K1090, Y1091, S1094, G1097, and H1098 of SEQ ID NO:331) forming a double-stranded molecule. The antisense nucleic acids interfere with the endogenous behavior of the RNA (e.g., a nucleic acid coding for one or more amino acids corresponding to E1105, C1101, E1078, S1079, A1082, I1083, K1090, Y1091, S1094, G1097, and H1098 of SEQ ID NO:331) and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994)J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “irreversible covalent bond” is used in accordance with its plain ordinary meaning in the art and refers to the resulting association between atoms or molecules of (e.g., electrophilic chemical moiety and nucleophilic moiety) wherein the probability of dissociation is low. In embodiments, the irreversible covalent bond does not easily dissociate under normal biological conditions. In embodiments, the irreversible covalent bond is formed through a chemical reaction between two species (e.g., electrophilic chemical moiety and nucleophilic moiety).

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; top sentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or r1L.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Gurin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

The term “reticulon 4 activity” as used herein refers to the biological activity of the protein. In embodiments, reticulon 4 activity includes endoplasmic reticulum (ER) tubule formation. Reticulon 4 activity may be quantified by measuring tubular ER network formation, ER morphology, mitosis rate, nuclear envelope assembly, nuclear envelope disassembly, or cell death.

The term “reticulon 4 protein-reticulon 4 inhibitor complex” as used herein refers to a reticulon 4 protein bonded (e.g., covalently bonded) to a Reticulon 4 inhibitor (e.g., a compound described herein).

### II. Compounds

In an aspect is provided a compound having the formula:

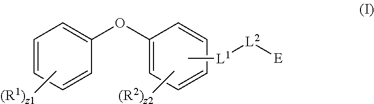

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl.

The symbol z1 is an integer from 0 to 5.

R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(OR^{2C}—C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Two adjacent R² substituents may optionally be joined to form a substituted or unsubstituted cycloalkyl, substituted or unsubstituted heterocycloalkyl, substituted or unsubstituted aryl, or substituted or unsubstituted heteroaryl.

The symbol z2 is an integer from 0 to 4.

L¹ is a bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene.

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene.

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}—, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

E is an electrophilic moiety.

Each R^{1A}, R^{1B}, R^{1C}, R^{1D}, R^{2A}, R^{2B}, R^{2C}, R^{2D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A} and R^{1B} substituents bonded to the same nitrogen atom may optionally be joined to form a substituted or unsubstituted heterocycloalkyl or substituted or unsubstituted heteroaryl. R^{2A} and R^{2B} substituents bonded to the same nitrogen atom may optionally be joined to form a substituted or unsubstituted heterocycloalkyl or substituted or unsubstituted heteroaryl. R^{4A} and R^{4B} substituents bonded to the same nitrogen atom may optionally be joined to form a substituted or unsubstituted heterocycloalkyl or substituted or unsubstituted heteroaryl. R^{5A} and R^{5B} substituents bonded to the same nitrogen atom may optionally be joined to form a substituted or unsubstituted heterocycloalkyl or substituted or unsubstituted heteroaryl.

Each X, X¹, X², X⁴, and X⁵ is independently —F, —Cl, —Br, or —I.

The symbols n1, n2, n4, and n5 are independently an integer from 0 to 4.

The symbols m1, m2, m4, m5, v1, v2, v4, and v5 are independently an integer from 1 to 2.

In embodiments, the compound has the formula:

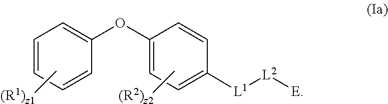

R¹, R², L¹, L², E, z1 and z2 are as described herein.

In embodiments, the compound has the formula:

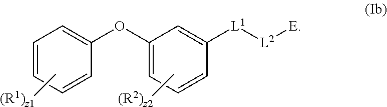

R¹, R², L¹, L², and E are as described herein.

In embodiments, the compound has the formula:

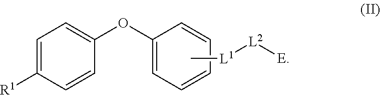

R¹, L¹, L², and E are as described herein.

In embodiments, the compound has the formula:

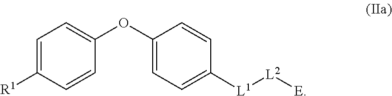

R¹, L¹, L², and E are as described herein.

In embodiments, the compound has the formula:

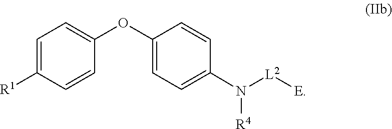

R¹, R⁴, L², and E are as described herein.

In embodiments, the compound has the formula:

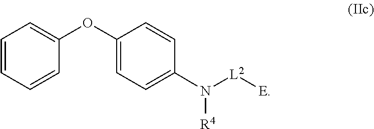

R⁴, L², and E are as described herein.

In embodiments, the compound has the formula:

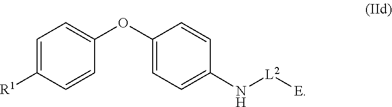

R¹, R⁴, L², and E are as described herein.

In embodiments, the compound has the formula:

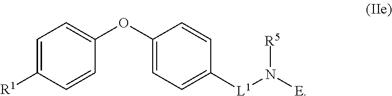

R¹, R⁵, L¹, and E are as described herein.

In embodiments, the compound has the formula:

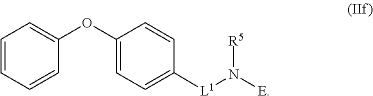

R⁵, L¹, and E are as described herein.

In embodiments, the compound has the formula:

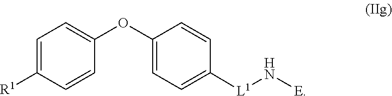

R¹, L², and E are as described herein.

In embodiments, the compound has the formula:

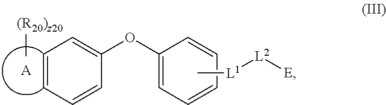

wherein R²⁰, L¹, L², and E are as described herein; two adjacent R¹ substituents form Ring A, wherein Ring A is a cycloalkyl, heterocycloalkyl, aryl, or heteroaryl. The symbol z20 is an integer from 0 to 8.

In embodiments, the compound has the formula:

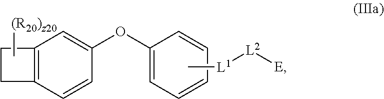

wherein R²⁰, z20, L¹, L², and E are as described herein.

In embodiments, the compound has the formula:

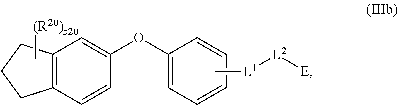

wherein R²⁰, z20, L¹, L², and E are as described herein.

In embodiments, the compound has the formula:

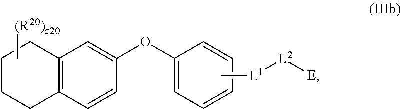

wherein R²⁰, z20, L¹, L², and E are as described herein.

In embodiments, the compound has the formula:

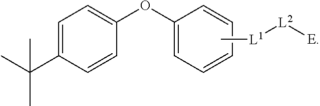

L¹, L², and E are as described herein.

In embodiments, R¹ is independently halogen, —CX¹₃, —CHX¹₂, —CH₂X¹, —OCX¹₃, —OCH₂X¹, —OCHX¹₂, —CN, —SR^{1D}, —NR^{1A}R^{1B}, —C(O)R^{1C}, —C(O)—OR^{1C}, —C(O)NR^{1A}R^{1B}, —OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ aryl, or substituted or unsubstituted 5 to 12 membered heteroaryl.

In embodiments, R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

In embodiments, two adjacent R¹ substituents are joined to form a substituted or unsubstituted cycloalkyl, substituted or unsubstituted heterocycloalkyl, substituted or unsubstituted aryl, or substituted or unsubstituted heteroaryl. In embodiments, two adjacent R¹ substituents are joined to form an unsubstituted cycloalkyl. In embodiments, two adjacent R¹ substituents are joined to form an unsubstituted C₃-C₆ cycloalkyl.

In embodiments, R¹ is independently —Cl. In embodiments, R¹ is independently halogen. In embodiments, R¹ is independently unsubstituted methyl. In embodiments, R¹ is independently unsubstituted ethyl. In embodiments, R¹ is independently unsubstituted propyl. In embodiments, R¹ is independently unsubstituted isopropyl. In embodiments, R¹ is independently unsubstituted n-propyl. In embodiments, R¹ is independently unsubstituted butyl. In embodiments, R¹ is independently unsubstituted n-butyl. In embodiments, R¹ is independently unsubstituted t-butyl. In embodiments, R¹ is independently unsubstituted pentyl. In embodiments, R¹ is independently unsubstituted n-pentyl. In embodiments, R¹ is independently unsubstituted hexyl. In embodiments, R¹ is independently unsubstituted n-hexyl. In embodiments, R¹ is independently unsubstituted heptyl. In embodiments, R¹ is independently unsubstituted n-heptyl. In embodiments, R¹ is independently unsubstituted octyl. In embodiments, R¹ is independently unsubstituted n-octyl. In embodiments, R¹ is independently unsubstituted benzyl. In embodiments, R¹ is independently unsubstituted C₁-C₈ alkyl. In embodiments, R¹ is independently halo-substituted methyl. In embodiments, R¹ is independently halo-substituted ethyl. In embodiments, R¹ is independently halo-substituted isopropyl. In embodiments, R¹ is independently halo-substituted n-propyl. In embodiments, R¹ is independently halo-substituted n-butyl. In embodiments, R¹ is independently halo-substituted t-butyl. In embodiments, R¹ is independently halo-substituted n-pentyl. In embodiments, R¹ is independently halo-substituted benzyl. In embodiments, R¹ is independently halo-substituted C₁-C₈ alkyl. In embodiments, R¹ is independently unsubstituted 2 to 6 membered heteroalkyl. In embodiments, R¹ is independently unsubstituted 2 to 7 membered heteroalkyl. In embodiments, R¹ is independently unsubstituted 2 to 8 membered heteroalkyl. In embodiments, R¹ is independently unsubstituted 2 to 9 membered heteroalkyl. In embodiments, R¹ is independently unsubstituted 2 to 10 membered heteroalkyl. In embodiments, R¹ is independently unsubstituted 3 to 10 membered heteroalkyl. In embodiments, R¹ is independently unsubstituted 4 to 10 membered heteroalkyl. In embodiments, R¹ is independently unsubstituted 5 to 10 membered heteroalkyl. In embodiments, R¹ is independently unsubstituted 6 to 10 membered heteroalkyl. In embodiments, R¹ is independently unsubstituted 7 to 10 membered heteroalkyl. In embodiments, R¹ is independently unsubstituted 8 to 10 membered heteroalkyl. In embodiments, R¹ is independently unsubstituted 6 to 10 membered heteroalkyl. In embodiments, R¹ is independently unsubstituted 7 to 9 membered heteroalkyl.

In embodiments, two adjacent R¹ substituents are joined to form an unsubstituted C₃-C₆ cycloalkyl. In embodiments, two adjacent R¹ substituents are joined to form an unsubstituted C₄-C₆ cycloalkyl. In embodiments, two adjacent R¹ substituents are joined to form an unsubstituted C₃-C₅ cycloalkyl. In embodiments, two adjacent R¹ substituents are joined to form an unsubstituted C₅-C₆ cycloalkyl. In embodiments, two adjacent R¹ substituents are joined to form an unsubstituted C₄ cycloalkyl.

In embodiments, R¹ is independently unsubstituted 5 membered heteroaryl. In embodiments, R¹ is independently unsubstituted 6 membered heteroaryl. In embodiments, R¹ is independently unsubstituted pyridyl. In embodiments, R¹ is independently unsubstituted 2-pyridyl. In embodiments, R¹ is independently unsubstituted 3-pyridyl. In embodiments, R¹ is independently unsubstituted 4-pyridyl. In embodiments, R¹ is independently unsubstituted pyridazinyl. In embodiments, R¹ is independently unsubstituted pyrimidinyl. In embodiments, R¹ is independently unsubstituted pyrazinyl. In embodiments, R¹ is independently unsubstituted triazinyl. In embodiments, R¹ is independently unsubstituted pyrrolyl. In embodiments, R¹ is independently unsubstituted 2-pyrrolyl. In embodiments, R¹ is independently unsubstituted 3-pyrrolyl. In embodiments, R¹ is independently unsubstituted furanyl. In embodiments, R¹ is independently unsubstituted 2-furanyl. In embodiments, R¹ is independently unsubstituted 3-furanyl. In embodiments, R¹ is independently unsubstituted thienyl. In embodiments, R¹ is independently unsubstituted 2-thienyl. In embodiments, R¹ is independently unsubstituted 3-thienyl. In embodiments, R¹ is independently unsubstituted pyrazolyl. In embodiments, R¹ is independently unsubstituted isoxazolyl. In embodiments, R¹ is independently unsubstituted isothiazolyl. In embodiments, R¹ is independently unsubstituted imidazolyl. In embodiments, R¹ is independently unsubstituted oxazolyl. In embodiments, R¹ is independently unsubstituted thiazolyl. In embodiments, R¹ is independently unsubstituted phenyl. In embodiments, R¹ is independently unsubstituted biphenyl. In embodiments, R¹ is independently unsubstituted 2-biphenyl. In embodiments, R¹ is independently unsubstituted 3-biphenyl. In embodiments, R¹ is independently unsubstituted 4-biphenyl.

In embodiments, R¹ is independently —CX¹₃. In embodiments, R¹ is independently —CHX¹₂. In embodiments, R¹ is independently —CH₂X¹. In embodiments, R¹ is independently —OCX¹₃. In embodiments, R¹ is independently —OCH₂X¹. In embodiments, R¹ is independently —OCHX¹₂. In embodiments, R¹ is independently —CN. In embodiments, R¹ is independently —SOₙ₁R^{1D}. In embodiments, R¹ is independently —SOᵥ₁NR^{1A}R^{1B}. In embodiments, R¹ is independently —NHC(O)NR^{1A}R^{1B}. In embodiments, R¹ is independently —N(O)ₘ₁. In embodiments, R¹ is independently —NR^{1A}R^{1B}. In embodiments, R¹ is independently —C(O)R^{1C}. In embodiments, R¹ is independently —C(O)—OR^{1C}. In embodiments, R¹ is independently —C(O)NR^{1A}R^{1B}. In embodiments, R¹ is independently —OR^{1D}. In embodiments, R¹ is independently —NR^{1A}SO₂R^{1D}. In embodiments, R¹ is independently —NR^{1A}C(O)R^{1C}. In embodiments, R¹ is independently —NR^{1A}C(O)OR^{1C}. In embodiments, R¹ is independently —NR^{1A}OR^{1C}. In embodiments, R¹ is independently —OH. In embodiments, R¹ is independently —NH₂. In embodiments, R¹ is independently —COOH. In embodiments, R¹ is independently —CONH₂. In embodiments, R¹ is independently —NO₂. In embodiments, R¹ is independently —SH. In embodiments, R¹ is independently halogen. In embodiments, R¹ is independently —F. In embodiments, R¹ is independently —Cl. In embodiments, R¹ is independently —Br. In embodiments, R¹ is independently —I. In embodiments, R¹ is independently —CF₃. In embodiments, R¹ is independently —CHF₂. In embodiments, R¹ is independently —CH₂F. In embodiments, R¹ is independently —OCF₃. In embodiments, R¹ is independently —OCH₂F. In embodiments, R¹ is independently —OCHF₂. In embodiments, R¹ is independently —OCH₃. In embodiments, R¹ is independently —OCH₂CH₃. In embodiments, R¹ is independently —OCH₂CH₂CH₃. In embodiments, R¹ is independently —OCH(CH₃)₂. In embodiments, R¹ is independently —OC(CH₃)₃. In embodiments, R¹ is independently —SCH₃. In embodiments, R¹ is independently —SCH₂CH₃. In embodiments, R¹ is independently —SCH₂CH₂CH₃. In embodiments, R¹ is independently —SCH(CH₃)₂. In embodiments, R¹ is independently —SC(CH₃)₃. In embodiments, R¹ is independently —CH₃. In embodiments, R¹ is independently —CH₂CH₃. In embodiments, R¹ is independently —CH₂CH₂CH₃. In embodiments, R¹ is independently —CH(CH₃)₂. In embodiments, R¹ is independently —C(CH₃)₃.

In embodiments, R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12, 5 to 10 membered, 5 to 9 membered, or 5 to 6 membered). In embodiments, R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12, 5 to 10 membered, 5 to 9 membered, or 5 to 6 membered).

In embodiments, R¹ is independently substituted or unsubstituted alkyl (e.g., C₁-C₈, C₁-C₆, C₁-C₄, or C₁-C₂). In embodiments, R¹ is independently substituted alkyl (e.g., C₁-C₈, C₁-C₆, C₁-C₄, or C₁-C₂). In embodiments, R¹ is independently unsubstituted alkyl (e.g., C₁-C₈, C₁-C₆, C₁-C₄, or C₁-C₂). In embodiments, R¹ is independently unsubstituted methyl. In embodiments, R¹ is independently unsubstituted ethyl. In embodiments, R¹ is independently unsubstituted propyl. In embodiments, R¹ is independently unsubstituted isopropyl. In embodiments, R¹ is independently unsubstituted tert-butyl. In embodiments, R¹ is independently substituted or unsubstituted heteroalkyl (e.g., 2 to 8 membered, 2 to 6 membered, 4 to 6 membered, 2 to 3 membered, or 4 to 5 membered). In embodiments, R¹ is independently substituted heteroalkyl (e.g., 2 to 8 membered, 2 to 6 membered, 4 to 6 membered, 2 to 3 membered, or 4 to 5 membered). In embodiments, R¹ is independently unsubstituted heteroalkyl (e.g., 2 to 8 membered, 2 to 6 membered, 4 to 6 membered, 2 to 3 membered, or 4 to 5 membered). In embodiments, R¹ is independently substituted or unsubstituted cycloalkyl (e.g., C₃-C₈, C₃-C₆, C₄-C₆, or C₅-C₆). In embodiments, R¹ is independently substituted cycloalkyl (e.g., C₃-C₈, C₃-C₆, C₄-C₆, or C₅-C₆). In embodiments, R¹ is independently unsubstituted cycloalkyl (e.g., C₃-C₈, C₃-C₆, C₄-C₆, or C₅-C₆). In embodiments, R¹ is independently substituted or unsubstituted heterocycloalkyl (e.g., 3 to 8 membered, 3 to 6 membered, 4 to 6 membered, 4 to 5 membered, or 5 to 6 membered). In embodiments, R¹ is independently substituted heterocycloalkyl (e.g., 3 to 8 membered, 3 to 6 membered, 4 to 6 membered, 4 to 5 membered, or 5 to 6 membered). In embodiments, R¹ is independently unsubstituted heterocycloalkyl (e.g., 3 to 8 membered, 3 to 6 membered, 4 to 6 membered, 4 to 5 membered, or 5 to 6 membered). In embodiments, R¹ is independently substituted or unsubstituted aryl (e.g., C₆-C₁₂, C₆-C₁₀, or phenyl). In embodiments, R¹ is independently substituted aryl (e.g., C₆-C₁₂, C₆-C₁₀, or phenyl). In embodiments, R¹ is independently unsubstituted aryl (e.g., C₆-C₁₂, C₆-C₁₀, or phenyl). In embodiments, R¹ is independently substituted or unsubstituted heteroaryl (e.g., 5 to 12 membered, 5 to 10 membered, 5 to 9 membered, or 5 to 6 membered). In embodiments, R¹ is independently substituted heteroaryl (e.g., 5 to 12 membered, 5 to 10 membered, 5 to 9 membered, or 5 to 6 membered). In embodiments, R¹ is independently unsubstituted heteroaryl (e.g., 5 to 12 membered, 5 to 10 membered, 5 to 9 membered, or 5 to 6 membered).

In embodiments, two adjacent R¹ substituents may optionally be joined to form a substituted or unsubstituted cycloalkyl (e.g., C₃-C₈, C₃-C₆, C₄-C₆, or C₅-C₆). In embodiments, two adjacent R¹ substituents may optionally be joined to form a substituted cycloalkyl (e.g., C₃-C₈, C₃-C₆, C₄-C₆, or C₅-C₆). In embodiments, two adjacent R¹ substituents may optionally be joined to form an unsubstituted cycloalkyl (e.g., C₃-C₈, C₃-C₆, C₄-C₆, or C₅-C₆). In embodiments, two adjacent R¹ substituents may optionally be joined to form a substituted or unsubstituted heterocycloalkyl (e.g., 3 to 8 membered, 3 to 6 membered, 4 to 6 membered, 4 to 5 membered, or 5 to 6 membered). In embodiments, two adjacent R¹ substituents may optionally be joined to form a substituted heterocycloalkyl (e.g., 3 to 8 membered, 3 to 6 membered, 4 to 6 membered, 4 to 5 membered, or 5 to 6 membered). In embodiments, two adjacent R¹ substituents may optionally be joined to form an unsubstituted heterocycloalkyl (e.g., 3 to 8 membered, 3 to 6 membered, 4 to 6 membered, 4 to 5 membered, or 5 to 6 membered). In embodiments, two adjacent R¹ substituents may optionally be joined to form a substituted or unsubstituted aryl (e.g., C₆-C₁₂, C₆-C₁₀, or phenyl). In embodiments, two adjacent R¹ substituents may optionally be joined to form a substituted aryl (e.g., C₆-C₁₂, C₆-C₁₀, or phenyl). In embodiments, two adjacent R¹ substituents may optionally be joined to form an unsubstituted aryl (e.g., C₆-C₁₂, C₆-C₁₀, or phenyl). In embodiments, two adjacent R¹ substituents may optionally be joined to form a substituted or unsubstituted heteroaryl (e.g., 5 to 12 membered, 5 to 10 membered, 5 to 9 membered, or 5 to 6 membered). In embodiments, two adjacent R¹ substituents may optionally be joined to form a substituted heteroaryl (e.g., 5 to 12 membered, 5 to 10 membered, 5 to 9 membered, or 5 to 6 membered). In embodiments, two adjacent R¹ substituents may optionally be joined to form an unsubstituted heteroaryl (e.g., 5 to 12 membered, 5 to 10 membered, 5 to 9 membered, or 5 to 6 membered).

In embodiments, R^{1A} is independently hydrogen. In embodiments, R^{1A} is independently —CX^{1A}₃. In embodiments, R^{1A} is independently —CHX^{1A}₂. In embodiments, R^{1A} is independently —CH₂X^{1A}. In embodiments, R^{1A} is independently —CN. In embodiments, R^{1A} is independently —COOH. In embodiments, R^{1A} is independently —CONH₂. In embodiments, X^{1A} is independently —F, —Cl, —Br, or —I.

In embodiments, R^{1A} is independently substituted or unsubstituted alkyl (e.g., C₁-C₈, C₁-C₆, C₁-C₄, or C₁-C₂). In embodiments, R^{1A} is independently substituted alkyl (e.g., C₁-C₈, C₁-C₆, C₁-C₄, or C₁-C₂). In embodiments, R^{1A} is independently unsubstituted alkyl (e.g., C₁-C₈, C₁-C₆, C₁-C₄, or C₁-C₂). In embodiments, R^{1A} is independently unsubstituted methyl. In embodiments, R^{1A} is independently unsubstituted ethyl. In embodiments, R^{1A} is independently unsubstituted propyl. In embodiments, R^{1A} is independently unsubstituted isopropyl. In embodiments, R^{1A} is independently unsubstituted tert-butyl. In embodiments, R^{1A} is independently substituted or unsubstituted heteroalkyl (e.g., 2 to 8 membered, 2 to 6 membered, 4 to 6 membered, 2 to 3 membered, or 4 to 5 membered). In embodiments, R^{1A} is independently substituted heteroalkyl (e.g., 2 to 8 membered, 2 to 6 membered, 4 to 6 membered, 2 to 3 membered, or 4 to 5 membered). In embodiments, R^{1A} is independently unsubstituted heteroalkyl (e.g., 2 to 8 membered, 2 to 6 membered, 4 to 6 membered, 2 to 3 membered, or 4 to 5 membered). In embodiments, R^{1A} is independently substituted or unsubstituted cycloalkyl (e.g., C₃-C₈, C₃-C₆, C₄-C₆, or C₅-C₆). In embodiments, R^{1A} is independently substituted cycloalkyl (e.g., C₃-C₈, C₃-C₆, C₄-C₆, or C₅-C₆). In embodiments, R^{1A} is independently unsubstituted cycloalkyl (e.g., C₃-C₈, C₃-C₆, C₄-C₆, or C₅-C₆). In embodiments, R^{1A} is independently substituted or unsubstituted heterocycloalkyl (e.g., 3 to 8 membered, 3 to 6 membered, 4 to 6 membered, 4 to 5 membered, or 5 to 6 membered). In embodiments, R^{1A} is independently substituted heterocycloalkyl (e.g., 3 to 8 membered, 3 to 6 membered, 4 to 6 membered, 4 to 5 membered, or 5 to 6 membered). In embodiments, R^{1A} is independently unsubstituted heterocycloalkyl (e.g., 3 to 8 membered, 3 to 6 membered, 4 to 6 membered, 4 to 5 membered, or 5 to 6 membered). In embodiments, R^{1A} is independently substituted or unsubstituted aryl (e.g., C₆-C₁₂, C₆-C₁₀, or phenyl). In embodiments, R^{1A} is independently substituted aryl (e.g., C₆-C₁₂, C₆-C₁₀, or phenyl). In embodiments, R^{1A} is independently unsubstituted aryl (e.g., C₆-C₁₂, C₆-C₁₀, or phenyl). In embodiments, R^{1A} is independently substituted or unsubstituted heteroaryl (e.g., 5 to 12 membered, 5 to 10 membered, 5 to 9 membered, or 5 to 6 membered). In embodiments, R^{1A} is independently substituted heteroaryl (e.g., 5 to 12 membered, 5 to 10 membered, 5 to 9 membered, or 5 to 6 membered). In embodiments, R^{1A} is independently unsubstituted heteroaryl (e.g., 5 to 12 membered, 5 to 10 membered, 5 to 9 membered, or 5 to 6 membered).

In embodiments, R^{1B} is independently hydrogen. In embodiments, R^{1B} is independently —CX^{1B}₃. In embodiments, R^{1B} is independently —CHX^{1B}₂. In embodiments, R^{1B} is independently —CH₂X^{1B}. In embodiments, R^{1B} is independently —CN. In embodiments, R^{1B} is independently —COOH. In embodiments, R^{1B} is independently —CONH₂. In embodiments, X^{1B} is independently —F, —Cl, —Br, or —I.

In embodiments, R^{1B} is independently substituted or unsubstituted alkyl (e.g., C₁-C₈, C₁-C₆, C₁-C₄, or C₁-C₂). In embodiments, R^{1B} is independently substituted alkyl (e.g., C₁-C₈, C₁-C₆, C₁-C₄, or C₁-C₂). In embodiments, R^{1B} is independently unsubstituted alkyl (e.g., C₁-C₈, C₁-C₆, C₁-C₄, or C₁-C₂). In embodiments, R^{1B} is independently unsubstituted methyl. In embodiments, R^{1B} is independently unsubstituted ethyl. In embodiments, R^{1B} is independently unsubstituted propyl. In embodiments, R^{1B} is independently unsubstituted isopropyl. In embodiments, R^{1B} is independently unsubstituted tert-butyl. In embodiments, R^{1B} is independently substituted or unsubstituted heteroalkyl (e.g., 2 to 8 membered, 2 to 6 membered, 4 to 6 membered, 2 to 3 membered, or 4 to 5 membered). In embodiments, R^{1B} is independently substituted heteroalkyl (e.g., 2 to 8 membered, 2 to 6 membered, 4 to 6 membered, 2 to 3 membered, or 4 to 5 membered). In embodiments, R^{1B} is independently unsubstituted heteroalkyl (e.g., 2 to 8 membered, 2 to 6 membered, 4 to 6 membered, 2 to 3 membered, or 4 to 5 membered). In embodiments, R^{1B} is independently substituted or unsubstituted cycloalkyl (e.g., C₃-C₈, C₃-C₆, C₄-C₆, or C₅-C₆). In embodiments, R^{1B} is independently substituted cycloalkyl (e.g., C₃-C₈, C₃-C₆, C₄-C₆, or C₅-C₆). In embodiments, R^{1B} is independently unsubstituted cycloalkyl (e.g., C₃-C₈, C₃-C₆, C₄-C₆, or C₅-C₆). In embodiments, R^{1B} is independently substituted or unsubstituted heterocycloalkyl (e.g., 3 to 8 membered, 3 to 6 membered, 4 to 6 membered, 4 to 5 membered, or 5 to 6 membered). In embodiments, R^{1B} is independently substituted heterocycloalkyl (e.g., 3 to 8 membered, 3 to 6 membered, 4 to 6 membered, 4 to 5 membered, or 5 to 6 membered). In embodiments, R^{1B} is independently unsubstituted heterocycloalkyl (e.g., 3 to 8 membered, 3 to 6 membered, 4 to 6 membered, 4 to 5 membered, or 5 to 6 membered). In embodiments, R^{1B} is independently substituted or unsubstituted aryl (e.g., C₆-C₁₂, C₆-C₁₀, or phenyl). In embodiments, R^{1B} is independently substituted aryl (e.g., C₆-C₁₂, C₆-C₁₀, or phenyl). In embodiments, R^{1B} is independently unsubstituted aryl (e.g., C₆-C₁₂, C₆-C₁₀, or phenyl). In embodiments, R^{1B} is independently substituted or unsubstituted heteroaryl (e.g., 5 to 12 membered, 5 to 10 membered, 5 to 9 membered, or 5 to 6 membered). In embodiments, R^{1B} is independently substituted heteroaryl (e.g., 5 to 12 membered, 5 to 10 membered, 5 to 9 membered, or 5 to 6 membered). In embodiments, R^{1B} is independently unsubstituted heteroaryl (e.g., 5 to 12 membered, 5 to 10 membered, 5 to 9 membered, or 5 to 6 membered).

In embodiments, R^{1A} and R^{1B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{1A} and R^{1B} substituents bonded to the same nitrogen atom may be joined to form a substituted or unsubstituted heteroaryl (e.g., 5 to 12 membered, 5 to 10 membered, 5 to 9 membered, or 5 to 6 membered). In embodiments, R^{1A} and R^{1B} substituents bonded to the same nitrogen atom may be joined to form a substituted heteroaryl (e.g., 5 to 12 membered, 5 to 10 membered, 5 to 9 membered, or 5 to 6 membered). In embodiments, R^{1A} and R^{1B} substituents bonded to the same nitrogen atom may be joined to form an unsubstituted heteroaryl (e.g., 5 to 12 membered, 5 to 10 membered, 5 to 9 membered, or 5 to 6 membered).

In embodiments, R^{1C} is independently hydrogen. In embodiments, R^{1C} is independently —CX^{1C}₃. In embodiments, R^{1C} is independently —CHX^{1C}₂. In embodiments, R^{1C} is independently —CH₂X^{1C}. In embodiments, R^{1C} is independently —CN. In embodiments, R^{1C} is independently —COOH. In embodiments, R^{1C} is independently —CONH₂. In embodiments, X^{1C} is independently —F, —Cl, —Br, or —I.

In embodiments, R^{1C} is independently substituted or unsubstituted alkyl (e.g., C₁-C₈, C₁-C₆, C₁-C₄, or C₁-C₂). In embodiments, R^{1C} is independently substituted alkyl (e.g., C₁-C₈, C₁-C₆, C₁-C₄, or C₁-C₂). In embodiments, R^{1C} is independently unsubstituted alkyl (e.g., C₁-C₈, C₁-C₆, C₁-C₄, or C₁-C₂). In embodiments, R^{1C} is independently unsubstituted methyl. In embodiments, R^{1C} is independently unsubstituted ethyl. In embodiments, R^{1C} is independently unsubstituted propyl. In embodiments, R^{1C} is independently unsubstituted isopropyl. In embodiments, R^{1C} is independently unsubstituted tert-butyl. In embodiments, R^{1C} is independently substituted or unsubstituted heteroalkyl (e.g., 2 to 8 membered, 2 to 6 membered, 4 to 6 membered, 2 to 3 membered, or 4 to 5 membered). In embodiments, R^{1C} is independently substituted heteroalkyl (e.g., 2 to 8 membered, 2 to 6 membered, 4 to 6 membered, 2 to 3 membered, or 4 to 5 membered). In embodiments, R^{1C} is independently unsubstituted heteroalkyl (e.g., 2 to 8 membered, 2 to 6 membered, 4 to 6 membered, 2 to 3 membered, or 4 to 5 membered). In embodiments, R^{1C} is independently substituted or unsubstituted cycloalkyl (e.g., C₃-C₈, C₃-C₆, C₄-C₆, or C₅-C₆). In embodiments, R^{1C} is independently substituted cycloalkyl (e.g., C₃-C₈, C₃-C₆, C₄-C₆, or C₅-C₆). In embodiments, R^{1C} is independently unsubstituted cycloalkyl (e.g., C₃-C₈, C₃-C₆, C₄-C₆, or C₅-C₆). In embodiments, R^{1C} is independently substituted or unsubstituted heterocycloalkyl (e.g., 3 to 8 membered, 3 to 6 membered, 4 to 6 membered, 4 to 5 membered, or 5 to 6 membered). In embodiments, R^{1C} is independently substituted heterocycloalkyl (e.g., 3 to 8 membered, 3 to 6 membered, 4 to 6 membered, 4 to 5 membered, or 5 to 6 membered). In embodiments, R^{1C} is independently unsubstituted heterocycloalkyl (e.g., 3 to 8 membered, 3 to 6 membered, 4 to 6 membered, 4 to 5 membered, or 5 to 6 membered). In embodiments, R^{1C} is independently substituted or unsubstituted aryl (e.g., C₆-C₁₂, C₆-C₁₀, or phenyl). In embodiments, R^{1C} is independently substituted aryl (e.g., C₆-C₁₂, C₆-C₁₀, or phenyl). In embodiments, R^{1C} is independently unsubstituted aryl (e.g., C₆-C₁₂, C₆-C₁₀, or phenyl). In embodiments, R^{1C} is independently substituted or unsubstituted heteroaryl (e.g., 5 to 12 membered, 5 to 10 membered, 5 to 9 membered, or 5 to 6 membered). In embodiments, R^{1C} is independently substituted heteroaryl (e.g., 5 to 12 membered, 5 to 10 membered, 5 to 9 membered, or 5 to 6 membered). In embodiments, R^{1C} is independently unsubstituted heteroaryl (e.g., 5 to 12 membered, 5 to 10 membered, 5 to 9 membered, or 5 to 6 membered).

In embodiments, R^{1D} is independently hydrogen. In embodiments, R^{1D} is independently —CX^{1D}₃. In embodiments, R^{1D} is independently —CHX^{1D}₂. In embodiments, R^{1D} is independently —CH₂X^{1D}. In embodiments, R^{1D} is independently —CN. In embodiments, R^{1D} is independently —COOH. In embodiments, R^{1D} is independently —CONH₂. In embodiments, X^{1C} is independently —F, —Cl, —Br, or —I.

In embodiments, R^{1D} is independently substituted or unsubstituted alkyl (e.g., C₁-C₈, C₁-C₆, C₁-C₄, or C₁-C₂). In embodiments, R^{1D} is independently substituted alkyl (e.g., C₁-C₈, C₁-C₆, C₁-C₄, or C₁-C₂). In embodiments, R^{1D} is independently unsubstituted alkyl (e.g., C₁-C₈, C₁-C₆, C₁-C₄, or C₁-C₂). In embodiments, R^{1D} is independently unsubstituted methyl. In embodiments, R^{1D} is independently unsubstituted ethyl. In embodiments, R^{1D} is independently unsubstituted propyl. In embodiments, R^{1D} is independently unsubstituted isopropyl. In embodiments, R^{1D} is independently unsubstituted tert-butyl. In embodiments, R^{1D} is independently substituted or unsubstituted heteroalkyl (e.g., 2 to 8 membered, 2 to 6 membered, 4 to 6 membered, 2 to 3 membered, or 4 to 5 membered). In embodiments, R^{1D} is independently substituted heteroalkyl (e.g., 2 to 8 membered, 2 to 6 membered, 4 to 6 membered, 2 to 3 membered, or 4 to 5 membered). In embodiments, R^{1D} is independently unsubstituted heteroalkyl (e.g., 2 to 8 membered, 2 to 6 membered, 4 to 6 membered, 2 to 3 membered, or 4 to 5 membered). In embodiments, R^{1D} is independently substituted or unsubstituted cycloalkyl (e.g., C₃-C₈, C₃-C₆, C₄-C₆, or C₅-C₆). In embodiments, R^{1D} is independently substituted cycloalkyl (e.g., C₃-C₈, C₃-C₆, C₄-C₆, or C₅-C₆). In embodiments, R^{1D} is independently unsubstituted cycloalkyl (e.g., C₃-C₈, C₃-C₆, C₄-C₆, or C₅-C₆). In embodiments, R^{1D} is independently substituted or unsubstituted heterocycloalkyl (e.g., 3 to 8 membered, 3 to 6 membered, 4 to 6 membered, 4 to 5 membered, or 5 to 6 membered). In embodiments, R^{1D} is independently substituted heterocycloalkyl (e.g., 3 to 8 membered, 3 to 6 membered, 4 to 6 membered, 4 to 5 membered, or 5 to 6 membered). In embodiments, R^{1D} is independently unsubstituted heterocycloalkyl (e.g., 3 to 8 membered, 3 to 6 membered, 4 to 6 membered, 4 to 5 membered, or 5 to 6 membered). In embodiments, R^{1D} is independently substituted or unsubstituted aryl (e.g., C₆-C₁₂, C₆-C₁₀, or phenyl). In embodiments, R^{1D} is independently substituted aryl (e.g., C₆-C₁₂, C₆-C₁₀, or phenyl). In embodiments, R^{1D} is independently unsubstituted aryl (e.g., C₆-C₁₂, C₆-C₁₀, or phenyl). In embodiments, R^{1D} is independently substituted or unsubstituted heteroaryl (e.g., 5 to 12 membered, 5 to 10 membered, 5 to 9 membered, or 5 to 6 membered). In embodiments, R^{1D} is independently substituted heteroaryl (e.g., 5 to 12 membered, 5 to 10 membered, 5 to 9 membered, or 5 to 6 membered). In embodiments, R^{1D} is independently unsubstituted heteroaryl (e.g., 5 to 12 membered, 5 to 10 membered, 5 to 9 membered, or 5 to 6 membered).

In embodiments, R¹ is independently halogen, —CX¹₃, —CHX¹₂, —CH₂X¹, —OCX¹₃, —OCH₂X¹, —OCHX¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²⁰-substituted or unsubstituted alkyl (e.g., C₁-C₈, C₁-C₆, C₁-C₄, or C₁-C₂), R²⁰-substituted or unsubstituted heteroalkyl (e.g., 2 to 8 membered, 2 to 6 membered, 4 to 6 membered, 2 to 3 membered, or 4 to 5 membered), R²⁰-substituted or unsubstituted cycloalkyl (e.g., C₃-C₈, C₃-C₆, C₄-C₆, or C₅-C₆), R²⁰-substituted or unsubstituted heterocycloalkyl (e.g., 3 to 8 membered, 3 to 6 membered, 4 to 6 membered, 4 to 5 membered, or 5 to 6 membered), R²⁰-substituted or unsubstituted aryl (e.g., C₆-C₁₂, C₆-C₁₀, or phenyl), or R²⁰-substituted or unsubstituted heteroaryl (e.g., 5 to 12 membered, 5 to 10 membered, 5 to 9 membered, or 5 to 6 membered). In embodiments, R¹ is independently halogen, —CX¹₃, —CHX¹₂, —CH₂X¹, —OCX¹₃, —OCH₂X¹, —OCHX¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X¹ is independently —F, —Cl, —Br, or —I. In embodiments, R¹ is independently hydrogen. In embodiments, R¹ is independently unsubstituted methyl. In embodiments, R¹ is independently unsubstituted ethyl. In embodiments, R¹ is independently halogen, —CX¹₃, —CHX¹₂, —CH₂X¹, —OCX¹₃, —OCH₂X¹, —OCHX¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²⁰-substituted or unsubstituted alkyl (e.g., C₁-C₈, C₁-C₆, C₁-C₄, or C₁-C₂), R²⁰-substituted or unsubstituted heteroalkyl (e.g., 2 to 8 membered, 2 to 6 membered, 4 to 6 membered, 2 to 3 membered, or 4 to 5 membered), R²⁰-substituted or unsubstituted cycloalkyl (e.g., C₃-C₈, C₃-C₆, C₄-C₆, or C₅-C₆), R²⁰-substituted or unsubstituted heterocycloalkyl (e.g., 3 to 8 membered, 3 to 6 membered, 4 to 6 membered, 4 to 5 membered, or 5 to 6 membered), R²⁰-substituted or unsubstituted aryl (e.g., C₆-C₁₂, C₆-C₁₀, or phenyl), or R²⁰-substituted or unsubstituted heteroaryl (e.g., 5 to 12 membered, 5 to 10 membered, 5 to 9 membered, or 5 to 6 membered). In embodiments, R¹ is independently halogen, —CX¹₃, —CHX¹₂, —CH₂X¹, —OCX¹₃, —OCH₂X¹, —OCHX¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, two adjacent R¹ substituents may optionally be joined to form a R²⁰-substituted or unsubstituted cycloalkyl (e.g., C₃-C₈, C₃-C₆, C₄-C₆, or C₅-C₆). In embodiments, two adjacent R¹ substituents may optionally be joined to form a R²⁰-substituted cycloalkyl (e.g., C₃-C₈, C₃-C₆, C₄-C₆, or C₅-C₆). In embodiments, two adjacent R¹ substituents may optionally be joined to form an unsubstituted cycloalkyl (e.g., C₃-C₈, C₃-C₆, C₄-C₆, or C₅-C₆). In embodiments, two adjacent R¹ substituents may optionally be joined to form a R²⁰-substituted or unsubstituted heterocycloalkyl (e.g., 3 to 8 membered, 3 to 6 membered, 4 to 6 membered, 4 to 5 membered, or 5 to 6 membered). In embodiments, two adjacent R¹ substituents may optionally be joined to form a R²⁰-substituted heterocycloalkyl (e.g., 3 to 8 membered, 3 to 6 membered, 4 to 6 membered, 4 to 5 membered, or 5 to 6 membered). In embodiments, two adjacent R¹ substituents may optionally be joined to form an unsubstituted heterocycloalkyl (e.g., 3 to 8 membered, 3 to 6 membered, 4 to 6 membered, 4 to 5 membered, or 5 to 6 membered). In embodiments, two adjacent R¹ substituents may optionally be joined to form a R²⁰-substituted or unsubstituted aryl (e.g., C₆-C₁₂, C₆-C₁₀, or phenyl). In embodiments, two adjacent R¹ substituents may optionally be joined to form a R²⁰-substituted aryl (e.g., C₆-C₁₂, C₆-C₁₀, or phenyl). In embodiments, two adjacent R¹ substituents may optionally be joined to form an unsubstituted aryl (e.g., C₆-C₁₂, C₆-C₁₀, or phenyl). In embodiments, two adjacent R¹ substituents may optionally be joined to form a R²⁰-substituted or unsubstituted heteroaryl (e.g., 5 to 12 membered, 5 to 10 membered, 5 to 9 membered, or 5 to 6 membered). In embodiments, two adjacent R¹ substituents may optionally be joined to form a R²⁰-substituted heteroaryl (e.g., 5 to 12 membered, 5 to 10 membered, 5 to 9 membered, or 5 to 6 membered). In embodiments, two adjacent R¹ substituents may optionally be joined to form an unsubstituted heteroaryl (e.g., 5 to 12 membered, 5 to 10 membered, 5 to 9 membered, or 5 to 6 membered).

R²⁰ is independently oxo,

halogen, —CX²⁰₃, —CHX²⁰₂, —CH₂X²⁰, —OCX²⁰₃, —OCH₂X²⁰, —OCHX²⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²¹-substituted or unsubstituted alkyl (e.g., C₁-C₈, C₁-C₆, C₁-C₄, or C₁-C₂), R²¹-substituted or unsubstituted heteroalkyl (e.g., 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R²¹-substituted or unsubstituted heterocycloalkyl (e.g., 3 to 8 membered, 3 to 6 membered, 4 to 6 membered, 4 to 5 membered, or 5 to 6 membered), R²¹-substituted or unsubstituted aryl (e.g., C₆-C₁₂, C₆-C₁₀, or phenyl), or R²¹-substituted or unsubstituted heteroaryl (e.g., 5 to 12 membered, 5 to 10 membered, 5 to 9 membered, or 5 to 6 membered). In embodiments, R²⁰ is independently oxo,
halogen, —CX²⁰₃, —CHX²⁰₂, —CH₂X²⁰, —OCX²⁰₃, —OCH₂X²⁰, —OCHX²⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, or phenyl), or unsubstituted heteroaryl (e.g., 5 to 12 membered, 5 to 10 membered, 5 to 9 membered, or 5 to 6 membered). X²⁰ is independently —F, —Cl, —Br, or —I. In embodiments, R²⁰ is independently unsubstituted methyl. In embodiments, R²⁰ is independently unsubstituted ethyl.

R²¹ is independently oxo,

halogen, —CX²¹₃, —CHX²¹₂, —CH₂X²¹, —OCX²¹₃, —OCH₂X²¹, —OCHX²¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²²-substituted or unsubstituted alkyl (e.g., C₁-C₈, C₁-C₆, C₁-C₄, or C₁-C₂), R²²-substituted or unsubstituted heteroalkyl (e.g., 2 to 8 membered, 2 to 6 membered, 4 to 6 membered, 2 to 3 membered, or 4 to 5 membered), R²²-substituted or unsubstituted cycloalkyl (e.g., C₃-C₈, C₃-C₆, C₄-C₆, or C₅-C₆), R²²-substituted or unsubstituted heterocycloalkyl (e.g., 3 to 8 membered, 3 to 6 membered, 4 to 6 membered, 4 to 5 membered, or 5 to 6 membered), R²²-substituted or unsubstituted aryl (e.g., C₆-C₁₂, C₆-C₁₀, or phenyl), or R²²-substituted or unsubstituted heteroaryl (e.g., 5 to 12 membered, 5 to 10 membered, 5 to 9 membered, or 5 to 6 membered). In embodiments, R²¹ is independently oxo,
halogen, —CX²¹₃, —CHX²¹₂, —CH₂X²¹, —OCX²¹₃, —OCH₂X²¹, —OCHX²¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X²¹ is independently —F, —Cl, —Br, or —I. In embodiments, R²¹ is independently unsubstituted methyl. In embodiments, R²¹ is independently unsubstituted ethyl.

R²² is independently oxo,

halogen, —CX²²₃, —CHX²²₂, —CH₂X²², —OCX²²₃, —OCH₂X²², —OCHX²²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X²² is independently —F, —Cl, —Br, or —I. In embodiments, R²² is independently unsubstituted methyl. In embodiments, R²² is independently unsubstituted ethyl.

In embodiments, R^{1A} is independently

hydrogen, —CX^{1A}₃, —CHX^{1A}₂, —CH₂X^{1A}, —CN, —COOH, —CONH₂, R^{20A}-substituted or unsubstituted alkyl (e.g., C₁-C₈, C₁-C₆, C₁-C₄, or C₁-C₂), R^{20A}-substituted or unsubstituted heteroalkyl (e.g., 2 to 8 membered, 2 to 6 membered, 4 to 6 membered, 2 to 3 membered, or 4 to 5 membered), R^{20A}-substituted or unsubstituted cycloalkyl (e.g., C₃-C₈, C₃-C₆, C₄-C₆, or C₅-C₆), R^{20A}-substituted or unsubstituted heterocycloalkyl (e.g., 3 to 8 membered, 3 to 6 membered, 4 to 6 membered, 4 to 5 membered, or 5 to 6 membered), R^{20A}-substituted or unsubstituted aryl (e.g., C₆-C₁₂, C₆-C₁₀, or phenyl), or R^{20A}-substituted or unsubstituted heteroaryl (e.g., 5 to 12 membered, 5 to 10 membered, 5 to 9 membered, or 5 to 6 membered). In embodiments, R^{1A} is independently hydrogen, —CX^{1A}₃, —CHX^{1A}₂, —CH₂X^{1A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{1A} is independently —F, —Cl, —Br, or —I. In embodiments, R^{1A} is independently hydrogen. In embodiments, R^{1A} is independently unsubstituted methyl. In embodiments, R^{1A} is independently unsubstituted ethyl.

In embodiments, R^{1A} and R^{1B} substituents bonded to the same nitrogen atom may optionally be joined to form a R^{20A}-substituted or unsubstituted heterocycloalkyl (e.g., 3 to 8 membered, 3 to 6 membered, 4 to 6 membered, 4 to 5 membered, or 5 to 6 membered) or R^{20A}-substituted or unsubstituted heteroaryl (e.g., 5 to 12 membered, 5 to 10 membered, 5 to 9 membered, or 5 to 6 membered). In embodiments, R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 12 membered, 5 to 10 membered, 5 to 9 membered, or 5 to 6 membered). In embodiments, R^{1A} and R^{1B} substituents bonded to the same nitrogen atom may optionally be joined to form a R^{20A}-substituted or unsubstituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{20A} is independently oxo,

halogen, —CX^{20A}₃, —CHX^{20A}₂, —CH₂X^{20A}, —OCX^{20A}₃, —OCH₂X^{20A}, —OCHX^{20A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{21A}-substituted or unsubstituted alkyl (e.g., C₁-C₈, C₁-C₆, C₁-C₄, or C₁-C₂), R^{21A}-substituted or unsubstituted heteroalkyl (e.g., 2 to 8 membered, 2 to 6 membered, 4 to 6 membered, 2 to 3 membered, or 4 to 5 membered), R^{21A}-substituted or unsubstituted cycloalkyl (e.g., C₃-C₈, C₃-C₆, C₄-C₆, or C₅-C₆), R^{21A}-substituted or unsubstituted heterocycloalkyl (e.g., 3 to 8 membered, 3 to 6 membered, 4 to 6 membered, 4 to 5 membered, or 5 to 6 membered), R^{21A}-substituted or unsubstituted aryl (e.g., C₆-C₁₂, C₆-C₁₀, or phenyl), or R^{21A}-substituted or unsubstituted heteroaryl (e.g., 5 to 12 membered, 5 to 10 membered, 5 to 9 membered, or 5 to 6 membered). In embodiments, R^{20A} is independently oxo, halogen, —CX^{20A}₃, —CHX^{20A}₂, —CH₂X^{20A}, —OCX^{20A}₃, —OCH₂X^{20A}, —OCHX^{20A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{20A} is independently —F, —Cl, —Br, or —I. In embodiments, R^{20A} is independently unsubstituted methyl. In embodiments, R^{20A} is independently unsubstituted ethyl.

R^{21A} is independently oxo,

halogen, —CX^{21A}₃, —CHX^{21A}₂, —CH₂X^{21A}, —OCX^{21A}₃, —OCH₂X^{21A}, —OCHX^{21A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{22A}-substituted or unsubstituted alkyl (e.g., C₁-C₈, C₁-C₆, C₁-C₄, or C₁-C₂), R^{22A}-substituted or unsubstituted heteroalkyl (e.g., 2 to 8 membered, 2 to 6 membered, 4 to 6 membered, 2 to 3 membered, or 4 to 5 membered), R^{22A}-substituted or unsubstituted cycloalkyl (e.g., C₃-C₈, C₃-C₆, C₄-C₆, or C₅-C₆), R^{22A}-substituted or unsubstituted heterocycloalkyl (e.g., 3 to 8 membered, 3 to 6 membered, 4 to 6 membered, 4 to 5 membered, or 5 to 6 membered), R^{22A}-substituted or unsubstituted aryl (e.g., C₆-C₁₂, C₆-C₁₀, or phenyl), or R^{22A}-substituted or unsubstituted heteroaryl (e.g., 5 to 12 membered, 5 to 10 membered, 5 to 9 membered, or 5 to 6 membered). In embodiments, R^{21A} is independently oxo,
halogen, —CX^{21A}₃, —CHX^{21A}₂, —CH₂X^{21A}, —OCX^{21A}₃, —OCH₂X^{21A}, —OCHX^{21A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{21A} is independently —F, —Cl, —Br, or —I. In embodiments, R^{21A} is independently unsubstituted methyl. In embodiments, R^{21A} is independently unsubstituted ethyl.

R^{22A} is independently oxo,

halogen, —CX^{22A}₃, —CHX^{22A}₂, —CH₂X^{22A}, —OCX^{22A}₃, —OCH₂X^{22A}, —OCHX^{22A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{22A} is independently —F, —Cl, —Br, or —I. In embodiments, R^{22A} is independently unsubstituted methyl. In embodiments, R^{22A} is independently unsubstituted ethyl.

In embodiments, R^{1B} is independently hydrogen, —CX^{1B}₃, —CHX^{1B}₂, —CH₂X^{1B}, —CN, —COOH, —CONH₂, R^{20B}-substituted or unsubstituted alkyl (e.g., C₁-C₈, C₁-C₆, C₁-C₄, or C₁-C₂), R^{20B}-substituted or unsubstituted heteroalkyl (e.g., 2 to 8 membered, 2 to 6 membered, 4 to 6 membered, 2 to 3 membered, or 4 to 5 membered), R^{20B}-substituted or unsubstituted cycloalkyl (e.g., C₃-C₈, C₃-C₆, C₄-C₆, or C₅-C₆), R^{20B}-substituted or unsubstituted heterocycloalkyl (e.g., 3 to 8 membered, 3 to 6 membered, 4 to 6 membered, 4 to 5 membered, or 5 to 6 membered), R^{20B}-substituted or unsubstituted aryl (e.g., C₆-C₁₂, C₆-C₁₀, or phenyl), or R^{20B}-substituted or unsubstituted heteroaryl (e.g., 5 to 12 membered, 5 to 10 membered, 5 to 9 membered, or 5 to 6 membered). In embodiments, R^{1B} is independently hydrogen, —CX^{1B}₃, —CHX^{1B}₂, —CH₂X^{1B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{1B} is independently —F, —Cl, —Br, or —I. In embodiments, R^{1B} is independently hydrogen. In embodiments, R^{1B} is independently unsubstituted methyl. In embodiments, R^{1B} is independently unsubstituted ethyl.

In embodiments, R^{1A} and R^{1B} substituents bonded to the same nitrogen atom may optionally be joined to form a R^{20B}-substituted or unsubstituted heterocycloalkyl (e.g., 3 to 8 membered, 3 to 6 membered, 4 to 6 membered, 4 to 5 membered, or 5 to 6 membered) or R^{20B}-substituted or unsubstituted heteroaryl (e.g., 5 to 12 membered, 5 to 10 membered, 5 to 9 membered, or 5 to 6 membered). In embodiments, R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 12 membered, 5 to 10 membered, 5 to 9 membered, or 5 to 6 membered). In embodiments, R^{1A} and R^{1B} substituents bonded to the same nitrogen atom may optionally be joined to form a R^{20B}-substituted or unsubstituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{20B} is independently oxo,

halogen, —CX^{20B}₃, —CHX^{20B}₂, —CH₂X^{20B}, —OXC^{20B}₃, —OCH₂X^{20B}₃, —OCHX^{20B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{21B}-substituted or unsubstituted alkyl (e.g., C₁-C₈, C₁-C₆, C₁-C₄, or C₁-C₂), R^{21B}-substituted or unsubstituted heteroalkyl (e.g., 2 to 8 membered, 2 to 6 membered, 4 to 6 membered, 2 to 3 membered, or 4 to 5 membered), R^{21B}-substituted or unsubstituted cycloalkyl (e.g., C₃-C₈, C₃-C₆, C₄-C₆, or C₅-C₆), R^{21B}-substituted or unsubstituted heterocycloalkyl (e.g., 3 to 8 membered, 3 to 6 membered, 4 to 6 membered, 4 to 5 membered, or 5 to 6 membered), R^{21B}-substituted or unsubstituted aryl (e.g., C₆-C₁₂, C₆-C₁₀, or phenyl), or R^{21B}-substituted or unsubstituted heteroaryl (e.g., 5 to 12 membered, 5 to 10 membered, 5 to 9 membered, or 5 to 6 membered). In embodiments, R^{20B} is independently oxo,
halogen, —CX^{20B}₃, —CHX^{20B}₂, —CH₂X^{20B}, —OCX^{20B}₃, —OCH₂X^{20B}, —OCHX^{20B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{20B} is independently —F, —Cl, —Br, or —I. In embodiments, R^{20B} is independently unsubstituted methyl. In embodiments, R^{20B} is independently unsubstituted ethyl.

R^{21B} is independently oxo,

halogen, —CX^{21B}₃, —CHX^{21B}₂, —CH₂X^{21B}, —OCX^{21B}₃, —OCH₂X^{21B}, —OCHX^{21B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{22B}-substituted or unsubstituted alkyl (e.g., C₁-C₈, C₁-C₆, C₁-C₄, or C₁-C₂), R^{22B}-substituted or unsubstituted heteroalkyl (e.g., 2 to 8 membered, 2 to 6 membered, 4 to 6 membered, 2 to 3 membered, or 4 to 5 membered), R^{22B}-substituted or unsubstituted cycloalkyl (e.g., C₃-C₈, C₃-C₆, C₄-C₆, or C₅-C₆), R^{22B}-substituted or unsubstituted heterocycloalkyl (e.g., 3 to 8 membered, 3 to 6 membered, 4 to 6 membered, 4 to 5 membered, or 5 to 6 membered), R^{22B}-substituted or unsubstituted aryl (e.g., C₆-C₁₂, C₆-C₁₀, or phenyl), or R^{22B}-substituted or unsubstituted heteroaryl (e.g., 5 to 12 membered, 5 to 10 membered, 5 to 9 membered, or 5 to 6 membered). In embodiments, R^{21B} is independently oxo,
halogen, —CX^{21B}₃, —CHX^{21B}₂, —CH₂X^{21B}, —OCX^{21B}₃, —OCH₂X^{21B}, —OCHX^{21B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{21B} is independently —F, —Cl, —Br, or —I. In embodiments, R^{21B} is independently unsubstituted methyl. In embodiments, R^{21B} is independently unsubstituted ethyl.

R^{22B} is independently oxo,

halogen, —CX^{22B}₃, —CHX^{22B}₂, —CH₂X^{22B}, —OCX^{22B}₃, —OCH₂X^{22B}, —OCHX^{22B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{22B} is independently —F, —Cl, —Br, or —I. In embodiments, R^{22B} is independently unsubstituted methyl. In embodiments, R^{22B} is independently unsubstituted ethyl.

In embodiments, R^{1C} is independently hydrogen, —CX^{1C}₃, —CHX^{1C}₂, —CH₂X^{1C}, —CN, —COOH, —CONH₂, R^{20C}-substituted or unsubstituted alkyl (e.g., C₁-C₈, C₁-C₆, C₁-C₄, or C₁-C₂), R^{20C}-substituted or unsubstituted heteroalkyl (e.g., 2 to 8 membered, 2 to 6 membered, 4 to 6 membered, 2 to 3 membered, or 4 to 5 membered), R^{20C}-substituted or unsubstituted cycloalkyl (e.g., C₃-C₈, C₃-C₆, C₄-C₆, or C₅-C₆), R^{20C}-substituted or unsubstituted heterocycloalkyl (e.g., 3 to 8 membered, 3 to 6 membered, 4 to 6 membered, 4 to 5 membered, or 5 to 6 membered), R^{20C}-substituted or unsubstituted aryl (e.g., C₆-C₁₂, C₆-C₁₀, or phenyl), or R^{20C}-substituted or unsubstituted heteroaryl (e.g., 5 to 12 membered, 5 to 10 membered, 5 to 9 membered, or 5 to 6 membered). In embodiments, R^{1C} is independently hydrogen, —CX^{1C}₃, —CHX^{1C}₂, —CH₂X^{1C}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{1C} is independently —F, —Cl, —Br, or —I. In embodiments, R^{1C} is independently hydrogen. In embodiments, R^{1C} is independently unsubstituted methyl. In embodiments, R^{1C} is independently unsubstituted ethyl.

R^{20C} is independently oxo,

halogen, —CX^{20C}₃, —CHX²n, —CH₂X^{20C}, —OCX²n, —OCH₂X^{20C}, —OCHX^{20C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{21C}-substituted or unsubstituted alkyl (e.g., C₁-C₈, C₁-C₆, C₁-C₄, or C₁-C₂), R^{21C}-substituted or unsubstituted heteroalkyl (e.g., 2 to 8 membered, 2 to 6 membered, 4 to 6 membered, 2 to 3 membered, or 4 to 5 membered), R^{21C}-substituted or unsubstituted cycloalkyl (e.g., C₃-C₈, C₃-C₆, C₄-C₆, or C₅-C₆), R^{21C}-substituted or unsubstituted heterocycloalkyl (e.g., 3 to 8 membered, 3 to 6 membered, 4 to 6 membered, 4 to 5 membered, or 5 to 6 membered), R^{21C}-substituted or unsubstituted aryl (e.g., C₆-C₁₂, C₆-C₁₀, or phenyl), or R^{21C}-substituted or unsubstituted heteroaryl (e.g., 5 to 12 membered, 5 to 10 membered, 5 to 9 membered, or 5 to 6 membered). In embodiments, R^{20C} is independently oxo,
halogen, —CX^{20C}₃, —CHX^{20C}₂, —CH₂X^{20C}, —OCX^{20C}₃, —OCH₂X^{20C}, —OCHX^{20C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{20C} is independently —F, —Cl, —Br, or —I. In embodiments, R^{20C} is independently unsubstituted methyl. In embodiments, R^{20C} is independently unsubstituted ethyl.

R^{21C} is independently oxo,

halogen, —CX^{21C}₃, —CHX^{21C}₂, —CH₂X^{21C}, —OCX^{21C}₃, —OCH₂X^{21C}, —OCHX^{21C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{22C}-substituted or unsubstituted alkyl (e.g., C₁-C₈, C₁-C₆, C₁-C₄, or C₁-C₂), R^{22C}-substituted or unsubstituted heteroalkyl (e.g., 2 to 8 membered, 2 to 6 membered, 4 to 6 membered, 2 to 3 membered, or 4 to 5 membered), R^{22C}-substituted or unsubstituted cycloalkyl (e.g., C₃-C₈, C₃-C₆, C₄-C₆, or C₅-C₆), R^{22C}-substituted or unsubstituted heterocycloalkyl (e.g., 3 to 8 membered, 3 to 6 membered, 4 to 6 membered, 4 to 5 membered, or 5 to 6 membered), R^{22C}-substituted or unsubstituted aryl (e.g., C₆-C₁₂, C₆-C₁₀, or phenyl), or R^{22C}-substituted or unsubstituted heteroaryl (e.g., 5 to 12 membered, 5 to 10 membered, 5 to 9 membered, or 5 to 6 membered). In embodiments, R^{21C} is independently oxo,
halogen, —CX^{21C}₃, —CHX^{21C}₂, —CH₂X^{21C}, —OCX^{21C}₃, —OCH₂X^{21C}, —OCHX^{21C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{21C} is independently —F, —Cl, —Br, or —I. In embodiments, R^{21C} is independently unsubstituted methyl. In embodiments, R^{21C} is independently unsubstituted ethyl.

R^{22C} is independently oxo,

halogen, —CX^{22C}₃, —CHX^{22C}₂, —CH₂X^{22C}, —OCX^{22C}₃, —OCH₂X^{22C}, —OCHX^{22C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{22C} is independently —F, —Cl, —Br, or —I. In embodiments, R^{22C} is independently unsubstituted methyl. In embodiments, R^{22C} is independently unsubstituted ethyl.

In embodiments, R^{1D} is independently hydrogen, —CX^{1D}₃, —CHX^{1D}₂, —CH₂X^{1D}, —CN, —COOH, —CONH₂, R^{20D}-substituted or unsubstituted alkyl (e.g., C₁-C₈, C₁-C₆, C₁-C₄, or C₁-C₂), R^{20D}-substituted or unsubstituted heteroalkyl (e.g., 2 to 8 membered, 2 to 6 membered, 4 to 6 membered, 2 to 3 membered, or 4 to 5 membered), R^{20D}-substituted or unsubstituted cycloalkyl (e.g., C₃-C₈, C₃-C₆, C₄-C₆, or C₅-C₆), R^{20D}-substituted or unsubstituted heterocycloalkyl (e.g., 3 to 8 membered, 3 to 6 membered, 4 to 6 membered, 4 to 5 membered, or 5 to 6 membered), R^{20D}-substituted or unsubstituted aryl (e.g., C₆-C₁₂, C₆-C₁₀, or phenyl), or R^{20D}-substituted or unsubstituted heteroaryl (e.g., 5 to 12 membered, 5 to 10 membered, 5 to 9 membered, or 5 to 6 membered). In embodiments, R^{1D} is independently hydrogen, —CX^{1D}₃, —CHX^{1D}₂, —CH₂X^{1D}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{1D} is independently —F, —Cl, —Br, or —I. In embodiments, R^{1D} is independently hydrogen. In embodiments, R^{1D} is independently unsubstituted methyl. In embodiments, R^{1D} is independently unsubstituted ethyl.

R^{20D} is independently oxo,

halogen, —CX^{20D}₃, —CHX^{20D}₂, —CH₂X^{20D}, —OCX^{20D}₃, —OCH₂X^{20D}, —OCHX^{20D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{21D}-substituted or unsubstituted alkyl (e.g., C₁-C₈, C₁-C₆, C₁-C₄, or C₁-C₂), R^{21D}-substituted or unsubstituted heteroalkyl (e.g., 2 to 8 membered, 2 to 6 membered, 4 to 6 membered, 2 to 3 membered, or 4 to 5 membered), R^{21D}-substituted or unsubstituted cycloalkyl (e.g., C₃-C₈, C₃-C₆, C₄-C₆, or C₅-C₆), R^{21D}-substituted or unsubstituted heterocycloalkyl (e.g., 3 to 8 membered, 3 to 6 membered, 4 to 6 membered, 4 to 5 membered, or 5 to 6 membered), R^{21D}-substituted or unsubstituted aryl (e.g., C₆-C₁₂, C₆-C₁₀, or phenyl), or R^{21D} substituted or unsubstituted heteroaryl (e.g., 5 to 12 membered, 5 to 10 membered, 5 to 9 membered, or 5 to 6 membered). In embodiments, R^{20D} is independently oxo,
halogen, —CX^{20D}₃, —CHX^{20D}₂, —CH₂X^{20D}, —OCX^{20D}₃, —OCH₂X^{20D}, —OCHX^{20D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{20D} is independently —F, —Cl, —Br, or —I. In embodiments, R^{20D} is independently unsubstituted methyl. In embodiments, R^{20D} is independently unsubstituted ethyl.

R^{21D} is independently oxo,

halogen, —CX^{21D}₃, —CHX^{21D}₂, —CH₂X^{21D}, —OCX^{21D}₃, —OCH₂X^{21D}, —OCHX^{21D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{22D}-substituted or unsubstituted alkyl (e.g., C₁-C₈, C₁-C₆, C₁-C₄, or C₁-C₂), R^{22D}-substituted or unsubstituted heteroalkyl (e.g., 2 to 8 membered, 2 to 6 membered, 4 to 6 membered, 2 to 3 membered, or 4 to 5 membered), R^{22D}-substituted or unsubstituted cycloalkyl (e.g., C₃-C₈, C₃-C₆, C₄-C₆, or C₅-C₆), R^{22D}-substituted or unsubstituted heterocycloalkyl (e.g., 3 to 8 membered, 3 to 6 membered, 4 to 6 membered, 4 to 5 membered, or 5 to 6 membered), R^{22D}-substituted or unsubstituted aryl (e.g., C₆-C₁₂, C₆-C₁₀, or phenyl), or R^{22D}-substituted or unsubstituted heteroaryl (e.g., 5 to 12 membered, 5 to 10 membered, 5 to 9 membered, or 5 to 6 membered). In embodiments, R^{21D} is independently oxo,
halogen, —CX^{21D}₃, —CHX^{21D}₂, —CH₂X^{21D}, —OCX^{21D}₃, —OCH₂X^{21D}, —OCHX^{21D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{21D} is independently —F, —Cl, —Br, or —I. In embodiments, R^{21D} is independently unsubstituted methyl. In embodiments, R^{21D} is independently unsubstituted ethyl.

R^{22D} is independently oxo,

halogen, —CX^{22D}₃, —CHX^{22D}₂, —CH₂X^{22D}, —OCX^{22D}₃, —OCH₂X^{22D}, —OCHX^{22D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{22D} is independently —F, —Cl, —Br, or —I. In embodiments, R^{22D} is independently unsubstituted methyl. In embodiments, R^{22D} is independently unsubstituted ethyl.

In embodiments, z1 is 0. In embodiments, z1 is 1. In embodiments, z1 is 2. In embodiments, z1 is 3. In embodiments, z1 is 4. In embodiments, z1 is 5.

In embodiments, R² is independently halogen, —CX²₃, —CHX²₂, —CH₂X², —OCX²₃, —OCH₂X², —OCHX²₂, —CN, —SR^{2D}, —NR^{2A}R^{2B}, —C(O)R^{2C}, —C(O)OR^{2C}, —C(O)NR^{2A}R^{2B}, —OR^{2D}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently halogen, —CX²₃, —CHX²₂, —CH₂X², —OCX²₃, —OCH₂X², —OCHX²₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ aryl, or substituted or unsubstituted 5 to 12 membered heteroaryl.

In embodiments, R² is independently halogen, —CX²₃, —CHX²₂, —CH₂X², —OCX²₃, —OCH₂X², —OCHX²₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

In embodiments, two adjacent R² substituents are joined to form a substituted or unsubstituted cycloalkyl, substituted or unsubstituted heterocycloalkyl, substituted or unsubstituted aryl, or substituted or unsubstituted heteroaryl. In embodiments, two adjacent R² substituents are joined to form an unsubstituted cycloalkyl. In embodiments, two adjacent R² substituents are joined to form an unsubstituted C₃-C₆ cycloalkyl.

In embodiments, R² is independently —CX²₃. In embodiments, R² is independently —CHX²₂. In embodiments, R² is independently —CH₂X². In embodiments, R² is independently —OCX²₃. In embodiments, R² is independently —OCH₂X². In embodiments, R² is independently —OCHX²₂. In embodiments, R² is independently —CN. In embodiments, R² is independently —SOₙ₂R^{2D}. In embodiments, R² is independently —SOᵥ₂NR^{2A}R^{2B}. In embodiments, R² is independently —NHC(O)NR^{2A}R^{2B}. In embodiments, R² is independently —N(O)ₘ₂. In embodiments, R² is independently —NR^{2A}R^{2B}. In embodiments, R² is independently —C(O)R^{2C}. In embodiments, R² is independently —C(O)—OR^{2C}. In embodiments, R² is independently —C(O)NR^{2A}R^{2B}. In embodiments, R² is independently —OR^{2D}. In embodiments, R² is independently —NR^{2A}SO₂R^{2D}. In embodiments, R² is independently —NR^{2A}C(O)R^{2C}. In embodiments, R² is independently —NR^{2A}C(O)OR^{2C}. In embodiments, R² is independently —NR^{2A}OR^{2C}. In embodiments, R² is independently —OH. In embodiments, R² is independently —NH₂. In embodiments, R² is independently —COOH. In embodiments, R² is independently —CONH₂. In embodiments, R² is independently —NO₂. In embodiments, R² is independently —SH. In embodiments, R² is independently halogen. In embodiments, R² is independently —F. In embodiments, R² is independently —Cl. In embodiments, R² is independently —Br. In embodiments, R² is independently —I. In embodiments, R² is independently —CF₃. In embodiments, R² is independently —CHF₂. In embodiments, R² is independently —CH₂F. In embodiments, R² is independently —OCF₃. In embodiments, R² is independently —OCH₂F. In embodiments, R² is independently —OCHF₂. In embodiments, R² is independently —OCH₃. In embodiments, R² is independently —OCH₂CH₃. In embodiments, R² is independently —OCH₂CH₂CH₃. In embodiments, R² is independently —OCH(CH₃)₂. In embodiments, R² is independently —OC(CH₃)₃. In embodiments, R² is independently —SCH₃. In embodiments, R² is independently —SCH₂CH₃. In embodiments, R² is independently —SCH₂CH₂CH₃. In embodiments, R² is independently —SCH(CH₃)₂. In embodiments, R² is independently —SC(CH₃)₃. In embodiments, R² is independently —CH₃. In embodiments, R² is independently —CH₂CH₃. In embodiments, R² is independently —CH₂CH₂CH₃. In embodiments, R² is independently —CH(CH₃)₂. In embodiments, R² is independently —C(CH₃)₃.

In embodiments, R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12, 5 to 10 membered, 5 to 9 membered, or 5 to 6 membered). In embodiments, R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}C, R^{2A}OR^{2C}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12, 5 to 10 membered, 5 to 9 membered, or 5 to 6 membered).

In embodiments, R² is independently substituted or unsubstituted alkyl (e.g., C₁-C₈, C₁-C₆, C₁-C₄, or C₁-C₂). In embodiments, R² is independently substituted alkyl (e.g., C₁-C₈, C₁-C₆, C₁-C₄, or C₁-C₂). In embodiments, R² is independently unsubstituted alkyl (e.g., C₁-C₈, C₁-C₆, C₁-C₄, or C₁-C₂). In embodiments, R² is independently unsubstituted methyl. In embodiments, R² is independently unsubstituted ethyl. In embodiments, R² is independently unsubstituted propyl. In embodiments, R² is independently unsubstituted isopropyl. In embodiments, R² is independently unsubstituted tert-butyl. In embodiments, R² is independently substituted or unsubstituted heteroalkyl (e.g., 2 to 8 membered, 2 to 6 membered, 4 to 6 membered, 2 to 3 membered, or 4 to 5 membered). In embodiments, R² is independently substituted heteroalkyl (e.g., 2 to 8 membered, 2 to 6 membered, 4 to 6 membered, 2 to 3 membered, or 4 to 5 membered). In embodiments, R² is independently unsubstituted heteroalkyl (e.g., 2 to 8 membered, 2 to 6 membered, 4 to 6 membered, 2 to 3 membered, or 4 to 5 membered). In embodiments, R² is independently substituted or unsubstituted cycloalkyl (e.g., C₃-C₈, C₃-C₆, C₄-C₆, or C₅-C₆). In embodiments, R² is independently substituted cycloalkyl (e.g., C₃-C₈, C₃-C₆, C₄-C₆, or C₅-C₆). In embodiments, R² is independently unsubstituted cycloalkyl (e.g., C₃-C₈, C₃-C₆, C₄-C₆, or C₅-C₆). In embodiments, R² is independently substituted or unsubstituted heterocycloalkyl (e.g., 3 to 8 membered, 3 to 6 membered, 4 to 6 membered, 4 to 5 membered, or 5 to 6 membered). In embodiments, R² is independently substituted heterocycloalkyl (e.g., 3 to 8 membered, 3 to 6 membered, 4 to 6 membered, 4 to 5 membered, or 5 to 6 membered). In embodiments, R² is independently unsubstituted heterocycloalkyl (e.g., 3 to 8 membered, 3 to 6 membered, 4 to 6 membered, 4 to 5 membered, or 5 to 6 membered). In embodiments, R² is independently substituted or unsubstituted aryl (e.g., C₆-C₁₂, C₆-C₁₀, or phenyl). In embodiments, R² is independently substituted aryl (e.g., C₆-C₁₂, C₆-C₁₀, or phenyl). In embodiments, R² is independently unsubstituted aryl (e.g., C₆-C₁₂, C₆-C₁₀, or phenyl). In embodiments, R² is independently substituted or unsubstituted heteroaryl (e.g., 5 to 12 membered, 5 to 10 membered, 5 to 9 membered, or 5 to 6 membered). In embodiments, R² is independently substituted heteroaryl (e.g., 5 to 12 membered, 5 to 10 membered, 5 to 9 membered, or 5 to 6 membered). In embodiments, R² is independently unsubstituted heteroaryl (e.g., 5 to 12 membered, 5 to 10 membered, 5 to 9 membered, or 5 to 6 membered).

In embodiments, two adjacent R² substituents may optionally be joined to form a substituted or unsubstituted cycloalkyl (e.g., C₃-C₈, C₃-C₆, C₄-C₆, or C₅-C₆). In embodiments, two adjacent R² substituents may optionally be joined to form a substituted cycloalkyl (e.g., C₃-C₈, C₃-C₆, C₄-C₆, or C₅-C₆). In embodiments, two adjacent R² substituents may optionally be joined to form an unsubstituted cycloalkyl (e.g., C₃-C₈, C₃-C₆, C₄-C₆, or C₅-C₆). In embodiments, two adjacent R² substituents may optionally be joined to form a substituted or unsubstituted heterocycloalkyl (e.g., 3 to 8 membered, 3 to 6 membered, 4 to 6 membered, 4 to 5 membered, or 5 to 6 membered). In embodiments, two adjacent R² substituents may optionally be joined to form a substituted heterocycloalkyl (e.g., 3 to 8 membered, 3 to 6 membered, 4 to 6 membered, 4 to 5 membered, or 5 to 6 membered). In embodiments, two adjacent R² substituents may optionally be joined to form an unsubstituted heterocycloalkyl (e.g., 3 to 8 membered, 3 to 6 membered, 4 to 6 membered, 4 to 5 membered, or 5 to 6 membered). In embodiments, two adjacent R² substituents may optionally be joined to form a substituted or unsubstituted aryl (e.g., C₆-C₁₂, C₆-C₁₀, or phenyl). In embodiments, two adjacent R^{2 substituents may optionally be joined to form a substituted aryl (e.g., C}₆-C₁₂, C₆-C₁₀, or phenyl). In embodiments, two adjacent R² substituents may optionally be joined to form an unsubstituted aryl (e.g., C₆-C₁₂, C₆-C₁₀, or phenyl). In embodiments, two adjacent R² substituents may optionally be joined to form a substituted or unsubstituted heteroaryl (e.g., 5 to 12 membered, 5 to 10 membered, 5 to 9 membered, or 5 to 6 membered). In embodiments, two adjacent R² substituents may optionally be joined to form a substituted heteroaryl (e.g., 5 to 12 membered, 5 to 10 membered, 5 to 9 membered, or 5 to 6 membered). In embodiments, two adjacent R² substituents may optionally be joined to form an unsubstituted heteroaryl (e.g., 5 to 12 membered, 5 to 10 membered, 5 to 9 membered, or 5 to 6 membered).

In embodiments, R^{2A} is independently hydrogen. In embodiments, R^{2A} is independently —CX^{2A3}. In embodiments, R^{2A} is independently —CHX^{2A}₂. In embodiments, R^{2A} is independently —CH₂X^{2A}. In embodiments, R^{2A} is independently —CN. In embodiments, R^{2A} is independently —COOH. In embodiments, R^{2A} is independently —CONH₂. In embodiments, X^{2A} is independently —F, —Cl, —Br, or —I.

In embodiments, R^{2A} is independently substituted or unsubstituted alkyl (e.g., C₁-C₈, C₁-C₆, C₁-C₄, or C₁-C₂). In embodiments, R^{2A} is independently substituted alkyl (e.g., C₁-C₈, C₁-C₆, C₁-C₄, or C₁-C₂). In embodiments, R^{2A} is independently unsubstituted alkyl (e.g., C₁-C₈, C₁-C₆, C₁-C₄, or C₁-C₂). In embodiments, R^{2A} is independently unsubstituted methyl. In embodiments, R^{2A} is independently unsubstituted ethyl. In embodiments, R^{2A} is independently unsubstituted propyl. In embodiments, R^{2A} is independently unsubstituted isopropyl. In embodiments, R^{2A} is independently unsubstituted tert-butyl. In embodiments, R^{2A} is independently substituted or unsubstituted heteroalkyl (e.g., 2 to 8 membered, 2 to 6 membered, 4 to 6 membered, 2 to 3 membered, or 4 to 5 membered). In embodiments, R^{2A} is independently substituted heteroalkyl (e.g., 2 to 8 membered, 2 to 6 membered, 4 to 6 membered, 2 to 3 membered, or 4 to 5 membered). In embodiments, R^{2A} is independently unsubstituted heteroalkyl (e.g., 2 to 8 membered, 2 to 6 membered, 4 to 6 membered, 2 to 3 membered, or 4 to 5 membered). In embodiments, R^{2A} is independently substituted or unsubstituted cycloalkyl (e.g., C₃-C₈, C₃-C₆, C₄-C₆, or C₅-C₆). In embodiments, R^{2A} is independently substituted cycloalkyl (e.g., C₃-C₈, C₃-C₆, C₄-C₆, or C₅-C₆). In embodiments, R^{2A} is independently unsubstituted cycloalkyl (e.g., C₃-C₈, C₃-C₆, C₄-C₆, or C₅-C₆). In embodiments, R^{2A} is independently substituted or unsubstituted heterocycloalkyl (e.g., 3 to 8 membered, 3 to 6 membered, 4 to 6 membered, 4 to 5 membered, or 5 to 6 membered). In embodiments, R^{2A} is independently substituted heterocycloalkyl (e.g., 3 to 8 membered, 3 to 6 membered, 4 to 6 membered, 4 to 5 membered, or 5 to 6 membered). In embodiments, R^{2A} is independently unsubstituted heterocycloalkyl (e.g., 3 to 8 membered, 3 to 6 membered, 4 to 6 membered, 4 to 5 membered, or 5 to 6 membered). In embodiments, R^{2A} is independently substituted or unsubstituted aryl (e.g., C₆-C₁₂, C₆-C₁₀, or phenyl). In embodiments, R^{2A} is independently substituted aryl (e.g., C₆-C₁₂, C₆-C₁₀, or phenyl). In embodiments, R^{2A} is independently unsubstituted aryl (e.g., C₆-C₁₂, C₆-C₁₀, or phenyl). In embodiments, R^{2A} is independently substituted or unsubstituted heteroaryl (e.g., 5 to 12 membered, 5 to 10 membered, 5 to 9 membered, or 5 to 6 membered). In embodiments, R^{2A} is independently substituted heteroaryl (e.g., 5 to 12 membered, 5 to 10 membered, 5 to 9 membered, or 5 to 6 membered). In embodiments, R^{2A} is independently unsubstituted heteroaryl (e.g., 5 to 12 membered, 5 to 10 membered, 5 to 9 membered, or 5 to 6 membered).

In embodiments, R^{2B} is independently hydrogen. In embodiments, R^{2B} is independently —CX^{2B3}. In embodiments, R^{2B} is independently —CHX^{2B}₂. In embodiments, R^{2B} is independently —CH₂X^{2B}. In embodiments, R^{2B} is independently —CN. In embodiments, R^{2B} is independently —COOH. In embodiments, R^{2B} is independently —CONH₂. In embodiments, X^{2B} is independently —F, —Cl, —Br, or —I.

In embodiments, R^{2B} is independently substituted or unsubstituted alkyl (e.g., C₁-C₈, C₁-C₆, C₁-C₄, or C₁-C₂). In embodiments, R^{2B} is independently substituted alkyl (e.g., C₁-C₈, C₁-C₆, C₁-C₄, or C₁-C₂). In embodiments, R^{2B} is independently unsubstituted alkyl (e.g., C₁-C₈, C₁-C₆, C₁-C₄, or C₁-C₂). In embodiments, R^{2B} is independently unsubstituted methyl. In embodiments, R^{2B} is independently unsubstituted ethyl. In embodiments, R^{2B} is independently unsubstituted propyl. In embodiments, R^{2B} is independently unsubstituted isopropyl. In embodiments, R^{2B} is independently unsubstituted tert-butyl. In embodiments, R^{2B} is independently substituted or unsubstituted heteroalkyl (e.g., 2 to 8 membered, 2 to 6 membered, 4 to 6 membered, 2 to 3 membered, or 4 to 5 membered). In embodiments, R^{2B} is independently substituted heteroalkyl (e.g., 2 to 8 membered, 2 to 6 membered, 4 to 6 membered, 2 to 3 membered, or 4 to 5 membered). In embodiments, R^{2B} is independently unsubstituted heteroalkyl (e.g., 2 to 8 membered, 2 to 6 membered, 4 to 6 membered, 2 to 3 membered, or 4 to 5 membered). In embodiments, R^{2B} is independently substituted or unsubstituted cycloalkyl (e.g., C₃-C₈, C₃-C₆, C₄-C₆, or C₅-C₆). In embodiments, R^{2B} is independently substituted cycloalkyl (e.g., C₃-C₈, C₃-C₆, C₄-C₆, or C₅-C₆). In embodiments, R^{2B} is independently unsubstituted cycloalkyl (e.g., C₃-C₈, C₃-C₆, C₄-C₆, or C₅-C₆). In embodiments, R^{2B} is independently substituted or unsubstituted heterocycloalkyl (e.g., 3 to 8 membered, 3 to 6 membered, 4 to 6 membered, 4 to 5 membered, or 5 to 6 membered). In embodiments, R^{2B} is independently substituted heterocycloalkyl (e.g., 3 to 8 membered, 3 to 6 membered, 4 to 6 membered, 4 to 5 membered, or 5 to 6 membered). In embodiments, R^{2B} is independently unsubstituted heterocycloalkyl (e.g., 3 to 8 membered, 3 to 6 membered, 4 to 6 membered, 4 to 5 membered, or 5 to 6 membered). In embodiments, R^{2B} is independently substituted or unsubstituted aryl (e.g., C₆-C₁₂, C₆-C₁₀, or phenyl). In embodiments, R^{2B} is independently substituted aryl (e.g., C₆-C₁₂, C₆-C₁₀, or phenyl). In embodiments, R^{2B} is independently unsubstituted aryl (e.g., C₆-C₁₂, C₆-C₁₀, or phenyl). In embodiments, R^{2B} is independently substituted or unsubstituted heteroaryl (e.g., 5 to 12 membered, 5 to 10 membered, 5 to 9 membered, or 5 to 6 membered). In embodiments, R^{2B} is independently substituted heteroaryl (e.g., 5 to 12 membered, 5 to 10 membered, 5 to 9 membered, or 5 to 6 membered). In embodiments, R^{2B} is independently unsubstituted heteroaryl (e.g., 5 to 12 membered, 5 to 10 membered, 5 to 9 membered, or 5 to 6 membered).

In embodiments, R^{2A} and R^{2B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{2A} and R^{2B} substituents bonded to the same nitrogen atom may be joined to form a substituted or unsubstituted heteroaryl (e.g., 5 to 12 membered, 5 to 10 membered, 5 to 9 membered, or 5 to 6 membered). In embodiments, R^{2A} and R^{2B} substituents bonded to the same nitrogen atom may be joined to form a substituted heteroaryl (e.g., 5 to 12 membered, 5 to 10 membered, 5 to 9 membered, or 5 to 6 membered). In embodiments, R^{2A} and R^{2B} substituents bonded to the same nitrogen atom may be joined to form an unsubstituted heteroaryl (e.g., 5 to 12 membered, 5 to 10 membered, 5 to 9 membered, or 5 to 6 membered).

In embodiments, R^{2C} is independently hydrogen. In embodiments, R^{2C} is independently —CX^{2C}₃. In embodiments, R^{2C} is independently —CHX^{2C}₂. In embodiments, R^{2C} is independently —CH₂X^{2C}. In embodiments, R^{2C} is independently —CN. In embodiments, R^{2C} is independently —COOH. In embodiments, R^{2C} is independently —CONH₂. In embodiments, X^{2C} is independently —F, —Cl, —Br, or —I.

In embodiments, R^{2C} is independently substituted or unsubstituted alkyl (e.g., C₁-C₈, C₁-C₆, C₁-C₄, or C₁-C₂). In embodiments, R^{2C} is independently substituted alkyl (e.g., C₁-C₈, C₁-C₆, C₁-C₄, or C₁-C₂). In embodiments, R^{2C} is independently unsubstituted alkyl (e.g., C₁-C₈, C₁-C₆, C₁-C₄, or C₁-C₂). In embodiments, R^{2C} is independently unsubstituted methyl. In embodiments, R^{2C} is independently unsubstituted ethyl. In embodiments, R^{2C} is independently unsubstituted propyl. In embodiments, R^{2C} is independently unsubstituted isopropyl. In embodiments, R^{2C} is independently unsubstituted tert-butyl. In embodiments, R^{2C} is independently substituted or unsubstituted heteroalkyl (e.g., 2 to 8 membered, 2 to 6 membered, 4 to 6 membered, 2 to 3 membered, or 4 to 5 membered). In embodiments, R^{2C} is independently substituted heteroalkyl (e.g., 2 to 8 membered, 2 to 6 membered, 4 to 6 membered, 2 to 3 membered, or 4 to 5 membered). In embodiments, R^{2C} is independently unsubstituted heteroalkyl (e.g., 2 to 8 membered, 2 to 6 membered, 4 to 6 membered, 2 to 3 membered, or 4 to 5 membered). In embodiments, R^{2C} is independently substituted or unsubstituted cycloalkyl (e.g., C₃-C₈, C₃-C₆, C₄-C₆, or C₅-C₆). In embodiments, R^{2C} is independently substituted cycloalkyl (e.g., C₃-C₈, C₃-C₆, C₄-C₆, or C₅-C₆). In embodiments, R^{2C} is independently unsubstituted cycloalkyl (e.g., C₃-C₈, C₃-C₆, C₄-C₆, or C₅-C₆). In embodiments, R^{2C} is independently substituted or unsubstituted heterocycloalkyl (e.g., 3 to 8 membered, 3 to 6 membered, 4 to 6 membered, 4 to 5 membered, or 5 to 6 membered). In embodiments, R^{2C} is independently substituted heterocycloalkyl (e.g., 3 to 8 membered, 3 to 6 membered, 4 to 6 membered, 4 to 5 membered, or 5 to 6 membered). In embodiments, R^{2C} is independently unsubstituted heterocycloalkyl (e.g., 3 to 8 membered, 3 to 6 membered, 4 to 6 membered, 4 to 5 membered, or 5 to 6 membered). In embodiments, R^{2C} is independently substituted or unsubstituted aryl (e.g., C₆-C₁₂, C₆-C₁₀, or phenyl). In embodiments, R^{2C} is independently substituted aryl (e.g., C₆-C₁₂, C₆-C₁₀, or phenyl). In embodiments, R^{2C} is independently unsubstituted aryl (e.g., C₆-C₁₂, C₆-C₁₀, or phenyl). In embodiments, R^{2C} is independently substituted or unsubstituted heteroaryl (e.g., 5 to 12 membered, 5 to 10 membered, 5 to 9 membered, or 5 to 6 membered). In embodiments, R^{2C} is independently substituted heteroaryl (e.g., 5 to 12 membered, 5 to 10 membered, 5 to 9 membered, or 5 to 6 membered). In embodiments, R^{2C} is independently unsubstituted heteroaryl (e.g., 5 to 12 membered, 5 to 10 membered, 5 to 9 membered, or 5 to 6 membered).

In embodiments, R^{2D} is independently hydrogen. In embodiments, R^{2D} is independently —CX^{2D}₃. In embodiments, R^{2D} is independently —CHX^{2D}₂. In embodiments, R^{2D} is independently —CH₂X^{2D}. In embodiments, R^{2D} is independently —CN. In embodiments, R^{2D} is independently —COOH. In embodiments, R^{2D} is independently —CONH₂. In embodiments, X^{2D} is independently —F, —Cl, —Br, or —I.

In embodiments, R^{2D} is independently substituted or unsubstituted alkyl (e.g., C₁-C₈, C₁-C₆, C₁-C₄, or C₁-C₂). In embodiments, R^{2D} is independently substituted alkyl (e.g., C₁-C₈, C₁-C₆, C₁-C₄, or C₁-C₂). In embodiments, R^{2D} is independently unsubstituted alkyl (e.g., C₁-C₈, C₁-C₆, C₁-C₄, or C₁-C₂). In embodiments, R^{2D} is independently unsubstituted methyl. In embodiments, R^{2D} is independently unsubstituted ethyl. In embodiments, R^{2D} is independently unsubstituted propyl. In embodiments, R^{2D} is independently unsubstituted isopropyl. In embodiments, R^{2D} is independently unsubstituted tert-butyl. In embodiments, R^{2D} is independently substituted or unsubstituted heteroalkyl (e.g., 2 to 8 membered, 2 to 6 membered, 4 to 6 membered, 2 to 3 membered, or 4 to 5 membered). In embodiments, R^{2D} is independently substituted heteroalkyl (e.g., 2 to 8 membered, 2 to 6 membered, 4 to 6 membered, 2 to 3 membered, or 4 to 5 membered). In embodiments, R^{2D} is independently unsubstituted heteroalkyl (e.g., 2 to 8 membered, 2 to 6 membered, 4 to 6 membered, 2 to 3 membered, or 4 to 5 membered). In embodiments, R^{2D} is independently substituted or unsubstituted cycloalkyl (e.g., C₃-C₈, C₃-C₆, C₄-C₆, or C₅-C₆). In embodiments, R^{2D} is independently substituted cycloalkyl (e.g., C₃-C₈, C₃-C₆, C₄-C₆, or C₅-C₆). In embodiments, R^{2D} is independently unsubstituted cycloalkyl (e.g., C₃-C₈, C₃-C₆, C₄-C₆, or C₅-C₆). In embodiments, R^{2D} is independently substituted or unsubstituted heterocycloalkyl (e.g., 3 to 8 membered, 3 to 6 membered, 4 to 6 membered, 4 to 5 membered, or 5 to 6 membered). In embodiments, R^{2D} is independently substituted heterocycloalkyl (e.g., 3 to 8 membered, 3 to 6 membered, 4 to 6 membered, 4 to 5 membered, or 5 to 6 membered). In embodiments, R^{2D} is independently unsubstituted heterocycloalkyl (e.g., 3 to 8 membered, 3 to 6 membered, 4 to 6 membered, 4 to 5 membered, or 5 to 6 membered). In embodiments, R^{2D} is independently substituted or unsubstituted aryl (e.g., C₆-C₁₂, C₆-C₁₀, or phenyl). In embodiments, R^{2D} is independently substituted aryl (e.g., C₆-C₁₂, C₆-C₁₀, or phenyl). In embodiments, R^{2D} is independently unsubstituted aryl (e.g., C₆-C₁₂, C₆-C₁₀, or phenyl). In embodiments, R^{2D} is independently substituted or unsubstituted heteroaryl (e.g., 5 to 12 membered, 5 to 10 membered, 5 to 9 membered, or 5 to 6 membered). In embodiments, R^{2D} is independently substituted heteroaryl (e.g., 5 to 12 membered, 5 to 10 membered, 5 to 9 membered, or 5 to 6 membered). In embodiments, R^{2D} is independently unsubstituted heteroaryl (e.g., 5 to 12 membered, 5 to 10 membered, 5 to 9 membered, or 5 to 6 membered).

In embodiments, R² is independently halogen, —CX²₃, —CHX²₂, —CH₂X², —OCX²₃, —OCH₂X², —OCHX²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²³-substituted or unsubstituted alkyl (e.g., C₁-C₈, C₁-C₆, C₁-C₄, or C₁-C₂), R²³-substituted or unsubstituted heteroalkyl (e.g., 2 to 8 membered, 2 to 6 membered, 4 to 6 membered, 2 to 3 membered, or 4 to 5 membered), R²³-substituted or unsubstituted cycloalkyl (e.g., C₃-C₈, C₃-C₆, C₄-C₆, or C₅-C₆), R²³-substituted or unsubstituted heterocycloalkyl (e.g., 3 to 8 membered, 3 to 6 membered, 4 to 6 membered, 4 to 5 membered, or 5 to 6 membered), R²³-substituted or unsubstituted aryl (e.g., C₆-C₁₂, C₆-C₁₀, or phenyl), or R²³-substituted or unsubstituted heteroaryl (e.g., 5 to 12 membered, 5 to 10 membered, 5 to 9 membered, or 5 to 6 membered). In embodiments, R² is independently halogen, —CX²₃, —CHX²₂, —CH₂X², —OCX²₃, —OCH₂X², —OCHX²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X² is independently —F, —Cl, —Br, or —I. In embodiments, R² is independently unsubstituted methyl. In embodiments, R² is independently unsubstituted ethyl. In embodiments, R² is independently

halogen, —CX²₃, —CHX²₂, —CH₂X², —OCX²₃, —OCH₂X², —OCHX²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²³-substituted or unsubstituted alkyl (e.g., C₁-C₈, C₁-C₆, C₁-C₄, or C₁-C₂), R²³-substituted or unsubstituted heteroalkyl (e.g., 2 to 8 membered, 2 to 6 membered, 4 to 6 membered, 2 to 3 membered, or 4 to 5 membered), R²³-substituted or unsubstituted cycloalkyl (e.g., C₃-C₈, C₃-C₆, C₄-C₆, or C₅-C₆), R²³-substituted or unsubstituted heterocycloalkyl (e.g., 3 to 8 membered, 3 to 6 membered, 4 to 6 membered, 4 to 5 membered, or 5 to 6 membered), R²³-substituted or unsubstituted aryl (e.g., C₆-C₁₂, C₆-C₁₀, or phenyl), or R²³-substituted or unsubstituted heteroaryl (e.g., 5 to 12 membered, 5 to 10 membered, 5 to 9 membered, or 5 to 6 membered). In embodiments, R² is independently halogen, —CX²₃, —CHX²₂, —CH₂X², —OCX²₃, —OCH₂X², —OCHX²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, two adjacent R² substituents may optionally be joined to form a R²³-substituted or unsubstituted cycloalkyl (e.g., C₃-C₈, C₃-C₆, C₄-C₆, or C₅-C₆). In embodiments, two adjacent R² substituents may optionally be joined to form a R²³-substituted cycloalkyl (e.g., C₃-C₈, C₃-C₆, C₄-C₆, or C₅-C₆). In embodiments, two adjacent R² substituents may optionally be joined to form an unsubstituted cycloalkyl (e.g., C₃-C₈, C₃-C₆, C₄-C₆, or C₅-C₆). In embodiments, two adjacent R² substituents may optionally be joined to form a R²³-substituted or unsubstituted heterocycloalkyl (e.g., 3 to 8 membered, 3 to 6 membered, 4 to 6 membered, 4 to 5 membered, or 5 to 6 membered). In embodiments, two adjacent R² substituents may optionally be joined to form a R²³-substituted heterocycloalkyl (e.g., 3 to 8 membered, 3 to 6 membered, 4 to 6 membered, 4 to 5 membered, or 5 to 6 membered). In embodiments, two adjacent R² substituents may optionally be joined to form an unsubstituted heterocycloalkyl (e.g., 3 to 8 membered, 3 to 6 membered, 4 to 6 membered, 4 to 5 membered, or 5 to 6 membered). In embodiments, two adjacent R² substituents may optionally be joined to form a R²³-substituted or unsubstituted aryl (e.g., C₆-C₁₂, C₆-C₁₀, or phenyl). In embodiments, two adjacent R² substituents may optionally be joined to form a R²³-substituted aryl (e.g., C₆-C₁₂, C₆-C₁₀, or phenyl). In embodiments, two adjacent R² substituents may optionally be joined to form an unsubstituted aryl (e.g., C₆-C₁₂, C₆-C₁₀, or phenyl). In embodiments, two adjacent R² substituents may optionally be joined to form a R²³-substituted or unsubstituted heteroaryl (e.g., 5 to 12 membered, 5 to 10 membered, 5 to 9 membered, or 5 to 6 membered). In embodiments, two adjacent R² substituents may optionally be joined to form a R²³-substituted heteroaryl (e.g., 5 to 12 membered, 5 to 10 membered, 5 to 9 membered, or 5 to 6 membered). In embodiments, two adjacent R² substituents may optionally be joined to form an unsubstituted heteroaryl (e.g., 5 to 12 membered, 5 to 10 membered, 5 to 9 membered, or 5 to 6 membered).

R²³ is independently oxo,

halogen, —CX²³₃, —CHX²³₂, —CH₂X²³, —OCX²³₃, —OCH₂X²³, —OCHX²³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²⁴-substituted or unsubstituted alkyl (e.g., C₁-C₈, C₁-C₆, C₁-C₄, or C₁-C₂), R²⁴-substituted or unsubstituted heteroalkyl (e.g., 2 to 8 membered, 2 to 6 membered, 4 to 6 membered, 2 to 3 membered, or 4 to 5 membered), R²⁴-substituted or unsubstituted cycloalkyl (e.g., C₃-C₈, C₃-C₆, C₄-C₆, or C₅-C₆), R²⁴-substituted or unsubstituted heterocycloalkyl (e.g., 3 to 8 membered, 3 to 6 membered, 4 to 6 membered, 4 to 5 membered, or 5 to 6 membered), R²⁴-substituted or unsubstituted aryl (e.g., C₆-C₁₂, C₆-C₁₀, or phenyl), or R²⁴-substituted or unsubstituted heteroaryl (e.g., 5 to 12 membered, 5 to 10 membered, 5 to 9 membered, or 5 to 6 membered). In embodiments, R²³ is independently oxo,
halogen, —CX²³₃, —CHX²³₂, —CH₂X²³, —OCX²³₃, —OCH₂X²³, —OCHX²³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X²³ is independently —F, —Cl, —Br, or —I. In embodiments, R²³ is independently unsubstituted methyl. In embodiments, R²³ is independently unsubstituted ethyl.

R²⁴ is independently oxo,

halogen, —CX²⁴₃, —CHX²⁴₂, —CH₂X²⁴, —OCX²⁴₃, —OCH₂X²⁴, —OCHX²⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²⁵-substituted or unsubstituted alkyl (e.g., C₁-C₈, C₁-C₆, C₁-C₄, or C₁-C₂), R²⁵-substituted or unsubstituted heteroalkyl (e.g., 2 to 8 membered, 2 to 6 membered, 4 to 6 membered, 2 to 3 membered, or 4 to 5 membered), R²⁵-substituted or unsubstituted cycloalkyl (e.g., C₃-C₈, C₃-C₆, C₄-C₆, or C₅-C₆), R²⁵-substituted or unsubstituted heterocycloalkyl (e.g., 3 to 8 membered, 3 to 6 membered, 4 to 6 membered, 4 to 5 membered, or 5 to 6 membered), R²⁵-substituted or unsubstituted aryl (e.g., C₆-C₁₂, C₆-C₁₀, or phenyl), or R²⁵-substituted or unsubstituted heteroaryl (e.g., 5 to 12 membered, 5 to 10 membered, 5 to 9 membered, or 5 to 6 membered). In embodiments, R²⁴ is independently oxo,
halogen, —CX²⁴₃, —CHX²⁴₂, —CH₂X²⁴, —OCX²⁴₃, —OCH₂X²⁴, —OCHX²⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X²⁴ is independently —F, —Cl, —Br, or —I. In embodiments, R²⁴ is independently unsubstituted methyl. In embodiments, R²⁴ is independently unsubstituted ethyl.

R²⁵ is independently oxo,

halogen, —CX²⁵₃, —CHX²⁵₂, —CH₂X²⁵, —OCX²⁵₃, —OCH₂X²⁵, —OCHX²⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X²⁵ is independently —F, —Cl, —Br, or —I. In embodiments, R²⁵ is independently unsubstituted methyl. In embodiments, R²⁵ is independently unsubstituted ethyl.

In embodiments, R^{2A} is independently hydrogen, —CX^{2A}₃, —CHX^{2A}₂, —CH₂X^{2A}, —CN, —COOH, —CONH₂, R^{23A}-substituted or unsubstituted alkyl (e.g., C₁-C₈, C₁-C₆, C₁-C₄, or C₁-C₂), R^{23A}-substituted or unsubstituted heteroalkyl (e.g., 2 to 8 membered, 2 to 6 membered, 4 to 6 membered, 2 to 3 membered, or 4 to 5 membered), R^{23A}-substituted or unsubstituted cycloalkyl (e.g., C₃-C₈, C₃-C₆, C₄-C₆, or C₅-C₆), R^{23A}-substituted or unsubstituted heterocycloalkyl (e.g., 3 to 8 membered, 3 to 6 membered, 4 to 6 membered, 4 to 5 membered, or 5 to 6 membered), R^{23A}-substituted or unsubstituted aryl (e.g., C₆-C₁₂, C₆-C₁₀, or phenyl), or R^{23A}-substituted or unsubstituted heteroaryl (e.g., 5 to 12 membered, 5 to 10 membered, 5 to 9 membered, or 5 to 6 membered). In embodiments, R^{2A} is independently hydrogen, —CX^{2A}₃, —CHX^{2A}₂, —CH₂X^{2A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{2A} is independently —F, —Cl, —Br, or —I. In embodiments, R^{2A} is independently hydrogen. In embodiments, R^{2A} is independently unsubstituted methyl. In embodiments, R^{2A} is independently unsubstituted ethyl.

In embodiments, R^{2A} and R^{2B} substituents bonded to the same nitrogen atom may optionally be joined to form a R^{23A}-substituted or unsubstituted heterocycloalkyl (e.g., 3 to 8 membered, 3 to 6 membered, 4 to 6 membered, 4 to 5 membered, or 5 to 6 membered) or R^{23A}-substituted or unsubstituted heteroaryl (e.g., 5 to 12 membered, 5 to 10 membered, 5 to 9 membered, or 5 to 6 membered). In embodiments, R^{2A} and R^{2B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 12 membered, 5 to 10 membered, 5 to 9 membered, or 5 to 6 membered). In embodiments, R^{2A} and R^{2B} substituents bonded to the same nitrogen atom may optionally be joined to form a R^{23A}-substituted or unsubstituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{23A} is independently oxo,

halogen, —CX^{23A}₃, —CHX^{23A}₂, —CH₂X^{23A}, —OCX^{23A}₃, —OCH₂X^{23A}, —OCHX^{23A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{24A}-substituted or unsubstituted alkyl (e.g., C₁-C₈, C₁-C₆, C₁-C₄, or C₁-C₂), R^{24A}-substituted or unsubstituted heteroalkyl (e.g., 2 to 8 membered, 2 to 6 membered, 4 to 6 membered, 2 to 3 membered, or 4 to 5 membered), R^{24A}-substituted or unsubstituted cycloalkyl (e.g., C₃-C₈, C₃-C₆, C₄-C₆, or C₅-C₆), R^{24A}-substituted or unsubstituted heterocycloalkyl (e.g., 3 to 8 membered, 3 to 6 membered, 4 to 6 membered, 4 to 5 membered, or 5 to 6 membered), R^{24A}-substituted or unsubstituted aryl (e.g., C₆-C₁₂, C₆-C₁₀, or phenyl), or R^{24A}-substituted or unsubstituted heteroaryl (e.g., 5 to 12 membered, 5 to 10 membered, 5 to 9 membered, or 5 to 6 membered). In embodiments, R^{23A} is independently oxo,
halogen, —CX^{23A}₃, —CHX^{23A}₂, —CH₂X^{23A}, —OCX^{23A}₃, —OCH₂X^{23A}, —OCHX^{23A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{23A} is independently —F, —Cl, —Br, or —I. In embodiments, R^{23A} is independently unsubstituted methyl. In embodiments, R^{23A} is independently unsubstituted ethyl.

R^{24A} is independently oxo,

halogen, —CX^{24A}₃, —CHX^{24A}₂, —CH₂X^{24A}, —OCX^{24A}₃, —OCH₂X^{24A}, —OCHX^{24A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{25A}-substituted or unsubstituted alkyl (e.g., C₁-C₈, C₁-C₆, C₁-C₄, or C₁-C₂), R^{25A}-substituted or unsubstituted heteroalkyl (e.g., 2 to 8 membered, 2 to 6 membered, 4 to 6 membered, 2 to 3 membered, or 4 to 5 membered), R^{25A}-substituted or unsubstituted cycloalkyl (e.g., C₃-C₈, C₃-C₆, C₄-C₆, or C₅-C₆), R^{25A}-substituted or unsubstituted heterocycloalkyl (e.g., 3 to 8 membered, 3 to 6 membered, 4 to 6 membered, 4 to 5 membered, or 5 to 6 membered), R^{25A}-substituted or unsubstituted aryl (e.g., C₆-C₁₂, C₆-C₁₀, or phenyl), or R^{25A}-substituted or unsubstituted heteroaryl (e.g., 5 to 12 membered, 5 to 10 membered, 5 to 9 membered, or 5 to 6 membered). In embodiments, R^{24A} is independently oxo,
halogen, —CX^{24A}₃, —CHX^{24A}₂, —CH₂X^{24A}, —OCX^{24A}₃, —OCH₂X^{24A}, —OCHX^{24A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{24A} is independently —F, —Cl, —Br, or —I. In embodiments, R^{24A} is independently unsubstituted methyl. In embodiments, R^{24A} is independently unsubstituted ethyl.

R^{25A} is independently oxo,

halogen, —CX^{25A}₃, —CHX^{25A}₂, —CH₂X^{25A}, —OCX^{25A}₃, —OCH₂X^{25A}, —OCHX^{25A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{25A} is independently —F, —Cl, —Br, or —I. In embodiments, R^{25A} is independently unsubstituted methyl. In embodiments, R^{25A} is independently unsubstituted ethyl.

In embodiments, R^{2B} is independently hydrogen, —CX^{2B}₃, —CHX^{2B}₂, —CH₂X^{2B}, —CN, —COOH, —CONH₂, R^{23B}-substituted or unsubstituted alkyl (e.g., C₁-C₈, C₁-C₆, C₁-C₄, or C₁-C₂), R^{23B}-substituted or unsubstituted heteroalkyl (e.g., 2 to 8 membered, 2 to 6 membered, 4 to 6 membered, 2 to 3 membered, or 4 to 5 membered), R^{23B}-substituted or unsubstituted cycloalkyl (e.g., C₃-C₈, C₃-C₆, C₄-C₆, or C₅-C₆), R^{23B}-substituted or unsubstituted heterocycloalkyl (e.g., 3 to 8 membered, 3 to 6 membered, 4 to 6 membered, 4 to 5 membered, or 5 to 6 membered), R^{23B}-substituted or unsubstituted aryl (e.g., C₆-C₁₂, C₆-C₁₀, or phenyl), or R^{23B}-substituted or unsubstituted heteroaryl (e.g., 5 to 12 membered, 5 to 10 membered, 5 to 9 membered, or 5 to 6 membered). In embodiments, R^{2B} is independently hydrogen, —CX^{2B}₃, —CHX^{2B}₂, —CH₂X^{2B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{2B} is independently —F, —Cl, —Br, or —I. In embodiments, R^{2B} is independently hydrogen. In embodiments, R^{2B} is independently unsubstituted methyl. In embodiments, R^{2B} is independently unsubstituted ethyl.

In embodiments, R^{2A} and R^{2B} substituents bonded to the same nitrogen atom may optionally be joined to form a R^{23B}-substituted or unsubstituted heterocycloalkyl (e.g., 3 to 8 membered, 3 to 6 membered, 4 to 6 membered, 4 to 5 membered, or 5 to 6 membered) or R^{23B}-substituted or unsubstituted heteroaryl (e.g., 5 to 12 membered, 5 to 10 membered, 5 to 9 membered, or 5 to 6 membered). In embodiments, R^{2A} and R^{2B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 12 membered, 5 to 10 membered, 5 to 9 membered, or 5 to 6 membered). In embodiments, R^{2A} and R^{2B} substituents bonded to the same nitrogen atom may optionally be joined to form a R^{23B}-substituted or unsubstituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{23B} is independently oxo,

halogen, —CX^{23B}₃, —CHX^{23B}₂, —CH₂X^{23B}, —OCX^{23B}₃, —OCH₂X^{23B}, —OCHX^{23B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{24B}-substituted or unsubstituted alkyl (e.g., C₁-C₈, C₁-C₆, C₁-C₄, or C₁-C₂), R^{24B}-substituted or unsubstituted heteroalkyl (e.g., 2 to 8 membered, 2 to 6 membered, 4 to 6 membered, 2 to 3 membered, or 4 to 5 membered), R^{24B}-substituted or unsubstituted cycloalkyl (e.g., C₃-C₈, C₃-C₆, C₄-C₆, or C₅-C₆), R^{24B}-substituted or unsubstituted heterocycloalkyl (e.g., 3 to 8 membered, 3 to 6 membered, 4 to 6 membered, 4 to 5 membered, or 5 to 6 membered), R^{24B}-substituted or unsubstituted aryl (e.g., C₆-C₁₂, C₆-C₁₀, or phenyl), or R^{24B}-substituted or unsubstituted heteroaryl (e.g., 5 to 12 membered, 5 to 10 membered, 5 to 9 membered, or 5 to 6 membered). In embodiments, R^{23B} is independently oxo,
halogen, —CX^{23B}₃, —CHX^{23B}₂, —CH₂X^{23B}, —OCX^{23B}₃, —OCH₂X^{23B}, —OCHX^{23B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{23B} is independently —F, —Cl, —Br, or —I. In embodiments, R^{23B} is independently unsubstituted methyl. In embodiments, R^{23B} is independently unsubstituted ethyl.

R^{24B} is independently oxo,

halogen, —CX^{24B}₃, —CHX^{24B}₂, —CH₂X^{24B}, —OCX^{24B}₃, —OCH₂X^{24B}, —OCHX^{24B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{25B}-substituted or unsubstituted alkyl (e.g., C₁-C₈, C₁-C₆, C₁-C₄, or C₁-C₂), R^{25B}-substituted or unsubstituted heteroalkyl (e.g., 2 to 8 membered, 2 to 6 membered, 4 to 6 membered, 2 to 3 membered, or 4 to 5 membered), R^{25B}-substituted or unsubstituted cycloalkyl (e.g., C₃-C₈, C₃-C₆, C₄-C₆, or C₅-C₆), R^{25B}-substituted or unsubstituted heterocycloalkyl (e.g., 3 to 8 membered, 3 to 6 membered, 4 to 6 membered, 4 to 5 membered, or 5 to 6 membered), R^{25B}-substituted or unsubstituted aryl (e.g., C₆-C₁₂, C₆-C₁₀, or phenyl), or R^{25B}-substituted or unsubstituted heteroaryl (e.g., 5 to 12 membered, 5 to 10 membered, 5 to 9 membered, or 5 to 6 membered). In embodiments, R^{24B} is independently oxo,
halogen, —CX^{24B}₃, —CHX^{24B}₂, —CH₂X^{24B}, —OCX^{24B}₃, —OCH₂X^{24B}, —OCHX^{24B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{24B} is independently —F, —Cl, —Br, or —I. In embodiments, R^{24B} is independently unsubstituted methyl. In embodiments, R^{24B} is independently unsubstituted ethyl.

R^{25B} is independently oxo,

halogen, —CX^{25B}₃, —CHX^{25B}₂, —CH₂X^{25B}, —OCX^{25B}₃, —OCH₂X^{25B}, —OCHX^{25B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{25B} is independently —F, —Cl, —Br, or —I. In embodiments, R^{25B} is independently unsubstituted methyl. In embodiments, R^{25B} is independently unsubstituted ethyl.

In embodiments, R^{2C} is independently hydrogen, —CX^{2C}₃, —CHX^{2C}₂, —CH₂X^{2C}, —CN, —COOH, —CONH₂, R^{23C}-substituted or unsubstituted alkyl (e.g., C₁-C₈, C₁-C₆, C₁-C₄, or C₁-C₂), R^{23C}-substituted or unsubstituted heteroalkyl (e.g., 2 to 8 membered, 2 to 6 membered, 4 to 6 membered, 2 to 3 membered, or 4 to 5 membered), R^{23C}-substituted or unsubstituted cycloalkyl (e.g., C₃-C₈, C₃-C₆, C₄-C₆, or C₅-C₆), R^{23C}-substituted or unsubstituted heterocycloalkyl (e.g., 3 to 8 membered, 3 to 6 membered, 4 to 6 membered, 4 to 5 membered, or 5 to 6 membered), R^{23C}-substituted or unsubstituted aryl (e.g., C₆-C₁₂, C₆-C₁₀, or phenyl), or R^{23C}-substituted or unsubstituted heteroaryl (e.g., 5 to 12 membered, 5 to 10 membered, 5 to 9 membered, or 5 to 6 membered). In embodiments, R^{2C} is independently hydrogen, —CX^{2C}₃, —CHX^{2C}₂, —CH₂X^{2C}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{2C} is independently —F, —Cl, —Br, or —I. In embodiments, R^{2C} is independently hydrogen. In embodiments, R^{2C} is independently unsubstituted methyl. In embodiments, R^{2C} is independently unsubstituted ethyl.

R^{23C} is independently oxo,

halogen, —CX^{23C}₃, —CHX^{23C}₂, —CH₂X^{23C}, —OCX^{23C}₃, —OCH₂X^{23C}, —OCHX^{23C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{24C}-substituted or unsubstituted alkyl (e.g., C₁-C₈, C₁-C₆, C₁-C₄, or C₁-C₂), R^{24C}-substituted or unsubstituted heteroalkyl (e.g., 2 to 8 membered, 2 to 6 membered, 4 to 6 membered, 2 to 3 membered, or 4 to 5 membered), R^{24C}-substituted or unsubstituted cycloalkyl (e.g., C₃-C₈, C₃-C₆, C₄-C₆, or C₅-C₆), R^{24C}-substituted or unsubstituted heterocycloalkyl (e.g., 3 to 8 membered, 3 to 6 membered, 4 to 6 membered, 4 to 5 membered, or 5 to 6 membered), R^{24C}-substituted or unsubstituted aryl (e.g., C₆-C₁₂, C₆-C₁₀, or phenyl), or R^{24C}-substituted or unsubstituted heteroaryl (e.g., 5 to 12 membered, 5 to 10 membered, 5 to 9 membered, or 5 to 6 membered). In embodiments, R^{23C} is independently oxo,
halogen, —CX^{23C}₃, —CHX^{23C}₂, —CH₂X^{23C}, —OCX^{23C}₃, —OCH₂X^{23C}, —OCHX^{23C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{23C} is independently —F, —Cl, —Br, or —I. In embodiments,

R^{23C} is independently unsubstituted methyl. In embodiments, R^{23C} is independently unsubstituted ethyl.

R^{24C} is independently oxo,

halogen, —CX^{24C}₃, —CHX^{24C}₂, —CH₂X^{24C}, —OCX^{24C}₃, —OCH₂X^{24C}, —OCHX^{24C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{25C}-substituted or unsubstituted alkyl (e.g., C₁-C₈, C₁-C₆, C₁-C₄, or C₁-C₂), R^{25C}-substituted or unsubstituted heteroalkyl (e.g., 2 to 8 membered, 2 to 6 membered, 4 to 6 membered, 2 to 3 membered, or 4 to 5 membered), R^{25C}-substituted or unsubstituted cycloalkyl (e.g., C₃-C₈, C₃-C₆, C₄-C₆, or C₅-C₆), R^{25C}-substituted or unsubstituted heterocycloalkyl (e.g., 3 to 8 membered, 3 to 6 membered, 4 to 6 membered, 4 to 5 membered, or 5 to 6 membered), R^{25C}-substituted or unsubstituted aryl (e.g., C₆-C₁₂, C₆-C₁₀, or phenyl), or R^{25C}-substituted or unsubstituted heteroaryl (e.g., 5 to 12 membered, 5 to 10 membered, 5 to 9 membered, or 5 to 6 membered). In embodiments, R^{24C} is independently oxo,
halogen, —CX^{24C}₃, —CHX^{24C}₂, —CH₂X^{24C}, —OCX^{24C}₃, —OCH₂X^{24C}, —OCHX^{24C}₂, —CN, —OH, —NH₂, —COOH, —CONH₂, —NO₂, —SH, —SO₃H, —SO₄H, —SO₂H₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{24C} is independently —F, —Cl, —Br, or —I. In embodiments,

R^{24C} is independently unsubstituted methyl. In embodiments, R^{24C} is independently unsubstituted ethyl.

R^{25C} is independently oxo,

halogen, —CX^{25C}₃, —CHX^{25C}₂, —CH₂X^{25C}, —OCX^{25C}₃, —OCH₂X^{25C}, —OCHX^{25C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{25C} is independently —F, —Cl, —Br, or —I. In embodiments, R^{25C} is independently unsubstituted methyl. In embodiments, R^{25C} is independently unsubstituted ethyl.

In embodiments, R^{2D} is independently hydrogen, —CX^{2D}₃, —CHX^{2D}₂, —CH₂X^{2D}, —CN, —COOH, —CONH₂, R^{23D}-substituted or unsubstituted alkyl (e.g., C₁-C₈, C₁-C₆, C₁-C₄, or C₁-C₂), R^{23D}-substituted or unsubstituted heteroalkyl (e.g., 2 to 8 membered, 2 to 6 membered, 4 to 6 membered, 2 to 3 membered, or 4 to 5 membered), R^{23D}-substituted or unsubstituted cycloalkyl (e.g., C₃-C₈, C₃-C₆, C₄-C₆, or C₅-C₆), R^{23D}-substituted or unsubstituted heterocycloalkyl (e.g., 3 to 8 membered, 3 to 6 membered, 4 to 6 membered, 4 to 5 membered, or 5 to 6 membered), R^{23D}-substituted or unsubstituted aryl (e.g., C₆-C₁₂, C₆-C₁₀, or phenyl), or R^{23D}-substituted or unsubstituted heteroaryl (e.g., 5 to 12 membered, 5 to 10 membered, 5 to 9 membered, or 5 to 6 membered). In embodiments, R^{2D} is independently hydrogen, —CX^{2D}₃, —CHX^{2D}₂, —CH₂X^{2D}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{2D} is independently —F, —Cl, —Br, or —I. In embodiments, R^{2D} is independently hydrogen. In embodiments, R^{2D} is independently unsubstituted methyl. In embodiments, R^{2D} is independently unsubstituted ethyl.

R^{23D} is independently oxo,

halogen, —CX^{23D}₃, —CHX^{23D}₂, —CH₂X^{23D}, —OCX^{23D}₃, —OCH₂X^{23D}, —OCHX^{23D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{24D}-substituted or unsubstituted alkyl (e.g., C₁-C₈, C₁-C₆, C₁-C₄, or C₁-C₂), R^{24D}-substituted or unsubstituted heteroalkyl (e.g., 2 to 8 membered, 2 to 6 membered, 4 to 6 membered, 2 to 3 membered, or 4 to 5 membered), R^{24D}-substituted or unsubstituted cycloalkyl (e.g., C₃-C₈, C₃-C₆, C₄-C₆, or C₅-C₆), R^{24D}-substituted or unsubstituted heterocycloalkyl (e.g., 3 to 8 membered, 3 to 6 membered, 4 to 6 membered, 4 to 5 membered, or 5 to 6 membered), R^{24D}-substituted or unsubstituted aryl (e.g., C₆-C₁₂, C₆-C₁₀, or phenyl), or R^{24D}-substituted or unsubstituted heteroaryl (e.g., 5 to 12 membered, 5 to 10 membered, 5 to 9 membered, or 5 to 6 membered). In embodiments, R^{23D} is independently oxo,
halogen, —CX^{23D}₃, —CHX^{23D}₂, —CH₂X^{23D}, —OCX^{23D}₃, —OCH₂X^{23D}, —OCHX^{23D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{23D} is independently —F, —Cl, —Br, or —I. In embodiments,

R^{23D} is independently unsubstituted methyl. In embodiments, R^{23D} is independently unsubstituted ethyl.

R^{24D} is independently oxo,

halogen, —CX^{24D}₃, —CHX^{24D}₂, —CH₂X^{24D}, —OCX^{24D}₃, —OCH₂X^{24D}, —OCHX^{24D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{25D}-substituted or unsubstituted alkyl (e.g., C₁-C₈, C₁-C₆, C₁-C₄, or C₁-C₂), R^{25D}-substituted or unsubstituted heteroalkyl (e.g., 2 to 8 membered, 2 to 6 membered, 4 to 6 membered, 2 to 3 membered, or 4 to 5 membered), R^{25D}-substituted or unsubstituted cycloalkyl (e.g., C₃-C₈, C₃-C₆, C₄-C₆, or C₅-C₆), R^{25D}-substituted or unsubstituted heterocycloalkyl (e.g., 3 to 8 membered, 3 to 6 membered, 4 to 6 membered, 4 to 5 membered, or 5 to 6 membered), R^{25D}-substituted or unsubstituted aryl (e.g., C₆-C₁₂, C₆-C₁₀, or phenyl), or R^{25D}-substituted or unsubstituted heteroaryl (e.g., 5 to 12 membered, 5 to 10 membered, 5 to 9 membered, or 5 to 6 membered). In embodiments, R^{24D} is independently oxo,
halogen, —CX^{24D}₃, —CHX^{24D}₂, —CH₂X^{24D}, —OCX^{24D}₃, —OCH₂X^{24D}, —OCHX^{24D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{24D} is independently —F, —Cl, —Br, or —I. In embodiments, R^{24D} is independently unsubstituted methyl. In embodiments, R^{24D} is independently unsubstituted ethyl.

R^{25D} is independently oxo,

halogen, —CX^{25D}₃, —CHX^{25D}₂, —CH₂X^{25D}, —OCX^{25D}₃, —OCH₂X^{25D}, —OCHX^{25D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{25D} is independently —F, —Cl, —Br, or —I. In embodiments, R^{25D} is independently unsubstituted methyl. In embodiments, R^{25D} is independently unsubstituted ethyl.

In embodiments, z2 is 0. In embodiments, z2 is 1. In embodiments, z2 is 2. In embodiments, z2 is 3. In embodiments, z2 is 4.

In embodiments, L¹ is a bond, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to 6 membered heteroarylene. In embodiments, L¹ is a bond.

In embodiments, 12 is a bond. In embodiments, L¹ is —S(O)₂—. In embodiments, L¹ is —NR⁴—. In embodiments, L¹ is —O—. In embodiments, L¹ is —S—. In embodiments, L¹ is —C(O)—. In embodiments, L¹ is —C(O)NR⁴—. In embodiments, L¹ is —NR⁴C(O)—. In embodiments, L¹ is —NR⁴C(O)NH—. In embodiments, L¹ is —NHC(O)NR⁴—. In embodiments, L¹ is —C(O)O—. In embodiments, L¹ is —OC(O)—. In embodiments, L¹ is —NH—. In embodiments, L¹ is —C(O)NH—. In embodiments, L¹ is —NHC(O)—. In embodiments, L¹ is —NHC(O)NH—. In embodiments, L¹ is —CH₂—. In embodiments, L¹ is OCH₂—. In embodiments, L¹ is —CH₂O—. In embodiments, L¹ is CH₂CH₂—. In embodiments, L¹ is —NHCH₂—. In embodiments, L¹ is —CH₂NH—. In embodiments, L¹ is a bond.

In embodiments, L¹ is a bond, —S(O)₂—, —NR⁴—, —O—, —S—, —C(O)—, —C(O)NR⁴, —NR⁴C(O)—, —NR⁴C(O)NH—, —NHC(O)NR⁴—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹ is independently substituted or unsubstituted alkylene (e.g., C₁-C₈, C₁-C₆, C₁-C₄, or C₁-C₂). In embodiments, L¹ is independently substituted alkylene (e.g., C₁-C₈, C₁-C₆, C₁-C₄, or C₁-C₂). In embodiments, L¹ is independently unsubstituted alkylene (e.g., C₁-C₈, C₁-C₄, or C₁-C₂). In embodiments, L¹ is independently unsubstituted methylene. In embodiments, L¹ is independently unsubstituted ethylene. In embodiments, L¹ is independently unsubstituted propylene. In embodiments, L¹ is independently unsubstituted isopropylene. In embodiments, L¹ is independently unsubstituted tert-butylene. In embodiments, L¹ is independently substituted or unsubstituted heteroalkylene (e.g., 2 to 8 membered, 2 to 6 membered, 4 to 6 membered, 2 to 3 membered, or 4 to 5 membered). In embodiments, L¹ is independently substituted heteroalkylene (e.g., 2 to 8 membered, 2 to 6 membered, 4 to 6 membered, 2 to 3 membered, or 4 to 5 membered). In embodiments, L¹ is independently unsubstituted heteroalkylene (e.g., 2 to 8 membered, 2 to 6 membered, 4 to 6 membered, 2 to 3 membered, or 4 to 5 membered). In embodiments, L¹ is independently substituted or unsubstituted cycloalkylene (e.g., C₃-C₈, C₃-C₆, C₄-C₆, or C₅-C₆). In embodiments, L¹ is independently substituted cycloalkylene (e.g., C₃-C₈, C₃-C₆, C₄-C₆, or C₅-C₆). In embodiments, L¹ is independently unsubstituted cycloalkylene (e.g., C₃-C₈, C₃-C₆, C₄-C₆, or C₅-C₆). In embodiments, L¹ is independently substituted or unsubstituted heterocycloalkylene (e.g., 3 to 8 membered, 3 to 6 membered, 4 to 6 membered, 4 to 5 membered, or 5 to 6 membered). In embodiments, L¹ is independently substituted heterocycloalkylene (e.g., 3 to 8 membered, 3 to 6 membered, 4 to 6 membered, 4 to 5 membered, or 5 to 6 membered). In embodiments, L¹ is independently unsubstituted heterocycloalkylene (e.g., 3 to 8 membered, 3 to 6 membered, 4 to 6 membered, 4 to 5 membered, or 5 to 6 membered). In embodiments, L¹ is independently substituted or unsubstituted arylene (e.g., C₆-C₁₀ or phenylene). In embodiments, L¹ is independently substituted arylene (e.g., C₆-C₁₀ or phenylene). In embodiments, L¹ is independently unsubstituted arylene (e.g., C₆-C₁₀ or phenylene). In embodiments, L¹ is independently substituted or unsubstituted heteroarylene (e.g., 5 to 10 membered, 5 to 9 membered, or 5 to 6 membered). In embodiments, L¹ is independently substituted heteroarylene (e.g., 5 to 10 membered, 5 to 9 membered, or 5 to 6 membered). In embodiments, L¹ is independently unsubstituted heteroarylene (e.g., 5 to 10 membered, 5 to 9 membered, or 5 to 6 membered).

In embodiments, L¹ is independently bond, —S(O)₂—, —N(R⁴)—, —O—, —S—, —C(O)—, —C(O)N(R⁴)—, —N(R⁴)C(O)—, —N(R⁴)C(O)NH—, —NHC(O) N(R⁴)—, —C(O)O—, —OC(O)—, R³⁵-substituted or unsubstituted alkylene (e.g., C₁-C₈, C₁-C₆, C₁-C₄, or C₁-C₂), R³⁵-substituted or unsubstituted heteroalkylene (e.g., 2 to 8 membered, 2 to 6 membered, 4 to 6 membered, 2 to 3 membered, or 4 to 5 membered), R³⁵-substituted or unsubstituted cycloalkylene (e.g., C₃-C₈, C₃-C₆, C₄-C₆, or C₅-C₆), R³⁵-substituted or unsubstituted heterocycloalkylene (e.g., 3 to 8 membered, 3 to 6 membered, 4 to 6 membered, 4 to 5 membered, or 5 to 6 membered), R³⁵-substituted or unsubstituted arylene (e.g., C₆-C₁₀ or phenylene), or R³⁵-substituted or unsubstituted heteroarylene (e.g., 5 to 10 membered, 5 to 9 membered, or 5 to 6 membered). In embodiments, L¹ is independently bond, —S(O)₂—, —N(R⁴)—, —O—, —S—, —C(O)—, —C(O)N(R⁴)—, —N(R⁴)C(O)—, —N(R⁴)C(O)NH—, —NHC(O) N(R⁴)—, —C(O)O—, —OC(O)—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, 2 to 3 membered, or 4 to 5 membered), unsubstituted cycloalkylene (e.g., C₃-C₈, C₄-C₆, or C₅-C₆), unsubstituted heterocycloalkylene (e.g., 3 to 8 membered, 3 to 6 membered, 4 to 6 membered, 4 to 5 membered, or 5 to 6 membered), unsubstituted arylene (e.g., C₆-C₁₀ or phenylene), or unsubstituted heteroarylene (e.g., 5 to 10 membered, 5 to 9 membered, or 5 to 6 membered). In embodiments, L¹ is independently unsubstituted methylene. In embodiments, L¹ is independently unsubstituted ethylene. In embodiments, L¹ is independently methyl-substituted methylene.

R³⁵ is independently oxo,

halogen, —CX³⁵₃, —CHX³⁵₂, —CH₂X³⁵, —OCX³⁵₃, —OCH₂X³⁵, —OCHX³⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³⁶-substituted or unsubstituted alkyl (e.g., C₁-C₈, C₁-C₆, C₁-C₄, or C₁-C₂), R³⁶-substituted or unsubstituted heteroalkyl (e.g., 2 to 8 membered, 2 to 6 membered, 4 to 6 membered, 2 to 3 membered, or 4 to 5 membered), R³⁶-substituted or unsubstituted cycloalkyl (e.g., C₃-C₈, C₄-C₆, or C₅-C₆), R³⁶-substituted or unsubstituted heterocycloalkyl (e.g., 3 to 8 membered, 3 to 6 membered, 4 to 6 membered, 4 to 5 membered, or 5 to 6 membered), R³⁶-substituted or unsubstituted aryl (e.g., C₆-C₁₀ or phenyl), or R³⁶-substituted or unsubstituted heteroaryl (e.g., 5 to 10 membered, 5 to 9 membered, or 5 to 6 membered). In embodiments, R³⁵ is independently oxo,
halogen, —CX³⁵₃, —CHX³⁵₂, —CH₂X³⁵, —OCX³⁵₃, —OCH₂X³⁵, —OCHX³⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁵ is independently —F, —Cl, —Br, or —I. In embodiments, R³⁵ is independently unsubstituted methyl. In embodiments, R³⁵ is independently unsubstituted ethyl.

R³⁶ is independently oxo,

halogen, —CX³⁶₃, —CHX³⁶₂, —CH₂X³⁶, —OCX³⁶₃, —OCH₂X³⁶, —OCHX³⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³⁷-substituted or unsubstituted alkyl (e.g., C₁-C₈, C₁-C₆, C₁-C₄, or C₁-C₂), R³⁷-substituted or unsubstituted heteroalkyl (e.g., 2 to 8 membered, 2 to 6 membered, 4 to 6 membered, 2 to 3 membered, or 4 to 5 membered), R³⁷-substituted or unsubstituted cycloalkyl (e.g., C₃-C₈, C₃-C₆, C₄-C₆, or C₅-C₆), R³⁷-substituted or unsubstituted heterocycloalkyl (e.g., 3 to 8 membered, 3 to 6 membered, 4 to 6 membered, 4 to 5 membered, or 5 to 6 membered), R³⁷-substituted or unsubstituted aryl (e.g., C₆-C₁₀ or phenyl), or R³⁷-substituted or unsubstituted heteroaryl (e.g., 5 to 10 membered, 5 to 9 membered, or 5 to 6 membered). In embodiments, R³⁶ is independently oxo,
halogen, —CX³⁶₃, —CHX³⁶₂, —CH₂X³⁶, —OCX³⁶₃, —OCH₂X³⁶, —OCHX³⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁶ is independently —F, —Cl, —Br, or —I. In embodiments, R³⁶ is independently unsubstituted methyl. In embodiments, R³⁶ is independently unsubstituted ethyl.

R³⁷ is independently oxo,

halogen, —CX³⁷₃, —CHX³⁷₂, —CH₂X³⁷, —OCX³⁷₃, —OCH₂X³⁷, —OCHX³⁷₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁷ is independently —F, —Cl, —Br, or —I. In embodiments, R³⁷ is independently unsubstituted methyl. In embodiments, R³⁷ is independently unsubstituted ethyl.

In embodiments, R⁴ is independently hydrogen, —CX⁴₃, —CHX⁴₂, —CH₂X⁴, —OCX⁴₃, —OCH₂X⁴, —OCHX⁴₂, —CN, —C(O)R^{4A}, —C(O)OR^{4A}, —C(O)NR^{4A}R^{4B}OR^{4A}—, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁴ is independently hydrogen. In embodiments, R⁴ is independently —CX⁴₃. In embodiments, R⁴ is independently —CHX⁴₂. In embodiments, R⁴ is independently —CH₂X⁴. In embodiments, R⁴ is independently —CN. In embodiments, R⁴ is independently —C(O)R^{4A}. In embodiments, R⁴ is independently —C(O)—OR^{4A}. In embodiments, R⁴ is independently —C(O)NR^{4A}R^{4B}. In embodiments, R⁴ is independently —COOH. In embodiments, R⁴ is independently —CONH₂. In embodiments, R⁴ is independently —CF₃. In embodiments, R⁴ is independently —CHF₂. In embodiments, R⁴ is independently —CH₂F. In embodiments, R⁴ is independently —CH₃. In embodiments, R⁴ is independently —CH₂CH₃. In embodiments, R⁴ is independently —CH₂CH₂CH₃. In embodiments, R⁴ is independently —CH(CH₃)₂. In embodiments, R⁴ is independently —C(CH₃)₃.

In embodiments, R⁴ is independently unsubstituted methyl. In embodiments, R⁴ is independently unsubstituted ethyl. In embodiments, R⁴ is independently unsubstituted propyl. In embodiments, R⁴ is independently unsubstituted isopropyl. In embodiments, R⁴ is independently unsubstituted n-propyl. In embodiments, R⁴ is independently unsubstituted butyl. In embodiments, R⁴ is independently unsubstituted n-butyl. In embodiments, R⁴ is independently unsubstituted t-butyl. In embodiments, R⁴ is independently unsubstituted pentyl. In embodiments, R⁴ is independently unsubstituted n-pentyl. In embodiments, R⁴ is independently unsubstituted hexyl. In embodiments, R⁴ is independently unsubstituted n-hexyl. In embodiments, R⁴ is independently unsubstituted heptyl. In embodiments, R⁴ is independently unsubstituted n-heptyl. In embodiments, R⁴ is independently unsubstituted octyl. In embodiments, R⁴ is independently unsubstituted n-octyl. In embodiments, R⁴ is independently unsubstituted benzyl. In embodiments, R⁴ is independently unsubstituted C₁-C₈ alkyl. In embodiments, R⁴ is independently halo-substituted methyl. In embodiments, R⁴ is independently halo-substituted ethyl. In embodiments, R⁴ is independently halo-substituted isopropyl. In embodiments, R⁴ is independently halo-substituted n-propyl. In embodiments, R⁴ is independently halo-substituted n-butyl. In embodiments, R⁴ is independently halo-substituted t-butyl. In embodiments, R¹ is independently halo-substituted n-pentyl. In embodiments, R⁴ is independently halo-substituted benzyl. In embodiments, R⁴ is independently halo-substituted C₁-C₈ alkyl. In embodiments, R⁴ is independently unsubstituted 2 to 6 membered heteroalkyl. In embodiments, R⁴ is independently unsubstituted 2 to 7 membered heteroalkyl. In embodiments, R⁴ is independently unsubstituted 2 to 8 membered heteroalkyl. In embodiments, R⁴ is independently unsubstituted 2 to 9 membered heteroalkyl. In embodiments, R⁴ is independently unsubstituted 2 to 10 membered heteroalkyl. In embodiments, R⁴ is independently unsubstituted 3 to 10 membered heteroalkyl. In embodiments, R⁴ is independently unsubstituted 4 to 10 membered heteroalkyl. In embodiments, R⁴ is independently unsubstituted 5 to 10 membered heteroalkyl. In embodiments, R⁴ is independently unsubstituted 6 to 10 membered heteroalkyl. In embodiments, R⁴ is independently unsubstituted 7 to 10 membered heteroalkyl. In embodiments, R⁴ is independently unsubstituted 8 to 10 membered heteroalkyl. In embodiments, R⁴ is independently unsubstituted 6 to 10 membered heteroalkyl. In embodiments, R⁴ is independently unsubstituted 7 to 9 membered heteroalkyl.

In embodiments, R⁴ is independently substituted or unsubstituted alkyl (e.g., C₁-C₈, C₁-C₆, C₁-C₄, or C₁-C₂). In embodiments, R⁴ is independently substituted alkyl (e.g., C₁-C₈, C₁-C₆, C₁-C₄, or C₁-C₂). In embodiments, R⁴ is independently unsubstituted alkyl (e.g., C₁-C₈, C₁-C₆, C₁-C₄, or C₁-C₂). In embodiments, R⁴ is independently unsubstituted methyl. In embodiments, R⁴ is independently unsubstituted ethyl. In embodiments, R⁴ is independently unsubstituted propyl. In embodiments, R⁴ is independently unsubstituted isopropyl. In embodiments, R⁴ is independently unsubstituted tert-butyl. In embodiments, R⁴ is independently substituted or unsubstituted heteroalkyl (e.g., 2 to 8 membered, 2 to 6 membered, 4 to 6 membered, 2 to 3 membered, or 4 to 5 membered). In embodiments, R⁴ is independently substituted heteroalkyl (e.g., 2 to 8 membered, 2 to 6 membered, 4 to 6 membered, 2 to 3 membered, or 4 to 5 membered). In embodiments, R⁴ is independently unsubstituted heteroalkyl (e.g., 2 to 8 membered, 2 to 6 membered, 4 to 6 membered, 2 to 3 membered, or 4 to 5 membered). In embodiments, R⁴ is independently substituted or unsubstituted cycloalkyl (e.g., C₃-C₈, C₃-C₆, C₄-C₆, or C₅-C₆). In embodiments, R⁴ is independently substituted cycloalkyl (e.g., C₃-C₈, C₃-C₆, C₄-C₆, or C₅-C₆). In embodiments, R⁴ is independently unsubstituted cycloalkyl (e.g., C₃-C₈, C₃-C₆, C₄-C₆, or C₅-C₆). In embodiments, R⁴ is independently substituted or unsubstituted heterocycloalkyl (e.g., 3 to 8 membered, 3 to 6 membered, 4 to 6 membered, 4 to 5 membered, or 5 to 6 membered). In embodiments, R⁴ is independently substituted heterocycloalkyl (e.g., 3 to 8 membered, 3 to 6 membered, 4 to 6 membered, 4 to 5 membered, or 5 to 6 membered). In embodiments, R⁴ is independently unsubstituted heterocycloalkyl (e.g., 3 to 8 membered, 3 to 6 membered, 4 to 6 membered, 4 to 5 membered, or 5 to 6 membered). In embodiments, R⁴ is independently substituted or unsubstituted aryl (e.g., C₆-C₁₀ or phenyl). In embodiments, R⁴ is independently substituted aryl (e.g., C₆-C₁₀ or phenyl). In embodiments, R⁴ is independently unsubstituted aryl (e.g., C₆-C₁₀ or phenyl). In embodiments, R⁴ is independently substituted or unsubstituted heteroaryl (e.g., 5 to 10 membered, 5 to 9 membered, or 5 to 6 membered). In embodiments, R⁴ is independently substituted heteroaryl (e.g., 5 to 10 membered, 5 to 9 membered, or 5 to 6 membered). In embodiments, R⁴ is independently unsubstituted heteroaryl (e.g., 5 to 10 membered, 5 to 9 membered, or 5 to 6 membered).

In embodiments, R^{4A} is independently hydrogen. In embodiments, R^{4A} is independently —CX^{4A}₃. In embodiments, R^{4A} is independently —CHX^{4A}₂. In embodiments, R^{4A} is independently —CH₂X^{4A}. In embodiments, R^{4A} is independently —CN. In embodiments, R^{4A} is independently —COOH. In embodiments, R^{4A} is independently —CONH₂. In embodiments, X^{4A} is independently —F, —Cl, —Br, or —I.

In embodiments, R^{4A} is independently substituted or unsubstituted alkyl (e.g., C₁-C₈, C₁-C₆, C₁-C₄, or C₁-C₂). In embodiments, R^{4A} is independently substituted alkyl (e.g., C₁-C₈, C₁-C₆, C₁-C₄, or C₁-C₂). In embodiments, R^{4A} is independently unsubstituted alkyl (e.g., C₁-C₈, C₁-C₆, C₁-C₄, or C₁-C₂). In embodiments, R^{4A} is independently unsubstituted methyl. In embodiments, R^{4A} is independently unsubstituted ethyl. In embodiments, R^{4A} is independently unsubstituted propyl. In embodiments, R^{4A} is independently unsubstituted isopropyl. In embodiments, R^{4A} is independently unsubstituted tert-butyl. In embodiments, R^{4A} is independently substituted or unsubstituted heteroalkyl (e.g., 2 to 8 membered, 2 to 6 membered, 4 to 6 membered, 2 to 3 membered, or 4 to 5 membered). In embodiments, R^{4A} is independently substituted heteroalkyl (e.g., 2 to 8 membered, 2 to 6 membered, 4 to 6 membered, 2 to 3 membered, or 4 to 5 membered). In embodiments, R^{4A} is independently unsubstituted heteroalkyl (e.g., 2 to 8 membered, 2 to 6 membered, 4 to 6 membered, 2 to 3 membered, or 4 to 5 membered). In embodiments, R^{4A} is independently substituted or unsubstituted cycloalkyl (e.g., C₃-C₈, C₃-C₆, C₄-C₆, or C₅-C₆). In embodiments, R^{4A} is independently substituted cycloalkyl (e.g., C₃-C₈, C₃-C₆, C₄-C₆, or C₅-C₆). In embodiments, R^{4A} is independently unsubstituted cycloalkyl (e.g., C₃-C₈, C₃-C₆, C₄-C₆, or C₅-C₆). In embodiments, R^{4A} is independently substituted or unsubstituted heterocycloalkyl (e.g., 3 to 8 membered, 3 to 6 membered, 4 to 6 membered, 4 to 5 membered, or 5 to 6 membered). In embodiments, R^{4A} is independently substituted heterocycloalkyl (e.g., 3 to 8 membered, 3 to 6 membered, 4 to 6 membered, 4 to 5 membered, or 5 to 6 membered). In embodiments, R^{4A} is independently unsubstituted heterocycloalkyl (e.g., 3 to 8 membered, 3 to 6 membered, 4 to 6 membered, 4 to 5 membered, or 5 to 6 membered). In embodiments, R^{4A} is independently substituted or unsubstituted aryl (e.g., C₆-C₁₀ or phenyl). In embodiments, R^{4A} is independently substituted aryl (e.g., C₆-C₁₀ or phenyl). In embodiments, R^{4A} is independently unsubstituted aryl (e.g., C₆-C₁₀ or phenyl). In embodiments, R^{4A} is independently substituted or unsubstituted heteroaryl (e.g., 5 to 10 membered, 5 to 9 membered, or 5 to 6 membered). In embodiments, R^{4A} is independently substituted heteroaryl (e.g., 5 to 10 membered, 5 to 9 membered, or 5 to 6 membered). In embodiments, R^{4A} is independently unsubstituted heteroaryl (e.g., 5 to 10 membered, 5 to 9 membered, or 5 to 6 membered).

In embodiments, R^{4B} is independently hydrogen. In embodiments, R^{4B} is independently —CX^{4B}₃. In embodiments, R^{4B} is independently —CHX^{4B}₂. In embodiments, R^{4B} is independently —CH₂X^{4B}. In embodiments, R^{4B} is independently —CN. In embodiments, R^{4B} is independently —COOH. In embodiments, R^{4B} is independently —CONH₂. In embodiments, X^{4B} is independently —F, —Cl, —Br, or —I.

In embodiments, R^{4B} is independently substituted or unsubstituted alkyl (e.g., C₁-C₈, C₁-C₆, C₁-C₄, or C₁-C₂). In embodiments, R^{4B} is independently substituted alkyl (e.g., C₁-C₈, C₁-C₆, C₁-C₄, or C₁-C₂). In embodiments, R^{4B} is independently unsubstituted alkyl (e.g., C₁-C₈, C₁-C₆, C₁-C₄, or C₁-C₂). In embodiments, R^{4B} is independently unsubstituted methyl. In embodiments, R^{4B} is independently unsubstituted ethyl. In embodiments, R^{4B} is independently unsubstituted propyl. In embodiments, R^{4B} is independently unsubstituted isopropyl. In embodiments, R^{4B} is independently unsubstituted tert-butyl. In embodiments, R^{4B} is independently substituted or unsubstituted heteroalkyl (e.g., 2 to 8 membered, 2 to 6 membered, 4 to 6 membered, 2 to 3 membered, or 4 to 5 membered). In embodiments, R^{4B} is independently substituted heteroalkyl (e.g., 2 to 8 membered, 2 to 6 membered, 4 to 6 membered, 2 to 3 membered, or 4 to 5 membered). In embodiments, R^{4B} is independently unsubstituted heteroalkyl (e.g., 2 to 8 membered, 2 to 6 membered, 4 to 6 membered, 2 to 3 membered, or 4 to 5 membered). In embodiments, R^{4B} is independently substituted or unsubstituted cycloalkyl (e.g., C₃-C₈, C₃-C₆, C₄-C₆, or C₅-C₆). In embodiments, R^{4B} is independently substituted cycloalkyl (e.g., C₃-C₈, C₃-C₆, C₄-C₆, or C₅-C₆). In embodiments, R^{4B} is independently unsubstituted cycloalkyl (e.g., C₃-C₈, C₃-C₆, C₄-C₆, or C₅-C₆). In embodiments, R^{4B} is independently substituted or unsubstituted heterocycloalkyl (e.g., 3 to 8 membered, 3 to 6 membered, 4 to 6 membered, 4 to 5 membered, or 5 to 6 membered). In embodiments, R^{4B} is independently substituted heterocycloalkyl (e.g., 3 to 8 membered, 3 to 6 membered, 4 to 6 membered, 4 to 5 membered, or 5 to 6 membered). In embodiments, R^{4B} is independently unsubstituted heterocycloalkyl (e.g., 3 to 8 membered, 3 to 6 membered, 4 to 6 membered, 4 to 5 membered, or 5 to 6 membered). In embodiments, R^{4B} is independently substituted or unsubstituted aryl (e.g., C₆-C₁₀ or phenyl). In embodiments, R^{4B} is independently substituted aryl (e.g., C₆-C₁₀ or phenyl). In embodiments, R^{4B} is independently unsubstituted aryl (e.g., C₆-C₁₀ or phenyl). In embodiments, R^{4B} is independently substituted or unsubstituted heteroaryl (e.g., 5 to 10 membered, 5 to 9 membered, or 5 to 6 membered). In embodiments, R^{4B} is independently substituted heteroaryl (e.g., 5 to 10 membered, 5 to 9 membered, or 5 to 6 membered). In embodiments, R^{4B} is independently unsubstituted heteroaryl (e.g., 5 to 10 membered, 5 to 9 membered, or 5 to 6 membered).

In embodiments, R^{4A} and R^{4B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{4A} and R^{4B} substituents bonded to the same nitrogen atom may be joined to form a substituted or unsubstituted heteroaryl (e.g., 5 to 10 membered, 5 to 9 membered, or 5 to 6 membered). In embodiments, R^{4A} and R^{4B} substituents bonded to the same nitrogen atom may be joined to form a substituted heteroaryl (e.g., 5 to 10 membered, 5 to 9 membered, or 5 to 6 membered). In embodiments, R^{4A} and R^{4B} substituents bonded to the same nitrogen atom may be joined to form an unsubstituted heteroaryl (e.g., 5 to 10 membered, 5 to 9 membered, or 5 to 6 membered).

In embodiments, R⁴ is independently hydrogen, —CX⁴₃, —CHX⁴₂, —CH₂X⁴, —CN, —COOH, —CONH₂, R²⁹-substituted or unsubstituted alkyl (e.g., C₁-C₈, C₁-C₆, C₁-C₄, or C₁-C₂), R²⁹-substituted or unsubstituted heteroalkyl (e.g., 2 to 8 membered, 2 to 6 membered, 4 to 6 membered, 2 to 3 membered, or 4 to 5 membered), R²⁹-substituted or unsubstituted cycloalkyl (e.g., C₃-C₈, C₃-C₆, C₄-C₆, or C₅-C₆), R²⁹-substituted or unsubstituted heterocycloalkyl (e.g., 3 to 8 membered, 3 to 6 membered, 4 to 6 membered, 4 to 5 membered, or 5 to 6 membered), R²⁹-substituted or unsubstituted aryl (e.g., C₆-C₁₀ or phenyl), or R²⁹-substituted or unsubstituted heteroaryl (e.g., 5 to 10 membered, 5 to 9 membered, or 5 to 6 membered). In embodiments, R⁴ is independently hydrogen, —CX⁴₃, —CHX⁴₂, —CH₂X⁴, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X⁴ is independently —F, —Cl, —Br, or —I. In embodiments, R⁴ is independently hydrogen. In embodiments, R⁴ is independently unsubstituted methyl. In embodiments, R⁴ is independently unsubstituted ethyl.

R²⁹ is independently oxo,

halogen, —CX²⁹₃, —CHX²⁹₂, —CH₂X²⁹, —OCX²⁹₃, —OCH₂X²⁹, —OCHX²⁹₂, —CN, —OH, —NH₂, —COOH, —CONH₂, —NO₂, —SH, 03H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³⁰-substituted or unsubstituted alkyl (e.g., C₁-C₈, C₁-C₆, C₁-C₄, or C₁-C₂), R³⁰-substituted or unsubstituted heteroalkyl (e.g., 2 to 8 membered, 2 to 6 membered, 4 to 6 membered, 2 to 3 membered, or 4 to 5 membered), R³⁰-substituted or unsubstituted cycloalkyl (e.g., C₃-C₈, C₃-C₆, C₄-C₆, or C₅-C₆), R³⁰-substituted or unsubstituted heterocycloalkyl (e.g., 3 to 8 membered, 3 to 6 membered, 4 to 6 membered, 4 to 5 membered, or 5 to 6 membered), R³⁰-substituted or unsubstituted aryl (e.g., C₆-C₁₀ or phenyl), or R³⁰-substituted or unsubstituted heteroaryl (e.g., 5 to 10 membered, 5 to 9 membered, or 5 to 6 membered). In embodiments, R²⁹ is independently oxo,
halogen, —CX²⁹₃, —CHX²⁹₂, —CH₂X²⁹, —OCX²⁹₃, —OCH₂X²⁹, —OCHX²⁹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²⁹ is independently —F, —Cl, —Br, or —I. In embodiments, R²⁹ is independently unsubstituted methyl. In embodiments, R²⁹ is independently unsubstituted ethyl.

R³⁰ is independently oxo,

halogen, —CX³⁰₃, —CHX³⁰₂, —CH₂X³⁰, —OCX³⁰₃, —OCH₂X³⁰, —OCHX³⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³¹-substituted or unsubstituted heteroaryl (e.g., 5 to 10 membered, 5 to 9 membered, or 5 to 6 membered). In embodiments, R³⁰ is independently oxo,
halogen, —CX³⁰₃, —CHX³⁰₂, —CH₂X³⁰, —OCX³⁰₃, —OCH₂X³⁰, —OCHX³⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁰ is independently —F, —Cl, —Br, or —I. In embodiments, R³⁰ is independently unsubstituted methyl. In embodiments, R³⁰ is independently unsubstituted ethyl.

R³¹ is independently oxo,

halogen, —CX³¹₃, —CHX³¹₂, —CH₂X³¹, —OCX³¹₃, —OCH₂X³¹, —OCHX³¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³¹ is independently —F, —Cl, —Br, or —I. In embodiments, R³¹ is independently unsubstituted methyl. In embodiments, R³¹ is independently unsubstituted ethyl.

In embodiments, R^{4A} is independently hydrogen, —CX^{4A}₃, —CHX^{4A}₂, —CH₂X^{4A}, —CN, —COOH, —CONH₂, R^{29A}-substituted or unsubstituted alkyl (e.g., C₁-C₈, C₁-C₄, or C₁-C₂), R^{29A}-substituted or unsubstituted heteroalkyl (e.g., 2 to 8 membered, 2 to 6 membered, 4 to 6 membered, 2 to 3 membered, or 4 to 5 membered), R^{29A}-substituted or unsubstituted cycloalkyl (e.g., C₃-C₈, C₃-C₆, C₄-C₆, or C₅-C₆), R^{29A}-substituted or unsubstituted heterocycloalkyl (e.g., 3 to 8 membered, 3 to 6 membered, 4 to 6 membered, 4 to 5 membered, or 5 to 6 membered), R^{29A}-substituted or unsubstituted aryl (e.g., C₆-C₁₀ or phenyl), or R^{29A}-substituted or unsubstituted heteroaryl (e.g., 5 to 10 membered, 5 to 9 membered, or 5 to 6 membered). In embodiments, R^{4A} is independently hydrogen, —CX^{4A}₃, —CHX^{4A}₂, —CH₂X^{4A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{4A} is independently —F, —Cl, —Br, or —I. In embodiments, R^{4A} is independently hydrogen. In embodiments, R^{4A} is independently unsubstituted methyl. In embodiments, R^{4A} is independently unsubstituted ethyl.

In embodiments, R^{4A} and R^{4B} substituents bonded to the same nitrogen atom may optionally be joined to form a R^{29A}-substituted or unsubstituted heterocycloalkyl (e.g., 3 to 8 membered, 3 to 6 membered, 4 to 6 membered, 4 to 5 membered, or 5 to 6 membered) or R^{29A}-substituted or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form a R^{29A}-substituted or unsubstituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{29A} is independently oxo,

halogen, —CX^{29A}₃, —CHX^{29A}₂, —CH₂X^{29A}, —OCX^{29A}₃, —OCH₂X^{29A}, —OCHX^{29A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{30A}-substituted or unsubstituted alkyl (e.g., C₁-C₈, C₁-C₆, C₁-C₄, or C₁-C₂), R^{30A}-substituted or unsubstituted heteroalkyl (e.g., 2 to 8 membered, 2 to 6 membered, 4 to 6 membered, 2 to 3 membered, or 4 to 5 membered), R^{30A}-substituted or unsubstituted cycloalkyl (e.g., C₃-C₈, C₃-C₆, C₄-C₆, or C₅-C₆), R^{30A}-substituted or unsubstituted heterocycloalkyl (e.g., 3 to 8 membered, 3 to 6 membered, 4 to 6 membered, 4 to 5 membered, or 5 to 6 membered), R^{30A}-substituted or unsubstituted aryl (e.g., C₆-C₁₀ or phenyl), or R^{30A}-substituted or unsubstituted heteroaryl (e.g., 5 to 10 membered, 5 to 9 membered, or 5 to 6 membered). In embodiments, R^{29A} is independently oxo,
halogen, —CX^{29A}₃, —CHX^{29A}₂, —CH₂X^{29A}, —OCX^{29A}₃, —OCH₂X^{29A}, —OCHX^{29A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{29A} is independently —F, —Cl, —Br, or —I. In embodiments, R^{29A} is independently unsubstituted methyl. In embodiments, R^{29A} is independently unsubstituted ethyl.

R^{30A} is independently oxo,

halogen, —CX^{30A}₃, —CHX^{30A}₂, —CH₂X^{30A}, —OCX^{30A}₃, —OCH₂X^{30A}, —OCHX^{30A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{31A}-substituted or unsubstituted alkyl (e.g., C₁-C₈, C₁-C₆, C₁-C₄, or C₁-C₂), R^{31A}-substituted or unsubstituted heteroalkyl (e.g., 2 to 8 membered, 2 to 6 membered, 4 to 6 membered, 2 to 3 membered, or 4 to 5 membered), R^{31A}-substituted or unsubstituted cycloalkyl (e.g., C₃-C₈, C₃-C₆, C₄-C₆, or C₅-C₆), R^{31A}-substituted or unsubstituted heterocycloalkyl (e.g., 3 to 8 membered, 3 to 6 membered, 4 to 6 membered, 4 to 5 membered, or 5 to 6 membered), R^{31A}-substituted or unsubstituted aryl (e.g., C₆-C₁₀ or phenyl), or R^{31A}-substituted or unsubstituted heteroaryl (e.g., 5 to 10 membered, 5 to 9 membered, or 5 to 6 membered). In embodiments, R^{30A} is independently oxo,
halogen, —CX^{30A}₃, —CHX^{30A}₂, —CH₂X^{30A}, —OCX^{30A}₃, —OCH₂X^{30A}, —OCHX^{30A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{30A} is independently —F, —Cl, —Br, or —I. In embodiments, R^{30A} is independently unsubstituted methyl. In embodiments, R^{30A} is independently unsubstituted ethyl.

R^{31A} is independently oxo,

halogen, —CX^{31A}₃, —CHX^{31A}₂, —CH₂X^{31A}, —OCX^{31A}₃, —OCH₂X^{31A}, —OCHX^{31A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{31A} is independently —F, —Cl, —Br, or —I. In embodiments, R^{31A} is independently unsubstituted methyl. In embodiments, R^{31A} is independently unsubstituted ethyl.

In embodiments, R^{4B} is independently hydrogen, —CX^{4B}₃, —CHX^{4B}₂, —CH₂X^{4B}, —CN, —COOH, —CONH₂, R^{29B}-substituted or unsubstituted alkyl (e.g., C₁-C₈, C₁-C₆, C₁-C₄, or C₁-C₂), R^{29B}-substituted or unsubstituted heteroalkyl (e.g., 2 to 8 membered, 2 to 6 membered, 4 to 6 membered, 2 to 3 membered, or 4 to 5 membered), R^{29B}-substituted or unsubstituted cycloalkyl (e.g., C₃-C₈, C₃-C₆, C₄-C₆, or C₅-C₆), R^{29B}-substituted or unsubstituted heterocycloalkyl (e.g., 3 to 8 membered, 3 to 6 membered, 4 to 6 membered, 4 to 5 membered, or 5 to 6 membered), R^{29B}-substituted or unsubstituted aryl (e.g., C₆-C₁₀ or phenyl), or R^{29B}-substituted or unsubstituted heteroaryl (e.g., 5 to 10 membered, 5 to 9 membered, or 5 to 6 membered). In embodiments, R^{4B} is independently hydrogen, —CX^{4B}₃, —CHX^{4B}₂, —CH₂X^{4B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{4B} is independently —F, —Cl, —Br, or —I. In embodiments, R^{4B} is independently hydrogen. In embodiments, R^{4B} is independently unsubstituted methyl. In embodiments, R^{4B} is independently unsubstituted ethyl.

In embodiments, R^{4A} and R^{4B} substituents bonded to the same nitrogen atom may optionally be joined to form a R^{29B}-substituted or unsubstituted heterocycloalkyl (e.g., 3 to 8 membered, 3 to 6 membered, 4 to 6 membered, 4 to 5 membered, or 5 to 6 membered) or R^{29B}-substituted or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form a R^{29B}-substituted or unsubstituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{29B} is independently oxo,

halogen, —CX^{29B}₃, —CHX^{29B}₂, —CH₂X^{29B}, —OCX^{29B}₃, —OCH₂X^{29B}, —OCHX^{29B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{30B}-substituted or unsubstituted alkyl (e.g., C₁-C₈, C₁-C₆, C₁-C₄, or C₁-C₂), R^{30B}-substituted or unsubstituted heteroalkyl (e.g., 2 to 8 membered, 2 to 6 membered, 4 to 6 membered, 2 to 3 membered, or 4 to 5 membered), R^{30B}-substituted or unsubstituted cycloalkyl (e.g., C₃-C₈, C₃-C₆, C₄-C₆, or C₅-C₆), R^{30B}-substituted or unsubstituted heterocycloalkyl (e.g., 3 to 8 membered, 3 to 6 membered, 4 to 6 membered, 4 to 5 membered, or 5 to 6 membered), R^{30B}-substituted or unsubstituted aryl (e.g., C₆-C₁₀ or phenyl), or R^{30B}-substituted or unsubstituted heteroaryl (e.g., 5 to 10 membered, 5 to 9 membered, or 5 to 6 membered). In embodiments, R^{29B} is independently oxo,
halogen, —CX^{29B}₃, —CHX^{29B}₂, —CH₂X^{29B}, —OCX^{29B}₃, —OCH₂X^{29B}, —OCHX^{29B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{29B} is independently —F, —Cl, —Br, or —I. In embodiments, R^{29B} is independently unsubstituted methyl. In embodiments, R^{29B} is independently unsubstituted ethyl.

R^{30B} is independently oxo,

halogen, —CX^{30B}₃, —CHX^{30B}₂, —CH₂X^{30B}, —OCX^{30B}₃, —OCH₂X^{30B}, —OCHX^{30B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{31B}-substituted or unsubstituted alkyl (e.g., C₁-C₈, C₁-C₆, C₁-C₄, or C₁-C₂), R^{31B}-substituted or unsubstituted heteroalkyl (e.g., 2 to 8 membered, 2 to 6 membered, 4 to 6 membered, 2 to 3 membered, or 4 to 5 membered), R^{31B}-substituted or unsubstituted cycloalkyl (e.g., C₃-C₈, C₃-C₆, C₄-C₆, or C₅-C₆), R^{31B}-substituted or unsubstituted heterocycloalkyl (e.g., 3 to 8 membered, 3 to 6 membered, 4 to 6 membered, 4 to 5 membered, or 5 to 6 membered), R^{31B}-substituted or unsubstituted aryl (e.g., C₆-C₁₀ or phenyl), or R^{31B}-substituted or unsubstituted heteroaryl (e.g., 5 to 10 membered, 5 to 9 membered, or 5 to 6 membered). In embodiments, R^{30B} is independently oxo,
halogen, —CX^{30B}₃, —CHX^{30B}₂, —CH₂X^{30B}, —OCX^{30B}₃, —OCH₂X^{30B}, —OCHX^{30B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{30B} is independently —F, —Cl, —Br, or —I. In embodiments, R^{30B} is independently unsubstituted methyl. In embodiments, R^{30B} is independently unsubstituted ethyl.

R^{31B} is independently oxo,

halogen, —CX^{31B}₃, —CHX^{31B}₂, —CH₂X^{31B}, —OCX^{31B}₃, —OCH₂X^{31B}, —OCHX^{31B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{31B} is independently —F, —Cl, —Br, or —I. In embodiments, R^{31B} is independently unsubstituted methyl. In embodiments, R^{31B} is independently unsubstituted ethyl.

In embodiments, L² is —NR⁵— or substituted or unsubstituted heterocycloalkylene including a ring nitrogen bonded directly to E. In embodiments, L² is —NR⁵—.

In embodiments, L² is a bond. In embodiments, L² is —S(O)₂—. In embodiments, L² is —NR⁵—. In embodiments, L² is —O—. In embodiments, L² is —S—. In embodiments, L² is —C(O)—. In embodiments, L² is —C(O)NR⁵—. In embodiments, L² is —NR⁵C(O)—. In embodiments, L² is —NR⁵C(O)NH—. In embodiments, L² is —NHC(O)NR⁵—. In embodiments, L² is —C(O)O—. In embodiments, L² is —OC(O)—. In embodiments, L² is —NH—. In embodiments, L² is —C(O)NH—. In embodiments, L² is —NHC(O)—. In embodiments, L² is —NHC(O)NH—. In embodiments, L² is —CH₂—. In embodiments, L² is OCH₂—. In embodiments, L² is —CH₂O—. In embodiments, L² is —NHCH₂—. In embodiments, L² is —CH₂NH—.

In embodiments, L² is a bond, —S(O)₂—, —NR⁵—, —O—, —S—, —C(O)—, —C(O)NR⁵—, —NR⁴C(O)—, —NR⁵C(O)NH—, —NHC(O)NR⁵—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L² is independently substituted or unsubstituted alkylene (e.g., C₁-C₈, C₁-C₆, C₁-C₄, or C₁-C₂). In embodiments, L² is independently substituted alkylene (e.g., C₁-C₈, C₁-C₆, C₁-C₄, or C₁-C₂). In embodiments, L² is independently unsubstituted alkylene (e.g., C₁-C₈, C₁-C₆, C₁-C₄, or C₁-C₂). In embodiments, L² is independently unsubstituted methylene. In embodiments, L² is independently unsubstituted ethylene. In embodiments, L² is independently unsubstituted propylene. In embodiments, L² is independently unsubstituted isopropylene. In embodiments, L² is independently unsubstituted tert-butylene. In embodiments, L² is independently substituted or unsubstituted heteroalkylene (e.g., 2 to 8 membered, 2 to 6 membered, 4 to 6 membered, 2 to 3 membered, or 4 to 5 membered). In embodiments, L² is independently substituted heteroalkylene (e.g., 2 to 8 membered, 2 to 6 membered, 4 to 6 membered, 2 to 3 membered, or 4 to 5 membered). In embodiments, L² is independently unsubstituted heteroalkylene (e.g., 2 to 8 membered, 2 to 6 membered, 4 to 6 membered, 2 to 3 membered, or 4 to 5 membered). In embodiments, L² is independently substituted or unsubstituted cycloalkylene (e.g., C₃-C₈, C₃-C₆, C₄-C₆, or C₅-C₆). In embodiments, L² is independently substituted cycloalkylene (e.g., C₃-C₈, C₃-C₆, C₄-C₆, or C₅-C₆). In embodiments, L² is independently unsubstituted cycloalkylene (e.g., C₃-C₈, C₃-C₆, C₄-C₆, or C₅-C₆). In embodiments, L² is independently substituted or unsubstituted heterocycloalkylene (e.g., 3 to 8 membered, 3 to 6 membered, 4 to 6 membered, 4 to 5 membered, or 5 to 6 membered). In embodiments, L² is independently substituted heterocycloalkylene (e.g., 3 to 8 membered, 3 to 6 membered, 4 to 6 membered, 4 to 5 membered, or 5 to 6 membered). In embodiments, L² is independently unsubstituted heterocycloalkylene (e.g., 3 to 8 membered, 3 to 6 membered, 4 to 6 membered, 4 to 5 membered, or 5 to 6 membered). In embodiments, L² is independently substituted or unsubstituted arylene (e.g., C₆-C₁₀ or phenylene). In embodiments, L² is independently substituted arylene (e.g., C₆-C₁₀ or phenylene). In embodiments, L² is independently unsubstituted arylene (e.g., C₆-C₁₀ or phenylene). In embodiments, L² is independently substituted or unsubstituted heteroarylene (e.g., 5 to 10 membered, 5 to 9 membered, or 5 to 6 membered). In embodiments, L² is independently substituted heteroarylene (e.g., 5 to 10 membered, 5 to 9 membered, or 5 to 6 membered). In embodiments, L² is independently unsubstituted heteroarylene (e.g., 5 to 10 membered, 5 to 9 membered, or 5 to 6 membered).

In embodiments, L² is independently bond, —S(O)₂—, —N(R⁵)—, —O—, —S—, —C(O)—, —C(O)N(R⁵)—, —N(R⁵)C(O)—, —N(R⁵)C(O)NH—, —NHC(O) N(R⁵)—, —C(O)O—, —OC(O)—, R³⁸-substituted or unsubstituted alkylene (e.g., C₁-C₈, C₁-C₆, C₁-C₄, or C₁-C₂), R³⁸-substituted or unsubstituted heteroalkylene (e.g., 2 to 8 membered, 2 to 6 membered, 4 to 6 membered, 2 to 3 membered, or 4 to 5 membered), R³⁸-substituted or unsubstituted cycloalkylene (e.g., C₃-C₈, C₃-C₆, C₄-C₆, or C₅-C₆), R³⁸-substituted or unsubstituted heterocycloalkylene (e.g., 3 to 8 membered, 3 to 6 membered, 4 to 6 membered, 4 to 5 membered, or 5 to 6 membered), R³⁸-substituted or unsubstituted arylene (e.g., C₆-C₁₀ or phenylene), or R³⁸-substituted or unsubstituted heteroarylene (e.g., 5 to 10 membered, 5 to 9 membered, or 5 to 6 membered). In embodiments, L² is independently bond, —S(O)₂—, —N(R⁵)—, —O—, —S—, —C(O)—, —C(O)N(R⁵)—, —N(R⁵)C(O)—, —N(R⁵)C(O)NH—, —NHC(O) N(R⁵)—, —C(O)O—, —OC(O)—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, 2 to 3 membered, or 4 to 5 membered), unsubstituted cycloalkylene (e.g., C₃-C₈, C₃-C₆, C₄-C₆, or C₅-C₆), unsubstituted heterocycloalkylene (e.g., 3 to 8 membered, 3 to 6 membered, 4 to 6 membered, 4 to 5 membered, or 5 to 6 membered), unsubstituted arylene (e.g., C₆-C₁₀ or phenylene), or unsubstituted heteroarylene (e.g., 5 to 10 membered, 5 to 9 membered, or 5 to 6 membered). In embodiments, L² is independently unsubstituted methylene. In embodiments, L² is independently unsubstituted ethylene. In embodiments, L² is independently methyl-substituted methylene.

R³⁸ is independently oxo,

halogen, —CX³⁸₃, —CHX³⁸₂, —CH₂X³⁸, —OCX³⁸₃, —OCH₂X³⁸, —OCHX³⁸₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³⁹-substituted or unsubstituted alkyl (e.g., C₁-C₈, C₁-C₆, C₁-C₄, or C₁-C₂), R³⁹-substituted or unsubstituted heteroalkyl (e.g., 2 to 8 membered, 2 to 6 membered, 4 to 6 membered, 2 to 3 membered, or 4 to 5 membered), R³⁹-substituted or unsubstituted cycloalkyl (e.g., C₃-C₈, C₃-C₆, C₄-C₆, or C₅-C₆), R³⁹-substituted or unsubstituted heterocycloalkyl (e.g., 3 to 8 membered, 3 to 6 membered, 4 to 6 membered, 4 to 5 membered, or 5 to 6 membered), R³⁹-substituted or unsubstituted aryl (e.g., C₆-C₁₀ or phenyl), or R³⁹-substituted or unsubstituted heteroaryl (e.g., 5 to 10 membered, 5 to 9 membered, or 5 to 6 membered). In embodiments, R³⁸ is independently oxo,
halogen, —CX³⁸₃, —CHX³⁸₂, —CH₂X³⁸, —OCX³⁸₃, —OCH₂X³⁸, —OCHX³⁸₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁸ is independently —F, —Cl, —Br, or —I. In embodiments, R³⁸ is independently unsubstituted methyl. In embodiments, R³⁸ is independently unsubstituted ethyl.

R³⁹ is independently oxo,

halogen, —CX³⁹₃, —CHX³⁹₂, —CH₂X³⁹, —OCX³⁹₃, —OCH₂X³⁹, —OCHX³⁹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R⁴⁰-substituted or unsubstituted alkyl (e.g., C₁-C₈, C₁-C₆, C₁-C₄, or C₁-C₂), R⁴⁰-substituted or unsubstituted heteroalkyl (e.g., 2 to 8 membered, 2 to 6 membered, 4 to 6 membered, 2 to 3 membered, or 4 to 5 membered), R⁴⁰-substituted or unsubstituted cycloalkyl (e.g., C₃-C₈, C₃-C₆, C₄-C₆, or C₅-C₆), R⁴⁰-substituted or unsubstituted heterocycloalkyl (e.g., 3 to 8 membered, 3 to 6 membered, 4 to 6 membered, 4 to 5 membered, or 5 to 6 membered), R⁴⁰-substituted or unsubstituted aryl (e.g., C₆-C₁₀ or phenyl), or R⁴⁰-substituted or unsubstituted heteroaryl (e.g., 5 to 10 membered, 5 to 9 membered, or 5 to 6 membered). In embodiments, R³⁹ is independently oxo,
halogen, —CX³⁹₃, —CHX³⁹₂, —CH₂X³⁹, —OCX³⁹₃, —OCH₂X³⁹, —OCHX³⁹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁹ is independently —F, —Cl, —Br, or —I. In embodiments, R³⁹ is independently unsubstituted methyl. In embodiments, R³⁹ is independently unsubstituted ethyl.

R⁴⁰ is independently oxo,

halogen, —CX⁴⁰₃, —CHX⁴⁰₂, —CH₂X⁴⁰, —OCX⁴⁰₃, —OCH₂X⁴⁰, —OCHX⁴⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X⁴⁰ is independently —F, —Cl, —Br, or —I. In embodiments, R⁴⁰ is independently unsubstituted methyl. In embodiments, R⁴⁰ is independently unsubstituted ethyl.

In embodiments, R⁵ is hydrogen, substituted or unsubstituted C₁-C₆ alkyl, or substituted or unsubstituted 2 to 6 membered heteroalkyl. In embodiments, R⁵ is hydrogen or unsubstituted C₁-C₃ alkyl. In embodiments, R⁵ is hydrogen, unsubstituted methyl, unsubstituted ethyl, unsubstituted hexyl, or unsubstituted benzyl. In embodiments, R⁵ is hydrogen.

In embodiments, R⁵ is independently unsubstituted methyl. In embodiments, R⁵ is independently unsubstituted ethyl. In embodiments, R⁵ is independently unsubstituted propyl. In embodiments, R⁵ is independently unsubstituted isopropyl. In embodiments, R⁵ is independently unsubstituted n-propyl. In embodiments, R⁵ is independently unsubstituted butyl. In embodiments, R⁵ is independently unsubstituted n-butyl. In embodiments, R⁵ is independently unsubstituted t-butyl. In embodiments, R⁵ is independently unsubstituted pentyl. In embodiments, R⁵ is independently unsubstituted n-pentyl. In embodiments, R⁵ is independently unsubstituted hexyl. In embodiments, R⁵ is independently unsubstituted n-hexyl. In embodiments, R⁵ is independently unsubstituted heptyl. In embodiments, R⁵ is independently unsubstituted n-heptyl. In embodiments, R⁵ is independently unsubstituted octyl. In embodiments, R⁵ is independently unsubstituted n-octyl. In embodiments, R⁵ is independently unsubstituted benzyl. In embodiments, R⁵ is independently unsubstituted C₁-C₈ alkyl. In embodiments, R⁵ is independently halo-substituted methyl. In embodiments, R⁵ is independently halo-substituted ethyl. In embodiments, R⁵ is independently halo-substituted isopropyl. In embodiments, R⁵ is independently halo-substituted n-propyl. In embodiments, R⁵ is independently halo-substituted n-butyl. In embodiments, R⁵ is independently halo-substituted t-butyl. In embodiments, R¹ is independently halo-substituted n-pentyl. In embodiments, R⁵ is independently halo-substituted benzyl. In embodiments, R⁵ is independently halo-substituted C₁-C₈ alkyl. In embodiments, R⁵ is independently unsubstituted 2 to 6 membered heteroalkyl. In embodiments, R⁵ is independently unsubstituted 2 to 7 membered heteroalkyl. In embodiments, R⁵ is independently unsubstituted 2 to 8 membered heteroalkyl. In embodiments, R⁵ is independently unsubstituted 2 to 9 membered heteroalkyl. In embodiments, R⁵ is independently unsubstituted 2 to 10 membered heteroalkyl. In embodiments, R⁵ is independently unsubstituted 3 to 10 membered heteroalkyl. In embodiments, R⁵ is independently unsubstituted 4 to 10 membered heteroalkyl. In embodiments, R⁵ is independently unsubstituted 5 to 10 membered heteroalkyl. In embodiments, R⁵ is independently unsubstituted 6 to 10 membered heteroalkyl. In embodiments, R⁵ is independently unsubstituted 7 to 10 membered heteroalkyl. In embodiments, R⁵ is independently unsubstituted 8 to 10 membered heteroalkyl. In embodiments, R⁵ is independently unsubstituted 6 to 10 membered heteroalkyl. In embodiments, R⁵ is independently unsubstituted 7 to 9 membered heteroalkyl.

In embodiments, R⁵ is independently hydrogen, —CX⁵₃, —CHX⁵₂, —CH₂X⁵, —OCX⁵₃, —OCH₂X⁵, —OCHX⁵₂, —CN, —C(O)R^{5A}, —C(O)OR^{5A}, —C(O)NR^{5A}R^{5B}, —OR^{5A}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁵ is independently hydrogen. In embodiments, R⁵ is independently —CX⁵₃. In embodiments, R⁵ is independently —CHX⁵₂. In embodiments, R⁵ is independently —CH₂X⁵. In embodiments, R⁵ is independently —CN. In embodiments, R⁵ is independently —C(O)R^{5A}. In embodiments, R⁵ is independently —C(O)—OR^{5A}. In embodiments, R⁵ is independently —C(O)NR^{5A}R^{5B}. In embodiments, R⁵ is independently —COOH. In embodiments, R⁵ is independently —CONH₂. In embodiments, R⁵ is independently —CF₃. In embodiments, R⁵ is independently —CHF₂. In embodiments, R⁵ is independently —CH₂F. In embodiments, R⁵ is independently —CH₃. In embodiments, R⁵ is independently —CH₂CH₃. In embodiments, R⁵ is independently —CH₂CH₂CH₃. In embodiments, R⁵ is independently —CH(CH₃)₂. In embodiments, R⁵ is independently —C(CH₃)₃.

In embodiments, R⁵ is independently substituted or unsubstituted alkyl (e.g., C₁-C₈, C₁-C₆, C₁-C₄, or C₁-C₂). In embodiments, R⁵ is independently substituted alkyl (e.g., C₁-C₈, C₁-C₄, or C₁-C₂). In embodiments, R⁵ is independently unsubstituted alkyl (e.g., C₁-C₈, C₁-C₆, C₁-C₄, or C₁-C₂). In embodiments, R⁵ is independently unsubstituted methyl. In embodiments, R⁵ is independently unsubstituted ethyl. In embodiments, R⁵ is independently unsubstituted propyl. In embodiments, R⁵ is independently unsubstituted isopropyl. In embodiments, R⁵ is independently unsubstituted tert-butyl. In embodiments, R⁵ is independently substituted or unsubstituted heteroalkyl (e.g., 2 to 8 membered, 2 to 6 membered, 4 to 6 membered, 2 to 3 membered, or 4 to 5 membered). In embodiments, R⁵ is independently substituted heteroalkyl (e.g., 2 to 8 membered, 2 to 6 membered, 4 to 6 membered, 2 to 3 membered, or 4 to 5 membered). In embodiments, R⁵ is independently unsubstituted heteroalkyl (e.g., 2 to 8 membered, 2 to 6 membered, 4 to 6 membered, 2 to 3 membered, or 4 to 5 membered). In embodiments, R⁵ is independently substituted or unsubstituted cycloalkyl (e.g., C₃-C₈, C₃-C₆, C₄-C₆, or C₅-C₆). In embodiments, R⁵ is independently substituted cycloalkyl (e.g., C₃-C₈, C₃-C₆, C₄-C₆, or C₅-C₆). In embodiments, R⁵ is independently unsubstituted cycloalkyl (e.g., C₃-C₈, C₃-C₆, C₄-C₆, or C₅-C₆). In embodiments, R⁵ is independently substituted or unsubstituted heterocycloalkyl (e.g., 3 to 8 membered, 3 to 6 membered, 4 to 6 membered, 4 to 5 membered, or 5 to 6 membered). In embodiments, R⁵ is independently substituted heterocycloalkyl (e.g., 3 to 8 membered, 3 to 6 membered, 4 to 6 membered, 4 to 5 membered, or 5 to 6 membered). In embodiments, R⁵ is independently unsubstituted heterocycloalkyl (e.g., 3 to 8 membered, 3 to 6 membered, 4 to 6 membered, 4 to 5 membered, or 5 to 6 membered). In embodiments, R⁵ is independently substituted or unsubstituted aryl (e.g., C₆-C₁₀ or phenyl). In embodiments, R⁵ is independently substituted aryl (e.g., C₆-C₁₀ or phenyl). In embodiments, R⁵ is independently unsubstituted aryl (e.g., C₆-C₁₀ or phenyl). In embodiments, R⁵ is independently substituted or unsubstituted heteroaryl (e.g., 5 to 10 membered, 5 to 9 membered, or 5 to 6 membered). In embodiments, R⁵ is independently substituted heteroaryl (e.g., 5 to 10 membered, 5 to 9 membered, or 5 to 6 membered). In embodiments, R⁵ is independently unsubstituted heteroaryl (e.g., 5 to 10 membered, 5 to 9 membered, or 5 to 6 membered).

In embodiments, R^{5A} is independently hydrogen. In embodiments, R^{5A} is independently —CX^{5A}₃. In embodiments, R^{5A} is independently —CHX^{5A}₂. In embodiments, R^{5A} is independently —CH₂X^{5A}. In embodiments, R^{5A} is independently —CN. In embodiments, R^{5A} is independently —COOH. In embodiments, R^{5A} is independently —CONH₂. In embodiments, X^{5A} is independently —F, —Cl, —Br, or —I.

In embodiments, R^{5A} is independently substituted or unsubstituted alkyl (e.g., C₁-C₈, C₁-C₆, C₁-C₄, or C₁-C₂). In embodiments, R^{5A} is independently substituted alkyl (e.g., C₁-C₈, C₁-C₆, C₁-C₄, or C₁-C₂). In embodiments, R^{5A} is independently unsubstituted alkyl (e.g., C₁-C₈, C₁-C₆, C₁-C₄, or C₁-C₂). In embodiments, R^{5A} is independently unsubstituted methyl. In embodiments, R^{5A} is independently unsubstituted ethyl. In embodiments, R^{5A} is independently unsubstituted propyl. In embodiments, R^{5A} is independently unsubstituted isopropyl. In embodiments, R^{5A} is independently unsubstituted tert-butyl. In embodiments, R^{5A} is independently substituted or unsubstituted heteroalkyl (e.g., 2 to 8 membered, 2 to 6 membered, 4 to 6 membered, 2 to 3 membered, or 4 to 5 membered). In embodiments, R^{5A} is independently substituted heteroalkyl (e.g., 2 to 8 membered, 2 to 6 membered, 4 to 6 membered, 2 to 3 membered, or 4 to 5 membered). In embodiments, R^{5A} is independently unsubstituted heteroalkyl (e.g., 2 to 8 membered, 2 to 6 membered, 4 to 6 membered, 2 to 3 membered, or 4 to 5 membered). In embodiments, R^{5A} is independently substituted or unsubstituted cycloalkyl (e.g., C₃-C₈, C₃-C₆, C₄-C₆, or C₅-C₆). In embodiments, R^{5A} is independently substituted cycloalkyl (e.g., C₃-C₈, C₃-C₆, C₄-C₆, or C₅-C₆). In embodiments, R^{5A} is independently unsubstituted cycloalkyl (e.g., C₃-C₈, C₃-C₆, C₄-C₆, or C₅-C₆). In embodiments, R^{5A} is independently substituted or unsubstituted heterocycloalkyl (e.g., 3 to 8 membered, 3 to 6 membered, 4 to 6 membered, 4 to 5 membered, or 5 to 6 membered). In embodiments, R^{5A} is independently substituted heterocycloalkyl (e.g., 3 to 8 membered, 3 to 6 membered, 4 to 6 membered, 4 to 5 membered, or 5 to 6 membered). In embodiments, R^{5A} is independently unsubstituted heterocycloalkyl (e.g., 3 to 8 membered, 3 to 6 membered, 4 to 6 membered, 4 to 5 membered, or 5 to 6 membered). In embodiments, R^{5A} is independently substituted or unsubstituted aryl (e.g., C₆-C₁₀ or phenyl). In embodiments, R^{5A} is independently substituted aryl (e.g., C₆-C₁₀ or phenyl). In embodiments, R^{5A} is independently unsubstituted aryl (e.g., C₆-C₁₀ or phenyl). In embodiments, R^{5A} is independently substituted or unsubstituted heteroaryl (e.g., 5 to 10 membered, 5 to 9 membered, or 5 to 6 membered). In embodiments, R^{5A} is independently substituted heteroaryl (e.g., 5 to 10 membered, 5 to 9 membered, or 5 to 6 membered). In embodiments, R^{5A} is independently unsubstituted heteroaryl (e.g., 5 to 10 membered, 5 to 9 membered, or 5 to 6 membered).

In embodiments, R^{5B} is independently hydrogen. In embodiments, R^{5B} is independently —CX^{5B}₃. In embodiments, R^{5B} is independently —CHX^{5B}₂. In embodiments, R^{5B} is independently —CH₂X^{5B}. In embodiments, R^{5B} is independently —CN. In embodiments, R^{5B} is independently —COOH. In embodiments, R^{5B} is independently —CONH₂. In embodiments, X^{5B} is independently —F, —Cl, —Br, or —I.

In embodiments, R^{5B} is independently substituted or unsubstituted alkyl (e.g., C₁-C₈, C₁-C₆, C₁-C₄, or C₁-C₂). In embodiments, R^{5B} is independently substituted alkyl (e.g., C₁-C₈, C₁-C₆, C₁-C₄, or C₁-C₂). In embodiments, R^{5B} is independently unsubstituted alkyl (e.g., C₁-C₈, C₁-C₆, C₁-C₄, or C₁-C₂). In embodiments, R^{5B} is independently unsubstituted methyl. In embodiments, R^{5B} is independently unsubstituted ethyl. In embodiments, R^{5B} is independently unsubstituted propyl. In embodiments, R^{5B} is independently unsubstituted isopropyl. In embodiments, R^{5B} is independently unsubstituted tert-butyl. In embodiments, R^{5B} is independently substituted or unsubstituted heteroalkyl (e.g., 2 to 8 membered, 2 to 6 membered, 4 to 6 membered, 2 to 3 membered, or 4 to 5 membered). In embodiments, R^{5B} is independently substituted heteroalkyl (e.g., 2 to 8 membered, 2 to 6 membered, 4 to 6 membered, 2 to 3 membered, or 4 to 5 membered). In embodiments, R^{5B} is independently unsubstituted heteroalkyl (e.g., 2 to 8 membered, 2 to 6 membered, 4 to 6 membered, 2 to 3 membered, or 4 to 5 membered). In embodiments, R^{5B} is independently substituted or unsubstituted cycloalkyl (e.g., C₃-C₈, C₃-C₆, C₄-C₆, or C₅-C₆). In embodiments, R^{5B} is independently substituted cycloalkyl (e.g., C₃-C₈, C₃-C₆, C₄-C₆, or C₅-C₆). In embodiments, R^{5B} is independently unsubstituted cycloalkyl (e.g., C₃-C₈, C₃-C₆, C₄-C₆, or C₅-C₆). In embodiments, R^{5B} is independently substituted or unsubstituted heterocycloalkyl (e.g., 3 to 8 membered, 3 to 6 membered, 4 to 6 membered, 4 to 5 membered, or 5 to 6 membered). In embodiments, R^{5B} is independently substituted heterocycloalkyl (e.g., 3 to 8 membered, 3 to 6 membered, 4 to 6 membered, 4 to 5 membered, or 5 to 6 membered). In embodiments, R^{5B} is independently unsubstituted heterocycloalkyl (e.g., 3 to 8 membered, 3 to 6 membered, 4 to 6 membered, 4 to 5 membered, or 5 to 6 membered). In embodiments, R^{5B} is independently substituted or unsubstituted aryl (e.g., C₆-C₁₀ or phenyl). In embodiments, R^{5B} is independently substituted aryl (e.g., C₆-C₁₀ or phenyl). In embodiments, R^{5B} is independently unsubstituted aryl (e.g., C₆-C₁₀ or phenyl). In embodiments, R^{5B} is independently substituted or unsubstituted heteroaryl (e.g., 5 to 10 membered, 5 to 9 membered, or 5 to 6 membered). In embodiments, R^{5B} is independently substituted heteroaryl (e.g., 5 to 10 membered, 5 to 9 membered, or 5 to 6 membered). In embodiments, R^{5B} is independently unsubstituted heteroaryl (e.g., 5 to 10 membered, 5 to 9 membered, or 5 to 6 membered).

In embodiments, R^{5A} and R^{5B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{5A} and R^{5B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{5A} and R^{5B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{5A} and R^{5B} substituents bonded to the same nitrogen atom may be joined to form a substituted or unsubstituted heteroaryl (e.g., 5 to 10 membered, 5 to 9 membered, or 5 to 6 membered). In embodiments, R^{5A} and R^{5B} substituents bonded to the same nitrogen atom may be joined to form a substituted heteroaryl (e.g., 5 to 10 membered, 5 to 9 membered, or 5 to 6 membered). In embodiments, R^{5A} and R^{5B} substituents bonded to the same nitrogen atom may be joined to form an unsubstituted heteroaryl (e.g., 5 to 10 membered, 5 to 9 membered, or 5 to 6 membered).

In embodiments, R⁵ is independently hydrogen, —CX⁵₃, —CHX⁵₂, —CH₂X⁵, —CN, —COOH, —CONH₂, R³²-substituted or unsubstituted alkyl (e.g., C₁-C₈, C₁-C₆, C₁-C₄, or C₁-C₂), R³²-substituted or unsubstituted heteroalkyl (e.g., 2 to 8 membered, 2 to 6 membered, 4 to 6 membered, 2 to 3 membered, or 4 to 5 membered), R³²-substituted or unsubstituted cycloalkyl (e.g., C₃-C₈, C₃-C₆, C₄-C₆, or C₅-C₆), R³²-substituted or unsubstituted heterocycloalkyl (e.g., 3 to 8 membered, 3 to 6 membered, 4 to 6 membered, 4 to 5 membered, or 5 to 6 membered), R³²-substituted or unsubstituted aryl (e.g., C₆-C₁₀ or phenyl), or R³²-substituted or unsubstituted heteroaryl (e.g., 5 to 10 membered, 5 to 9 membered, or 5 to 6 membered). In embodiments, R⁵ is independently hydrogen, —CX⁵₃, —CHX⁵₂, —CH₂X⁵, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X⁵ is independently —F, —Cl, —Br, or —I. In embodiments, R⁵ is independently hydrogen. In embodiments, R⁵ is independently unsubstituted methyl. In embodiments, R⁵ is independently unsubstituted ethyl.

R³² is independently oxo,

halogen, —CX³²₃, —CHX³²₂, —CH₂X³², —OCX³²₃, —OCH₂X³², —OCHX³²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³³-substituted or unsubstituted alkyl (e.g., C₁-C₈, C₁-C₆, C₁-C₄, or C₁-C₂), R³³-substituted or unsubstituted heteroalkyl (e.g., 2 to 8 membered, 2 to 6 membered, 4 to 6 membered, 2 to 3 membered, or 4 to 5 membered), R³³-substituted or unsubstituted cycloalkyl (e.g., C₃-C₈, C₃-C₆, C₄-C₆, or C₅-C₆), R³³-substituted or unsubstituted heterocycloalkyl (e.g., 3 to 8 membered, 3 to 6 membered, 4 to 6 membered, 4 to 5 membered, or 5 to 6 membered), R³³-substituted or unsubstituted aryl (e.g., C₆-C₁₀ or phenyl), or R³³-substituted or unsubstituted heteroaryl (e.g., 5 to 10 membered, 5 to 9 membered, or 5 to 6 membered). In embodiments, R³² is independently oxo,
halogen, —CX³²₃, —CHX³²₂, —CH₂X³², —OCX³²₃, —OCH₂X³², —OCHX³²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³² is independently —F, —Cl, —Br, or —I. In embodiments, R³² is independently unsubstituted methyl. In embodiments, R³² is independently unsubstituted ethyl.

R³³ is independently oxo,

halogen, —CX³³₃, —CHX³³₂, —CH₂X³³, —OCX³³₃, —OCH₂X³³, —OCHX³³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³⁴-substituted or unsubstituted alkyl (e.g., C₁-C₈, C₁-C₆, C₁-C₄, or C₁-C₂), R³⁴-substituted or unsubstituted heteroalkyl (e.g., 2 to 8 membered, 2 to 6 membered, 4 to 6 membered, 2 to 3 membered, or 4 to 5 membered), R³⁴-substituted or unsubstituted cycloalkyl (e.g., C₃-C₈, C₃-C₆, C₄-C₆, or C₅-C₆), R³⁴-substituted or unsubstituted heterocycloalkyl (e.g., 3 to 8 membered, 3 to 6 membered, 4 to 6 membered, 4 to 5 membered, or 5 to 6 membered), R³⁴-substituted or unsubstituted aryl (e.g., C₆-C₁₀ or phenyl), or R³⁴-substituted or unsubstituted heteroaryl (e.g., 5 to 10 membered, 5 to 9 membered, or 5 to 6 membered). In embodiments, R³³ is independently oxo,
halogen, —CX³³₃, —CHX³³₂, —CH₂X³³, —OCX³³₃, —OCH₂X³³, —OCHX³³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³³ is independently —F, —Cl, —Br, or —I. In embodiments, R³³ is independently unsubstituted methyl. In embodiments, R³³ is independently unsubstituted ethyl.

R³⁴ is independently oxo,

halogen, —CX³⁴₃, —CHX³⁴₂, —CH₂X³⁴, —OCX³⁴₃, —OCH₂X³⁴, —OCHX³⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁴ is independently —F, —Cl, —Br, or —I. In embodiments, R³⁴ is independently unsubstituted methyl. In embodiments, R³⁴ is independently unsubstituted ethyl.

In embodiments, R^{5A} is independently hydrogen, —CX^{5A}₃, —CHX^{5A}₂, —CH₂X^{5A}, —CN, —COOH, —CONH₂, R^{32A}-substituted or unsubstituted alkyl (e.g., C₁-C₈, C₁-C₆, C₁-C₄, or C₁-C₂), R^{32A}-substituted or unsubstituted heteroalkyl (e.g., 2 to 8 membered, 2 to 6 membered, 4 to 6 membered, 2 to 3 membered, or 4 to 5 membered), R^{32A}-substituted or unsubstituted cycloalkyl (e.g., C₃-C₈, C₃-C₆, C₄-C₆, or C₅-C₆), R^{32A}-substituted or unsubstituted heterocycloalkyl (e.g., 3 to 8 membered, 3 to 6 membered, 4 to 6 membered, 4 to 5 membered, or 5 to 6 membered), R^{32A}-substituted or unsubstituted aryl (e.g., C₆-C₁₀ or phenyl), or R^{32A}-substituted or unsubstituted heteroaryl (e.g., 5 to 10 membered, 5 to 9 membered, or 5 to 6 membered). In embodiments, R^{5A} is independently hydrogen, —CX^{5A}₃, —CHX^{5A}₂, —CH₂X^{5A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{5A} is independently —F, —Cl, —Br, or —I. In embodiments, R^{5A} is independently hydrogen. In embodiments, R^{5A} is independently unsubstituted methyl. In embodiments, R^{5A} is independently unsubstituted ethyl.

In embodiments, R^{5A} and R^{5B} substituents bonded to the same nitrogen atom may optionally be joined to form a R^{32A}-substituted or unsubstituted heterocycloalkyl (e.g., 3 to 8 membered, 3 to 6 membered, 4 to 6 membered, 4 to 5 membered, or 5 to 6 membered) or R^{32A}-substituted or unsubstituted heteroaryl (e.g., 5 to 10 membered, 5 to 9 membered, or 5 to 6 membered). In embodiments, R^{5A} and R^{5B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{5A} and R^{5B} substituents bonded to the same nitrogen atom may optionally be joined to form a R^{32A}-substituted or unsubstituted heterocycloalkyl (e.g., 3 to 8 membered, 3 to 6 membered, 4 to 6 membered, 4 to 5 membered, or 5 to 6 membered). In embodiments, R^{5A} and R^{5B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{32A} is independently oxo,

halogen, —CX^{32A}₃, —CHX^{32A}₂, —CH₂X^{32A}, —OCX^{32A}₃, —OCH₂X^{32A}, —OCHX^{32A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{33A}-substituted or unsubstituted alkyl (e.g., C₁-C₈, C₁-C₆, C₁-C₄, or C₁-C₂), R^{33A}-substituted or unsubstituted heteroalkyl (e.g., 2 to 8 membered, 2 to 6 membered, 4 to 6 membered, 2 to 3 membered, or 4 to 5 membered), R^{33A}-substituted or unsubstituted cycloalkyl (e.g., C₃-C₈, C₃-C₆, C₄-C₆, or C₅-C₆), R^{33A}-substituted or unsubstituted heterocycloalkyl (e.g., 3 to 8 membered, 3 to 6 membered, 4 to 6 membered, 4 to 5 membered, or 5 to 6 membered), R^{33A}-substituted or unsubstituted aryl (e.g., C₆-C₁₀ or phenyl), or R^{33A}-substituted or unsubstituted heteroaryl (e.g., 5 to 10 membered, 5 to 9 membered, or 5 to 6 membered). In embodiments, R^{32A} is independently oxo,
halogen, —CX^{32A}₃, —CHX^{32A}₂, —CH₂X^{32A}, —OCX^{32A}₃, —OCH₂X^{32A}, —OCHX^{32A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{32A} is independently —F, —Cl, —Br, or —I. In embodiments, R^{32A} is independently unsubstituted methyl. In embodiments, R^{32A} is independently unsubstituted ethyl.

R^{33A} is independently oxo,

halogen, —CX^{33A}₃, —CHX^{33A}₂, —CH₂X^{33A}, —OCX^{33A}₃, —OCH₂X^{33A}, —OCHX^{33A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{34A}-substituted or unsubstituted alkyl (e.g., C₁-C₈, C₁-C₆, C₁-C₄, or C₁-C₂), R^{34A}-substituted or unsubstituted heteroalkyl (e.g., 2 to 8 membered, 2 to 6 membered, 4 to 6 membered, 2 to 3 membered, or 4 to 5 membered), R^{34A}-substituted or unsubstituted cycloalkyl (e.g., C₃-C₈, C₃-C₆, C₄-C₆, or C₅-C₆), R^{34A}-substituted or unsubstituted heterocycloalkyl (e.g., 3 to 8 membered, 3 to 6 membered, 4 to 6 membered, 4 to 5 membered, or 5 to 6 membered), R^{34A}-substituted or unsubstituted aryl (e.g., C₆-C₁₀ or phenyl), or R^{34A}-substituted or unsubstituted heteroaryl (e.g., 5 to 10 membered, 5 to 9 membered, or 5 to 6 membered). In embodiments, R^{33A} is independently oxo,
halogen, —CX^{33A}₃, —CHX^{33A}₂, —CH₂X^{33A}, —OCX^{33A}₃, —OCH₂X^{33A}, —OCHX^{33A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{33A} is independently —F, —Cl, —Br, or —I. In embodiments, R^{33A} is independently unsubstituted methyl. In embodiments, R^{33A} is independently unsubstituted ethyl.

R^{34A} is independently oxo,

halogen, —CX^{34A}₃, —CHX^{34A}₂, —CH₂X^{34A}, —OCX^{34A}₃, —OCH₂X^{34A}, —OCHX^{34A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{34A} is independently —F, —Cl, —Br, or —I. In embodiments, R^{34A} is independently unsubstituted methyl. In embodiments, R^{34A} is independently unsubstituted ethyl.

In embodiments, R^{5B} is independently hydrogen, —CX^{5B}₃, —CHX^{5B}₂, —CH₂X^{5B}, —CN, —COOH, —CONH₂, R^{32B}-substituted or unsubstituted alkyl (e.g., C₁-C₈, C₁-C₆, C₁-C₄, or C₁-C₂), R^{32B}-substituted or unsubstituted heteroalkyl (e.g., 2 to 8 membered, 2 to 6 membered, 4 to 6 membered, 2 to 3 membered, or 4 to 5 membered), R^{32B}-substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), R^{32B}-substituted or unsubstituted aryl (e.g., C₆-C₁₀ or phenyl), or R^{32B}-substituted or unsubstituted heteroaryl (e.g., 5 to 10 membered, 5 to 9 membered, or 5 to 6 membered). In embodiments, R^{5B} is independently hydrogen, —CX^{5B}₃, —CHX^{5B}₂, —CH₂X^{5B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{5B} is independently —F, —Cl, —Br, or —I. In embodiments, R^{5B} is independently hydrogen. In embodiments, R^{5B} is independently unsubstituted methyl. In embodiments, R^{5B} is independently unsubstituted ethyl.

In embodiments, R^{5A} and R^{5B} substituents bonded to the same nitrogen atom may optionally be joined to form a R^{32B}-substituted or unsubstituted heterocycloalkyl (e.g., 3 to 8 membered, 3 to 6 membered, 4 to 6 membered, 4 to 5 membered, or 5 to 6 membered) or R^{32B}-substituted or unsubstituted heteroaryl (e.g., 5 to 10 membered, 5 to 9 membered, or 5 to 6 membered). In embodiments, R^{5A} and R^{5B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{5A} and R^{5B} substituents bonded to the same nitrogen atom may optionally be joined to form a R^{32B}-substituted or unsubstituted heterocycloalkyl (e.g., 3 to 8 membered, 3 to 6 membered, 4 to 6 membered, 4 to 5 membered, or 5 to 6 membered). In embodiments, R^{5A} and R^{5B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{32B} is independently oxo,

halogen, —CX^{32B}₃, —CHX^{32B}₂, —CH₂X^{32B}, —OCX^{32B}₃, —OCH₂X^{32B}, —OCHX^{32B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{33B}-substituted or unsubstituted alkyl (e.g., C₁-C₈, C₁-C₆, C₁-C₄, or C₁-C₂), R^{33B}-substituted or unsubstituted heteroalkyl (e.g., 2 to 8 membered, 2 to 6 membered, 4 to 6 membered, 2 to 3 membered, or 4 to 5 membered), R^{33B}-substituted or unsubstituted cycloalkyl (e.g., C₃-C₈, C₃-C₆, C₄-C₆, or C₅-C₆), R^{33B}-substituted or unsubstituted heterocycloalkyl (e.g., 3 to 8 membered, 3 to 6 membered, 4 to 6 membered, 4 to 5 membered, or 5 to 6 membered), R^{33B}-substituted or unsubstituted aryl (e.g., C₆-C₁₀ or phenyl), or R^{33B}-substituted or unsubstituted heteroaryl (e.g., 5 to 10 membered, 5 to 9 membered, or 5 to 6 membered). In embodiments, R^{32B} is independently oxo,
halogen, —CX^{32B}₃, —CHX^{32B}₂, —CH₂X^{32B}, —OCX^{32B}₃, —OCH₂X^{32B}, —OCHX^{32B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{32B} is independently —F, —Cl, —Br, or —I. In embodiments, R^{32B} is independently unsubstituted methyl. In embodiments, R^{32B} is independently unsubstituted ethyl.

R^{33B} is independently oxo,

halogen, —CX^{33B}₃, —CHX^{33B}₂, —CH₂X^{33B}, —OCX^{33B}₃, —OCH₂X^{33B}, —OCHX^{33B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{34B}-substituted or unsubstituted alkyl (e.g., C₁-C₈, C₁-C₆, C₁-C₄, or C₁-C₂), R^{34B}-substituted or unsubstituted heteroalkyl (e.g., 2 to 8 membered, 2 to 6 membered, 4 to 6 membered, 2 to 3 membered, or 4 to 5 membered), R^{34B}-substituted or unsubstituted cycloalkyl (e.g., C₃-C₈, C₃-C₆, C₄-C₆, or C₅-C₆), R^{34B}-substituted or unsubstituted heterocycloalkyl (e.g., 3 to 8 membered, 3 to 6 membered, 4 to 6 membered, 4 to 5 membered, or 5 to 6 membered), R^{34B}-substituted or unsubstituted aryl (e.g., C₆-C₁₀ or phenyl), or R^{34B}-substituted or unsubstituted heteroaryl (e.g., 5 to 10 membered, 5 to 9 membered, or 5 to 6 membered). In embodiments, R^{33B} is independently oxo,
halogen, —CX^{33B}₃, —CHX^{33B}₂, —CH₂X^{33B}, —OCX^{33B}₃, —OCH₂X^{33B}, —OCHX^{33B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{33B} is independently —F, —Cl, —Br, or —I. In embodiments, R^{33B} is independently unsubstituted methyl. In embodiments, R^{33B} is independently unsubstituted ethyl.

R^{34B} is independently oxo,

halogen, —CX^{34B}₃, —CHX^{34B}₂, —CH₂X^{34B}, —OCX^{34B}₃, —OCH₂X^{34B}, —OCHX^{34B}₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{34B} is independently —F, —Cl, —Br, or —I. In embodiments, R^{34B} is independently unsubstituted methyl. In embodiments, R^{34B} is independently unsubstituted ethyl.

In embodiments, X is —F. In embodiments, X is —Cl. In embodiments, X is —Br. In embodiments, X is —I. In embodiments, X¹ is —F. In embodiments, X¹ is —Cl. In embodiments, X¹ is —Br. In embodiments, X¹ is —I. In embodiments, X² is —F. In embodiments, X² is —Cl. In embodiments, X² is —Br. In embodiments, X² is —I. In embodiments, X⁴ is —F. In embodiments, X⁴ is —Cl. In embodiments, X⁴ is —Br. In embodiments, X⁴ is —I. In embodiments, X⁵ is —F. In embodiments, X⁵ is —Cl. In embodiments, X⁵ is —Br. In embodiments, X⁵ is —I.

In embodiments, n1 is 0. In embodiments, n1 is 1. In embodiments, n1 is 2. In embodiments, n1 is 3. In embodiments, n1 is 4. In embodiments, n2 is 0. In embodiments, n2 is 1. In embodiments, n2 is 2. In embodiments, n2 is 3. In embodiments, n2 is 4. In embodiments, n4 is 0. In embodiments, n4 is 1. In embodiments, n4 is 2. In embodiments, n4 is 3. In embodiments, n4 is 4. In embodiments, n5 is 0. In embodiments, n5 is 1. In embodiments, n5 is 2. In embodiments, n5 is 3. In embodiments, n5 is 4.

In embodiments, m1 is 1. In embodiments, m1 is 2. In embodiments, m2 is 1. In embodiments, m2 is 2. In embodiments, m4 is 1. In embodiments, m4 is 2. In embodiments, m5 is 1. In embodiments, m5 is 2.

In embodiments, v1 is 1. In embodiments, v1 is 2. In embodiments, v2 is 1. In embodiments, v2 is 2. In embodiments, v4 is 1. In embodiments, v4 is 2. In embodiments, v5 is 1. In embodiments, v5 is 2.

In embodiments, E is a covalent cysteine modifier moiety.

In embodiments, E is:

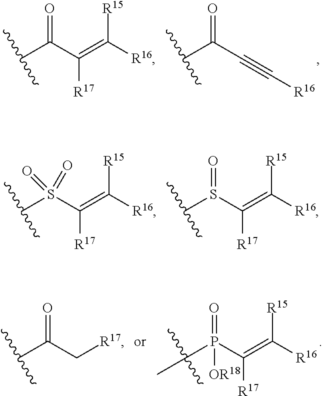

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

Each R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, and R^{18D}, is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl. Each X, X¹⁵, X¹⁶, X¹⁷ and X¹⁸ is independently —F, —Cl, —Br, or —I. The symbols n15, n16, n17, v15, v16, and v17, are independently and integer from 0 to 4. The symbols m15, m16, and m17 are independently and integer between 1 and 2.

In embodiments, E is:

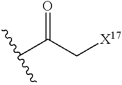

and X¹⁷ is —Cl. In embodiments, E is:

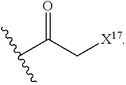

In embodiments, X¹⁷ is —Cl.

In embodiments, E is:

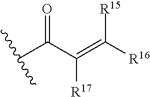

and R¹⁵, R¹⁶, and R¹⁷ are independently hydrogen. In embodiments, E is:

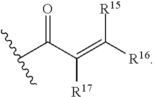

In embodiments, R¹⁵, R¹⁶, and R¹⁷ are independently hydrogen.

In embodiments, E is:

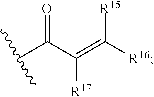

R¹⁵ is independently hydrogen; R¹⁶ is independently hydrogen or CH₂NR^{16A}R^{16B}; R¹⁷ is independently hydrogen; and R^{16A} and R^{16B} are independently hydrogen or unsubstituted alkyl. In embodiments, E is:

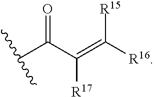

In embodiments, R¹⁵ is independently hydrogen. In embodiments, R¹⁶ is independently hydrogen or CH₂NR^{16A}R^{16B}. In embodiments, R¹⁷ is independently hydrogen. In embodiments, R^{16A} and R¹⁶ are independently hydrogen or unsubstituted alkyl. In embodiments, R^{16A} and R^{16B} are independently unsubstituted methyl.

In embodiments, E is:

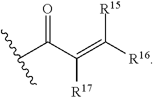

In embodiments, E is:

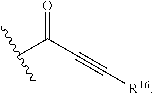

In embodiments, E is:

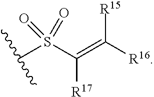

In embodiments, E is:

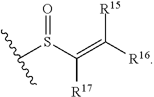

In embodiments, E is:

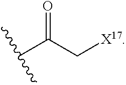

In embodiments, E is:

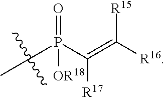

In embodiments, E is:

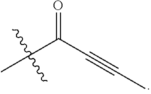 [SMILES: *C(=O)C#CC]

In embodiments, E is:

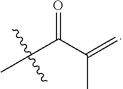 [SMILES: *C(=O)C(=C)C]

In embodiments, E is:

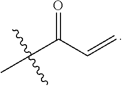 [SMILES: *C(=O)C=C]

In embodiments, E is:

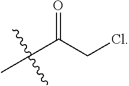 [SMILES: *C(=O)CCl]

X may independently be F. X may independently be —Cl. X may independently be —Br. X may independently be —I. X¹⁵ may independently be —F. X¹⁵ may independently be —Cl. X¹⁵ may independently be —Br. X¹⁵ may independently be —I. X¹⁶ may independently be —F. X¹⁶ may independently be —Cl. X¹⁶ may independently be —Br. X¹⁶ may independently be —I. X¹⁷ may independently be —F. X¹⁷ may independently be —Cl. X¹⁷ may independently be —Br. X¹⁷ may independently be —I. X¹⁸ may independently be —F. X¹⁸ may independently be —Cl. X¹⁸ may independently be —Br. X¹⁸ may independently be —I. n15 may independently be 0. n15 may independently be 1. n15 may independently be 2. n15 may independently be 3. n15 may independently be 4. n16 may independently be 0. n16 may independently be 1. n16 may independently be 2. n16 may independently be 3. n16 may independently be 4. n17 may independently be 0. n17 may independently be 1. n17 may independently be 2. n17 may independently be 3. n17 may independently be 4. v15 may independently be 0. v15 may independently be 1. v15 may independently be 2. v15 may independently be 3. v15 may independently be 4. v16 may independently be 0. v16 may independently be 1. v16 may independently be 2. v16 may independently be 3. v16 may independently be 4. v17 may independently be 0. v17 may independently be 1. v17 may independently be 2. v17 may independently be 3. v17 may independently be 4. m15 may independently be 1. m15 may independently be 2. m16 may independently be 1. m16 may independently be 2. m17 may independently be 1. m17 may independently be 2.

In embodiments, R¹⁵ is hydrogen. In embodiments, R¹⁵ is halogen. In embodiments, R¹⁵ is CX¹⁵₃. In embodiments, R¹⁵ is —CHX¹⁵₂. In embodiments, R¹⁵ is —CH₂X¹⁵. In embodiments, R¹⁵ is —CN. In embodiments, R¹⁵ is —SOₙ₁₅R^{15D}. In embodiments, R¹⁵ is —SOᵥ₁₅NR^{15A}R^{15B}. In embodiments, R¹⁵ is —NHNR^{15A}R^{15B}. In embodiments, R¹⁵ is —ONR^{15A}R^{15B}. In embodiments, R¹⁵ is —NHC═(O)NHNR^{15A}R^{15B}. In embodiments, R¹⁵ is —NHC(O)NR^{15A}R^{15B}. In embodiments, R¹⁵ is —N(O)ₘ₁₅. In embodiments, R¹⁵ is —NR^{15A}R^{15B}. In embodiments, R¹⁵ is —C(O)R^{15C}. In embodiments, R¹⁵ is —C(O)—OR^{15C}. In embodiments, R¹⁵ is —C(O)NR^{15A}R^{15B}. In embodiments, R¹⁵ is —OR^{15D}. In embodiments, R¹⁵ is —NR^{15A}SO₂R^{15D}. In embodiments, R¹⁵ is —NR^{15A}C(O)R^{15C}. In embodiments, R¹⁵ is —NR^{15A}C(O)OR^{15C}. In embodiments, R¹⁵ is —NR^{15A}OR^{15C}. In embodiments, R¹⁵ is —OCX¹⁵₃. In embodiments, R¹⁵ is —OCHX¹⁵₂. In embodiments, R¹⁵ is substituted or unsubstituted alkyl. In embodiments, R¹⁵ is substituted or unsubstituted heteroalkyl. In embodiments, R¹⁵ is substituted or unsubstituted cycloalkyl. In embodiments, R¹⁵ is substituted or unsubstituted heterocycloalkyl. In embodiments, R¹⁵ is substituted or unsubstituted aryl. In embodiments, R¹⁵ is substituted or unsubstituted heteroaryl. In embodiments, R¹⁵ is substituted alkyl. In embodiments, R¹⁵ is substituted heteroalkyl. In embodiments, R¹⁵ is substituted cycloalkyl. In embodiments, R¹⁵ is substituted heterocycloalkyl. In embodiments, R¹⁵ is substituted aryl. In embodiments, R¹⁵ is substituted heteroaryl. In embodiments, R¹⁵ is unsubstituted alkyl. In embodiments, R¹⁵ is unsubstituted heteroalkyl. In embodiments, R¹⁵ is unsubstituted cycloalkyl. In embodiments, R¹⁵ is unsubstituted heterocycloalkyl. In embodiments, R¹⁵ is unsubstituted aryl. In embodiments, R¹⁵ is unsubstituted heteroaryl. In embodiments, R¹⁵ is unsubstituted methyl. In embodiments, R¹⁵ is unsubstituted ethyl. In embodiments, R¹⁵ is unsubstituted propyl. In embodiments, R¹⁵ is unsubstituted isopropyl. In embodiments, R¹⁵ is unsubstituted butyl. In embodiments, R¹⁵ is unsubstituted tert-butyl.

In embodiments, R^{15A} is hydrogen. In embodiments, R^{15A} is —CX₃. In embodiments, R^{15A} is —CN. In embodiments, R^{15A} is —COOH. In embodiments, R^{15A} is —CONH₂. In embodiments, R^{15A} is —CHX₂. In embodiments, R^{15A} is —CH₂X. In embodiments, R^{15A} is unsubstituted methyl. In embodiments, R^{15A} is unsubstituted ethyl. In embodiments, R^{15A} is unsubstituted propyl. In embodiments, R^{15A} is unsubstituted isopropyl. In embodiments, R^{15A} is unsubstituted butyl. In embodiments, R^{15A} is unsubstituted tert-butyl.

In embodiments, R^{15B} is hydrogen. In embodiments, R^{15B} is —CX₃. In embodiments, R^{15B} is —CN. In embodiments, R^{15B} is —COOH. In embodiments, R^{15B} is —CONH₂. In embodiments, R^{15B} is —CHX₂. In embodiments, R^{15B} is —CH₂X. In embodiments, R^{15B} is unsubstituted methyl. In embodiments, R^{15B} is unsubstituted ethyl. In embodiments, R^{15B} is unsubstituted propyl. In embodiments, R^{15B} is unsubstituted isopropyl. In embodiments, R^{15B} is unsubstituted butyl. In embodiments, R^{15B} is unsubstituted tert-butyl.

In embodiments, R^{15C} is hydrogen. In embodiments, R^{15C} is —CX₃. In embodiments, R^{15C} is —CN. In embodiments, R^{15C} is —COOH. In embodiments, R^{15C} is —CONH₂. In embodiments, R^{15C} is —CHX₂. In embodiments, R^{15C} is —CH₂X. In embodiments, R^{15C} is unsubstituted methyl. In embodiments, R^{15C} is unsubstituted ethyl. In embodiments, R^{15C} is unsubstituted propyl. In embodiments, R^{15C} is unsubstituted isopropyl. In embodiments, R^{15C} is unsubstituted butyl. In embodiments, R^{15C} is unsubstituted tert-butyl.

In embodiments, R^{15D} is hydrogen. In embodiments, R^{15D} is —CX₃. In embodiments, R^{15D} is —CN. In embodiments, R^{15D} is —COOH. In embodiments, R^{15D} is —CONH₂. In embodiments, R^{15D} is —CHX₂. In embodiments, R^{15D} is —CH₂X. In embodiments, R^{15D} is unsubstituted methyl. In embodiments, R^{15D} is unsubstituted ethyl. In embodiments, R^{15D} is unsubstituted propyl. In embodiments, R^{15D} is unsubstituted isopropyl. In embodiments, R^{15D} is unsubstituted butyl. In embodiments, R^{15D} is unsubstituted tert-butyl.

In embodiments, R¹⁵ is independently hydrogen, oxo,

halogen, —CX¹⁵₃, —CHX¹⁵₂, —OCH₂X¹⁵, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁵₃, —OCHX¹⁵₂, R⁷²-substituted or unsubstituted alkyl, R⁷²-substituted or unsubstituted heteroalkyl, R⁷²-substituted or unsubstituted cycloalkyl, R⁷²-substituted or unsubstituted heterocycloalkyl, R⁷²-substituted or unsubstituted aryl, or R⁷²-substituted or unsubstituted heteroaryl. X¹⁵ is halogen. In embodiments, X¹⁵ is F.

R⁷² is independently oxo,

halogen, —CX⁷²₃, —CHX⁷²₂, —OCH₂X⁷², —OCHX⁷²₂, —CN, —OH, —NH₂, —COOH, —CONH₂, —NO₂, —S H, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷²₃, —OCHX⁷²₂, R⁷³-substituted or unsubstituted alkyl, R⁷³-substituted or unsubstituted heteroalkyl, R⁷³-substituted or unsubstituted cycloalkyl, R⁷³-substituted or unsubstituted heterocycloalkyl, R⁷³-substituted or unsubstituted aryl, or R⁷³-substituted or unsubstituted heteroaryl. X⁷² is halogen. In embodiments, X⁷² is F.

R⁷³ is independently oxo,

halogen, —CX⁷³₃, —CHX⁷³₂, —OCH₂X⁷³, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷³₃, —OCHX⁷³₂, R⁷⁴-substituted or unsubstituted alkyl, R⁷⁴-substituted or unsubstituted heteroalkyl, R⁷⁴-substituted or unsubstituted cycloalkyl, R⁷⁴-substituted or unsubstituted heterocycloalkyl, R⁷⁴-substituted or unsubstituted aryl, or R⁷⁴-substituted or unsubstituted heteroaryl. X⁷³ is halogen. In embodiments, X⁷³ is F.

In embodiments, R^{15A} is independently hydrogen, oxo,

halogen, —CX^{15A}₃, —CHX^{15A}₂, —OCH₂X^{15A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{15A}₃, —OCHX^{15A}₂, R^{72A}-substituted or unsubstituted alkyl, R^{72A}-substituted or unsubstituted heteroalkyl, R^{72A}-substituted or unsubstituted cycloalkyl, R^{72A}-substituted or unsubstituted heterocycloalkyl, R^{72A}-substituted or unsubstituted aryl, or R^{72A}-substituted or unsubstituted heteroaryl. X^{15A} is halogen. In embodiments, X^{15A} is F.

R^{72A} is independently oxo,

halogen, —CX^{72A}₃, —CHX^{72A}₂, —OCH₂X^{72A}, —OCHX^{72A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{72A}₃, —OCHX^{72A}₂, R^{73A}-substituted or unsubstituted alkyl, R^{73A}-substituted or unsubstituted heteroalkyl, R^{73A}-substituted or unsubstituted cycloalkyl, R^{73A}-substituted or unsubstituted heterocycloalkyl, R^{73A}-substituted or unsubstituted aryl, or R^{73A}-substituted or unsubstituted heteroaryl. X^{72A} is halogen. In embodiments, X^{72A} is F.

R^{73A} is independently oxo,

halogen, —CX^{73A}₃, —CHX^{73A}₂, —OCH₂X^{73A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{73A}₃, —OCHX^{73A}₂, R^{74A}-substituted or unsubstituted alkyl, R^{74A}-substituted or unsubstituted heteroalkyl, R^{74A}-substituted or unsubstituted cycloalkyl, R^{74A}-substituted or unsubstituted heterocycloalkyl, R^{74A}-substituted or unsubstituted aryl, or R^{74A}-substituted or unsubstituted heteroaryl. X^{73A} is halogen. In embodiments, X^{73A} is F.

In embodiments, R^{15B} is independently hydrogen, oxo,

halogen, —CX^{15B}₃, —CHX^{15B}₂, —OCH₂X^{15B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{15B}₃, —OCHX^{15B}₂, R^{72B}-substituted or unsubstituted alkyl, R^{72B}-substituted or unsubstituted heteroalkyl, R^{72B}-substituted or unsubstituted cycloalkyl, R^{72B}-substituted or unsubstituted heterocycloalkyl, R^{72B}-substituted or unsubstituted aryl, or R^{72B}-substituted or unsubstituted heteroaryl. X^{15B} is halogen. In embodiments, X^{15B} is F.

R^{72B} is independently oxo,

halogen, —CX^{72B}₃, —CHX^{72B}₂, —OCH₂X^{72B}, —OCHX^{72B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{72B}₃, —OCHX^{72B}₂, R^{73B}-substituted or unsubstituted alkyl, R^{73B}-substituted or unsubstituted heteroalkyl, R^{73B}-substituted or unsubstituted cycloalkyl, R^{73B}-substituted or unsubstituted heterocycloalkyl, R^{73B}-substituted or unsubstituted aryl, or R^{73B}-substituted or unsubstituted heteroaryl. X^{72B} is halogen. In embodiments, X^{72B} is F.

R^{73B} is independently oxo,

halogen, —CX^{73B}₃, —CHX^{73B}₂, —OCH₂X^{73B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{73B}₃, —OCHX^{73B}₂, R^{74B}-substituted or unsubstituted alkyl, R^{74B}-substituted or unsubstituted heteroalkyl, R^{74B}-substituted or unsubstituted cycloalkyl, R^{74B}-substituted or unsubstituted heterocycloalkyl, R^{74B}-substituted or unsubstituted aryl, or R^{74B}-substituted or unsubstituted heteroaryl. X^{73B} is halogen. In embodiments, X^{73B} is F.

In embodiments, R^{15C} is independently hydrogen, oxo,

halogen, —CX^{15C}₃, —CHX^{15C}₂, —OCH₂X^{15C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{15C}₃, —OCHX^{15C}₂, R^{72C}-substituted or unsubstituted alkyl, R^{72C}-substituted or unsubstituted heteroalkyl, R^{72C}-substituted or unsubstituted cycloalkyl, R^{72C}-substituted or unsubstituted heterocycloalkyl, R^{72C}-substituted or unsubstituted aryl, or R^{72C}-substituted or unsubstituted heteroaryl. X^{15C} is halogen. In embodiments, X^{15C} is F.

R^{72C} is independently oxo,

halogen, —CX^{72C}₃, —CHX^{72C}₂, —OCH₂X^{72C}, —OCHX^{72C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{72C}₃, —OCHX^{72C}₂, R^{73C}-substituted or unsubstituted alkyl, R^{73C}-substituted or unsubstituted heteroalkyl, R^{73C}-substituted or unsubstituted cycloalkyl, R^{73C}-substituted or unsubstituted heterocycloalkyl, R^{73C}-substituted or unsubstituted aryl, or R^{73C}-substituted or unsubstituted heteroaryl. X^{72C} is halogen. In embodiments, X^{72C} is F.

R^{73C} is independently oxo,

halogen, —CX^{73C}₃, —CHX^{73C}₂, —OCH₂X^{73C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{73C}₃, —OCHX^{73C}₂, R^{74C}-substituted or unsubstituted alkyl, R^{74C}-substituted or unsubstituted heteroalkyl, R^{74C}-substituted or unsubstituted cycloalkyl, R^{74C}-substituted or unsubstituted heterocycloalkyl, R^{74C}-substituted or unsubstituted aryl, or R^{74C}-substituted or unsubstituted heteroaryl. X^{73C} is halogen. In embodiments, X^{73C} is F.

In embodiments, R^{15D} is independently hydrogen, oxo,

halogen, —CX^{15D}₃, —CHX^{15D}₂, —OCH₂X^{15D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{15D}₃, —OCHX^{15D}₂, R^{72D}-substituted or unsubstituted alkyl, R^{72D}-substituted or unsubstituted heteroalkyl, R^{72D}-substituted or unsubstituted cycloalkyl, R^{72D}-substituted or unsubstituted heterocycloalkyl, R^{72D}-substituted or unsubstituted aryl, or R^{72D}-substituted or unsubstituted heteroaryl. X^{15D} is halogen. In embodiments, X^{15D} is F.

R^{72D} is independently oxo,

halogen, —CX^{72D}₃, —CHX^{72D}₂, —OCH₂X^{72D}, —OCHX^{72D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{72D}₃, —OCHX^{72D}₂, R^{73D}-substituted or unsubstituted alkyl, R^{73D}-substituted or unsubstituted heteroalkyl, R^{73D}-substituted or unsubstituted cycloalkyl, R^{73D}-substituted or unsubstituted heterocycloalkyl, R^{73D}-substituted or unsubstituted aryl, or R^{73D}-substituted or unsubstituted heteroaryl. X^{72D} is halogen. In embodiments, X^{72D} is F.

R^{73D} is independently oxo,

halogen, —CX^{73D}₃, —CHX^{73D}₂, —OCH₂X^{73D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{73D}₃, —OCHX^{73D}₂, R^{74D}-substituted or unsubstituted alkyl, R^{74D}-substituted or unsubstituted heteroalkyl, R^{74D}-substituted or unsubstituted cycloalkyl, R^{74D}-substituted or unsubstituted heterocycloalkyl, R^{74D}-substituted or unsubstituted aryl, or R^{74D}-substituted or unsubstituted heteroaryl. X^{73D} is halogen. In embodiments, X^{73D} is F.

In embodiments, R¹⁶ is hydrogen. In embodiments, R¹⁶ is halogen. In embodiments, R¹⁶ is —CX¹⁶₃. In embodiments, R¹⁶ is —CHX¹⁶₂. In embodiments, R¹⁶ is —CH₂X¹⁶. In embodiments, R¹⁶ is —CN. In embodiments, R¹⁶ is —SOₙ₁₆R^{16D}. In embodiments, R¹⁶ is —SOᵥ₁₆NR^{16A}R^{16B}. In embodiments, R¹⁶ is —NHNR^{16A}R^{16B}. In embodiments, R¹⁶ is —ONR^{16A}R^{16B}. In embodiments, R¹⁶ is —NHC═(O)NHNR^{16A}R^{16B}. In embodiments, R¹⁶ is —NHC(O)NR^{16A}R^{16B}. In embodiments, R¹⁶ is —N(O)ₘ₁₆. In embodiments, R¹⁶ is —NR^{16A}R^{16B}. In embodiments, R¹⁶ is —C(O)R^{16C}. In embodiments, R¹⁶ is —C(O)—OR^{16C}. In embodiments, R¹⁶ is —C(O)NR^{16A}R^{16B}. In embodiments, R¹⁶ is —OR^{16D}. In embodiments, R¹⁶ is —NR^{16A}SO₂R^{16D}. In embodiments, R¹⁶ is —NR^{16A}C(O)R^{16C}. In embodiments, R¹⁶ is —NR^{16A}C(O)OR^{16C}. In embodiments, R¹⁶ is —NR^{16A}OR^{16C}. In embodiments, R¹⁶ is —OCX¹⁶₃. In embodiments, R¹⁶ is —OCHX¹⁶₂. In embodiments, R¹⁶ is substituted or unsubstituted alkyl. In embodiments, R¹⁶ is substituted or unsubstituted heteroalkyl. In embodiments, R¹⁶ is substituted or unsubstituted cycloalkyl. In embodiments, R¹⁶ is substituted or unsubstituted heterocycloalkyl. In embodiments, R¹⁶ is substituted or unsubstituted aryl. In embodiments, R¹⁶ is substituted or unsubstituted heteroaryl. In embodiments, R¹⁶ is substituted alkyl. In embodiments, R¹⁶ is substituted heteroalkyl. In embodiments, R¹⁶ is substituted cycloalkyl. In embodiments, R¹⁶ is substituted heterocycloalkyl. In embodiments, R¹⁶ is substituted aryl. In embodiments, R¹⁶ is substituted heteroaryl. In embodiments, R¹⁶ is unsubstituted alkyl. In embodiments, R¹⁶ is unsubstituted heteroalkyl. In embodiments, R¹⁶ is unsubstituted cycloalkyl. In embodiments, R¹⁶ is unsubstituted heterocycloalkyl. In embodiments, R¹⁶ is unsubstituted aryl. In embodiments, R¹⁶ is unsubstituted heteroaryl. In embodiments, R¹⁶ is unsubstituted methyl. In embodiments, R¹⁶ is unsubstituted ethyl. In embodiments, R¹⁶ is unsubstituted propyl. In embodiments, R¹⁶ is unsubstituted isopropyl. In embodiments, R¹⁶ is unsubstituted butyl. In embodiments, R¹⁶ is unsubstituted tert-butyl.

In embodiments, R^{16A} is hydrogen. In embodiments, R^{16A} is —CX₃. In embodiments, R^{16A} is —CN. In embodiments, R^{16A} is —COOH. In embodiments, R^{16A} is —CONH₂. In embodiments, R^{16A} is —CHX₂. In embodiments, R^{16A} is —CH₂X. In embodiments, R^{16A} is unsubstituted methyl. In embodiments, R^{16A} is unsubstituted ethyl. In embodiments, R^{16A} is unsubstituted propyl. In embodiments, R^{16A} is unsubstituted isopropyl. In embodiments, R^{16A} is unsubstituted butyl. In embodiments, R^{16A} is unsubstituted tert-butyl.

In embodiments, R^{16B} is hydrogen. In embodiments, R^{16B} is —CX₃. In embodiments, R^{16B} is —CN. In embodiments, R^{16B} is —COOH. In embodiments, R^{16B} is —CONH₂. In embodiments, R^{16B} is —CHX₂. In embodiments, R^{16B} is —CH₂X. In embodiments, R^{16B} is unsubstituted methyl. In embodiments, R^{16B} is unsubstituted ethyl. In embodiments, R^{16B} is unsubstituted propyl. In embodiments, R^{16B} is unsubstituted isopropyl. In embodiments, R^{16B} is unsubstituted butyl. In embodiments, R^{16B} is unsubstituted tert-butyl.

In embodiments, R^{16C} is hydrogen. In embodiments, R^{16C} is —CX₃. In embodiments, R^{16C} is —CN. In embodiments, R^{16C} is —COOH. In embodiments, R^{16C} is —CONH₂. In embodiments, R^{16C} is —CHX₂. In embodiments, R^{16C} is —CH₂X. In embodiments, R^{16C} is unsubstituted methyl. In embodiments, R^{16C} is unsubstituted ethyl. In embodiments, R^{16C} is unsubstituted propyl. In embodiments, R^{16C} is unsubstituted isopropyl. In embodiments, R^{16C} is unsubstituted butyl. In embodiments, R^{16C} is unsubstituted tert-butyl.

In embodiments, R^{16D} is hydrogen. In embodiments, R^{16D} is —CX₃. In embodiments, R^{16D} is —CN. In embodiments, R^{16D} is —COOH. In embodiments, R^{16D} is —CONH₂. In embodiments, R^{16D} is —CHX₂. In embodiments, R^{16D} is —CH₂X. In embodiments, R^{16D} is unsubstituted methyl. In embodiments, R^{16D} is unsubstituted ethyl. In embodiments, R^{16D} is unsubstituted propyl. In embodiments, R^{16D} is unsubstituted isopropyl. In embodiments, R^{16D} is unsubstituted butyl. In embodiments, R^{16D} is unsubstituted tert-butyl.

In embodiments, R¹⁶ is independently hydrogen, oxo,

halogen, —CX¹⁶₃, —CHX¹⁶₂, —OCH₂X¹⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁶₃, —OCHX¹⁶₂, R⁷⁵-substituted or unsubstituted alkyl, R⁷⁵-substituted or unsubstituted heteroalkyl, R⁷⁵-substituted or unsubstituted cycloalkyl, R⁷⁵-substituted or unsubstituted heterocycloalkyl, R⁷⁵-substituted or unsubstituted aryl, or R⁷⁵-substituted or unsubstituted heteroaryl. X¹⁶ is halogen. In embodiments, X¹⁶ is F.

R⁷⁵ is independently oxo,

halogen, —CX⁷⁵₃, —CHX⁷⁵₂, —OCH₂X⁷⁵, —OCHX⁷⁵₂, —CN, —OH, —NH₂, —COOH, —CONH₂, —NO₂, —S H, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁵₃, —OCHX⁷⁵₂, R⁷⁶-substituted or unsubstituted alkyl, R⁷⁶-substituted or unsubstituted heteroalkyl, R⁷⁶-substituted or unsubstituted cycloalkyl, R⁷⁶-substituted or unsubstituted heterocycloalkyl, R⁷⁶-substituted or unsubstituted aryl, or R⁷⁶-substituted or unsubstituted heteroaryl. X⁷⁵ is halogen. In embodiments, X⁷⁵ is F.

R⁷⁶ is independently oxo,

halogen, —CX⁷⁶₃, —CHX⁷⁶₂, —OCH₂X⁷⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁶₃, —OCHX⁷⁶₂, R⁷⁷-substituted or unsubstituted alkyl, R⁷⁷-substituted or unsubstituted heteroalkyl, R⁷⁷-substituted or unsubstituted cycloalkyl, R⁷⁷-substituted or unsubstituted heterocycloalkyl, R⁷⁷-substituted or unsubstituted aryl, or R⁷⁷-substituted or unsubstituted heteroaryl. X⁷⁶ is halogen. In embodiments, X⁷⁶ is F.

In embodiments, R^{16A} is independently hydrogen, oxo,

halogen, —CX^{16A}₃, —CHX^{16A}₂, —OCH₂X^{16A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{16A}₃, —OCHX^{16A}₂, R^{75A}-substituted or unsubstituted alkyl, R^{75A}-substituted or unsubstituted heteroalkyl, R^{75A}-substituted or unsubstituted cycloalkyl, R^{75A}-substituted or unsubstituted heterocycloalkyl, R^{75A}-substituted or unsubstituted aryl, or R^{75A}-substituted or unsubstituted heteroaryl. X^{16A} is halogen. In embodiments, X^{16A} is F.

R^{75A} is independently oxo,

halogen, —CX^{75A}₃, —CHX^{75A}₂, —OCH₂X^{75A}, —OCHX^{75A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{75A}₃, —OCHX^{75A}₂, R^{76A}-substituted or unsubstituted alkyl, R^{76A}-substituted or unsubstituted heteroalkyl, R^{76A}-substituted or unsubstituted cycloalkyl, R^{76A}-substituted or unsubstituted heterocycloalkyl, R^{76A}-substituted or unsubstituted aryl, or R^{76A}-substituted or unsubstituted heteroaryl. X^{75A} is halogen. In embodiments, X^{75A} is F.

R^{76A} is independently oxo,

halogen, —CX^{76A}₃, —CHX^{76A}₂, —OCH₂X^{76A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{76A}₃, —OCHX^{76A}₂, R^{77A}-substituted or unsubstituted alkyl, R^{77A}-substituted or unsubstituted heteroalkyl, R^{77A}-substituted or unsubstituted cycloalkyl, R^{77A}-substituted or unsubstituted heterocycloalkyl, R^{77A}-substituted or unsubstituted aryl, or R^{77A}-substituted or unsubstituted heteroaryl. X^{76A} is halogen. In embodiments, X^{76A} is F.

In embodiments, R^{16B} is independently hydrogen, oxo,

halogen, —CX^{16B}₃, —CHX^{16B}₂, —OCH₂X^{16B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{16B}₃, —OCHX^{16B}₂, R^{75B}-substituted or unsubstituted alkyl, R^{75B}-substituted or unsubstituted heteroalkyl, R^{75B}-substituted or unsubstituted cycloalkyl, R^{75B}-substituted or unsubstituted heterocycloalkyl, R^{75B}-substituted or unsubstituted aryl, or R^{75B}-substituted or unsubstituted heteroaryl. X^{16B} is halogen. In embodiments, X^{16B} is F.

R^{75B} is independently oxo,

halogen, —CX^{75B}₃, —CHX^{75B}₂, —OCH₂X^{75B}, —OCHX^{75B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{75B}₃, —OCHX^{75B}₂, R^{76B}-substituted or unsubstituted alkyl, R^{76B}-substituted or unsubstituted heteroalkyl, R^{76B}-substituted or unsubstituted cycloalkyl, R^{76B}-substituted or unsubstituted heterocycloalkyl, R^{76B}-substituted or unsubstituted aryl, or R^{76B}-substituted or unsubstituted heteroaryl. X^{75B} is halogen. In embodiments, X^{75B} is F.

R^{76B} is independently oxo,

halogen, —CX^{76B}₃, —CHX^{76B}₂, —OCH₂X^{76B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{76B}₃, —OCHX^{76B}₂, R^{77B}-substituted or unsubstituted alkyl, R^{77B}-substituted or unsubstituted heteroalkyl, R^{77B}-substituted or unsubstituted cycloalkyl, R^{77B}-substituted or unsubstituted heterocycloalkyl, R^{77B}-substituted or unsubstituted aryl, or R^{77B}-substituted or unsubstituted heteroaryl. X^{76B} is halogen. In embodiments, X^{76B} is F.

In embodiments, R^{16C} is independently hydrogen, oxo,

halogen, —CX^{16C}₃, —CHX^{16C}₂, —OCH₂X^{16C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{16C}₃, —OCHX^{16C}₂, R^{75C}-substituted or unsubstituted alkyl, R^{75C}-substituted or unsubstituted heteroalkyl, R^{75C}-substituted or unsubstituted cycloalkyl, R^{75C}-substituted or unsubstituted heterocycloalkyl, R^{75C}-substituted or unsubstituted aryl, or R^{75C}-substituted or unsubstituted heteroaryl. X^{16C} is halogen. In embodiments, X^{16C} is F.

R^{75C} is independently oxo,

halogen, —CX^{75C}₃, —CHX^{75C}₂, —OCH₂X^{75C}, —OCHX^{75C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{75C}₃, —OCHX^{75C}₂, R^{76C}-substituted or unsubstituted alkyl, R^{76C}-substituted or unsubstituted heteroalkyl, R^{76C}-substituted or unsubstituted cycloalkyl, R^{76C}-substituted or unsubstituted heterocycloalkyl, R^{76C}-substituted or unsubstituted aryl, or R^{76C}-substituted or unsubstituted heteroaryl. X^{75C} is halogen. In embodiments, X^{75C} is F.

R^{76C} is independently oxo,

halogen, —CX^{76C}₃, —CHX^{76C}₂, —OCH₂X^{76C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{76C}₃, —OCHX^{76C}₂, R^{77C}-substituted or unsubstituted alkyl, R^{77C}-substituted or unsubstituted heteroalkyl, R^{77C}-substituted or unsubstituted cycloalkyl, R^{77C}-substituted or unsubstituted heterocycloalkyl, R^{77C}-substituted or unsubstituted aryl, or R^{77C}-substituted or unsubstituted heteroaryl. X^{76C} is halogen. In embodiments, X^{76C} is F.

In embodiments, R^{16D} is independently hydrogen, oxo,

halogen, —CX^{16D}₃, —CHX^{16D}₂, —OCH₂X^{16D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{16D}₃, —OCHX^{16D}₂, R^{75D}-substituted or unsubstituted alkyl, R^{75D}-substituted or unsubstituted heteroalkyl, R^{75D}-substituted or unsubstituted cycloalkyl, R^{75D}-substituted or unsubstituted heterocycloalkyl, R^{75D}-substituted or unsubstituted aryl, or R^{75D}-substituted or unsubstituted heteroaryl. X^{16D} is halogen. In embodiments, X^{16D} is F.

R^{75D} is independently oxo,

halogen, —CX^{75D}₃, —CHX^{75D}₂, —OCH₂X^{75D}, —OCHX^{75D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{75D}₃, —OCHX^{75D}₂, R^{76D}-substituted or unsubstituted alkyl, R^{76D}-substituted or unsubstituted heteroalkyl, R^{76D}-substituted or unsubstituted cycloalkyl, R^{76D}-substituted or unsubstituted heterocycloalkyl, R^{76D}-substituted or unsubstituted aryl, or R^{76D}-substituted or unsubstituted heteroaryl. X^{75D} is halogen. In embodiments, X^{75D} is F.

R^{76D} is independently oxo,

halogen, —CX^{76D}₃, —CHX^{76D}₂, —OCH₂X^{76D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{76D}₃, —OCHX^{76D}₂, R^{77D}-substituted or unsubstituted alkyl, R^{77D}-substituted or unsubstituted heteroalkyl, R^{77D}-substituted or unsubstituted cycloalkyl, R^{77D}-substituted or unsubstituted heterocycloalkyl, R^{77D}-substituted or unsubstituted aryl, or R^{77D}-substituted or unsubstituted heteroaryl. X^{76D} is halogen. In embodiments, X^{76D} is F.

In embodiments, R¹⁷ is hydrogen. In embodiments, R¹⁷ is halogen. In embodiments, R¹⁷ is CX¹⁷₃. In embodiments, R¹⁷ is —CHX¹⁷₂. In embodiments, R¹⁷ is —CH₂X¹⁷. In embodiments, R¹⁷ is —CN. In embodiments, R¹⁷ is —SOₙ₁₇R^{17D}. In embodiments, R¹⁷ is —SOᵥ₁₇NR^{17A}R^{17B}. In embodiments, R¹⁷ is —NHNR^{17A}R^{17B}. In embodiments, R¹⁷ is —ONR^{17A}R^{17B}. In embodiments, R¹⁷ is —NHC═(O)NHNR^{17A}R^{17B}. In embodiments, R¹⁷ is —NHC(O)NR^{17A}R^{17B}. In embodiments, R¹⁷ is —N(O)ₘ₁₇. In embodiments, R¹⁷ is —NR^{17A}R^{17B}. In embodiments, R¹⁷ is —C(O)R^{17C}. In embodiments, R¹⁷ is —C(O)—OR^{17C}. In embodiments, R¹⁷ is —C(O)NR^{17A}R^{17B}. In embodiments, R¹⁷ is —OR^{17C}. In embodiments, R¹⁷ is —NR^{17A}SO₂R^{17D}. In embodiments, R¹⁷ is —NR^{17A}C(O)R^{17C}. In embodiments, R¹⁷ is —NR^{17A}C(O)OR^{17C}. In embodiments, R¹⁷ is —NR^{17A}OR^{17C}. In embodiments, R¹⁷ is —OCX¹⁷₃. In embodiments, R¹⁷ is —OCHX¹⁷2. In embodiments, R¹⁷ is substituted or unsubstituted alkyl. In embodiments, R¹⁷ is substituted or unsubstituted heteroalkyl. In embodiments, R¹⁷ is substituted or unsubstituted cycloalkyl. In embodiments, R¹⁷ is substituted or unsubstituted heterocycloalkyl. In embodiments, R¹⁷ is substituted or unsubstituted aryl. In embodiments, R¹⁷ is substituted or unsubstituted heteroaryl. In embodiments, R¹⁷ is substituted alkyl. In embodiments, R¹⁷ is substituted heteroalkyl. In embodiments, R¹⁷ is substituted cycloalkyl. In embodiments, R¹⁷ is substituted heterocycloalkyl. In embodiments, R¹⁷ is substituted aryl. In embodiments, R¹⁷ is substituted heteroaryl. In embodiments, R¹⁷ is unsubstituted alkyl. In embodiments, R¹⁷ is unsubstituted heteroalkyl. In embodiments, R¹⁷ is unsubstituted cycloalkyl. In embodiments, R¹⁷ is unsubstituted heterocycloalkyl. In embodiments, R¹⁷ is unsubstituted aryl. In embodiments, R¹⁷ is unsubstituted heteroaryl. In embodiments, R¹⁷ is unsubstituted methyl. In embodiments, R¹⁷ is unsubstituted ethyl. In embodiments, R¹⁷ is unsubstituted propyl. In embodiments, R¹⁷ is unsubstituted isopropyl. In embodiments, R¹⁷ is unsubstituted butyl. In embodiments, R¹⁷ is unsubstituted tert-butyl.

In embodiments, R^{17A} is hydrogen. In embodiments, R^{17A} is —CX₃. In embodiments, R^{17A} is —CN. In embodiments, R^{17A} is —COOH. In embodiments, R^{17A} is —CONH₂. In embodiments, R^{17A} is —CHX₂. In embodiments, R^{17A} is —CH₂X. In embodiments, R^{17A} is unsubstituted methyl. In embodiments, R^{17A} is unsubstituted ethyl. In embodiments, R^{17A} is unsubstituted propyl. In embodiments, R^{17A} is unsubstituted isopropyl. In embodiments, R^{17A} is unsubstituted butyl. In embodiments, R^{17A} is unsubstituted tert-butyl.

In embodiments, R^{17B} is hydrogen. In embodiments, R^{17B} is —CX₃. In embodiments, R^{17B} is —CN. In embodiments, R^{17B} is —COOH. In embodiments, R^{17B} is —CONH₂. In embodiments, R^{17B} is —CHX₂. In embodiments, R^{17B} is —CH₂X. In embodiments, R^{17B} is unsubstituted methyl. In embodiments, R^{17B} is unsubstituted ethyl. In embodiments, R^{17B} is unsubstituted propyl. In embodiments, R^{17B} is unsubstituted isopropyl. In embodiments, R^{17B} is unsubstituted butyl. In embodiments, R^{17B} is unsubstituted tert-butyl.

In embodiments, R^{17C} is hydrogen. In embodiments, R^{17C} is —CX₃. In embodiments, R^{17C} is —CN. In embodiments, R^{17C} is —COOH. In embodiments, R^{17C} is —CONH₂. In embodiments, R^{17C} is —CHX₂. In embodiments, R^{17C} is —CH₂X. In embodiments, R^{17C} is unsubstituted methyl. In embodiments, R^{17C} is unsubstituted ethyl. In embodiments, R^{17C} is unsubstituted propyl. In embodiments, R^{17C} is unsubstituted isopropyl. In embodiments, R^{17C} is unsubstituted butyl. In embodiments, R^{17C} is unsubstituted tert-butyl.

In embodiments, R^{17D} is hydrogen. In embodiments, R^{17D} is —CX₃. In embodiments, R^{17D} is —CN. In embodiments, R^{17D} is —COOH. In embodiments, R^{17D} is —CONH₂. In embodiments, R^{17D} is —CHX₂. In embodiments, R^{17D} is —CH₂X. In embodiments, R^{17D} is unsubstituted methyl. In embodiments, R^{17D} is unsubstituted ethyl. In embodiments, R^{17D} is unsubstituted propyl. In embodiments, R^{17D} is unsubstituted isopropyl. In embodiments, R^{17D} is unsubstituted butyl. In embodiments, R^{17D} is unsubstituted tert-butyl.

In embodiments, R¹⁷ is independently hydrogen, oxo,

halogen, —CX¹⁷₃, —CHX¹⁷₂, —OCH₂X¹⁷, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁷₃, —OCHX¹⁷₂, R⁷⁸-substituted or unsubstituted alkyl, R⁷⁸-substituted or unsubstituted heteroalkyl, R⁷⁸-substituted or unsubstituted cycloalkyl, R⁷⁸-substituted or unsubstituted heterocycloalkyl, R⁷⁸-substituted or unsubstituted aryl, or R⁷⁸-substituted or unsubstituted heteroaryl. X¹⁷ is halogen. In embodiments, X¹⁷ is F.

R⁷⁸ is independently oxo,

halogen, —CX⁷⁸₃, —CHX⁷⁸₂, —OCH₂X⁷⁸, —OCHX⁷⁸₂, —CN, —OH, —NH₂, —COOH, —CONH₂, —NO₂, —S H, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁸₃, —OCHX⁷⁸₂, R⁷⁹-substituted or unsubstituted alkyl, R⁷⁹-substituted or unsubstituted heteroalkyl, R⁷⁹-substituted or unsubstituted cycloalkyl, R⁷⁹-substituted or unsubstituted heterocycloalkyl, R⁷⁹-substituted or unsubstituted aryl, or R⁷⁹-substituted or unsubstituted heteroaryl. X⁷⁸ is halogen. In embodiments, X⁷⁸ is F.

R⁷⁹ is independently oxo,

halogen, —CX⁷⁹₃, —CHX⁷⁹₂, —OCH₂X⁷⁹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁹₃, —OCHX⁷⁹₂, R⁸⁰-substituted or unsubstituted alkyl, R⁸⁰-substituted or unsubstituted heteroalkyl, R⁸⁰-substituted or unsubstituted cycloalkyl, R⁸⁰-substituted or unsubstituted heterocycloalkyl, R⁸⁰-substituted or unsubstituted aryl, or R⁸⁰-substituted or unsubstituted heteroaryl. X⁷⁹ is halogen. In embodiments, X⁷⁹ is F.

In embodiments, R^{17A} is independently hydrogen, oxo,

halogen, —CX^{17A}₃, —CHX^{17A}₂, —OCH₂X^{17A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{17A}₃, —OCHX^{17A}₂, R^{78A}-substituted or unsubstituted alkyl, R^{78A}-substituted or unsubstituted heteroalkyl, R^{78A}-substituted or unsubstituted cycloalkyl, R^{78A}-substituted or unsubstituted heterocycloalkyl, R^{78A}-substituted or unsubstituted aryl, or R^{78A}-substituted or unsubstituted heteroaryl. X^{17A} is halogen. In embodiments, X^{17A} is F.

R^{78A} is independently oxo,

halogen, —CX^{78A}₃, —CHX^{78A}₂, —OCH₂X^{78A}, —OCHX^{78A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{78A}₃, —OCHX^{78A}₂, R^{79A}-substituted or unsubstituted alkyl, R^{79A}-substituted or unsubstituted heteroalkyl, R^{79A}-substituted or unsubstituted cycloalkyl, R^{79A}-substituted or unsubstituted heterocycloalkyl, R^{79A}-substituted or unsubstituted aryl, or R^{79A}-substituted or unsubstituted heteroaryl. X^{78A} is halogen. In embodiments, X^{78A} is F.

R^{79A} is independently oxo,

halogen, —CX^{79A}₃, —CHX^{79A}₂, —OCH₂X^{79A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{79A}₃, —OCHX^{79A}₂, R^{80A}-substituted or unsubstituted alkyl, R^{80A}-substituted or unsubstituted heteroalkyl, R^{80A}-substituted or unsubstituted cycloalkyl, R^{80A}-substituted or unsubstituted heterocycloalkyl, R^{80A}-substituted or unsubstituted aryl, or R^{80A}-substituted or unsubstituted heteroaryl. X^{79A} is halogen. In embodiments, X^{79A} is F.

In embodiments, R^{17B} is independently hydrogen, oxo,

halogen, —CX^{17B}₃, —CHX^{17B}₂, —OCH₂X^{17B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{17B}₃, —OCHX^{17B}₂, R^{78B}-substituted or unsubstituted alkyl, R^{78B}-substituted or unsubstituted heteroalkyl, R^{78B}-substituted or unsubstituted cycloalkyl, R^{78B}-substituted or unsubstituted heterocycloalkyl, R^{78B}-substituted or unsubstituted aryl, or R^{78B}-substituted or unsubstituted heteroaryl. X^{17B} is halogen. In embodiments, X^{17B} is F.

R^{78B} is independently oxo,

halogen, —CX^{78B}₃, —CHX^{78B}₂, —OCH₂X^{78B}, —OCHX^{78B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{78B}₃, —OCHX^{78B}₂, R^{79B}-substituted or unsubstituted alkyl, R^{79B}-substituted or unsubstituted heteroalkyl, R^{79B}-substituted or unsubstituted cycloalkyl, R^{79B}-substituted or unsubstituted heterocycloalkyl, R^{79B}-substituted or unsubstituted aryl, or R^{79B}-substituted or unsubstituted heteroaryl. X^{78B} is halogen. In embodiments, X^{78B} is F.

R^{79B} is independently oxo,

halogen, —CX^{79B}₃, —CHX^{79B}₂, —OCH₂X^{79B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{79B}₃, —OCHX^{79B}₂, R^{80B}-substituted or unsubstituted alkyl, R^{80B}-substituted or unsubstituted heteroalkyl, R^{80B}-substituted or unsubstituted cycloalkyl, R^{80B}-substituted or unsubstituted heterocycloalkyl, R″^{B}-substituted or unsubstituted aryl, or R^{80B}-substituted or unsubstituted heteroaryl. X^{79B} is halogen. In embodiments, X^{79B} is F.

In embodiments, R^{17C} is independently hydrogen, oxo,

halogen, —CX^{17C}₃, —CHX^{17C}₂, —OCH₂X^{17C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{17C}₃, —OCHX^{17C}₂, R^{78C}-substituted or unsubstituted alkyl, R^{78C}-substituted or unsubstituted heteroalkyl, R^{78C}-substituted or unsubstituted cycloalkyl, R^{78C}-substituted or unsubstituted heterocycloalkyl, R^{78C}-substituted or unsubstituted aryl, or R^{78C}-substituted or unsubstituted heteroaryl. X^{17C} is halogen. In embodiments, X^{17C} is F.

R^{78C} is independently oxo,

halogen, —CX^{78C}₃, —CHX^{78C}₂, —OCH₂X^{78C}, —OCHX^{78C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{78C}₃, —OCHX^{78C}₂, R^{79C}-substituted or unsubstituted alkyl, R^{79C}-substituted or unsubstituted heteroalkyl, R^{79C}-substituted or unsubstituted cycloalkyl, R^{79C}-substituted or unsubstituted heterocycloalkyl, R^{79C}-substituted or unsubstituted aryl, or R^{79C}-substituted or unsubstituted heteroaryl. X^{78C} is halogen. In embodiments, X^{78C} is F.

R^{79C} is independently oxo,

halogen, —CX^{79C}₃, —CHX^{79C}₂, —OCH₂X^{79C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{79C}₃, —OCHX^{79C}₂, R^{80C}-substituted or unsubstituted alkyl, R^{80C}-substituted or unsubstituted heteroalkyl, R^{80C}-substituted or unsubstituted cycloalkyl, R^{80C}-substituted or unsubstituted heterocycloalkyl, R^{80C}-substituted or unsubstituted aryl, or R^{80C}-substituted or unsubstituted heteroaryl. X^{79C} is halogen. In embodiments, X^{79C} is F.

In embodiments, R^{17D} is independently hydrogen, oxo,

halogen, —CX^{17D}₃, —CHX^{17D}₂, —OCH₂X^{17D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{17D}₃, —OCHX^{17D}₂, R^{78D}-substituted or unsubstituted alkyl, R^{78D}-substituted or unsubstituted heteroalkyl, R^{78D}-substituted or unsubstituted cycloalkyl, R^{78D}-substituted or unsubstituted heterocycloalkyl, R^{78D}-substituted or unsubstituted aryl, or R^{78D}-substituted or unsubstituted heteroaryl. X^{17D} is halogen. In embodiments, X^{17D} is F.

R^{78D} is independently oxo,

halogen, —CX^{78D}₃, —CHX^{78D}₂, —OCH₂X^{78D}, —OCHX^{78D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{78D}₃, —OCHX^{78D}₂, R^{79D}-substituted or unsubstituted alkyl, R^{79D}-substituted or unsubstituted heteroalkyl, R^{79D}-substituted or unsubstituted cycloalkyl, R^{79D}-substituted or unsubstituted heterocycloalkyl, R^{79D}-substituted or unsubstituted aryl, or R^{79D}-substituted or unsubstituted heteroaryl. X^{78D} is halogen. In embodiments, X^{78D} is F.

R^{79D} is independently oxo,

halogen, —CX^{79D}₃, —CHX^{79D}₂, —OCH₂X^{79D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{79D}₃, —OCHX^{79D}₂, R^{80D}-substituted or unsubstituted alkyl, R^{80D}-substituted or unsubstituted heteroalkyl, R^{80D}-substituted or unsubstituted cycloalkyl, R^{80D}-substituted or unsubstituted heterocycloalkyl, R^{80D}-substituted or unsubstituted aryl, or R^{80D}-substituted or unsubstituted heteroaryl. X^{79D} is halogen. In embodiments, X^{79D} is F.

In embodiments, R¹⁸ is hydrogen. In embodiments, R¹⁸ is halogen. In embodiments, R¹⁸ is CX¹⁸₃. In embodiments, R¹⁸ is —CHX¹⁸₂. In embodiments, R¹⁸ is —CH₂X¹⁸. In embodiments, R¹⁸ is —CN. In embodiments, R¹⁸ is —SOₙ₁₈R^{18D}. In embodiments, R¹⁸ is —SOᵥ₁₈NR^{18A}R^{18B}. In embodiments, R¹⁸ is —NHNR^{18A}R^{18B}. In embodiments, R¹⁸ is —ONR^{18A}R^{18B}. In embodiments, R¹⁸ is —NHC═(O)NHNR^{18A}R^{18B}. In embodiments, R¹⁸ is —NHC(O)NR^{18A}R^{18B}. In embodiments, R¹⁸ is —N(O)ₘ₁₈. In embodiments, R¹⁸ is —NR^{18A}R^{18B}. In embodiments, R¹⁸ is —C(O)R^{18C}. In embodiments, R¹⁸ is —C(O)—OR^{18C}. In embodiments, R¹⁸ is —C(O)NR^{18A}R^{18B}. In embodiments, R¹⁸ is —OR^{18D}. In embodiments, R¹⁸ is —NR^{18A}SO₂R^{18D}. In embodiments, R¹⁸ is —NR^{18A}C(O)R^{18C}. In embodiments, R¹⁸ is —NR^{18A}C(O)OR^{18C}. In embodiments, R¹⁸ is —NR^{18A}OR^{18C}. In embodiments, R¹⁸ is —OCX¹⁸₃. In embodiments, R¹⁸ is —OCHX¹⁸₂. In embodiments, R¹⁸ is substituted or unsubstituted alkyl. In embodiments, R¹⁸ is substituted or unsubstituted heteroalkyl. In embodiments, R¹⁸ is substituted or unsubstituted cycloalkyl. In embodiments, R¹⁸ is substituted or unsubstituted heterocycloalkyl. In embodiments, R¹⁸ is substituted or unsubstituted aryl. In embodiments, R¹⁸ is substituted or unsubstituted heteroaryl. In embodiments, R¹⁸ is substituted alkyl. In embodiments, R¹⁸ is substituted heteroalkyl. In embodiments, R¹⁸ is substituted cycloalkyl. In embodiments, R¹⁸ is substituted heterocycloalkyl. In embodiments, R¹⁸ is substituted aryl. In embodiments, R¹⁸ is substituted heteroaryl. In embodiments, R¹⁸ is unsubstituted alkyl. In embodiments, R¹⁸ is unsubstituted heteroalkyl. In embodiments, R¹⁸ is unsubstituted cycloalkyl. In embodiments, R¹⁸ is unsubstituted heterocycloalkyl. In embodiments, R¹⁸ is unsubstituted aryl. In embodiments, R¹⁸ is unsubstituted heteroaryl. In embodiments, R¹⁸ is unsubstituted methyl. In embodiments, R¹⁸ is unsubstituted ethyl. In embodiments, R¹⁸ is unsubstituted propyl. In embodiments, R¹⁸ is unsubstituted isopropyl. In embodiments, R¹⁸ is unsubstituted butyl. In embodiments, R¹⁸ is unsubstituted tert-butyl.

In embodiments, R^{18A} is hydrogen. In embodiments, R^{18A} is —CX₃. In embodiments, R^{18A} is —CN. In embodiments, R^{18A} is —COOH. In embodiments, R^{18A} is —CONH₂. In embodiments, R^{18A} is —CHX₂. In embodiments, R^{18A} is —CH₂X. In embodiments, R^{18A} is unsubstituted methyl. In embodiments, R^{18A} is unsubstituted ethyl. In embodiments, R^{18A} is unsubstituted propyl. In embodiments, R^{18A} is unsubstituted isopropyl. In embodiments, R^{18A} is unsubstituted butyl. In embodiments, R^{18A} is unsubstituted tert-butyl.

In embodiments, R^{18B} is hydrogen. In embodiments, R^{18B} is —CX₃. In embodiments, R^{18B} is —CN. In embodiments, R^{18B} is —COOH. In embodiments, R^{18B} is —CONH₂. In embodiments, R^{18B} is —CHX₂. In embodiments, R^{18B} is —CH₂X. In embodiments, R^{18B} is unsubstituted methyl. In embodiments, R^{18B} is unsubstituted ethyl. In embodiments, R^{18B} is unsubstituted propyl. In embodiments, R^{18B} is unsubstituted isopropyl. In embodiments, R^{18B} is unsubstituted butyl. In embodiments, R^{18B} is unsubstituted tert-butyl.

In embodiments, R^{18B} is hydrogen. In embodiments, R^{18B} is —CX₃. In embodiments, R^{18B} is —CN. In embodiments, R^{18C} is —COOH. In embodiments, R^{18B} is —CONH₂. In embodiments, R^{18C} is —CHX₂. In embodiments, R^{18C} is —CH₂X. In embodiments, R^{18C} is unsubstituted methyl. In embodiments, R^{18C} is unsubstituted ethyl. In embodiments, R^{18C} is unsubstituted propyl. In embodiments, R^{18C} is unsubstituted isopropyl. In embodiments, R^{18C} is unsubstituted butyl. In embodiments, R^{18C} is unsubstituted tert-butyl.

In embodiments, R^{18D} is hydrogen. In embodiments, R^{18D} is —CX₃. In embodiments, R¹⁸ is —CN. In embodiments, R^{18D} is —COOH. In embodiments, R¹⁸ is —CONH₂. In embodiments, R^{18D} is —CHX₂. In embodiments, R^{18D} is —CH₂X. In embodiments, R^{18D} is unsubstituted methyl. In embodiments, R^{18D} is unsubstituted ethyl. In embodiments, R^{18D} is unsubstituted propyl. In embodiments, R^{18D} is unsubstituted isopropyl. In embodiments, R^{18D} is unsubstituted butyl. In embodiments, R^{18D} is unsubstituted tert-butyl.

In embodiments, R¹⁸ is independently hydrogen, oxo,

halogen, —CX¹⁸₃, —CHX¹⁸₂, —OCH₂X¹⁸, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁸₃, —OCHX¹⁸₂, R⁸¹-substituted or unsubstituted alkyl, R⁸¹-substituted or unsubstituted heteroalkyl, R⁸¹-substituted or unsubstituted cycloalkyl, R⁸¹-substituted or unsubstituted heterocycloalkyl, R⁸¹-substituted or unsubstituted aryl, or R⁸¹-substituted or unsubstituted heteroaryl. X¹⁸ is halogen. In embodiments, X¹⁸ is F.

R^{8′} is independently oxo,

halogen, —CX⁸¹₃, —CHX⁸¹₂, —OCH₂X⁸¹, —OCHX⁸¹₂, —CN, —OH, —NH₂, —COOH, —CONH₂, —NO₂, —S H, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁸¹₃, —OCHX⁸¹₂, R⁸²-substituted or unsubstituted alkyl, R⁸²-substituted or unsubstituted heteroalkyl, R⁸²-substituted or unsubstituted cycloalkyl, R⁸²-substituted or unsubstituted heterocycloalkyl, R⁸²-substituted or unsubstituted aryl, or R⁸²-substituted or unsubstituted heteroaryl. X⁸¹ is halogen. In embodiments, X⁸¹ is F.

R⁸² is independently oxo,

halogen, —CX⁸²₃, —CHX⁸²₂, —OCH₂X⁸², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁸²₃, —OCHX⁸²₂, R⁸³-substituted or unsubstituted alkyl, R⁸³-substituted or unsubstituted heteroalkyl, R⁸³-substituted or unsubstituted cycloalkyl, R⁸³-substituted or unsubstituted heterocycloalkyl, R⁸³-substituted or unsubstituted aryl, or R⁸³-substituted or unsubstituted heteroaryl. X⁸² is halogen. In embodiments, X⁸² is F.

In embodiments, R^{18A} is independently hydrogen, oxo,

halogen, —CX^{18A}₃, —CHX^{18A}₂, —OCH₂X^{18A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{18A}₃, —OCHX^{18A}₂, R^{81A}-substituted or unsubstituted alkyl, R^{81A}-substituted or unsubstituted heteroalkyl, R^{81A}-substituted or unsubstituted cycloalkyl, R^{81A}-substituted or unsubstituted heterocycloalkyl, R^{81A}-substituted or unsubstituted aryl, or R^{81A}-substituted or unsubstituted heteroaryl. X^{18A} is halogen. In embodiments, X^{18A} is F.

R^{81A} is independently oxo,

halogen, —CX^{81A}₃, —CHX^{81A}₂, —OCH₂X^{81A}, —OCHX^{81A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{81A}₃, —OCHX^{81A}₂, R^{82A}-substituted or unsubstituted alkyl, R^{82A}-substituted or unsubstituted heteroalkyl, R^{82A}-substituted or unsubstituted cycloalkyl, R^{82A}-substituted or unsubstituted heterocycloalkyl, R^{82A}-substituted or unsubstituted aryl, or R^{82A}-substituted or unsubstituted heteroaryl. X^{81A} is halogen. In embodiments, X^{81A} is F.

R^{82A} is independently oxo,

halogen, —CX^{82A}₃, —CHX^{82A}₂, —OCH₂X^{82A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{82A}₃, —OCHX^{82A}₂, R^{83A}-substituted or unsubstituted alkyl, R^{83A}-substituted or unsubstituted heteroalkyl, R^{83A}-substituted or unsubstituted cycloalkyl, R^{83A}-substituted or unsubstituted heterocycloalkyl, R^{83A}-substituted or unsubstituted aryl, or R^{83A}-substituted or unsubstituted heteroaryl. X^{82A} is halogen. In embodiments, X^{82A} is F.

In embodiments, R^{18B} is independently hydrogen, oxo,

halogen, —CX^{18B}₃, —CHX^{18B}₂, —OCH₂X^{18B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{18B}₃, —OCHX^{18B}₂, R^{81B}-substituted or unsubstituted alkyl, R^{81B}-substituted or unsubstituted heteroalkyl, R^{81B}-substituted or unsubstituted cycloalkyl, R^{81B}-substituted or unsubstituted heterocycloalkyl, R^{81B}-substituted or unsubstituted aryl, or R^{81B}-substituted or unsubstituted heteroaryl. X^{18B} is halogen. In embodiments, X^{18B} is F.

R^{81B} is independently oxo,

halogen, —CX^{81B}₃, —CHX^{81B}₂, —OCH₂X^{81B}, —OCHX^{81B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{81B}₃, —OCHX^{81B}₂, R^{82B}-substituted or unsubstituted alkyl, R^{82B}-substituted or unsubstituted heteroalkyl, R^{82B}-substituted or unsubstituted cycloalkyl, R^{82B}-substituted or unsubstituted heterocycloalkyl, R^{82B}-substituted or unsubstituted aryl, or R^{82B}-substituted or unsubstituted heteroaryl. X^{81B} is halogen. In embodiments, X^{81B} is F.

R^{82B} is independently oxo,

halogen, —CX^{82B}₃, —CHX^{82B}₂, —OCH₂X^{82B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{82B}₃, —OCHX^{82B}₂, R^{83B}-substituted or unsubstituted alkyl, R^{83B}-substituted or unsubstituted heteroalkyl, R^{83B}-substituted or unsubstituted cycloalkyl, R^{83B}-substituted or unsubstituted heterocycloalkyl, R^{83B}-substituted or unsubstituted aryl, or R^{83B}-substituted or unsubstituted heteroaryl. X^{82B} is halogen. In embodiments, X^{82B} is F.

In embodiments, R^{18C} is independently hydrogen, oxo,

halogen, —CX^{18C}₃, —CHX^{18C}₂, —OCH₂X^{18C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{18C}₃, —OCHX^{18C}₂, R^{81C}-substituted or unsubstituted alkyl, R^{81C}-substituted or unsubstituted heteroalkyl, R^{81C}-substituted or unsubstituted cycloalkyl, R^{81C}-substituted or unsubstituted heterocycloalkyl, R^{81C}-substituted or unsubstituted aryl, or R^{81C}-substituted or unsubstituted heteroaryl. X^{18C} is halogen. In embodiments, X^{18C} is F.

R^{81C} is independently oxo,

halogen, —CX^{81C}₃, —CHX^{81C}₂, —OCH₂X^{81C}, —OCHX^{81C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{81C}₃, —OCHX^{81C}₂, R^{82C}-substituted or unsubstituted alkyl, R^{82C}-substituted or unsubstituted heteroalkyl, R^{82C}-substituted or unsubstituted cycloalkyl, R^{82C}-substituted or unsubstituted heterocycloalkyl, R^{82C}-substituted or unsubstituted aryl, or R^{82C}-substituted or unsubstituted heteroaryl. X^{81C} is halogen. In embodiments, X^{81C} is F.

R^{82C} is independently oxo,

halogen, —CX^{82C}₃, —CHX^{82C}₂, —OCH₂X^{82C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{82C}₃, —OCHX^{82C}₂, R^{83C}-substituted or unsubstituted alkyl, R^{83C}-substituted or unsubstituted heteroalkyl, R^{83C}-substituted or unsubstituted cycloalkyl, R^{83C}-substituted or unsubstituted heterocycloalkyl, R^{83C}-substituted or unsubstituted aryl, or R^{83C}-substituted or unsubstituted heteroaryl. X^{82C} is halogen. In embodiments, X^{82C} is F.

In embodiments, R^{18D} is independently hydrogen, oxo,

halogen, —CX^{18D}₃, —CHX^{18D}₂, —OCH₂X^{18D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{18D}₃, —OCHX^{18D}₂, R^{81D}-substituted or unsubstituted alkyl, R^{81D} substituted or unsubstituted heteroalkyl, R^{81D} substituted or unsubstituted cycloalkyl, R^{81D}-substituted or unsubstituted heterocycloalkyl, R^{81D} substituted or unsubstituted aryl, or R^{81D}-substituted or unsubstituted heteroaryl. X^{18D} is halogen. In embodiments, X^{18D} is F.

R^{18D} is independently oxo,

halogen, —CX^{81D}₃, —CHX^{81D}₂, —OCH₂X^{81D}, —OCHX^{81D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{81D}₃, —OCHX^{81D}₂, R^{82D}-substituted or unsubstituted alkyl, R^{82D}-substituted or unsubstituted heteroalkyl, R^{82D}-substituted or unsubstituted cycloalkyl, R^{82D}-substituted or unsubstituted heterocycloalkyl, R^{82D}-substituted or unsubstituted aryl, or R^{82D}-substituted or unsubstituted heteroaryl. X^{81D} is halogen. In embodiments, X^{81D} is F.

R^{82D} is independently oxo,

halogen, —CX^{82D}₃, —CHX^{82D}₂, —OCH₂X^{82D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{82D}₃, —OCHX^{82D}₂, R^{83D}-substituted or unsubstituted alkyl, R^{83D}-substituted or unsubstituted heteroalkyl, R^{83D}-substituted or unsubstituted cycloalkyl, R^{83D}-substituted or unsubstituted heterocycloalkyl, R^{83D}-substituted or unsubstituted aryl, or R^{83D}-substituted or unsubstituted heteroaryl. X^{82D} is halogen. In embodiments, X^{82D} is F.

R⁷⁴, R⁷⁷, R⁸⁰, R⁸³, R^{74A}, R^{77A}, R^{80A}, R^{83A}, R^{74B}, R^{77B}, R^{80B}, R^{83B}, R^{74C}, R^{77C}, R^{80C}, R^{83C}, R^{74D}, R^{77D}, R^{80D}, R^{83D}, R⁸⁶, R⁸⁹, R⁹², and R⁹⁸ are independently hydrogen, oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. In R⁷⁴, R⁷⁷, R⁸⁰, R⁸³, R^{74A}, R^{77A}, R^{80A}, R^{83A}, R^{74B}, R^{77B}, R^{80B}, R^{83B}, R^{74C}, R^{77C}, R^{80C}, R^{83C}, R^{74D}, R^{77D}, R^{80D}, R^{83D}, R⁸⁶, R⁸⁹, R⁹², and R⁹⁸ are independently hydrogen, oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. In embodiments, R⁷⁴, R⁷⁷, R⁸⁰, R⁸³, R^{74A}, R^{77A}, R^{80A}, R^{83A}, R^{74B}, R^{77B}, R^{80B}, R^{83B}, R^{74C}, R^{77C}, R^{80C}, R^{83C}, R^{74D}, R^{77D}, R^{80D}, R^{83D}, R⁸⁶, R⁸⁹, R⁹², and R⁹⁸ are independently hydrogen, oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, unsubstituted C₁-C₈ alkyl, unsubstituted 2 to 8 membered heteroalkyl, unsubstituted C₃-C₈ cycloalkyl, unsubstituted 3 to 6 membered heterocycloalkyl, unsubstituted phenyl, or unsubstituted 5 to 6 membered heteroaryl.

In embodiments, R¹⁵, R¹⁶, R¹⁷, and R¹⁸ are hydrogen.

In embodiments, E is:

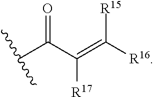

In embodiments, E is:

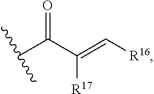

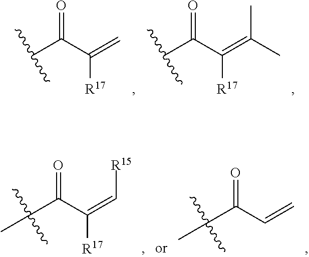

In embodiments, E is:

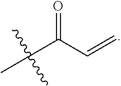 [SMILES: *C(=O)C=C]

In embodiments, E is:

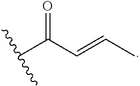 [SMILES: *C(=O)/C=C/C]

In embodiments, E is:

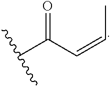 [SMILES: *C(=O)/C=C\C]

In embodiments, E is:

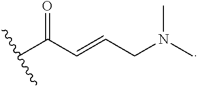 [SMILES: *C(=O)/C=C/CN(C)C]

In embodiments, E is:

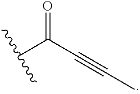 [SMILES: *C(=O)C#CC]

In some embodiments, a compound as described herein may include multiple instances of R¹ or R², and/or other variables. In such embodiments, each variable may optional be different and be appropriately labeled to distinguish each group for greater clarity. For example, where each R¹ and/or R², is different, they may be referred to, for example, as R^{1.1}, R^{1.2}, R^{1.3}, R^{1.4}, R^{1.5}, R^{2.1}, R^{2.2}, R^{2.3}, or R^{2.4}, respectively, wherein the definition of R¹ is assumed by R^{1.1}, R^{1.2}, R^{1.3}, R^{1.4}, R^{1.5}; and/or R² is assumed by R^{2.1}, R^{2.2}, R^{2.3}, R^{2.4}. The variables used within a definition of R¹ and/or R², and/or other variables that appear at multiple instances and are different may similarly be appropriately labeled to distinguish each group for greater clarity. In some embodiments, the compound is a compound described herein (e.g., in an aspect, embodiment, example, claim, table, scheme, drawing, or figure).

In embodiments, unless otherwise indicated, a compound described herein is a racemic mixture of all stereoisomers. In embodiments, unless otherwise indicated, a compound described herein is a racemic mixture of all enantiomers. In embodiments, unless otherwise indicated, a compound described herein is a racemic mixture of two opposite stereoisomers. In embodiments, unless otherwise indicated, a compound described herein is a racemic mixture of two opposite enantiomers. In embodiments, unless otherwise indicated, a compound described herein is a single stereoisomer. In embodiments, unless otherwise indicated, a compound described herein is a single enantiomer. In embodiments, the compound is a compound described herein (e.g., in an aspect, embodiment, example, figure, table, scheme, or claim).

In an aspect is provided a Reticulon 4 inhibitor. In embodiments, the Reticulon 4 inhibitor is a compound described herein. In embodiments, the Reticulon 4 inhibitor is an oligonucleotide (e.g., DNA, RNA, shRNA, or siRNA), protein (e.g., antibody, anti-Reticulon 4 antibody, anti-Reticulon 4 binding antibody fragment), or compound (e.g., compound described herein). In embodiments, the Reticulon 4 inhibitor contacts one or more amino acids corresponding to E1105, C1101, E1078, 51079, A1082, I1083, K1090, Y1091, S1094, G1097, and H1098 of human reticulon 4. In embodiments, the Reticulon 4 inhibitor covalently binds an amino acid corresponding to C1101 in human reticulon 4. In embodiments, the Reticulon 4 inhibitor contacts an amino acids corresponding to E1105, C1101, E1078, S1079, A1082, I1083, K1090, Y1091, S1094, G1097, and H1098 of human reticulon 4. In embodiments, the Reticulon 4 inhibitor contacts an amino acids corresponding to E1105 of human reticulon 4. In embodiments, the Reticulon 4 inhibitor contacts an amino acids corresponding to C1101 of human reticulon 4. In embodiments, the Reticulon 4 inhibitor contacts an amino acids corresponding to E1078 of human reticulon 4. In embodiments, the Reticulon 4 inhibitor contacts an amino acids corresponding to S1079 of human reticulon 4. In embodiments, the Reticulon 4 inhibitor contacts an amino acids corresponding to A1082 of human reticulon 4. In embodiments, the Reticulon 4 inhibitor contacts an amino acids corresponding to I1083 of human reticulon 4. In embodiments, the Reticulon 4 inhibitor contacts an amino acids corresponding to K1090 of human reticulon 4. In embodiments, the Reticulon 4 inhibitor contacts an amino acids corresponding to Y1091 of human reticulon 4. In embodiments, the Reticulon 4 inhibitor contacts an amino acids corresponding to S1094 of human reticulon 4. In embodiments, the Reticulon 4 inhibitor contacts an amino acids corresponding to G1097 of human reticulon 4. In embodiments, the Reticulon 4 inhibitor contacts an amino acids corresponding to H1098 of human reticulon 4.

In an aspect is provided a Reticulon 4 inhibitor. In embodiments, the Reticulon 4 inhibitor is a compound described herein. In embodiments, the Reticulon 4 inhibitor is an oligonucleotide (e.g., DNA, RNA, shRNA, or siRNA), protein (e.g., antibody, anti-Reticulon 4 antibody, anti-Reticulon 4 binding antibody fragment), or compound (e.g., compound described herein). In embodiments, the Reticulon 4 inhibitor contacts one or more amino acids corresponding to E1105, C1101, E1078, S1079, A1082, I1083, K1090, Y1091, S1094, G1097, and H1098 of SEQ ID NO:331. In embodiments, the Reticulon 4 inhibitor covalently binds an amino acid corresponding to C1101 in of SEQ ID NO:331. In embodiments, the Reticulon 4 inhibitor contacts an amino acids corresponding to E1105, C1101, E1078, S1079, A1082, I1083, K1090, Y1091, S1094, G1097, and H1098 of SEQ ID NO:331. In embodiments, the Reticulon 4 inhibitor contacts an amino acids corresponding to E1105 of SEQ ID NO:331. In embodiments, the Reticulon 4 inhibitor contacts an amino acids corresponding to C1101 of SEQ ID NO:331. In embodiments, the Reticulon 4 inhibitor contacts an amino acids corresponding to E1078 of SEQ ID NO:331. In embodiments, the Reticulon 4 inhibitor contacts an amino acids corresponding to S1079 of SEQ ID NO:331. In embodiments, the Reticulon 4 inhibitor contacts an amino acids corresponding to A1082 of SEQ ID NO:331. In embodiments, the Reticulon 4 inhibitor contacts an amino acids corresponding to I1083 of SEQ ID NO:331. In embodiments, the Reticulon 4 inhibitor contacts an amino acids corresponding to K1090 of SEQ ID NO:331. In embodiments, the Reticulon 4 inhibitor contacts an amino acids corresponding to Y1091 of SEQ ID NO:331. In embodiments, the Reticulon 4 inhibitor contacts an amino acids corresponding to S1094 of SEQ ID NO:331. In embodiments, the Reticulon 4 inhibitor contacts an amino acids corresponding to G1097 of SEQ ID NO:331. In embodiments, the Reticulon 4 inhibitor contacts an amino acids corresponding to H1098 of SEQ ID NO:331.

In embodiments, the compound has the formula:

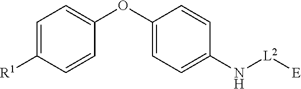

wherein R¹, L², and E are as described herein, including embodiments. In embodiments, the compound has the formula:

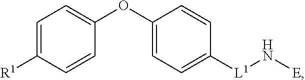

wherein R¹, L¹, and E are as described herein, including embodiments. In embodiments, R¹ is independently halogen, —CX¹₃, —CN, —OH, —NH₂, —COOH, —CONH₂, —NO₂, —SH, —OCX¹₃, —OCHX¹₂, —OCH₂X¹, —CHX¹₂, —CH₂X¹, substituted or unsubstituted C₁-C₈ alkyl, or substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl. In embodiments, R¹ is independently halogen, —CX¹₃, —CN, unsubstituted C₁-C₄ alkyl, or unsubstituted 2 to 4 membered heteroalkyl. In embodiments, R¹ is independently unsubstituted methyl, unsubstituted ethyl, unsubstituted isopropyl, or unsubstituted tert-butyl. In embodiments, R¹ is independently unsubstituted methyl. In embodiments, R¹ is independently unsubstituted ethyl. In embodiments, R¹ is independently unsubstituted propyl. In embodiments, R¹ is independently unsubstituted n-propyl. In embodiments, R¹ is independently unsubstituted isopropyl. In embodiments, R¹ is independently unsubstituted butyl. In embodiments, R¹ is independently unsubstituted n-butyl. In embodiments, R¹ is independently unsubstituted isobutyl. In embodiments, R¹ is independently unsubstituted tert-butyl. In embodiments, R¹ is independently unsubstituted pentyl. In embodiments, R¹ is independently unsubstituted hexyl. In embodiments, R¹ is independently unsubstituted heptyl. In embodiments, R¹ is independently unsubstituted octyl. In embodiments, R¹ is independently —CF₃. In embodiments, R¹ is independently —CCl₃. In embodiments, R¹ is independently unsubstituted phenyl. In embodiments, R¹ is independently unsubstituted pyridyl. In embodiments, R¹ is independently halogen. In embodiments, R¹ is independently —CN. In embodiments, R¹ is independently —OH. In embodiments, R¹ is independently —NH₂. In embodiments, R¹ is independently —COOH. In embodiments, R¹ is independently —CONH₂. In embodiments, R¹ is independently —NO₂. In embodiments, R¹ is independently —SH. In embodiments, R¹ is independently —SO₃H. In embodiments, R¹ is independently —SO₄H. In embodiments, R¹ is independently —SO₂NH₂. In embodiments, R¹ is independently —NHNH₂. In embodiments, R¹ is independently —ONH₂. In embodiments, R¹ is independently —NHC(O)NHNH₂. In embodiments, R¹ is independently —NHC(O)NH₂. In embodiments, R¹ is independently —NHSO₂H. In embodiments, R¹ is independently —NHC(O)H. In embodiments, R¹ is independently —NHC(O)OH. In embodiments, R¹ is independently —NHOH. In embodiments, R¹ is independently substituted or unsubstituted alkyl. In embodiments, R¹ is independently substituted or unsubstituted heteroalkyl. In embodiments, R¹ is independently substituted or unsubstituted cycloalkyl. In embodiments, R¹ is independently substituted or unsubstituted heterocycloalkyl. In embodiments, R¹ is independently substituted or unsubstituted aryl. In embodiments, R¹ is independently substituted or unsubstituted heteroaryl. In embodiments, R¹ is independently substituted alkyl. In embodiments, R¹ is independently substituted heteroalkyl. In embodiments, R¹ is independently substituted cycloalkyl. In embodiments, R¹ is independently substituted heterocycloalkyl. In embodiments, R¹ is independently substituted aryl. In embodiments, R¹ is independently substituted heteroaryl. In embodiments, R¹ is independently unsubstituted alkyl. In embodiments, R¹ is independently unsubstituted heteroalkyl. In embodiments, R¹ is independently unsubstituted cycloalkyl. In embodiments, R¹ is independently unsubstituted heterocycloalkyl. In embodiments, R¹ is independently unsubstituted aryl. In embodiments, R¹ is independently unsubstituted heteroaryl. In embodiments, R¹ is independently substituted or unsubstituted C₁-C₈ alkyl. In embodiments, R¹ is independently substituted or unsubstituted 2 to 8 membered heteroalkyl. In embodiments, R¹ is independently substituted or unsubstituted C₃-C₈ cycloalkyl. In embodiments, R¹ is independently substituted or unsubstituted 3 to 8 membered heterocycloalkyl. In embodiments, R¹ is independently substituted or unsubstituted C₆-C₁₀ aryl. In embodiments, R¹ is independently substituted or unsubstituted 5 to 10 membered heteroaryl. In embodiments, R¹ is independently substituted C₁-C₈ alkyl. In embodiments, R¹ is independently substituted 2 to 8 membered heteroalkyl. In embodiments, R¹ is independently substituted C₃-C₈ cycloalkyl. In embodiments, R¹ is independently substituted 3 to 8 membered heterocycloalkyl. In embodiments, R¹ is independently substituted C₆-C₁₀ aryl. In embodiments, R¹ is independently substituted 5 to 10 membered heteroaryl. In embodiments, R¹ is independently unsubstituted C₁-C₈ alkyl. In embodiments, R¹ is independently unsubstituted 2 to 8 membered heteroalkyl. In embodiments, R¹ is independently unsubstituted C₃-C₈ cycloalkyl. In embodiments, R¹ is independently unsubstituted 3 to 8 membered heterocycloalkyl. In embodiments, R¹ is independently unsubstituted C₆-C₁₀ aryl. In embodiments, R¹ is independently unsubstituted 5 to 10 membered heteroaryl. In embodiments, R¹ is independently substituted or unsubstituted C₁-C₄ alkyl. In embodiments, R¹ is independently substituted or unsubstituted 2 to 4 membered heteroalkyl. In embodiments, R¹ is independently substituted or unsubstituted C₃-C₆ cycloalkyl. In embodiments, R¹ is independently substituted or unsubstituted 3 to 6 membered heterocycloalkyl. In embodiments, R¹ is independently substituted or unsubstituted phenyl. In embodiments, R¹ is independently substituted or unsubstituted 5 to 6 membered heteroaryl. In embodiments, R¹ is independently substituted C₁-C₄ alkyl. In embodiments, R¹ is independently substituted 2 to 4 membered heteroalkyl. In embodiments, R¹ is independently substituted C₃-C₆ cycloalkyl. In embodiments, R¹ is independently substituted 3 to 6 membered heterocycloalkyl. In embodiments, R¹ is independently substituted phenyl. In embodiments, R¹ is independently substituted 5 to 6 membered heteroaryl. In embodiments, R¹ is independently unsubstituted C₁-C₄ alkyl. In embodiments, R¹ is independently unsubstituted 2 to 4 membered heteroalkyl. In embodiments, R¹ is independently unsubstituted C₃-C₆ cycloalkyl. In embodiments, R¹ is independently unsubstituted 3 to 6 membered heterocycloalkyl. In embodiments, R¹ is independently unsubstituted phenyl. In embodiments, R¹ is independently unsubstituted 5 to 6 membered heteroaryl. In embodiments, R¹ is independently —OH. In embodiments, R¹ is independently —NH₂. In embodiments, R¹ is independently —COOH. In embodiments, R¹ is independently —CONH₂. In embodiments, R¹ is independently —NO₂. In embodiments, R¹ is independently —SH. In embodiments, R¹ is independently —CF₃. In embodiments, R¹ is independently —CHF₂. In embodiments, R¹ is independently —CH₂F. In embodiments, R¹ is independently —OCF₃. In embodiments, R¹ is independently —OCH₂F. In embodiments, R¹ is independently —OCHF₂. In embodiments, R¹ is independently —OCH₃. In embodiments, R¹ is independently —OCH₂CH₃. In embodiments, R¹ is independently —OCH₂CH₂CH₃. In embodiments, R¹ is independently —OCH(CH₃)₂. In embodiments, R¹ is independently —OC(CH₃)₃. In embodiments, R¹ is independently —SCH₃. In embodiments, R¹ is independently —SCH₂CH₃. In embodiments, R¹ is independently —SCH₂CH₂CH₃. In embodiments, R¹ is independently —SCH(CH₃)₂. In embodiments, R¹ is independently —SC(CH₃)₃. In embodiments, R¹ is independently —CH₃. In embodiments, R¹ is independently —CH₂CH₃. In embodiments, R¹ is independently —CH₂CH₂CH₃. In embodiments, R¹ is independently —CH(CH₃)₂. In embodiments, R¹ is independently —C(CH₃)₃. In embodiments, R¹ is independently —F. In embodiments, R¹ is independently —Cl. In embodiments, R¹ is independently —Br. In embodiments, R¹ is independently —I.

In embodiments, R¹ is R²⁰-substituted or unsubstituted methyl. In embodiments, R¹ is R²⁰-substituted or unsubstituted C₂ alkyl. In embodiments, R¹ is R²⁰-substituted or unsubstituted C₃ alkyl. In embodiments, R¹ is R²⁰-substituted or unsubstituted C₄ alkyl. In embodiments, R¹ is R²⁰-substituted or unsubstituted C₅ alkyl. In embodiments, R¹ is R²⁰-substituted or unsubstituted C₆ alkyl. In embodiments, R¹ is R²⁰-substituted or unsubstituted C₇ alkyl. In embodiments, R¹ is R²⁰-substituted or unsubstituted C₈ alkyl. In embodiments, R¹ is R²⁰-substituted methyl. In embodiments, R¹ is R²⁰-substituted C₂ alkyl. In embodiments, R¹ is R²⁰-substituted C₃ alkyl. In embodiments, R¹ is R²⁰-substituted C₄ alkyl. In embodiments, R¹ is R²⁰-substituted C₅ alkyl. In embodiments, R¹ is R²⁰-substituted C₆ alkyl. In embodiments, R¹ is R²⁰-substituted C₇ alkyl. In embodiments, R¹ is R²⁰-substituted C₈ alkyl. In embodiments, R¹ is an unsubstituted methyl. In embodiments, R¹ is an unsubstituted C₂ alkyl. In embodiments, R¹ is an unsubstituted C₃ alkyl. In embodiments, R¹ is an unsubstituted C₄ alkyl. In embodiments, R¹ is an unsubstituted C₅ alkyl. In embodiments, R¹ is an unsubstituted C₆ alkyl. In embodiments, R¹ is an unsubstituted C₇ alkyl. In embodiments, R¹ is an unsubstituted C₈ alkyl.

In embodiments, the compound has the formula:

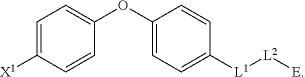

X¹, L¹, L², and E are as described herein.

In embodiments, the compound has the formula:

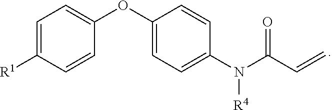

R¹ and R⁴ are as described herein.

In embodiments, the compound has the formula:

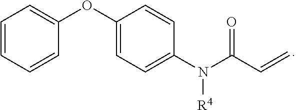

R⁴ is as described herein.

In embodiments, the compound has the formula:

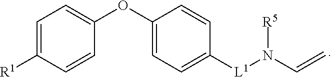

R¹, R⁵, and L¹ are as described herein.

In embodiments, the compound has the formula:

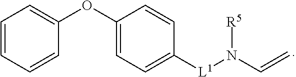

L¹ and R⁵ are as described herein.

In embodiments, R⁴ is independently substituted or unsubstituted alkyl. In embodiments, R⁴ is independently substituted or unsubstituted heteroalkyl. In embodiments, R⁴ is independently substituted or unsubstituted cycloalkyl. In embodiments, R⁴ is independently substituted or unsubstituted heterocycloalkyl. In embodiments, R⁴ is independently substituted or unsubstituted aryl. In embodiments, R⁴ is independently substituted or unsubstituted heteroaryl. In embodiments, R⁴ is independently substituted alkyl. In embodiments, R⁴ is independently substituted heteroalkyl. In embodiments, R⁴ is independently substituted cycloalkyl. In embodiments, R⁴ is independently substituted heterocycloalkyl. In embodiments, R⁴ is independently substituted aryl. In embodiments, R⁴ is independently substituted heteroaryl. In embodiments, R⁴ is independently unsubstituted alkyl. In embodiments, R⁴ is independently unsubstituted heteroalkyl. In embodiments, R⁴ is independently unsubstituted cycloalkyl. In embodiments, R⁴ is independently unsubstituted heterocycloalkyl. In embodiments, R⁴ is independently unsubstituted aryl. In embodiments, R⁴ is independently unsubstituted heteroaryl. In embodiments, R⁴ is independently substituted or unsubstituted C₁-C₈ alkyl. In embodiments, R⁴ is independently substituted or unsubstituted 2 to 8 membered heteroalkyl. In embodiments, R⁴ is independently substituted or unsubstituted C₃-C₈ cycloalkyl. In embodiments, R⁴ is independently substituted or unsubstituted 3 to 8 membered heterocycloalkyl. In embodiments, R⁴ is independently substituted or unsubstituted C₆-C₁₀ aryl. In embodiments, R⁴ is independently substituted or unsubstituted 5 to 10 membered heteroaryl. In embodiments, R⁴ is independently substituted C₁-C₈ alkyl. In embodiments, R⁴ is independently substituted 2 to 8 membered heteroalkyl. In embodiments, R⁴ is independently substituted C₃-C₈ cycloalkyl. In embodiments, R⁴ is independently substituted 3 to 8 membered heterocycloalkyl. In embodiments, R⁴ is independently substituted C₆-C₁₀ aryl. In embodiments, R⁴ is independently substituted 5 to 10 membered heteroaryl. In embodiments, R⁴ is independently unsubstituted C₁-C₈ alkyl. In embodiments, R⁴ is independently unsubstituted 2 to 8 membered heteroalkyl. In embodiments, R⁴ is independently unsubstituted C₃-C₈ cycloalkyl. In embodiments, R⁴ is independently unsubstituted 3 to 8 membered heterocycloalkyl. In embodiments, R⁴ is independently unsubstituted C₆-C₁₀ aryl. In embodiments, R⁴ is independently unsubstituted 5 to 10 membered heteroaryl. In embodiments, R⁴ is independently substituted or unsubstituted C₁-C₄ alkyl. In embodiments, R⁴ is independently substituted or unsubstituted 2 to 4 membered heteroalkyl. In embodiments, R⁴ is independently substituted or unsubstituted C₃-C₆ cycloalkyl. In embodiments, R⁴ is independently substituted or unsubstituted 3 to 6 membered heterocycloalkyl. In embodiments, R⁴ is independently substituted or unsubstituted phenyl. In embodiments, R⁴ is independently substituted or unsubstituted 5 to 6 membered heteroaryl. In embodiments, R⁴ is independently substituted C₁-C₄ alkyl. In embodiments, R⁴ is independently substituted 2 to 4 membered heteroalkyl. In embodiments, R⁴ is independently substituted C₃-C₆ cycloalkyl. In embodiments, R⁴ is independently substituted 3 to 6 membered heterocycloalkyl. In embodiments, R⁴ is independently substituted phenyl. In embodiments, R⁴ is independently substituted 5 to 6 membered heteroaryl. In embodiments, R⁴ is independently unsubstituted C₁-C₄ alkyl. In embodiments, R⁴ is independently unsubstituted 2 to 4 membered heteroalkyl. In embodiments, R⁴ is independently unsubstituted C₃-C₆ cycloalkyl. In embodiments, R⁴ is independently unsubstituted 3 to 6 membered heterocycloalkyl. In embodiments, R⁴ is independently unsubstituted phenyl. In embodiments, R⁴ is independently unsubstituted 5 to 6 membered heteroaryl.

In embodiments, R⁴ is R²⁹-substituted or unsubstituted methyl. In embodiments, R⁴ is R²⁹-substituted or unsubstituted C₂ alkyl. In embodiments, R⁴ is R²⁹-substituted or unsubstituted C₃ alkyl. In embodiments, R⁴ is R²⁹-substituted or unsubstituted C₄ alkyl. In embodiments, R⁴ is R²⁹-substituted or unsubstituted C₅ alkyl. In embodiments, R⁴ is R²⁹-substituted or unsubstituted C₆ alkyl. In embodiments, R⁴ is R²⁹-substituted or unsubstituted C₇ alkyl. In embodiments, R⁴ is R²⁹-substituted or unsubstituted C₈ alkyl. In embodiments, R⁴ is R²⁹-substituted methyl. In embodiments, R⁴ is R²⁹-substituted C₂ alkyl. In embodiments, R⁴ is R²⁹-substituted C₃ alkyl. In embodiments, R⁴ is R²⁹-substituted C₄ alkyl. In embodiments, R⁴ is R²⁹-substituted C₈ alkyl. In embodiments, R⁴ is R²⁹-substituted C₆ alkyl. In embodiments, R⁴ is R²⁹-substituted C₇ alkyl. In embodiments, R⁴ is R²⁹-substituted C₈ alkyl. In embodiments, R⁴ is an unsubstituted methyl. In embodiments, R⁴ is an unsubstituted C₂ alkyl. In embodiments, R⁴ is an unsubstituted C₃ alkyl. In embodiments, R⁴ is an unsubstituted C₄ alkyl. In embodiments, R⁴ is an unsubstituted C₅ alkyl. In embodiments, R⁴ is an unsubstituted C₆ alkyl. In embodiments, R⁴ is an unsubstituted C₇ alkyl. In embodiments, R⁴ is an unsubstituted C₈ alkyl.

In embodiments, R⁴ is independently —OH. In embodiments, R⁴ is independently —NH₂. In embodiments, R⁴ is independently —COOH. In embodiments, R⁴ is independently —CONH₂. In embodiments, R⁴ is independently —CF₃. In embodiments, R⁴ is independently —CHF₂. In embodiments, R⁴ is independently —CH₂F. In embodiments, R⁴ is independently —OCF₃. In embodiments, R⁴ is independently —OCH₂F. In embodiments, R⁴ is independently —OCHF₂. In embodiments, R⁴ is independently —OCH₃. In embodiments, R⁴ is independently —OCH₂CH₃. In embodiments, R⁴ is independently —OCH₂CH₂CH₃. In embodiments, R⁴ is independently —OCH(CH₃)₂. In embodiments, R⁴ is independently —OC(CH₃)₃. In embodiments, R⁴ is independently —SCH₃. In embodiments, R⁴ is independently —SCH₂CH₃. In embodiments, R⁴ is independently —SCH₂CH₂CH₃. In embodiments, R⁴ is independently —SCH(CH₃)₂. In embodiments, R⁴ is independently —SC(CH₃)₃. In embodiments, R⁴ is independently —CH₃. In embodiments, R⁴ is independently —CH₂CH₃. In embodiments, R⁴ is independently —CH₂CH₂CH₃. In embodiments, R⁴ is independently —CH(CH₃)₂. In embodiments, R⁴ is independently —C(CH₃)₃. In embodiments, R⁴ is independently hydrogen.

In an aspect is provided a compound having the formula:

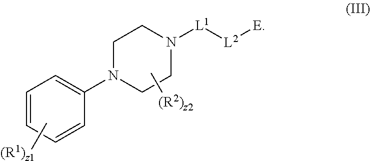

R¹, R², L¹, L², E, z1 and z2 are as described herein.

In embodiments, the compound has the formula:

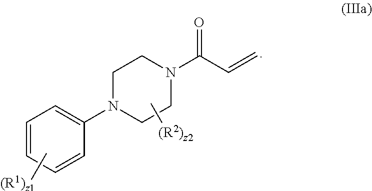

R¹, R², z1 and z2 are as described herein.

In embodiments, the compound has the formula:

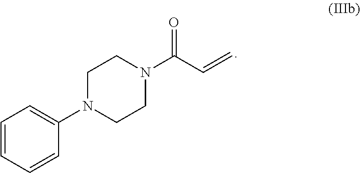 [SMILES: C=CC(=O)N1CCN(c2ccccc2)CC1]

In embodiments, the compound is a compound described herein, including in an aspect, embodiment, claim, figure, table, example, or scheme.

In embodiments, the compound has the formula:

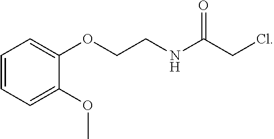 [SMILES: COc1ccccc1OCCNC(=O)CCl]

In embodiments, the compound has the formula:

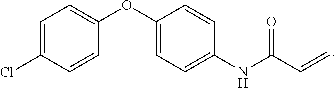 [SMILES: C=CC(=O)Nc1ccc(Oc2ccc(Cl)cc2)cc1]

In embodiments, the compound has the formula:

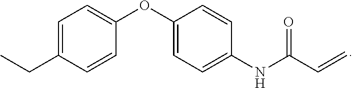 [SMILES: C=CC(=O)Nc1ccc(Oc2ccc(CC)cc2)cc1]

In embodiments, the compound has the formula:

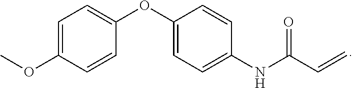 [SMILES: C=CC(=O)Nc1ccc(Oc2ccc(OC)cc2)cc1]

In embodiments, the compound has the formula:

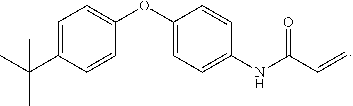 [SMILES: C=CC(=O)Nc1ccc(Oc2ccc(C(C)(C)C)cc2)cc1]

In embodiments, the compound has the formula:

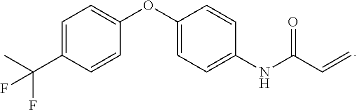 [SMILES: C=CC(=O)Nc1ccc(Oc2ccc(C(C)(F)F)cc2)cc1]

In embodiments, the compound has the formula:

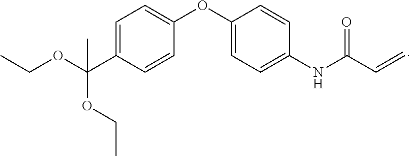 [SMILES: C=CC(=O)Nc1ccc(Oc2ccc(C(C)(OCC)OCC)cc2)cc1]

In embodiments, the compound has the formula:

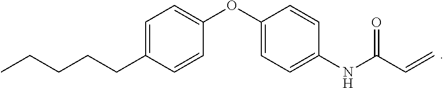 [SMILES: C=CC(=O)Nc1ccc(Oc2ccc(CCCCC)cc2)cc1]

In embodiments, the compound has the formula:

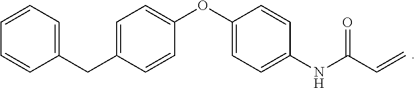 [SMILES: C=CC(=O)Nc1ccc(Oc2ccc(Cc3ccccc3)cc2)cc1]

In embodiments, the compound has the formula:

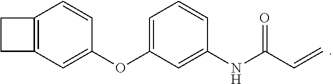 [SMILES: C=CC(=O)Nc1cccc(Oc2ccc3c(c2)CC3)c1]

In embodiments, the compound has the formula:

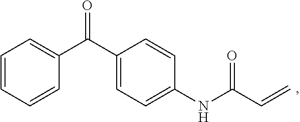 [SMILES: C=CC(=O)Nc1ccc(C(=O)c2ccccc2)cc1]

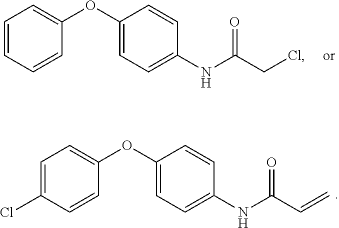 [SMILES: O=C(CCl)Nc1ccc(Oc2ccccc2)cc1 ; C=CC(=O)Nc1ccc(Oc2ccc(Cl)cc2)cc1]

In embodiments, the compound has the formula:

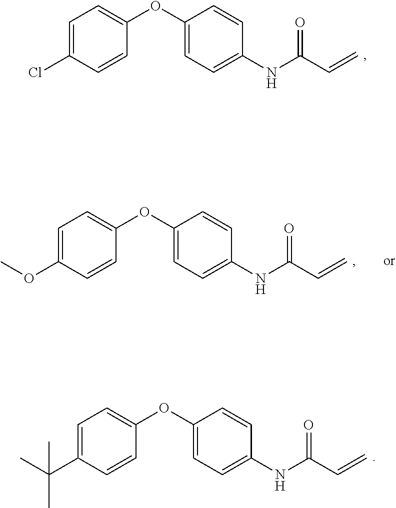

In embodiments, the compound has the formula:

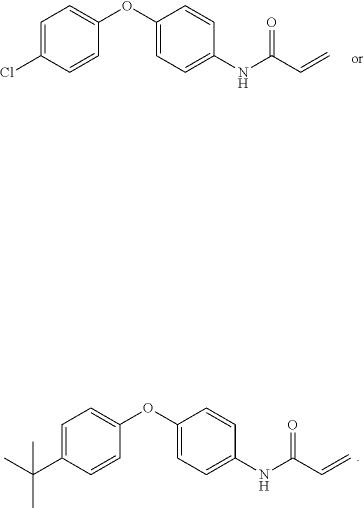

### III. Pharmaceutical Compositions

In an aspect is provided a pharmaceutical composition including a Reticulon 4 inhibitor and a pharmaceutically acceptable excipient. In embodiments, the Reticulon 4 inhibitor is a compound described herein. In embodiments, the Reticulon 4 inhibitor is an oligonucleotide (e.g., DNA, RNA, or siRNA), protein (e.g., antibody, anti-Reticulon 4 antibody, anti-Reticulon 4 binding antibody fragment), or compound (e.g., compound described herein). In embodiments, the Reticulon 4 inhibitor is included in a therapeutically effective amount.

In an aspect is provided a pharmaceutical composition including a compound described herein, or pharmaceutically acceptable salt thereof, and a pharmaceutically acceptable excipient.

In embodiments of the pharmaceutical compositions, the compound, or pharmaceutically acceptable salt thereof, is included in a therapeutically effective amount.

In embodiments of the pharmaceutical compositions, the pharmaceutical composition includes a second agent (e.g. therapeutic agent). In embodiments of the pharmaceutical compositions, the pharmaceutical composition includes a second agent (e.g. therapeutic agent) in a therapeutically effective amount. In embodiments of the pharmaceutical compositions, the second agent is an agent for treating cancer. In embodiments, the second agent is an anti-cancer agent. In embodiments, the second agent is a chemotherapeutic. In embodiments, the second agent is an anti-inflammatory agent.

### IV. Methods of Treatment

In an aspect is provided a method of treating cancer, the method including administering to a subject in need thereof an effective amount of a Reticulon 4 inhibitor. In embodiments, the Reticulon 4 inhibitor is a compound described herein. In embodiments, the Reticulon 4 inhibitor is an oligonucleotide (e.g., DNA, RNA, or siRNA), protein (e.g., antibody, anti-Reticulon 4 antibody, anti-Reticulon 4 binding antibody fragment), or compound (e.g., compound described herein). In embodiments, the Reticulon 4 inhibitor is included in a therapeutically effective amount. In embodiments, the Reticulon 4 inhibitor is an antisense nucleic acid.

In an aspect is provided a method of treating cancer including administering to a subject in need thereof an effective amount of a compound described herein. In embodiments, the cancer is colorectal cancer. In embodiments, the cancer is liver cancer. In embodiments, the cancer is hepatocellular cancer. In embodiments, the cancer is breast cancer. In embodiments, the cancer is estrogen receptor positive breast cancer. In embodiments, the cancer is estrogen receptor (ER) negative breast cancer. In embodiments, the cancer is tamoxifen resistant breast cancer. In embodiments, the cancer is HER2 negative breast cancer. In embodiments, the cancer is HER2 positive breast cancer. In embodiments, the cancer is low grade (well differentiated) breast cancer. In embodiments, the cancer is intermediate grade (moderately differentiated) breast cancer. In embodiments, the cancer is high grade (poorly differentiated) breast cancer. In embodiments, the cancer is stage 0 breast cancer. In embodiments, the cancer is stage I breast cancer. In embodiments, the cancer is stage II breast cancer. In embodiments, the cancer is stage III breast cancer. In embodiments, the cancer is stage IV breast cancer. In embodiments, the cancer is triple negative breast cancer.

In an aspect is provided a method of treating a neurodegenerative disease, the method including administering to a subject in need thereof an effective amount of a Reticulon 4 inhibitor. In an aspect is provided a method of treating nerve damage, the method including administering to a subject in need thereof an effective amount of a Reticulon 4 inhibitor. In an aspect is provided a method of treating a traumatic brain injury, the method including administering to a subject in need thereof an effective amount of a Reticulon 4 inhibitor. In an aspect is provided a method of treating a spinal cord injury, the method including administering to a subject in need thereof an effective amount of a Reticulon 4 inhibitor. In an aspect is provided a method of treating stroke, the method including administering to a subject in need thereof an effective amount of a Reticulon 4 inhibitor. In embodiments, the Reticulon 4 inhibitor is a compound described herein. In embodiments, the Reticulon 4 inhibitor is an oligonucleotide (e.g., DNA, RNA, or siRNA), protein (e.g., antibody, anti-Reticulon 4 antibody, anti-Reticulon 4 binding antibody fragment), or compound (e.g., compound described herein). In embodiments, the Reticulon 4 inhibitor is included in a therapeutically effective amount. In embodiments, the neurodegenerative disease is ALS. In embodiments, the neurodegenerative disease is multiple sclerosis.

In an aspect is provided a method of treating neurodegenerative disease including administering to a subject in need thereof an effective amount of a compound described herein. In an aspect is provided a method of treating nerve damage including administering to a subject in need thereof an effective amount of a compound described herein. In an aspect is provided a method of treating traumatic brain injury including administering to a subject in need thereof an effective amount of a compound described herein. In an aspect is provided a method of treating spinal cord injury including administering to a subject in need thereof an effective amount of a compound described herein. In an aspect is provided a method of treating stroke including administering to a subject in need thereof an effective amount of a compound described herein. In embodiments, the neurodegenerative disease is ALS. In embodiments, the neurodegenerative disease is multiple sclerosis.

In an aspect is provided a method of treating a disease associated with reticulon 4 activity including administering to a subject in need thereof an effective amount of a Reticulon 4 inhibitor. In embodiments, the Reticulon 4 inhibitor is a compound described herein. In embodiments, the Reticulon 4 inhibitor is an oligonucleotide (e.g., DNA, RNA, or siRNA), protein (e.g., antibody, anti-Reticulon 4 antibody, anti-Reticulon 4 binding antibody fragment), or compound (e.g., compound described herein). In embodiments, the disease is associated with aberrant reticulon 4 activity.

In an aspect is provided a method of increasing nerve growth (e.g., neurite growth, neuron growth), the method including administering to a subject (e.g., contacting the nerve or neurite) in need thereof an effective amount of a Reticulon 4 inhibitor. In embodiments, the Reticulon 4 inhibitor is a compound described herein. In embodiments, the Reticulon 4 inhibitor is an oligonucleotide (e.g., DNA, RNA, or siRNA), protein (e.g., antibody, anti-Reticulon 4 antibody, anti-Reticulon 4 binding antibody fragment), or compound (e.g., compound described herein). In embodiments, the Reticulon 4 inhibitor is included in a therapeutically effective amount.

In an aspect is provided a method of increasing nerve growth (e.g., neurite growth, neuron growth) including administering to a subject (e.g., contacting the nerve or neurite) in need thereof an effective amount of a compound described herein.

In embodiments, the method includes administering a second agent (e.g. therapeutic agent). In embodiments, the method includes administering a second agent (e.g. therapeutic agent) in a therapeutically effective amount. In embodiments, the second agent is an agent for treating cancer. In embodiments, the second agent is an anti-cancer agent. In embodiments, the second agent is a chemotherapeutic. In embodiments, the second agent is an agent for treating a neurodegenerative disease. In embodiments, the second agent is an agent for promoting nerve growth. In embodiments, the second agent is an agent for treating traumatic brain injury. In embodiments, the second agent is an agent for treating nerve damage. In embodiments, the second agent is an agent for treating spinal cord injury. In embodiments, the second agent is an agent for treating stroke.

### V. Methods of Inhibition

In an aspect is provided a method of inhibiting reticulon 4 activity including contacting the reticulon 4 with a Reticulon 4 inhibitor. In embodiments, the reticulon 4 is a human reticulon 4. In embodiments, the Reticulon 4 inhibitor is a compound described herein. In embodiments, the Reticulon 4 inhibitor is an oligonucleotide (e.g., DNA, RNA, or siRNA), protein (e.g., antibody, anti-Reticulon 4 antibody, anti-Reticulon 4 binding antibody fragment), or compound (e.g., compound described herein). In embodiments, the Reticulon 4 inhibitor is provided in a therapeutically effective amount.

In embodiments, the reticulon 4 is SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:331, SEQ ID NO:341, or SEQ ID NO:342. In embodiments, the reticulon 4 is SEQ ID NO:333. In embodiments, the reticulon 4 is SEQ ID NO:334. In embodiments, the reticulon 4 is SEQ ID NO:335. In embodiments, the reticulon 4 is SEQ ID NO:336. In embodiments, the reticulon 4 is SEQ ID NO:337. In embodiments, the reticulon 4 is SEQ ID NO:338. In embodiments, the reticulon 4 is SEQ ID NO:339. In embodiments, the reticulon 4 is SEQ ID NO:340. In embodiments, the reticulon 4 is SEQ ID NO:331. In embodiments, the reticulon 4 is SEQ ID NO:341. In embodiments, the reticulon 4 is SEQ ID NO:342.

In embodiments, the Reticulon 4 inhibitor contacts one or more amino acids corresponding to E1105, C1101, E1078, S1079, A1082, I1083, K1090, Y1091, S1094, G1097, and H1098 of human reticulon 4 (e.g., SEQ ID NO:331). In embodiments, the Reticulon 4 inhibitor contacts one or more amino acids corresponding to E1105, E1078, S1079, A1082, I1083, K1090, Y1091, S1094, G1097, and H1098 of SEQ ID NO:331. In embodiments, the Reticulon 4 inhibitor contacts one or more amino acids corresponding to E1105, E1078, S1079, A1082, I1083, K1090, Y1091, S1094, G1097, and H1098 of SEQ ID NO:331. In embodiments, the Reticulon 4 inhibitor covalently binds an amino acid corresponding to C1101 of SEQ ID NO:331 in human reticulon 4. In embodiments, the Reticulon 4 inhibitor contacts an amino acids corresponding to E1105, C1101, E1078, S1079, A1082, I1083, K1090, Y1091, S1094, G1097, and H1098 of SEQ ID NO:331. In embodiments, the Reticulon 4 inhibitor contacts an amino acids corresponding to E1105, E1078, S1079, A1082, I1083, K1090, Y1091, S1094, G1097, and H1098 of SEQ ID NO:331.

In embodiments, the Reticulon 4 inhibitor contacts an amino acids corresponding to E1105 of SEQ ID NO:331. In embodiments, the Reticulon 4 inhibitor contacts an amino acids corresponding to C1101 of SEQ ID NO:331. In embodiments, the Reticulon 4 inhibitor contacts an amino acids corresponding to E1078 of SEQ ID NO:331. In embodiments, the Reticulon 4 inhibitor contacts an amino acids corresponding to S1079 of SEQ ID NO:331. In embodiments, the Reticulon 4 inhibitor contacts an amino acids corresponding to A1082 of SEQ ID NO:331. In embodiments, the Reticulon 4 inhibitor contacts an amino acids corresponding to I1083 of SEQ ID NO:331. In embodiments, the Reticulon 4 inhibitor contacts an amino acids corresponding to K1090 of SEQ ID NO:331. In embodiments, the Reticulon 4 inhibitor contacts an amino acids corresponding to Y1091 of SEQ ID NO:331. In embodiments, the Reticulon 4 inhibitor contacts an amino acids corresponding to S1094 of SEQ ID NO:331. In embodiments, the Reticulon 4 inhibitor contacts an amino acids corresponding to G1097 of SEQ ID NO:331. In embodiments, the Reticulon 4 inhibitor contacts an amino acids corresponding to H1098 of SEQ ID NO:331.

In an aspect is provided a method of inhibiting reticulon 4 activity including contacting the reticulon 4 with a compound described herein. In embodiments, the reticulon 4 is a human reticulon 4. In embodiments, the compound is provided in an effective amount. In embodiments, the compound is provided in a therapeutically effective amount. In embodiments, the method includes contacting the reticulon 4 protein with an effective amount of a compound described herein. In embodiments, compound is covalently bonded to the amino acid corresponding to C1101 of SEQ ID NO:331. In embodiments, the compound contacts one or more amino acids corresponding to E1105, C1101, E1078, S1079, A1082, I1083, K1090, Y1091, S1094, G1097, and H1098 of SEQ ID NO:331. In embodiments, the compound contacts one or more amino acids corresponding to E1105, E1078, S1079, A1082, I1083, K1090, Y1091, S1094, G1097, and H1098 of SEQ ID NO:331. In embodiments, the compound covalently binds an amino acid corresponding to C1101 in SEQ ID NO:331. In embodiments, the compound contacts an amino acids corresponding to E1105, C1101, E1078, S1079, A1082, I1083, K1090, Y1091, S1094, G1097, and H1098 of SEQ ID NO:331. In embodiments, the compound contacts an amino acids corresponding to E1105, E1078, S1079, A1082, I1083, K1090, Y1091, S1094, G1097, and H1098 of SEQ ID NO:331. In embodiments, the compound contacts an amino acids corresponding to E1105 of SEQ ID NO:331. In embodiments, the compound contacts an amino acids corresponding to C1101 of SEQ ID NO:331. In embodiments, the compound contacts an amino acids corresponding to E1078 of SEQ ID NO:331. In embodiments, the compound contacts an amino acids corresponding to S1079 of SEQ ID NO:331. In embodiments, the compound contacts an amino acids corresponding to A1082 of SEQ ID NO:331. In embodiments, the compound contacts an amino acids corresponding to I1083 of SEQ ID NO:331. In embodiments, the compound contacts an amino acids corresponding to K1090 of SEQ ID NO:331. In embodiments, the compound contacts an amino acids corresponding to Y1091 of SEQ ID NO:331. In embodiments, the compound contacts an amino acids corresponding to S1094 of SEQ ID NO:331. In embodiments, the compound contacts an amino acids corresponding to G1097 of SEQ ID NO:331. In embodiments, the compound contacts an amino acids corresponding to H1098 of SEQ ID NO:331.

In embodiments, the compound contacts a cysteine in a sequence described herein. In embodiments, the sequence is (SEQ ID NO:1). In embodiments, the sequence is (SEQ ID NO:2). In embodiments, the sequence is (SEQ ID NO:3). In embodiments, the sequence is (SEQ ID NO:4). In embodiments, the sequence is (SEQ ID NO:5). In embodiments, the sequence is (SEQ ID NO:6). In embodiments, the sequence is (SEQ ID NO:7). In embodiments, the sequence is (SEQ ID NO:8). In embodiments, the sequence is (SEQ ID NO:9). In embodiments, the sequence is (SEQ ID NO:10). In embodiments, the sequence is (SEQ ID NO:11). In embodiments, the sequence is (SEQ ID NO:12). In embodiments, the sequence is (SEQ ID NO:13). In embodiments, the sequence is (SEQ ID NO:14). In embodiments, the sequence is (SEQ ID NO:15). In embodiments, the sequence is (SEQ ID NO:16). In embodiments, the sequence is (SEQ ID NO:17). In embodiments, the sequence is (SEQ ID NO:18). In embodiments, the sequence is (SEQ ID NO:19). In embodiments, the sequence is (SEQ ID NO:20). In embodiments, the sequence is (SEQ ID NO:21). In embodiments, the sequence is (SEQ ID NO:22). In embodiments, the sequence is (SEQ ID NO:23). In embodiments, the sequence is (SEQ ID NO:24). In embodiments, the sequence is (SEQ ID NO:25). In embodiments, the sequence is (SEQ ID NO:26). In embodiments, the sequence is (SEQ ID NO:27). In embodiments, the sequence is (SEQ ID NO:28). In embodiments, the sequence is (SEQ ID NO:29). In embodiments, the sequence is (SEQ ID NO:30). In embodiments, the sequence is (SEQ ID NO:31). In embodiments, the sequence is (SEQ ID NO:32). In embodiments, the sequence is (SEQ ID NO:33). In embodiments, the sequence is (SEQ ID NO:34). In embodiments, the sequence is (SEQ ID NO:35). In embodiments, the sequence is (SEQ ID NO:36). In embodiments, the sequence is (SEQ ID NO:37). In embodiments, the sequence is (SEQ ID NO:38). In embodiments, the sequence is (SEQ ID NO:39). In embodiments, the sequence is (SEQ ID NO:40). In embodiments, the sequence is (SEQ ID NO:41). In embodiments, the sequence is (SEQ ID NO:42). In embodiments, the sequence is (SEQ ID NO:43). In embodiments, the sequence is (SEQ ID NO:44). In embodiments, the sequence is (SEQ ID NO:45). In embodiments, the sequence is (SEQ ID NO:46). In embodiments, the sequence is (SEQ ID NO:47). In embodiments, the sequence is (SEQ ID NO:48). In embodiments, the sequence is (SEQ ID NO:49). In embodiments, the sequence is (SEQ ID NO:50). In embodiments, the sequence is (SEQ ID NO:51). In embodiments, the sequence is (SEQ ID NO:52). In embodiments, the sequence is (SEQ ID NO:53). In embodiments, the sequence is (SEQ ID NO:54). In embodiments, the sequence is (SEQ ID NO:55). In embodiments, the sequence is (SEQ ID NO:56). In embodiments, the sequence is (SEQ ID NO:57). In embodiments, the sequence is (SEQ ID NO:58). In embodiments, the sequence is (SEQ ID NO:59). In embodiments, the sequence is (SEQ ID NO:60). In embodiments, the sequence is (SEQ ID NO:61). In embodiments, the sequence is (SEQ ID NO:62). In embodiments, the sequence is (SEQ ID NO:63). In embodiments, the sequence is (SEQ ID NO:64). In embodiments, the sequence is (SEQ ID NO:65). In embodiments, the sequence is (SEQ ID NO:66). In embodiments, the sequence is (SEQ ID NO:67). In embodiments, the sequence is (SEQ ID NO:68). In embodiments, the sequence is (SEQ ID NO:69). In embodiments, the sequence is (SEQ ID NO:70). In embodiments, the sequence is (SEQ ID NO:71). In embodiments, the sequence is (SEQ ID NO:72). In embodiments, the sequence is (SEQ ID NO:73). In embodiments, the sequence is (SEQ ID NO:74). In embodiments, the sequence is (SEQ ID NO:75). In embodiments, the sequence is (SEQ ID NO:76). In embodiments, the sequence is (SEQ ID NO:77). In embodiments, the sequence is (SEQ ID NO:78). In embodiments, the sequence is (SEQ ID NO:79). In embodiments, the sequence is (SEQ ID NO:80). In embodiments, the sequence is (SEQ ID NO:81). In embodiments, the sequence is (SEQ ID NO:82). In embodiments, the sequence is (SEQ ID NO:83). In embodiments, the sequence is (SEQ ID NO:84). In embodiments, the sequence is (SEQ ID NO:85). In embodiments, the sequence is (SEQ ID NO:86). In embodiments, the sequence is (SEQ ID NO:87). In embodiments, the sequence is (SEQ ID NO:88). In embodiments, the sequence is (SEQ ID NO:89). In embodiments, the sequence is (SEQ ID NO:90). In embodiments, the sequence is (SEQ ID NO:91). In embodiments, the sequence is (SEQ ID NO:92). In embodiments, the sequence is (SEQ ID NO:93). In embodiments, the sequence is (SEQ ID NO:94). In embodiments, the sequence is (SEQ ID NO:95). In embodiments, the sequence is (SEQ ID NO:96). In embodiments, the sequence is (SEQ ID NO:97). In embodiments, the sequence is (SEQ ID NO:98). In embodiments, the sequence is (SEQ ID NO:99). In embodiments, the sequence is (SEQ ID NO:100). In embodiments, the sequence is (SEQ ID NO:101). In embodiments, the sequence is (SEQ ID NO:102). In embodiments, the sequence is (SEQ ID NO:103). In embodiments, the sequence is (SEQ ID NO:104). In embodiments, the sequence is (SEQ ID NO:105). In embodiments, the sequence is (SEQ ID NO:106). In embodiments, the sequence is (SEQ ID NO:107). In embodiments, the sequence is (SEQ ID NO:108). In embodiments, the sequence is (SEQ ID NO:109). In embodiments, the sequence is (SEQ ID NO:110). In embodiments, the sequence is (SEQ ID NO:111). In embodiments, the sequence is (SEQ ID NO:112). In embodiments, the sequence is (SEQ ID NO:113). In embodiments, the sequence is (SEQ ID NO:114). In embodiments, the sequence is (SEQ ID NO:115). In embodiments, the sequence is (SEQ ID NO:116). In embodiments, the sequence is (SEQ ID NO:117). In embodiments, the sequence is (SEQ ID NO:118). In embodiments, the sequence is (SEQ ID NO:119). In embodiments, the sequence is (SEQ ID NO:120). In embodiments, the sequence is (SEQ ID NO:121). In embodiments, the sequence is (SEQ ID NO:122). In embodiments, the sequence is (SEQ ID NO:123). In embodiments, the sequence is (SEQ ID NO:124). In embodiments, the sequence is (SEQ ID NO:125). In embodiments, the sequence is (SEQ ID NO:126). In embodiments, the sequence is (SEQ ID NO:127). In embodiments, the sequence is (SEQ ID NO:128). In embodiments, the sequence is (SEQ ID NO:129). In embodiments, the sequence is (SEQ ID NO:130). In embodiments, the sequence is (SEQ ID NO:131). In embodiments, the sequence is (SEQ ID NO:132). In embodiments, the sequence is (SEQ ID NO:133). In embodiments, the sequence is (SEQ ID NO:134). In embodiments, the sequence is (SEQ ID NO:135). In embodiments, the sequence is (SEQ ID NO:136). In embodiments, the sequence is (SEQ ID NO:137). In embodiments, the sequence is (SEQ ID NO:138). In embodiments, the sequence is (SEQ ID NO:139). In embodiments, the sequence is (SEQ ID NO:140). In embodiments, the sequence is (SEQ ID NO:141). In embodiments, the sequence is (SEQ ID NO:142). In embodiments, the sequence is (SEQ ID NO:143). In embodiments, the sequence is (SEQ ID NO:144). In embodiments, the sequence is (SEQ ID NO:145). In embodiments, the sequence is (SEQ ID NO:146). In embodiments, the sequence is (SEQ ID NO:147). In embodiments, the sequence is (SEQ ID NO:148). In embodiments, the sequence is (SEQ ID NO:149). In embodiments, the sequence is (SEQ ID NO:150). In embodiments, the sequence is (SEQ ID NO:151). In embodiments, the sequence is (SEQ ID NO:152). In embodiments, the sequence is (SEQ ID NO:153). In embodiments, the sequence is (SEQ ID NO:154). In embodiments, the sequence is (SEQ ID NO:155). In embodiments, the sequence is (SEQ ID NO:156). In embodiments, the sequence is (SEQ ID NO:157). In embodiments, the sequence is (SEQ ID NO:158). In embodiments, the sequence is (SEQ ID NO:159). In embodiments, the sequence is (SEQ ID NO:160). In embodiments, the sequence is (SEQ ID NO:161). In embodiments, the sequence is (SEQ ID NO:162). In embodiments, the sequence is (SEQ ID NO:163). In embodiments, the sequence is (SEQ ID NO:164). In embodiments, the sequence is (SEQ ID NO:165). In embodiments, the sequence is (SEQ ID NO:166). In embodiments, the sequence is (SEQ ID NO:167). In embodiments, the sequence is (SEQ ID NO:168). In embodiments, the sequence is (SEQ ID NO:169). In embodiments, the sequence is (SEQ ID NO:170). In embodiments, the sequence is (SEQ ID NO:171). In embodiments, the sequence is (SEQ ID NO:172). In embodiments, the sequence is (SEQ ID NO:173). In embodiments, the sequence is (SEQ ID NO:174). In embodiments, the sequence is (SEQ ID NO:175). In embodiments, the sequence is (SEQ ID NO:176). In embodiments, the sequence is (SEQ ID NO:177). In embodiments, the sequence is (SEQ ID NO:178). In embodiments, the sequence is (SEQ ID NO:179). In embodiments, the sequence is (SEQ ID NO:180). In embodiments, the sequence is (SEQ ID NO:181). In embodiments, the sequence is (SEQ ID NO:182). In embodiments, the sequence is (SEQ ID NO:183). In embodiments, the sequence is (SEQ ID NO:184). In embodiments, the sequence is (SEQ ID NO:185). In embodiments, the sequence is (SEQ ID NO:186). In embodiments, the sequence is (SEQ ID NO:187). In embodiments, the sequence is (SEQ ID NO:188). In embodiments, the sequence is (SEQ ID NO:189). In embodiments, the sequence is (SEQ ID NO:190). In embodiments, the sequence is (SEQ ID NO:191). In embodiments, the sequence is (SEQ ID NO:192). In embodiments, the sequence is (SEQ ID NO:193). In embodiments, the sequence is (SEQ ID NO:194). In embodiments, the sequence is (SEQ ID NO:195). In embodiments, the sequence is (SEQ ID NO:196). In embodiments, the sequence is (SEQ ID NO:197). In embodiments, the sequence is (SEQ ID NO:198). In embodiments, the sequence is (SEQ ID NO:199). In embodiments, the sequence is (SEQ ID NO:200). In embodiments, the sequence is (SEQ ID NO:201). In embodiments, the sequence is (SEQ ID NO:202). In embodiments, the sequence is (SEQ ID NO:203). In embodiments, the sequence is (SEQ ID NO:204). In embodiments, the sequence is (SEQ ID NO:205). In embodiments, the sequence is (SEQ ID NO:206). In embodiments, the sequence is (SEQ ID NO:207). In embodiments, the sequence is (SEQ ID NO:208). In embodiments, the sequence is (SEQ ID NO:209). In embodiments, the sequence is (SEQ ID NO:210). In embodiments, the sequence is (SEQ ID NO:211). In embodiments, the sequence is (SEQ ID NO:212). In embodiments, the sequence is (SEQ ID NO:213). In embodiments, the sequence is (SEQ ID NO:214). In embodiments, the sequence is (SEQ ID NO:215). In embodiments, the sequence is (SEQ ID NO:216). In embodiments, the sequence is (SEQ ID NO:217). In embodiments, the sequence is (SEQ ID NO:218). In embodiments, the sequence is (SEQ ID NO:219). In embodiments, the sequence is (SEQ ID NO:220). In embodiments, the sequence is (SEQ ID NO:221). In embodiments, the sequence is (SEQ ID NO:222). In embodiments, the sequence is (SEQ ID NO:223). In embodiments, the sequence is (SEQ ID NO:224). In embodiments, the sequence is (SEQ ID NO:225). In embodiments, the sequence is (SEQ ID NO:226). In embodiments, the sequence is (SEQ ID NO:227). In embodiments, the sequence is (SEQ ID NO:228). In embodiments, the sequence is (SEQ ID NO:229). In embodiments, the sequence is (SEQ ID NO:230). In embodiments, the sequence is (SEQ ID NO:231). In embodiments, the sequence is (SEQ ID NO:232). In embodiments, the sequence is (SEQ ID NO:233). In embodiments, the sequence is (SEQ ID NO:234). In embodiments, the sequence is (SEQ ID NO:235). In embodiments, the sequence is (SEQ ID NO:236). In embodiments, the sequence is (SEQ ID NO:237). In embodiments, the sequence is (SEQ ID NO:238). In embodiments, the sequence is (SEQ ID NO:239). In embodiments, the sequence is (SEQ ID NO:240). In embodiments, the sequence is (SEQ ID NO:241). In embodiments, the sequence is (SEQ ID NO:242). In embodiments, the sequence is (SEQ ID NO:243). In embodiments, the sequence is (SEQ ID NO:244). In embodiments, the sequence is (SEQ ID NO:245). In embodiments, the sequence is (SEQ ID NO:246). In embodiments, the sequence is (SEQ ID NO:247). In embodiments, the sequence is (SEQ ID NO:248). In embodiments, the sequence is (SEQ ID NO:249). In embodiments, the sequence is (SEQ ID NO:250). In embodiments, the sequence is (SEQ ID NO:251). In embodiments, the sequence is (SEQ ID NO:252). In embodiments, the sequence is (SEQ ID NO:253). In embodiments, the sequence is (SEQ ID NO:254). In embodiments, the sequence is (SEQ ID NO:255). In embodiments, the sequence is (SEQ ID NO:256). In embodiments, the sequence is (SEQ ID NO:257). In embodiments, the sequence is (SEQ ID NO:258). In embodiments, the sequence is (SEQ ID NO:259). In embodiments, the sequence is (SEQ ID NO:260). In embodiments, the sequence is (SEQ ID NO:261). In embodiments, the sequence is (SEQ ID NO:262). In embodiments, the sequence is (SEQ ID NO:263). In embodiments, the sequence is (SEQ ID NO:264). In embodiments, the sequence is (SEQ ID NO:265). In embodiments, the sequence is (SEQ ID NO:266). In embodiments, the sequence is (SEQ ID NO:267). In embodiments, the sequence is (SEQ ID NO:268). In embodiments, the sequence is (SEQ ID NO:269). In embodiments, the sequence is (SEQ ID NO:270). In embodiments, the sequence is (SEQ ID NO:271). In embodiments, the sequence is (SEQ ID NO:272). In embodiments, the sequence is (SEQ ID NO:273). In embodiments, the sequence is (SEQ ID NO:274). In embodiments, the sequence is (SEQ ID NO:275). In embodiments, the sequence is (SEQ ID NO:276). In embodiments, the sequence is (SEQ ID NO:277). In embodiments, the sequence is (SEQ ID NO:278). In embodiments, the sequence is (SEQ ID NO:279). In embodiments, the sequence is (SEQ ID NO:280). In embodiments, the sequence is (SEQ ID NO:281). In embodiments, the sequence is (SEQ ID NO:282). In embodiments, the sequence is (SEQ ID NO:283). In embodiments, the sequence is (SEQ ID NO:284). In embodiments, the sequence is (SEQ ID NO:285). In embodiments, the sequence is (SEQ ID NO:286). In embodiments, the sequence is (SEQ ID NO:287). In embodiments, the sequence is (SEQ ID NO:288). In embodiments, the sequence is (SEQ ID NO:289). In embodiments, the sequence is (SEQ ID NO:290). In embodiments, the sequence is (SEQ ID NO:291). In embodiments, the sequence is (SEQ ID NO:292). In embodiments, the sequence is (SEQ ID NO:293). In embodiments, the sequence is (SEQ ID NO:294). In embodiments, the sequence is (SEQ ID NO:295). In embodiments, the sequence is (SEQ ID NO:296). In embodiments, the sequence is (SEQ ID NO:297). In embodiments, the sequence is (SEQ ID NO:298). In embodiments, the sequence is (SEQ ID NO:299). In embodiments, the sequence is (SEQ ID NO:300). In embodiments, the sequence is (SEQ ID NO:301). In embodiments, the sequence is (SEQ ID NO:302). In embodiments, the sequence is (SEQ ID NO:303). In embodiments, the sequence is (SEQ ID NO:304). In embodiments, the sequence is (SEQ ID NO:305). In embodiments, the sequence is (SEQ ID NO:306). In embodiments, the sequence is (SEQ ID NO:307). In embodiments, the sequence is (SEQ ID NO:308). In embodiments, the sequence is (SEQ ID NO:309). In embodiments, the sequence is (SEQ ID NO:310). In embodiments, the sequence is (SEQ ID NO:311). In embodiments, the sequence is (SEQ ID NO:312). In embodiments, the sequence is (SEQ ID NO:313). In embodiments, the sequence is (SEQ ID NO:314). In embodiments, the sequence is (SEQ ID NO:315). In embodiments, the sequence is (SEQ ID NO:316). In embodiments, the sequence is (SEQ ID NO:317). In embodiments, the sequence is (SEQ ID NO:318). In embodiments, the sequence is (SEQ ID NO:319). In embodiments, the sequence is (SEQ ID NO:320). In embodiments, the sequence is (SEQ ID NO:321). In embodiments, the sequence is (SEQ ID NO:322). In embodiments, the sequence is (SEQ ID NO:323). In embodiments, the sequence is (SEQ ID NO:324). In embodiments, the sequence is (SEQ ID NO:325). In embodiments, the sequence is (SEQ ID NO:326). In embodiments, the sequence is (SEQ ID NO:327). In embodiments, the sequence is (SEQ ID NO:328). In embodiments, the sequence is (SEQ ID NO:329). In embodiments, the sequence is (SEQ ID NO:330). In embodiments, the sequence is (SEQ ID NO:331). In embodiments, the sequence is (SEQ ID NO:332). In embodiments, the sequence is (SEQ ID NO:333). In embodiments, the sequence is (SEQ ID NO:334). In embodiments, the sequence is (SEQ ID NO:335). In embodiments, the sequence is (SEQ ID NO:336). In embodiments, the sequence is (SEQ ID NO:337). In embodiments, the sequence is (SEQ ID NO:338). In embodiments, the sequence is (SEQ ID NO:339). In embodiments, the sequence is (SEQ ID NO:340). In embodiments, the sequence is (SEQ ID NO:341). In embodiments, the sequence is (SEQ ID NO:342). In embodiments, the sequence is (SEQ ID NO:343).

In embodiments, the inhibition is competitive inhibition. In embodiments, the inhibition is irreversible. In embodiments, the inhibition is reversible. In embodiments, the compound covalently binds to the reticulon 4 protein.

Where the compound covalently binds to the reticulon 4 a reticulon 4 protein (e.g., human reticulon 4) covalently bonded to a reticulon 4 inhibitor is formed (also referred to herein as a “reticulon 4-compound adduct”), as described below. In embodiments, the resulting covalent bond is reversible. Where the resulting covalent bond is reversible, the bonding reverses upon denaturation of the protein. Thus, in embodiments, the reversibility of a covalent bond between the compound and the reticulon 4 upon denaturation of the reticulon 4 avoids or decreases autoimmune response in a subject subsequent to administration of the compound (relative to irreversibility). Moreover, in embodiments, the reversibility of a covalent bond between the compound and the reticulon 4 upon denaturation of the reticulon 4 avoids or decreases the toxicity (e.g. liver toxicity) of the compound in a subject (relative to irreversibility).

In embodiments, the reticulon 4 activity is endoplasmic reticulum (ER) tubule formation. In embodiments, the reticulon 4 activity is an increase in endoplasmic reticulum (ER) tubule formation. In embodiments, the reticulon 4 activity is RTN 4 membrane associate. In embodiments, the reticulon 4 activity is RTN 4 membrane contact. In embodiments, the reticulon 4 activity is increasing ER tubule networks. In embodiments, the reticulon 4 activity is nuclear envelope assembly (e.g., during mitosis). In embodiments, the reticulon 4 activity is nuclear envelope formation (e.g., during mitosis). In embodiments, the reticulon 4 activity is nuclear envelope disassembly (e.g., during mitosis). In embodiments, the reticulon 4 activity is increasing cell division. In embodiments, the reticulon 4 activity is increasing the rate of cell divisional. In embodiments, the reticulon 4 activity is promoting cell division. In embodiments, the reticulon 4 activity is completing cell division. In embodiments, the reticulon 4 activity is maintaining natural nuclear envelope morphology (e.g., during mitosis). In embodiments, the reticulon 4 activity is maintaining natural interphase nuclear envelope morphology. In embodiments, the reticulon 4 activity is nuclear envelope remodeling (e.g., during mitosis). In embodiments, the reticulon 4 activity is inhibition of neurite cell growth. In embodiments, the reticulon 4 activity is neuron growth. In embodiments, the reticulon 4 activity is neuron survival. In embodiments, the reticulon 4 activity is neuron proliferation. In embodiments, the reticulon 4 activity is completing mitosis. In embodiments, the reticulon 4 activity is increasing the rate of mitosis (e.g, compared to lack of RTN 4 activity).

In an aspect is provided a method of inhibiting cell divisional (e.g., cancer cell division, cancer proliferation), the method including contacting a cell (e.g., cancer cell) with an effective amount of a Reticulon 4 inhibitor. In embodiments, the Reticulon 4 inhibitor is a compound described herein. In embodiments, the Reticulon 4 inhibitor is an oligonucleotide (e.g., DNA, RNA, or siRNA), protein (e.g., antibody, anti-Reticulon 4 antibody, anti-Reticulon 4 binding antibody fragment), or compound (e.g., compound described herein).

In an aspect is provided a method of inhibiting cell divisional (e.g., cancer cell division, cancer proliferation) including contacting a cell (e.g., cancer cell) with an effective amount of a compound described herein.

### VI. Reticulon 4 Protein

In an aspect is provided a reticulon 4 protein covalently bonded to a Reticulon 4 inhibitor (a reticulon 4 protein-reticulon 4 inhibitor complex). In embodiments, the reticulon 4 is a human reticulon 4. In embodiments, the reticulon 4 is has the sequence SEQ ID NO:331. In embodiments, the Reticulon 4 inhibitor is a compound described herein. In embodiments, the Reticulon 4 inhibitor is an oligonucleotide (e.g., DNA, RNA, or siRNA), protein (e.g., antibody, anti-Reticulon 4 antibody, anti-Reticulon 4 binding antibody fragment), or compound (e.g., compound described herein). In embodiments, the Reticulon 4 inhibitor is provided in a therapeutically effective amount. In embodiments, the Reticulon 4 inhibitor contacts one or more amino acids corresponding to E1105, C1101, E1078, S1079, A1082, I1083, K1090, Y1091, S1094, G1097, and H1098 of SEQ ID NO:331. In embodiments, the Reticulon 4 inhibitor covalently binds an amino acid corresponding to C1101 in SEQ ID NO:331. In embodiments, the Reticulon 4 inhibitor contacts an amino acids corresponding to E1105, C1101, E1078, S1079, A1082, I1083, K1090, Y1091, S1094, G1097, and H1098 of SEQ ID NO:331. In embodiments, the Reticulon 4 inhibitor contacts an amino acids corresponding to E1105 of SEQ ID NO:331. In embodiments, the Reticulon 4 inhibitor contacts an amino acids corresponding to C1101 of SEQ ID NO:331. In embodiments, the Reticulon 4 inhibitor contacts an amino acids corresponding to E1078 of SEQ ID NO:331. In embodiments, the Reticulon 4 inhibitor contacts an amino acids corresponding to S1079 of SEQ ID NO:331. In embodiments, the Reticulon 4 inhibitor contacts an amino acids corresponding to A1082 of SEQ ID NO:331. In embodiments, the Reticulon 4 inhibitor contacts an amino acids corresponding to I1083 of SEQ ID NO:331. In embodiments, the Reticulon 4 inhibitor contacts an amino acids corresponding to K1090 of SEQ ID NO:331. In embodiments, the Reticulon 4 inhibitor contacts an amino acids corresponding to Y1091 of SEQ ID NO:331. In embodiments, the Reticulon 4 inhibitor contacts an amino acids corresponding to S1094 of SEQ ID NO:331. In embodiments, the Reticulon 4 inhibitor contacts an amino acids corresponding to G1097 of SEQ ID NO:331. In embodiments, the Reticulon 4 inhibitor contacts an amino acids corresponding to H1098 of SEQ ID NO:331.

In an aspect is provided a reticulon 4 protein covalently bonded to a compound described herein. In embodiments, compound is covalently bonded to the amino acid corresponding to C1101 of SEQ ID NO:331. In embodiments, the compound contacts one or more amino acids corresponding to E1105, C1101, E1078, S1079, A1082, I1083, K1090, Y1091, S1094, G1097, and H1098 of SEQ ID NO:331. In embodiments, the compound covalently binds an amino acid corresponding to C1101 in SEQ ID NO:331. In embodiments, the compound contacts an amino acids corresponding to E1105, C1101, E1078, S1079, A1082, I1083, K1090, Y1091, S1094, G1097, and H1098 of SEQ ID NO:331. In embodiments, the compound contacts an amino acids corresponding to E1105 of SEQ ID NO:331. In embodiments, the compound contacts an amino acids corresponding to C1101 of SEQ ID NO:331. In embodiments, the compound contacts an amino acids corresponding to E1078 of SEQ ID NO:331. In embodiments, the compound contacts an amino acids corresponding to S1079 of SEQ ID NO:331. In embodiments, the compound contacts an amino acids corresponding to A1082 of SEQ ID NO:331. In embodiments, the compound contacts an amino acids corresponding to I1083 of SEQ ID NO:331. In embodiments, the compound contacts an amino acids corresponding to K1090 of SEQ ID NO:331. In embodiments, the compound contacts an amino acids corresponding to Y1091 of SEQ ID NO:331. In embodiments, the compound contacts an amino acids corresponding to S1094 of SEQ ID NO:331. In embodiments, the compound contacts an amino acids corresponding to G1097 of SEQ ID NO:331. In embodiments, the compound contacts an amino acids corresponding to H1098 of SEQ ID NO:331.

In embodiments, the compound is bonded to a cysteine residue of the reticulon 4 protein. In embodiments, the compound is covalently bonded to a cysteine residue of the reticulon 4 protein. In embodiments, the compound is reversibly covalently bonded to a cysteine residue of the reticulon 4 protein. In embodiments, the compound is irreversibly covalently bonded to a cysteine residue of the reticulon 4 protein. In embodiments, the cysteine residue corresponds to C1101 of SEQ ID NO:331.

In an embodiment, the reticulon 4 protein is covalently bonded (e.g., reversibly or irreversibly) to a portion of a compound described herein (e.g., portion of a reticulon 4 inhibitor or portion of a compound described herein).

In an aspect is provided a reticulon 4 protein (e.g., human reticulon 4) covalently bonded to a reticulon 4 inhibitor (e.g., reticulon 4 inhibitor, compound described herein, or a portion of a compound described herein).

In embodiments, the reticulon 4 protein (e.g., human reticulon 4) is covalently bonded to a reticulon 4 inhibitor (e.g., compound described herein or a portion of a compound described herein). In embodiments, the reticulon 4 protein (e.g., human reticulon 4) is irreversibly covalently bonded to a reticulon 4 inhibitor (e.g., compound described herein or a portion of a compound described herein). In embodiments, the reticulon 4 protein (e.g., human reticulon 4) is reversibly covalently bonded to a reticulon 4 inhibitor (e.g., compound described herein or a portion of a compound described herein). In embodiments, the reticulon 4 protein (e.g., human reticulon 4) is covalently bonded to a portion of a reticulon 4 inhibitor (e.g., compound described herein). In embodiments, the reticulon 4 protein (e.g., human reticulon 4) is irreversibly covalently bonded to a portion of a reticulon 4 inhibitor (e.g., compound described herein). In embodiments, the reticulon 4 protein (e.g., human reticulon 4) is reversibly covalently bonded to a portion of a reticulon 4 inhibitor (e.g., compound described herein). In embodiments, the reticulon 4 inhibitor (e.g., compound described herein) is bonded to a cysteine residue (e.g., Cys1101 of human reticulon 4 or cysteine corresponding to Cys1101 of human reticulon 4) of the reticulon 4 protein (e.g., human reticulon 4). In embodiments, the portion of a reticulon 4 inhibitor (e.g., compound described herein) is bonded to a cysteine residue (e.g., Cys1101 of SEQ ID NO:331 or cysteine corresponding to Cys1101 of SEQ ID NO:331) of the reticulon 4 protein (e.g., human reticulon 4).

In embodiments, the RTN4 protein covalently bonded to a RTN4 inhibitor or compound described herein is the product of a reaction between the RTN4 protein and a RTN4 inhibitor or compound described herein. It will be understood that the covalently bonded RTN4 protein and RTN4 inhibitor (e.g., compound described herein) are the remnants of the reactant RTN4 protein and RTN4 inhibitor or compound, wherein each reactant now participates in the covalent bond between the RTN4 protein and RTN4 inhibitor or compound. In embodiments of the covalently bonded RTN4 protein and compound described herein, the remnant of the E substitutent is a linker including a covalent bond between the RTN4 protein and the remainder of the compound described herein. It will be understood by a person of ordinary skill in the art that when a RTN4 protein is covalently bonded to a RTN4 inhibitor (e.g., compound described herein), the RTN4 inhibitor (e.g., compound described herein) forms a remnant of the pre-reacted RTN4 inhibitor (e.g., compound described herein) wherein a bond connects the remnant of the RTN4 inhibitor (e.g., compound described herein) to the remnant of the RTN4 protein (e.g., cysteine sulfur, sulfur of amino acid corresponding to C1101 of human RTN4, sulfur of C1101 of human RTN4). The remnant of the RTN4 inhibitor (compound described herein) may also be called a portion of the RTN4 inhibitor. In embodiments, the remnant of the E substituent is a linker selected from a bond, —S(O)₂—, —NH—, —O—, —S—, —C(O)—, —C(O)NH—, —NHC(O)—, —NHC(O)NH—, —NHC(O)NH—, —C(O)O—, —OC(O)—, —CH₂NH—, substituted (e.g., substituted with a substituent group, a size-limited substituent group, or lower substituent group) or unsubstituted alkylene (e.g., C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₀ or phenyl), or substituted (e.g., substituted with a substituent group, a size-limited substituent group, or lower substituent group) or unsubstituted heteroarylene (e.g., 5 to 10 membered, 5 to 9 membered, or 5 to 6 membered). As a non-limiting example, the RTN4 protein covalently bonded to a RTN4 inhibitor may have the formula:

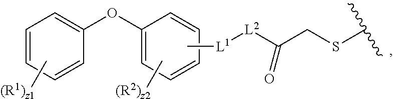

wherein S is the sulfur of a reticulon 4 protein cysteine (e.g., corresponding to C1101 of human reticulon 4), which is bonded to the remainder of the reticulon 4 protein and wherein R¹, R², L¹, L², z1, and z2 are as described herein. As a non-limiting example, the RTN4 protein covalently bonded to a RTN4 inhibitor may have the formula:

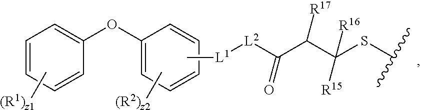

wherein S is the sulfur of a reticulon 4 protein cysteine (e.g., corresponding to C1101 of human reticulon 4), which is bonded to the remainder of the reticulon 4 protein and wherein R¹, R², R¹⁵, R¹⁷, L¹, L², z1, and z2 are as described herein. As a non-limiting example, the RTN4 protein covalently bonded to a RTN4 inhibitor may have the formula:

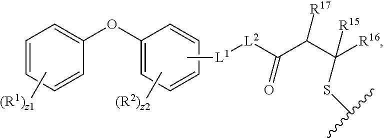

wherein S is the sulfur of a reticulon 4 protein cysteine (e.g., corresponding to C1101 of human reticulon 4), which is bonded to the remainder of the reticulon 4 protein and wherein R¹, R², R¹⁶, R¹⁷, L¹, L², z1, and z2 are as described herein.

### VII. Embodiments

### Embodiment P1

A compound having the formula:

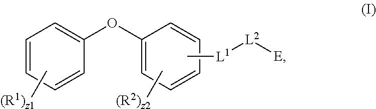

wherein, R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl; z1 is an integer from 0 to 5;

R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl; z2 is an integer from 0 to 4; L¹ is a bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene; R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene; R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; Each R^{1A}, R^{1B}, R^{1C}, R^{1D}, R^{2A}, R^{2B}, R^{2C}, R^{2D}, R^{4A}, R^{4B}, R^{5A}, and R^{5B} is independently

hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
each X, X¹, X², X⁴, and X⁵ is independently —F, —Cl, —Br, or —I;
n1, n2, n4, and n5 are independently an integer from 0 to 4; and
m1, m2, m4, m5, v1, v2, v4, and v5 are independently an integer from 1 to 2.

### Embodiment P2

The compound of embodiment P1 having the formula:

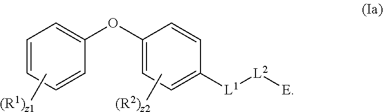

### Embodiment P3

The compound of embodiment P1 having the formula:

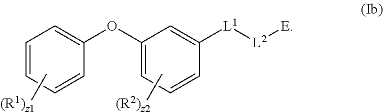

### Embodiment P4

The compound of embodiment P1 having the formula:

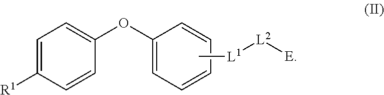

### Embodiment P5

The compound of embodiment P1 having the formula:

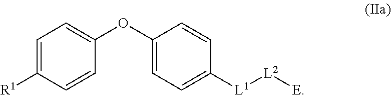

### Embodiment P6

The compound of one of embodiments P1 to P5, wherein R¹ is independently halogen, —CX¹₃, —CHX¹₂, —CH₂X¹, —OCX¹₃, —OCH₂X¹, —OCHX¹₂, —CN, —SR^{1D}, —NR^{1A}R^{1B}, —C(O)R^{1C}, —C(O)OR^{1C}, —C(O)NR^{1A}R^{1B}, —OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P7

The compound of one of embodiments P1 to P5, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ aryl, or substituted or unsubstituted 5 to 12 membered heteroaryl.

### Embodiment P8

The compound of one of embodiments P1 to P5, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

### Embodiment P9

The compound of one of embodiments P1 to P5, wherein R¹ is independently —Cl.

### Embodiment P10

The compound of embodiment P1, wherein two adjacent R¹ substituents are joined to form a substituted or unsubstituted cycloalkyl, substituted or unsubstituted heterocycloalkyl, substituted or unsubstituted aryl, or substituted or unsubstituted heteroaryl.

### Embodiment P11

The compound of embodiment P1, wherein two adjacent R¹ substituents are joined to form an unsubstituted cycloalkyl.

### Embodiment P12

The compound of embodiment P1, wherein two adjacent R¹ substituents are joined to form an unsubstituted C₃-C₆ cycloalkyl.

### Embodiment P13

The compound of one of embodiments P1 to P12, wherein L¹ is a bond, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to 6 membered heteroarylene.

### Embodiment P14

The compound of one of embodiments P1 to P12, wherein L¹ is a bond.

### Embodiment P15

The compound of one of embodiments P1 to P14, wherein L² is —NR⁵— or substituted or unsubstituted heterocycloalkylene comprising a ring nitrogen bonded directly to E.

### Embodiment P16

The compound of one of embodiments P1 to P14, wherein L² is —NR⁵—.

### Embodiment P17

The compound of embodiment P16, wherein R⁵ is hydrogen, substituted or unsubstituted C₁-C₆ alkyl, or substituted or unsubstituted 2 to 6 membered heteroalkyl.

### Embodiment P18

The compound of embodiment P16, wherein R⁵ is hydrogen or unsubstituted C₁-C₃ alkyl.

### Embodiment P19

The compound of embodiment P16, wherein R⁵ is hydrogen, unsubstituted methyl, unsubstituted ethyl, unsubstituted hexyl, or unsubstituted benzyl.

### Embodiment P20

The compound of embodiment P16, wherein R⁵ is hydrogen.

### Embodiment P21

The compound of one of embodiments P1 to P20, wherein E is a covalent cysteine modifier moiety.

### Embodiment P22

The compound of one of embodiments P1 to P20, wherein E is:

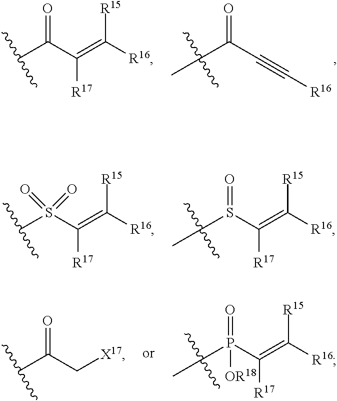

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁶ is independently hydrogen, halogen, CX¹⁶₃, —CHX¹⁶₂, —

CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B},
—NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B},

—NHC═(O)NHNR^{17A}R^{17B},
—NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, and R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;

each X, X¹⁵, X¹⁶, X¹⁷ and X¹⁸ is independently —F, —Cl, —Br, or —I;

n15, n16, n17, v15, v16, and v17, are independently an integer from 0 to 4; and

m15, m16, and m17 are independently and integer from 1 to 2.

### Embodiment P23

The compound of embodiment P22, wherein R¹⁵, R¹⁶, R¹⁷, and R¹⁸ are hydrogen.

### Embodiment P24

The compound of one of embodiments P22 to P23, wherein E is:

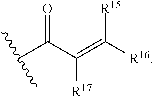

### Embodiment P25

A pharmaceutical composition comprising a Reticulon 4 inhibitor and a pharmaceutically acceptable excipient.

### Embodiment P26

A pharmaceutical composition comprising the compound of any one of embodiments P1 to P24 and a pharmaceutically acceptable excipient.

### Embodiment P27

A method of inhibiting reticulon 4 protein activity, said method comprising contacting the reticulon 4 protein with a Reticulon 4 inhibitor.

### Embodiment P28

The method of embodiment P27, wherein the Reticulon 4 inhibitor is an siRNA, antibody, or compound.

### Embodiment P29

The method of embodiment P30, wherein the Reticulon 4 inhibitor contacts one or more amino acids corresponding to E1105, C1101, E1078, 51079, A1082, I1083, K1090, Y1091, S1094, G1097, and H1098 of human reticulon 4.

### Embodiment P30

A method of inhibiting reticulon 4 protein activity, said method comprising contacting the reticulon 4 protein with an effective amount of a compound of one of embodiments P1 to P24.

### Embodiment P31

The method of embodiment P30, wherein the compound is covalently bonded to the amino acid corresponding to C1101 of human reticulon 4.

### Embodiment P32

The method of embodiment P30, wherein the compound contacts one or more amino acids corresponding to E1105, C1101, E1078, 51079, A1082, I1083, K1090,

Y1091, S1094, G1097, and H1098 of human reticulon 4.

### Embodiment P33

A method of treating cancer, said method comprising administering to a subject in need thereof an effective amount of a Reticulon 4 inhibitor.

### Embodiment P34

A method of treating cancer, said method comprising administering to a subject in need thereof an effective amount of a compound of one of embodiments P1 to P24.

### Embodiment P35

The method of one of embodiments P33 to P34, wherein the cancer is colorectal cancer.

### Embodiment P36

A reticulon 4 protein covalently bonded to a compound of one of embodiments P1 to P24.

### Embodiment P37

The Reticulon 4 protein of embodiment P36, wherein the compound is bonded to a cysteine residue of the protein.

### Embodiment P38

The reticulon 4 protein of embodiment P36, covalently bonded to a portion of a compound of one of embodiments 1 to 24.

### Embodiment P39

The reticulon 4 protein of embodiment P36, irreversibly covalently bonded to a portion of a compound of one of embodiments 1 to 24.

### Embodiment P40

The reticulon 4 protein of one of embodiments P36 to P39, wherein the compound or portion of the compound is covalently bonded to an amino acid corresponding to C1101 of human reticulon 4.

### VIII. Additional Embodiments

### Embodiment 1

A method of treating cancer, said method comprising administering to a subject in need thereof an effective amount of a compound having the

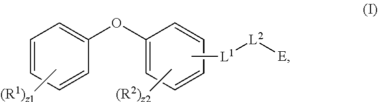

formula:

wherein, R¹ is independently halogen, —CX¹₃, —CHX¹₂, —CH₂X¹, —OCX¹₃, —OCH₂X¹, —OCHX¹₂, —CN, —SOₙ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 5;

R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(OR^{2C}—C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;
z2 is an integer from 0 to 4;

L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{2A}, R^{2B}, R^{2C}, R^{2D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X², X⁴, and X⁵ is independently —F, —Cl, —Br, or —I;

n1, n2, n4, and n5 are independently an integer from 0 to 4; and

m1, m2, m4, m5, v1, v2, v4, and v5 are independently an integer from 1 to 2.

### Embodiment 2

The method of embodiment 1, wherein the compound has the formula:

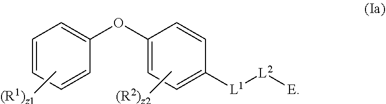

### Embodiment 3

The method of embodiment 1, wherein the compound has the formula:

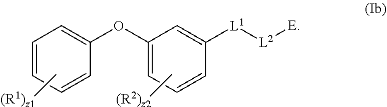

### Embodiment 4

The method of embodiment 1, wherein the compound has the formula:

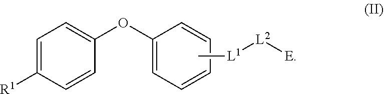

### Embodiment 5

The method of embodiment 1, wherein the compound has the formula:

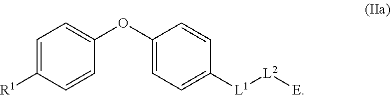

### Embodiment 6

The method of one of embodiments 1 to 5, wherein R¹ is independently halogen, —CX¹₃, —CHX¹₂, —CH₂X¹, —OCX¹₃, —OCH₂X¹, —OCHX¹₂, —CN, —SR^{1D}, —NR^{1A}R^{1B}, —C(O)R^{1C}, —C(O)OR^{1C}, —C(O)NR^{1A}R^{1B}, —OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 7

The method of one of embodiments 1 to 5, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ aryl, or substituted or unsubstituted 5 to 12 membered heteroaryl.

### Embodiment 8

The method of one of embodiments 1 to 5, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

### Embodiment 9

The method of one of embodiments 1 to 5, wherein R¹ is independently —Cl.

### Embodiment 10

The method of embodiment 1, wherein two adjacent R¹ substituents are joined to form a substituted or unsubstituted cycloalkyl, substituted or unsubstituted heterocycloalkyl, substituted or unsubstituted aryl, or substituted or unsubstituted heteroaryl.

### Embodiment 11

The method of embodiment 1, wherein two adjacent R¹ substituents are joined to form an unsubstituted cycloalkyl.

### Embodiment 12

The method of embodiment 1, wherein two adjacent R¹ substituents are joined to form an unsubstituted C₃-C₆ cycloalkyl.

### Embodiment 13

The method of one of embodiments 1 to 12, wherein L¹ is a bond, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to 6 membered heteroarylene.

### Embodiment 14

The method of one of embodiments 1 to 12, wherein L¹ is a bond.

### Embodiment 15

The method of one of embodiments 1 to 14, wherein L² is —NR⁵— or substituted or unsubstituted heterocycloalkylene comprising a ring nitrogen bonded directly to E.

### Embodiment 16

The method of one of embodiments 1 to 14, wherein L² is —NR⁵—.

### Embodiment 17

The method of embodiment 16, wherein R⁵ is hydrogen, substituted or unsubstituted C₁-C₆ alkyl, or substituted or unsubstituted 2 to 6 membered heteroalkyl.

### Embodiment 18

The method of embodiment 16, wherein R⁵ is hydrogen or unsubstituted C₁-C₃ alkyl.

### Embodiment 19

The method of embodiment 16, wherein R⁵ is hydrogen, unsubstituted methyl, unsubstituted ethyl, unsubstituted hexyl, or unsubstituted benzyl.

### Embodiment 20

The method of embodiment 16, wherein R⁵ is hydrogen.

### Embodiment 21

The method of one of embodiments 1 to 20, wherein E is a covalent cysteine modifier moiety.

### Embodiment 22

The method of one of embodiments 1 to 20, wherein E is:

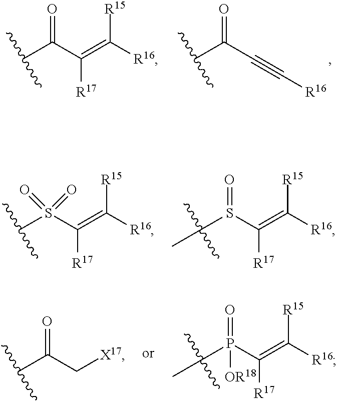

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B},

—NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, θNR^{16A}R^{16B},

—NHC═(O)NHNR^{16A}R^{16B},
—NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B},

—NHC═(O)NHNR^{17A}R^{17B},
—NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁸ is independently hydrogen, —CX¹⁸₃, —CHX¹⁸₂, —

CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, and R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;

each X, X¹⁵, X¹⁶, X¹⁷ and X¹⁸ is independently —F, —Cl, —Br, or —I;

n15, n16, n17, v15, v16, and v17, are independently an integer from 0 to 4; and

m15, m16, and m17 are independently and integer from 1 to 2.

### Embodiment 23

The method of embodiment 22, wherein R¹⁵, R¹⁶, R¹⁷, and R¹⁸ are hydrogen.

### Embodiment 24

The method of one of embodiments 22 to 23, wherein E is:

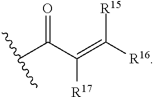

### Embodiment 25

The method of embodiment 1, having the formula:

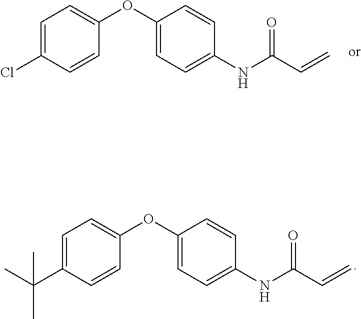

### Embodiment 26

The method of one of embodiments 1 to 25, wherein the cancer is colorectal cancer.

### Embodiment 27

The use of a compound for the preparation of a medicament for the treatment of cancer, wherein the compound has the formula:

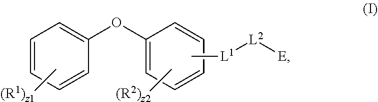

wherein,

R¹ is independently halogen, —CX¹₃, —CHX¹₂, —CH₂X¹, —OCX¹₃, —OCH₂X¹, —OCHX¹₂, —CN, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 5;

R² is independently halogen, —CX²₃, —CHX²₂, —CH₂X², —OCX²₃, —

OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(OR^{2C}—C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;
z2 is an integer from 0 to 4;
L¹ is a
   bond, —S(O)₂—, —NR⁴—, —O—, —S—, —C(O)—, —C(O)NR⁴—, —NR⁴C(O)NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{2A}, R^{2B}, R^{2C}, R^{2D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X², X⁴, and X⁵ is independently —F, —Cl, —Br, or —I;

n1, n2, n4, and n5 are independently an integer from 0 to 4; and

m1, m2, m4, m5, v1, v2, v4, and v5 are independently an integer from 1 to 2.

### Embodiment 28

The compound of embodiment 27, wherein the compound has the formula:

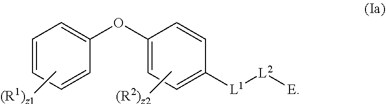

### Embodiment 29

The compound of embodiment 27, wherein the compound has the formula:

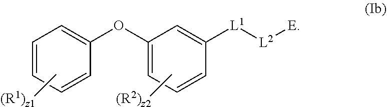

### Embodiment 30

The compound of embodiment 27, wherein the compound has the formula:

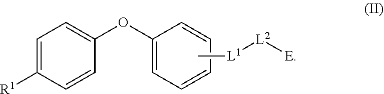

### Embodiment 31

The compound of embodiment 27, wherein the compound has the formula:

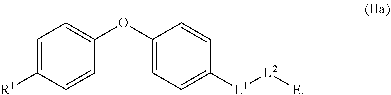

### Embodiment 32

The compound of embodiment 27, wherein the compound has the formula:

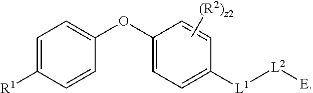

### Embodiment 33

The compound of one of embodiments 27 to 32, wherein R¹ is independently halogen, —CX¹₃, —CHX¹₂, —CH₂X¹, —OCX¹₃, —OCH₂X¹, —OCHX¹₂, —CN, —SR^{1D}, —NR^{1A}R^{1B}, —C(O)R^{1C}, —C(O)OR^{1C}, —C(O)NR^{1A}R^{1B}, —OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 34

The compound of one of embodiments 27 to 32, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ aryl, or substituted or unsubstituted 5 to 12 membered heteroaryl.

### Embodiment 35

The compound of one of embodiments 27 to 32, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

### Embodiment 36

The compound of one of embodiments 27 to 32, wherein R¹ is independently —Cl.

### Embodiment 37

The compound of embodiment 27, wherein two adjacent R¹ substituents are joined to form a substituted or unsubstituted cycloalkyl, substituted or unsubstituted heterocycloalkyl, substituted or unsubstituted aryl, or substituted or unsubstituted heteroaryl.

### Embodiment 38

The compound of embodiment 27, wherein two adjacent R¹ substituents are joined to form an unsubstituted cycloalkyl.

### Embodiment 39

The compound of embodiment 27, wherein two adjacent R¹ substituents are joined to form an unsubstituted C₃-C₆ cycloalkyl.

### Embodiment 40

The compound of one of embodiments 27 to 39, wherein L¹ is a bond, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to 6 membered heteroarylene.

### Embodiment 41

The compound of one of embodiments 27 to 39, wherein L¹ is a bond.

### Embodiment 42

The compound of one of embodiments 27 to 41, wherein L² is —NR⁵— or substituted or unsubstituted heterocycloalkylene comprising a ring nitrogen bonded directly to E.

### Embodiment 43

The compound of one of embodiments 27 to 41, wherein L² is —NR⁵—.

### Embodiment 44

The compound of embodiment 43, wherein R⁵ is hydrogen, substituted or unsubstituted C₁-C₆ alkyl, or substituted or unsubstituted 2 to 6 membered heteroalkyl.

### Embodiment 45

The compound of embodiment 43, wherein R⁵ is hydrogen or unsubstituted C₁-C₃ alkyl.

### Embodiment 46

The compound of embodiment 43, wherein R⁵ is hydrogen, unsubstituted methyl, unsubstituted ethyl, unsubstituted hexyl, or unsubstituted benzyl.

### Embodiment 47

The compound of embodiment 43, wherein R⁵ is hydrogen.

### Embodiment 48

The compound of one of embodiments 27 to 47, wherein E is a covalent cysteine modifier moiety.

### Embodiment 49

The compound of one of embodiments 27 to 47, wherein E is:

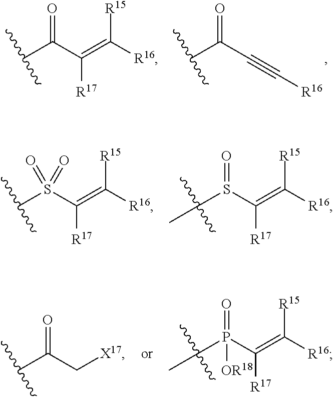

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B},

—NHC═(O)NHNR^{15A}R^{15B},
—NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁶ is independently hydrogen, halogen, CX¹⁶₃, —CHX¹⁶₂, —

CH₂X¹⁶, —CN, —SOₙ₁₆R^{16D}, —SOᵥ₁₆NR^{16A}R^{16B}, —NHNR^{16A}R^{16B}, —ONR^{16A}R^{16B},
—NHC═(O)NHNR^{16A}R^{16B},
—NHC(O)NR^{16A}R^{16B},
—NHC(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁷ is independently hydrogen, halogen, CX¹⁷₃, —CHX¹⁷₂,

—CH₂X¹⁷, —CN, —SOₙ₁₇R^{17D}, —SOᵥ₁₇NR^{17A}R^{17B}, —NHNR^{17A}R^{17B}, —ONR^{17A}R^{17B},
—NHC═(O)NHNR^{17A}R^{17B},
—NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, and R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;

each X, X¹⁵, X¹⁶, X¹⁷ and X¹⁸ is independently —F, —Cl, —Br, or —I;

n15, n16, n17, v15, v16, and v17, are independently an integer from 0 to 4; and

m15, m16, and m17 are independently and integer from 1 to 2.

### Embodiment 50

The compound of embodiment 49, wherein R¹⁵, R¹⁶, R¹⁷, and R¹⁸ are hydrogen.

### Embodiment 51

The compound of one of embodiments 49 to 50, wherein E is:

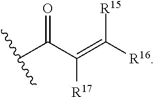

### Embodiment 52

The compound of embodiment 27, having the formula:

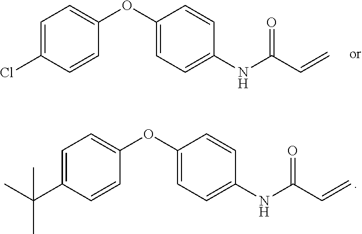

### Embodiment 53

A pharmaceutical composition comprising a Reticulon 4 inhibitor and a pharmaceutically acceptable excipient.

### Embodiment 54

The pharmaceutical composition of embodiment 53, wherein the Reticulon 4 inhibitor is the compound has the formula:

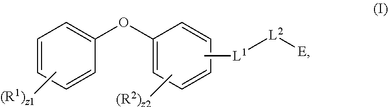

wherein,

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 5; R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;

z2 is an integer from 0 to 4;

L¹ is a

bond, —S(O)₂—, —NR⁴—, —O—, —S—, —C(O)—, —C(O)NR⁴, —, —NR⁴(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{2A}, R^{2B}, R^{2C}, R^{2D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X², X⁴, and X⁵ is independently —F, —Cl, —Br, or —I;

n1, n2, n4, and n5 are independently an integer from 0 to 4; and

m1, m2, m4, m5, v1, v2, v4, and v5 are independently an integer from 1 to 2.

### Embodiment 55

A method of inhibiting reticulon 4 protein activity, said method comprising contacting a reticulon 4 protein with an effective amount of a Reticulon 4 inhibitor, wherein said Reticulon 4 inhibitor contacts one or more amino acids corresponding to E1105, C1101, E1078, S1079, A1082, I1083, K1090, Y1091, S1094, G1097, and H1098 of SEQ ID NO:331.

### Embodiment 56

The method of embodiment 55, wherein the Reticulon 4 inhibitor is an antisense nucleic acid, antibody, or a compound.

### Embodiment 57

The method of embodiment 55 or 56, wherein said Reticulon 4 inhibitor is a compound having the formula:

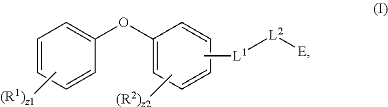

wherein,

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 5;

R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;

z2 is an integer from 0 to 4;

L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—, —OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{2A}, R^{2B}, R^{2C}, R^{2D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X², X⁴, and X⁵ is independently —F, —Cl, —Br, or —I;

n1, n2, n4, and n5 are independently an integer from 0 to 4; and

m1, m2, m4, m5, v1, v2, v4, and v5 are independently an integer from 1 to 2.

### Embodiment 58

The method of embodiment 57, wherein the compound is covalently bonded to the amino acid corresponding to C1101 of SEQ ID NO:331.

### Embodiment 59

A reticulon 4 protein covalently bonded to a compound having the formula:

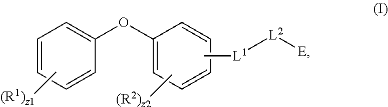

wherein,

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 5;

R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;

z2 is an integer from 0 to 4;

L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{2A}, R^{2B}, R^{2C}, R^{2D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X², X⁴, and X⁵ is independently —F, —Cl, —Br, or —I;

n1, n2, n4, and n5 are independently an integer from 0 to 4; and

m1, m2, m4, m5, v1, v2, v4, and v5 are independently an integer from 1 to 2;

wherein the reticulon 4 protein is covalently bonded to said compound through said reacted electrophilic moiety.

### Embodiment 60

The Reticulon 4 protein of embodiment 59, wherein the compound is bonded to a cysteine residue of the protein.

### Embodiment 61

The reticulon 4 protein of one of embodiments 59 to 60, wherein the compound is covalently bonded to an amino acid corresponding to C1101 of SEQ ID NO:331.

### Embodiment 62

A compound having the formula:

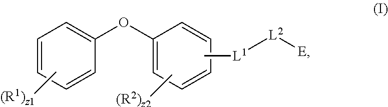

wherein,

R¹ is independently halogen, —CX¹₃, —CHX¹₂, —CH₂X¹, —OCX¹₃, —OCH₂X¹, —OCHX¹₂, —CN, —SOₙ₁NR^{1A}R^{1B}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 5;

R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;

z2 is an integer from 0 to 4;

L¹ is a

bond, —S(O)₂—, —NR⁴—, —O—, —S—, —C(O)—, —C(O)NR⁴—, —NR⁴C(O), —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{2A}, R^{2B}, R^{2C}, R^{2D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X², X⁴, and X⁵ is independently —F, —Cl, —Br, or —I;

n1, n2, n4, and n5 are independently an integer from 0 to 4; and

m1, m2, m4, m5, v1, v2, v4, and v5 are independently an integer from 1 to 2.

### Embodiment 63

The compound of embodiment 62, wherein the compound has the formula:

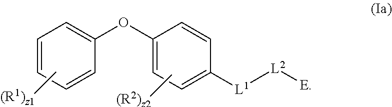

### Embodiment 64

The compound of embodiment 1, wherein the compound has the formula:

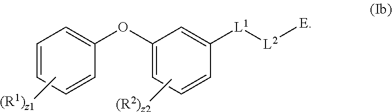

### Embodiment 65

The compound of embodiment 1, wherein the compound has the formula:

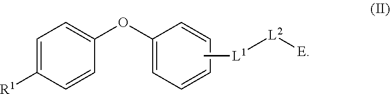

### Embodiment 66

The compound of embodiment 1, wherein the compound has the formula:

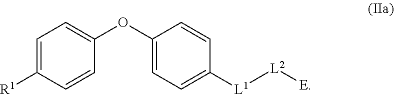

### Embodiment 67

The compound of one of embodiments 62 to 66, wherein R¹ is independently halogen, —CX¹₃, —CHX¹₂, —CH₂X¹, —OCX¹₃, —OCH₂X¹, —OCHX¹₂, —CN, —SR^{1D}, —NR^{1A}R^{1B}, —C(O)R^{1C}, —C(O)OR^{1C}, —C(O)NR^{1A}R^{1B}, —OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 68

The compound of one of embodiments 62 to 66, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ aryl, or substituted or unsubstituted 5 to 12 membered heteroaryl.

### Embodiment 69

The compound of one of embodiments 62 to 66, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

### Embodiment 70

The compound of one of embodiments 62 to 66, wherein R¹ is independently —Cl.

### Embodiment 71

The compound of embodiment 62, wherein two adjacent R¹ substituents are joined to form a substituted or unsubstituted cycloalkyl, substituted or unsubstituted heterocycloalkyl, substituted or unsubstituted aryl, or substituted or unsubstituted heteroaryl.

### Embodiment 72

The compound of embodiment 62, wherein two adjacent R¹ substituents are joined to form an unsubstituted cycloalkyl.

### Embodiment 73

The compound of embodiment 62, wherein two adjacent R¹ substituents are joined to form an unsubstituted C₃-C₆ cycloalkyl.

### Embodiment 74

The compound of one of embodiments 62 to 73, wherein L¹ is a bond, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to 6 membered heteroarylene.

### Embodiment 75

The compound of one of embodiments 62 to 73, wherein L¹ is a bond.

### Embodiment 76

The compound of one of embodiments 62 to 75, wherein L² is —NR⁵— or substituted or unsubstituted heterocycloalkylene comprising a ring nitrogen bonded directly to E.

### Embodiment 77

The compound of one of embodiments 62 to 75, wherein L² is —NR⁵—.

### Embodiment 78

The compound of embodiment 77, wherein R⁵ is hydrogen, substituted or unsubstituted C₁-C₆ alkyl, or substituted or unsubstituted 2 to 6 membered heteroalkyl.

### Embodiment 79

The compound of embodiment 77, wherein R⁵ is hydrogen or unsubstituted C₁-C₃ alkyl.

### Embodiment 80

The compound of embodiment 77, wherein R⁵ is hydrogen, unsubstituted methyl, unsubstituted ethyl, unsubstituted hexyl, or unsubstituted benzyl.

### Embodiment 81

The compound of embodiment 77, wherein R⁵ is hydrogen.

### Embodiment 82

The compound of one of embodiments 62 to 81, wherein E is a covalent cysteine modifier moiety.

### Embodiment 83

The compound of one of embodiments 62 to 81, wherein E is:

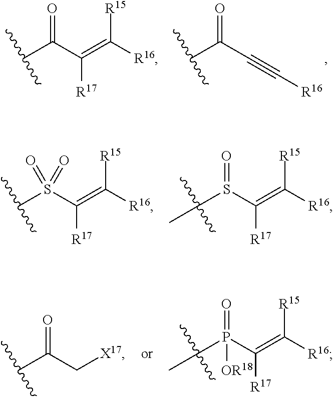

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B},

—NHC═(O)NHNR^{15A}R^{15B},
—NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B},

—NHC═(O)NHNR^{16A}R^{16B},
—NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B},

—NHC═(O)NHNR^{17A}R^{17B},
—NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃,
—OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, and R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;

each X, X¹⁵, X¹⁶, X¹⁷ and X¹⁸ is independently —F, —Cl, —Br, or —I;

n15, n16, n17, v15, v16, and v17, are independently an integer from 0 to 4; and

m15, m16, and m17 are independently and integer from 1 to 2.

### Embodiment 84

The compound of embodiment 83, wherein R¹⁵, R¹⁶, R¹⁷, and R¹⁸ are hydrogen.

### Embodiment 85

The compound of one of embodiments 82 to 83, wherein E is:

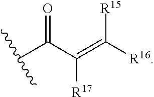

### Embodiment 86

The compound of embodiment 62, having the formula:

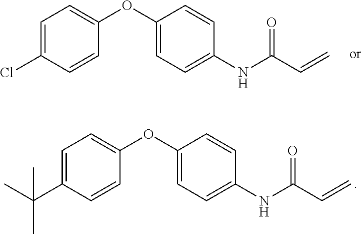

### IX. Examples

### I. Chemoproteomics-Enabled Covalent Ligand Screen Reveals a Cysteine Hotspot in Reticulon 4 that Impairs ER Morphology and Cancer Pathogenicity

Chemical genetics has arisen as a powerful approach for identifying novel anti-cancer agents. However, a major bottleneck in chemical genetics is identifying the targets of leads that arise from screens. Here, we generated and screened a library of cysteine-reactive fragment-based covalent ligands for agents that impair colorectal cancer pathogenicity and coupled the discovery of lead compounds with target identification using isotopic tandem orthogonal proteolysis-enabled activity-based protein profiling (isoTOP-ABPP) platforms. Through this coupled approach, we discovered a cysteine-reactive acrylamide DKM 3-30 that impaired colorectal cancer cell pathogenicity through targeting C1101 on reticulon 4 (RTN4). This protein has been established as a critical mediator of endoplasmic reticulum tubular network formation. We show here that covalent modification of C1101 on RTN4 by DKM 3-30 or genetic knockdown of RTN4 impairs endoplasmic reticulum and nuclear envelope morphology and colorectal cancer pathogenicity. RTN4 is a novel colorectal cancer therapeutic target and we determined a unique druggable hotspot within RTN4 that can be targeted by covalent ligands to impair colorectal cancer pathogenicity. Our results underscore the utility of coupling the screening of fragment-based covalent ligands with isoTOP-ABPP platforms for mining the proteome for novel druggable nodes that can be targeted for cancer therapy.

Traditional strategies for cancer target discovery oftentimes involve searching for proteins or genes that may be dysregulated or mutated in tumors, which may miss promising therapeutic targets that may not necessarily be changing in expression or activity. Screening chemical libraries for anti-cancer small-molecules using chemical genetics strategies have arisen as a powerful complementary approach to traditional target discovery approaches for mining druggable nodes that can be pharmacologically interrogated in cancer^{3,4}. However, a major challenge with chemical genetics is identifying the targets of leads that arise from screens. Oftentimes, lead compounds must be derivatized to either bear bioorthogonal and/or photoaffinity handles or conjugated to beads to facilitate chemoproteomic target identification⁴. However, these approaches oftentimes require additional synthetic efforts to make analogs of the lead molecule and alter the structure of the molecule, which hinder or may prevent target identification.

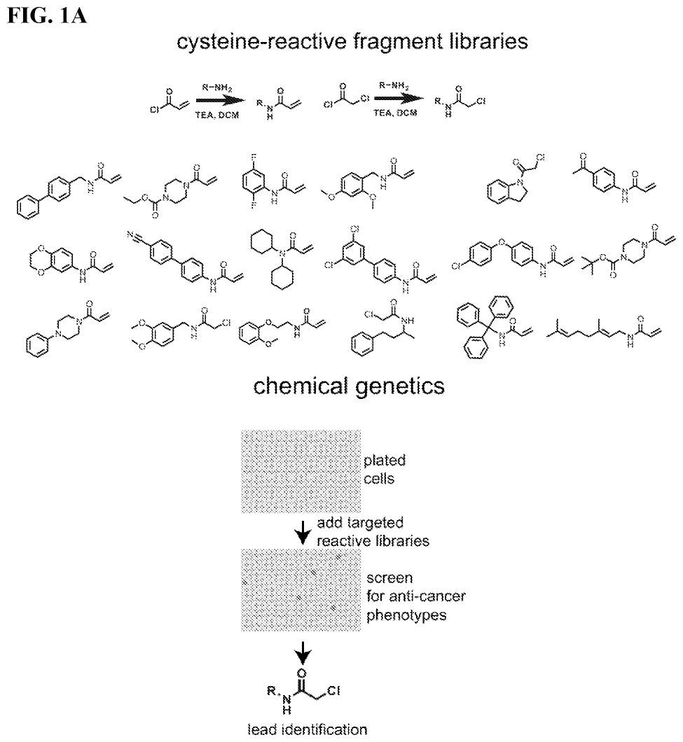

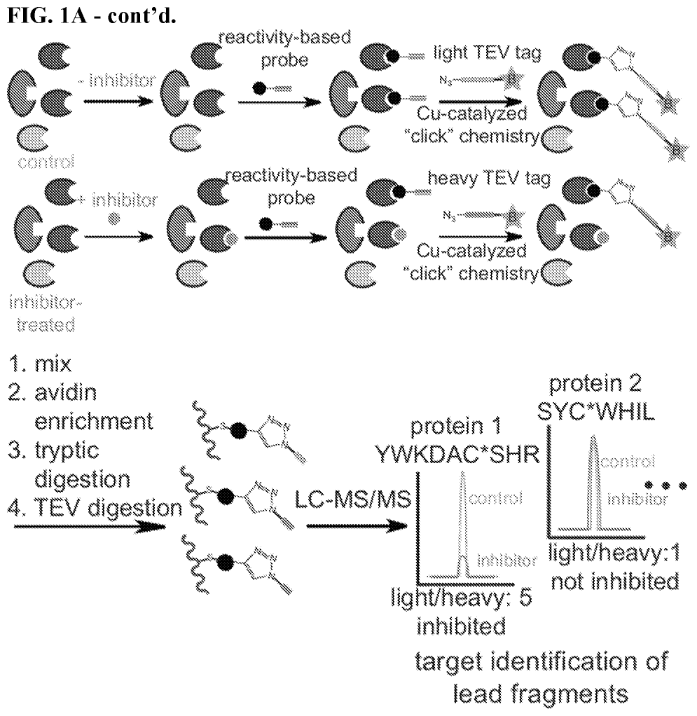

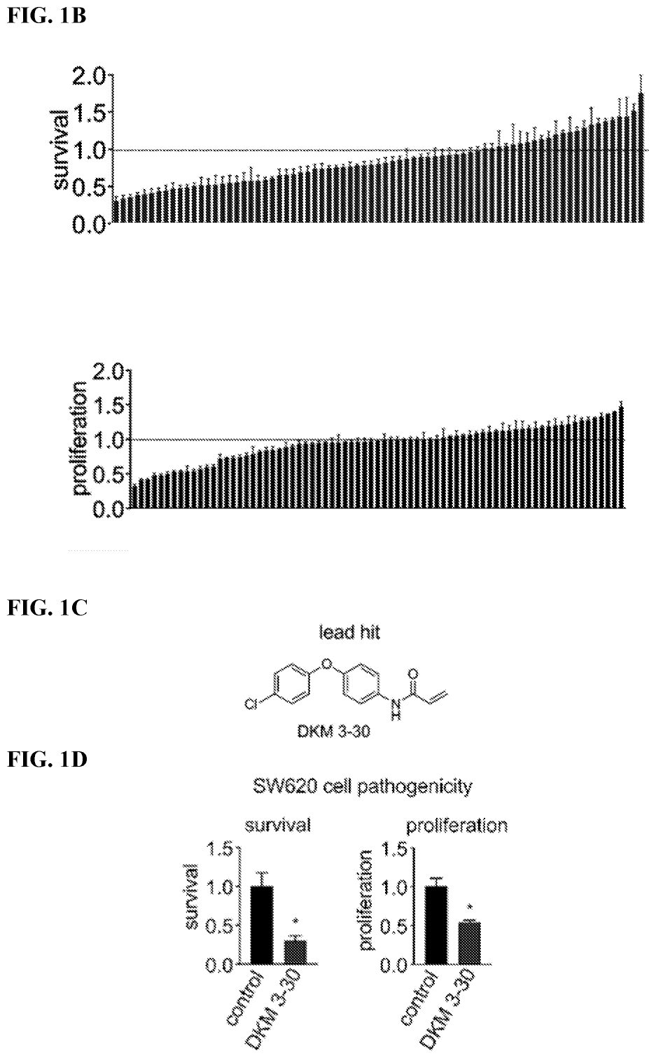

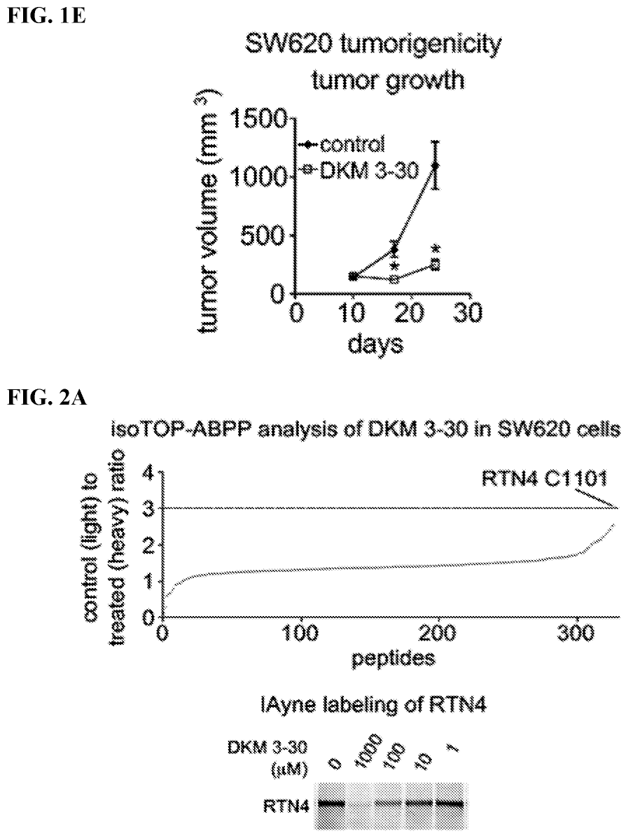

Here, we have generated a library of 75 cysteine-reactive fragment-based covalent ligands and coupled the screening of this library with an isotopic tandem orthogonal proteolysis-enabled activity-based protein profiling (isoTOP-ABPP) platform to rapidly couple the identification of covalent ligands that impair colorectal cancer pathogenicity with the identification of its direct targets and druggable hotspots within these targets (FIG. 1A). IsoTOP-ABPP uses reactivity-based chemical probes to map proteome-wide reactive, functional, and ligandable hotspots. When used in a competitive manner, covalent small-molecules can be competed against the binding of their corresponding reactivity-based probes to rapidly identify the targets of these molecules⁵⁻⁷. In this case, upon identifying a cysteine-reactive lead fragment, lead molecules can be competed against a broad cysteine-reactive probe to subsequently identify its targets and the specific sites of labeling.

There are several advantages to this overall approach. First, our library already introduces specific covalent interactions through the incorporation of cysteine-reactive acrylamide and chloroacetamide warheads, thus avoiding the necessity for introducing photoaffinity handles for target identification. Recent studies have shown that the reactivity of these scaffolds can be tempered and made to confer substantial selectivity through appending small-molecular weight fragments⁶. Also, because these compounds are small molecular weight fragment-based covalent ligands, they can sample more macromolecular protein space and enable interrogation of more druggable nodes, a notion explored by many pharmaceutical companies with fragment-based ligand discovery⁸. Second, the advantage of this approach is that the lead molecule itself can be directly competed against reactivity-based probes for target identification without the need for additional derivatization or synthetic efforts.

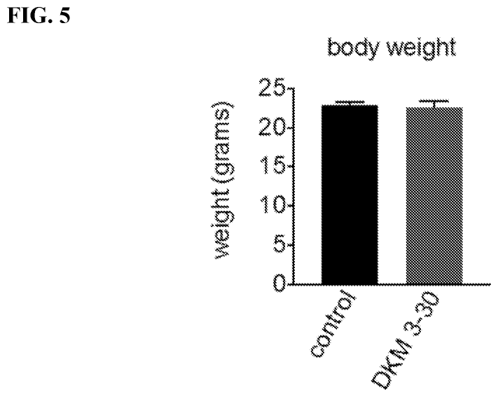

We screened our cysteine-reactive ligand library of acrylamides and chloroacetamides to identify compounds that impair colorectal cancer cell survival and proliferation in the highly metastatic and tumorigenic SW620 colorectal cancer cells (FIG. 1B, Table 1). We identified a lead acrylamide DKM 3-30 as a hit from this screen which significantly impaired both serum-free cell survival and proliferation in SW620 colorectal cancer cells (FIG. 1C, 1D). We next wanted to determine whether DKM 3-30 impaired tumor growth in vivo in immune-deficient mice without causing overt toxicity. We initiated daily intraperitoneal treatments with this compound ten days after subcutaneous injection of SW620 cells and establishment of tumors. Strikingly, we observed initial regression and a sustained impairment in tumor growth from daily treatment of DKM 3-30, without any changes in body weight or any signs of overt toxicity (FIG. 1E, FIG. 5). Taken together, our data suggested that DKM 3-30 significantly impaired SW620 colorectal cancer pathogenicity both in culture and in vivo.

TABLE 1
Covalent ligand screening data. SW620 cells were treated with either
DMSO or cysteine-reactive fragment (50 μM) for 48 h after which
serum-free cell survival or proliferation were assessed by Hoescht
staining. Shown are average and sem values from n = 3/group.

| SW620 survival | Ave | sem | p value |
|---|---|---|---|
| DKM 3-30 | 0.31 | 0.06 | 2.21E−02 |
| DKM 3-16 | 0.34 | 0.04 | 2.28E−02 |
| DKM 2-40 | 0.36 | 0.03 | 3.11E−03 |
| DKM 2-91 | 0.38 | 0.03 | 8.97E−04 |
| DKM 2-101 | 0.39 | 0.06 | 7.57E−03 |
| DKM 3-10 | 0.41 | 0.06 | 7.05E−03 |
| DKM 2-94 | 0.43 | 0.05 | 2.39E−03 |
| DKM 2-76 | 0.44 | 0.07 | 7.08E−03 |
| DKM 2-80 | 0.47 | 0.08 | 9.35E−03 |
| TRH 1-55 | 0.47 | 0.04 | 2.37E−03 |
| TRH 1-12 | 0.49 | 0.03 | 7.24E−03 |
| DKM 3-7 | 0.51 | 0.04 | 5.46E−02 |
| DKM 2-95 | 0.52 | 0.10 | 3.89E−02 |
| DKM 3-43 | 0.52 | 0.08 | 7.87E−02 |
| DKM 2-98 | 0.52 | 0.13 | 3.64E−02 |
| DKM 3-36 | 0.54 | 0.09 | 9.56E−02 |
| TRH 1-32 | 0.55 | 0.10 | 1.00E−01 |
| DKM 3-41 | 0.55 | 0.09 | 9.87E−02 |
| DKM 3-70 | 0.57 | 0.11 | 1.26E−01 |
| DKM 2-37 | 0.57 | 0.18 | 1.09E−01 |
| TRH 1-50 | 0.58 | 0.07 | 9.26E−02 |
| DKM 3-5 | 0.59 | 0.04 | 1.21E−02 |
| DKM 2-108 | 0.61 | 0.02 | 1.36E−02 |
| DKM 3-31 | 0.65 | 0.08 | 1.64E−01 |
| DKM 2-83 | 0.66 | 0.07 | 3.18E−02 |
| DKM 2-59 | 0.66 | 0.07 | 4.92E−02 |
| TRH 1-53 | 0.69 | 0.07 | 4.08E−02 |
| DKM 3-32 | 0.70 | 0.07 | 2.03E−01 |
| DKM 2-93 | 0.74 | 0.06 | 4.79E−02 |
| DKM 2-84 | 0.74 | 0.07 | 7.66E−02 |
| DKM 2-113 | 0.75 | 0.03 | 6.08E−02 |
| DKM 3-9 | 0.75 | 0.03 | 2.44E−01 |
| DKM 2-114 | 0.76 | 0.03 | 7.42E−02 |
| DKM 3-13 | 0.77 | 0.06 | 2.89E−01 |
| DKM 2-34 | 0.78 | 0.02 | 8.76E−02 |
| DKM 2-47 | 0.78 | 0.05 | 1.16E−01 |
| DKM 3-29 | 0.79 | 0.05 | 3.11E−01 |
| DKM 2-49 | 0.80 | 0.06 | 1.60E−01 |
| DKM 2-71 | 0.81 | 0.08 | 3.96E−01 |
| DKM 2-43 | 0.84 | 0.06 | 2.27E−01 |
| DKM 2-107 | 0.86 | 0.05 | 2.62E−01 |
| DKM 2-67 | 0.87 | 0.14 | 6.20E−01 |
| DKM 2-50 | 0.89 | 0.02 | 3.05E−01 |
| DKM 2-31 | 0.89 | 0.04 | 3.69E−01 |
| DKM 2-48 | 0.90 | 0.05 | 4.00E−01 |
| DKM 2-32 | 0.91 | 0.11 | 5.55E−01 |
| DKM 2-33 | 0.92 | 0.08 | 5.36E−01 |
| DKM 2-52 | 0.92 | 0.10 | 7.25E−01 |
| DKM 2-39 | 0.93 | 0.05 | 5.71E−01 |
| TRH 1-13 | 0.94 | 0.05 | 6.20E−01 |
| DKM 2-72 | 0.96 | 0.06 | 8.49E−01 |
| DKM 2-58 | 0.98 | 0.05 | 8.50E−01 |
| TRH 1-19 | 1.01 | 0.06 | 9.16E−01 |
| DKM 2-120 | 1.02 | 0.06 | 8.83E−01 |
| DKM 3-42 | 1.04 | 0.21 | 9.09E−01 |
| DKM 2-42 | 1.04 | 0.04 | 7.11E−01 |
| DKM 2-97 | 1.06 | 0.27 | 8.33E−01 |
| DKM 2-60 | 1.08 | 0.16 | 7.51E−01 |
| DKM 2-86 | 1.09 | 0.13 | 6.45E−01 |
| DKM 2-110 | 1.12 | 0.17 | 5.54E−01 |
| TRH 1-20 | 1.14 | 0.05 | 2.66E−01 |
| DKM 2-62 | 1.15 | 0.09 | 2.76E−01 |
| DKM 3-11 | 1.20 | 0.18 | 5.32E−01 |
| DKM 3-4 | 1.22 | 0.03 | 8.53E−02 |
| DKM 2-116 | 1.23 | 0.20 | 3.58E−01 |
| DKM 2-102 | 1.24 | 0.05 | 9.50E−02 |
| DKM 3-12 | 1.29 | 0.09 | 2.49E−01 |
| DKM 2-111 | 1.33 | 0.23 | 2.40E−01 |
| DKM 2-103 | 1.35 | 0.06 | 4.12E−02 |
| DKM 2-100 | 1.38 | 0.03 | 1.54E−02 |
| DKM 2-109 | 1.40 | 0.02 | 1.17E−02 |
| TRH 1-27 | 1.44 | 0.24 | 2.79E−01 |
| DKM 2-106 | 1.44 | 0.25 | 1.75E−01 |
| DKM 3-8 | 1.52 | 0.09 | 6.85E−02 |
| TRH 1-54 | 1.75 | 0.24 | 1.01E−01 |
| DKM 2-94 | 0.32 | 0.03 | 2.69E−04 |
| DKM 2-71 | 0.41 | 0.01 | 4.06E−06 |
| DKM 2-98 | 0.42 | 0.02 | 6.51E−06 |
| DKM 2-83 | 0.48 | 0.03 | 9.33E−04 |
| DKM 2-80 | 0.48 | 0.02 | 5.02E−04 |
| DKM 2-76 | 0.50 | 0.03 | 1.02E−03 |
| DKM 3-70 | 0.53 | 0.02 | 2.15E−05 |
| DKM 2-52 | 0.53 | 0.02 | 5.63E−05 |
| TRH 1-55 | 0.53 | 0.09 | 1.68E−02 |
| DKM 3-30 | 0.54 | 0.03 | 1.50E−02 |
| DKM 2-93 | 0.57 | 0.04 | 2.66E−03 |
| DKM 2-91 | 0.59 | 0.03 | 2.18E−03 |
| DKM 3-16 | 0.60 | 0.03 | 2.62E−02 |
| TRH 1-53 | 0.72 | 0.06 | 3.32E−02 |
| DKM 2-67 | 0.73 | 0.01 | 3.21E−06 |
| DKM 2-37 | 0.73 | 0.02 | 1.45E−03 |
| DKM 2-59 | 0.74 | 0.03 | 3.06E−03 |
| TRH 1-50 | 0.76 | 0.04 | 8.71E−03 |
| DKM 3-10 | 0.79 | 0.10 | 2.12E−01 |
| DKM 3-5 | 0.82 | 0.03 | 3.03E−03 |
| DKM 2-84 | 0.84 | 0.05 | 3.17E−02 |
| DKM 2-48 | 0.85 | 0.04 | 3.85E−02 |
| DKM 2-95 | 0.85 | 0.01 | 6.52E−02 |
| TRH 1-12 | 0.89 | 0.07 | 1.86E−01 |
| DKM 2-116 | 0.90 | 0.03 | 2.11E−02 |
| DKM 3-41 | 0.93 | 0.05 | 6.18E−01 |
| DKM 3-13 | 0.94 | 0.03 | 6.02E−01 |
| DKM 3-43 | 0.94 | 0.01 | 6.04E−01 |
| DKM 3-32 | 0.95 | 0.03 | 6.55E−01 |
| DKM 2-62 | 0.95 | 0.02 | 5.64E−02 |
| DKM 2-110 | 0.95 | 0.06 | 5.12E−01 |
| DKM 2-108 | 0.95 | 0.11 | 7.80E−01 |
| DKM 2-120 | 0.96 | 0.01 | 4.87E−02 |
| DKM 2-109 | 0.96 | 0.02 | 1.74E−01 |
| DKM 2-97 | 0.96 | 0.05 | 5.01E−01 |
| DKM 2-101 | 0.96 | 0.05 | 6.87E−01 |
| DKM 3-36 | 0.97 | 0.04 | 8.31E−01 |
| DKM 2-40 | 0.98 | 0.01 | 4.54E−01 |
| DKM 2-107 | 0.99 | 0.09 | 9.13E−01 |
| DKM 3-31 | 0.99 | 0.04 | 9.63E−01 |
| DKM 2-100 | 1.00 | 0.04 | 9.31E−01 |
| DKM 3-7 | 1.00 | 0.01 | 9.83E−01 |
| TRH 1-32 | 1.00 | 0.03 | 9.60E−01 |
| DKM 2-72 | 1.00 | 0.03 | 9.74E−01 |
| DKM 3-9 | 1.00 | 0.10 | 9.91E−01 |
| DKM 2-106 | 1.00 | 0.02 | 9.02E−01 |
| DKM 2-60 | 1.00 | 0.05 | 9.64E−01 |
| DKM 2-86 | 1.02 | 0.13 | 8.97E−01 |
| DKM 3-8 | 1.05 | 0.00 | 6.93E−01 |
| DKM 2-34 | 1.05 | 0.07 | 5.50E−01 |
| DKM 2-111 | 1.06 | 0.01 | 2.53E−02 |
| DKM 3-12 | 1.06 | 0.06 | 6.44E−01 |
| DKM 2-49 | 1.09 | 0.02 | 6.51E−02 |
| DKM 2-39 | 1.10 | 0.03 | 6.16E−02 |
| DKM 2-114 | 1.11 | 0.08 | 4.12E−01 |
| DKM 2-47 | 1.12 | 0.02 | 2.38E−02 |
| DKM 2-103 | 1.12 | 0.11 | 4.76E−01 |
| DKM 3-42 | 1.12 | 0.07 | 2.68E−01 |
| DKM 2-32 | 1.14 | 0.12 | 3.13E−01 |
| DKM 2-33 | 1.15 | 0.11 | 2.44E−01 |
| DKM 2-58 | 1.16 | 0.04 | 2.34E−02 |
| DKM 2-31 | 1.16 | 0.09 | 1.75E−01 |
| DKM 3-11 | 1.18 | 0.02 | 1.79E−01 |
| TRH 1-19 | 1.19 | 0.07 | 1.47E−01 |
| DKM 3-4 | 1.20 | 0.10 | 2.19E−01 |
| DKM 3-29 | 1.21 | 0.04 | 1.76E−02 |
| TRH 1-20 | 1.21 | 0.11 | 2.47E−01 |
| TRH 1-27 | 1.25 | 0.08 | 6.96E−02 |
| DKM 2-43 | 1.28 | 0.03 | 2.17E−03 |
| TRH 1-13 | 1.28 | 0.04 | 4.17E−03 |
| DKM 2-50 | 1.30 | 0.01 | 4.14E−04 |
| DKM 2-102 | 1.33 | 0.05 | 1.65E−02 |
| DKM 2-42 | 1.37 | 0.00 | 1.52E−04 |
| TRH 1-54 | 1.39 | 0.01 | 1.49E−06 |
| DKM 2-113 | 1.47 | 0.07 | 1.23E−02 |

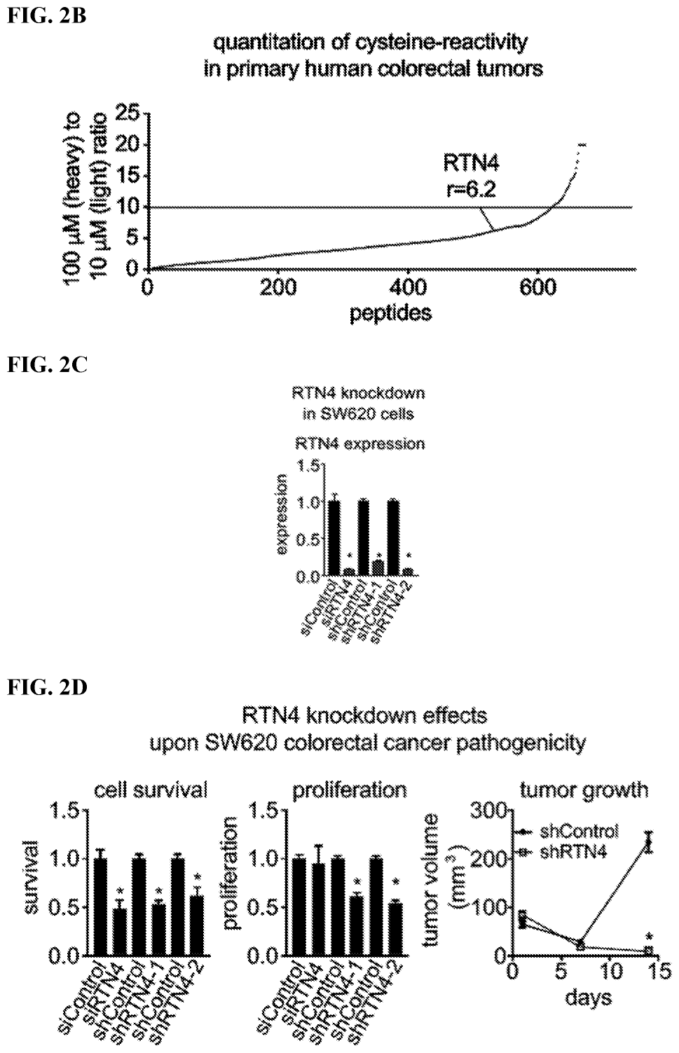

We next performed isoTOP-ABPP studies to identify the direct targets of these lead compounds. We competed either vehicle or DKM 3-30 against labeling of SW620 proteomes with a broad cysteine-reactive probe, iodoacetamide-alkyne (IAyne), followed by appending probe-labeled proteins with a biotin-azide tag bearing a TEV protease recognition site and an isotopically light (for vehicle-treated) or heavy (for fragment-treated) tags via copper catalyzed azide-alkyne cycloaddition (CuAAC)^{6,9}. We then combined control and treated proteomes in a 1:1 ratio, enriched probe-labeled proteins with avidin, and digested proteomes with trypsin. Avidin-enriched probe-modified tryptic peptides were released by TEV protease digestion for subsequent quantitative proteomic analysis. Through these studies, we identified the top hit for DKM 3-30 as C1101 in reticulon 4 (RTN4, Uniprot ID Q9NQC3-1) with a light to heavy ratio of 3.0 (FIG. 2A; Table 2). We further validated this hit by competing DKM 3-30 against IAyne labeling of pure human RTN4 protein using gel-based ABPP methods (FIG. 2A).

TABLE 2
IsoTOP-ABPP analyses of DKM 3-5 and DKM 3-30 in SW620 cells. SW620 cell proteomes
were pre-treated with DMSO or DKM 3-5 (50 μM) or DKM 3-30 (50 μM) for 30 min at
37° C. prior to labeling of proteomes with IAyne (100 μM) for 1h at room temperature.
Proteomes were then subjected to copper-catalyzed azide-alkyne cycloaddition with
a biotin-azide tag bearing an isotopically light (for DMSO-treated) versus heavy
(for ligand-treated) valine and a TEV protease recognition site. Proteomes were
then mixed in a 1: 1 ratio and probe-modified peptides were enriched and released
by TEV protease for subsequent quantitative proteomic analysis. The data was
filtered for only those probe-modified peptides that were identified in at least
2 out of 3 runs. Ratios for the same redundant probe-modified peptides and peptides
across runs were averaged. Top hits of covalent ligands were confirmed to have more
than one light to heavy ratio greater than 2. Shown below are the final consolidated
and averaged light to heavy ratios for those peptides only observed in at least 2 out of
3 biological replicates for isoTOP-ABPP studies with DKM 3-5 and DKM 3-30, respectively.

| | | Cys | Ave area | | # of |
|---|---|---|---|---|---|
| Peptide | Seq ID Number | Index | ratio | UniProt | runs |
| YSNSALGHVNC*TIK | (SEQ ID NO: 1) | C1101 | 3.04 | Q9NQC3 F8W914 | 3 |
| AYEYVEC*PIR | (SEQ ID NO: 2) | | 2.54 | P53701 | 2 |
| APPWVPAMGFTLAPS | (SEQ ID NO: 3) | C19 | 2.51 | B1AH87 P30536 | 2 |
| LGC*FVGSR | | | | | |
| YYGGAEVVDEIELLC*QR | (SEQ ID NO: 4) | | 2.43 | G3V5L0 | 2 |
| KVIGIEC*SSISDYAVK | (SEQ ID NO: 5) | C101 | 2.40 | Q99873 | 2 |
| TYDPSGDSTLPTC*SK | (SEQ ID NO: 6) | | 2.35 | Q9Y2X3 | 2 |
| FTTSC*MTGYSPQLQGLSSGGSGSYSPGVTYSPVSGYNK | (SEQ ID NO: 7) | C158 | 2.31 | Q965K2 Q965K2 | 2 |
| | | C157 | | | |
| SLVQNNCLSRPNIFLC*PEIEPK | (SEQ ID NO: 8) | C145 | 2.28 | Q8TAQ2 Q8TAQ2 | 2 |
| | | | | Q8TAQ2 F8VXC8 | |
| LWNTLGVC*K | (SEQ ID NO: 9) | | 2.22 | P63244 | 2 |
| SLPSAVYC*IEDK | (SEQ ID NO: 10) | C674 | 2.20 | O43290 | 2 |
| EGGQYGLVAAC*AAGGQGHAMIVEAYPK | (SEQ ID NO: 11) | C436 | 2.14 | P55084 P55084 | 2 |
| AAIGC*GIVESILNWVK | (SEQ ID NO: 12) | C486 | 2.14 | P11388 P11388 | 2 |
| | | C431 | | P11388 P11388 | |
| LC*PLKDEPWPIHPWEPGSFR | (SEQ ID NO: 13) | C105 | 2.11 | E7ETU7 H0Y9G6 | 2 |
| | | C78 | | E9PF06 P09001 | |
| VIEASDVVLEVLDARDPLGC*R | (SEQ ID NO: 14) | C144 | 2.08 | Q9BVP2 Q9BVP2 | 2 |
| YVAAAFPSAC*GK | (SEQ ID NO: 15) | C172 | 2.07 | B4DW73 Q16822 | 3 |
| VC*TLAIIDPGDSDIIR | (SEQ ID NO: 16) | C92 | 2.06 | P62888 E5RI99 | 2 |
| STPYEC*GFDPMSPAR | (SEQ ID NO: 17) | C39 | 2.01 | P03897 | 2 |
| VALEGLRPTIPPGISPHVC*K | (SEQ ID NO: 18) | C361 | 2.00 | Q13418 Q13418 | 2 |
| | | C453 | | A0A0A0MTH3 | |
| | | | | Q13418 | |
| LLEETGIC*VVPGSGFGQR | (SEQ ID NO: 19) | C477 | 1.94 | Q8TD30 | 2 |
| FGVIC*LEDLIHEIAFPGK | (SEQ ID NO: 20) | | 1.92 | Q6DKI1 | 2 |
| ASC*LYGQLPK | (SEQ ID NO: 21) | | 1.89 | P09211 | 3 |
| ATGHSGGGC*ISQGR | (SEQ ID NO: 22) | | 1.84 | I3L407 I3L139 | 3 |
| | | | | Q9HA64 I3L3W5 | |
| HVVC*AAETGSGK | (SEQ ID NO: 23) | | 1.82 | Q9NUL7 | 3 |
| STFFNVLTNSQASAENFPFC*TIDPNESRVPVPDER | (SEQ ID NO: 24) | | 1.78 | J3KQ32 Q9NTK5 | 2 |
| VDDEILGFISEATPLGGIQAASTESC*NQQLDLALCR | (SEQ ID NO: 25) | | 1.78 | P42166 | 2 |
| IDRYTQQGFGNLPIC*MAK | (SEQ ID NO: 26) | C841 | 1.77 | A0A087WVM4 | 2 |
| | | C906 | | Q6UB35 B7ZM99 | |
| | | C907 | | | |
| AVC*MLSNTTAIAEAWAR | (SEQ ID NO: 27) | C376 | 1.77 | P68366 Q13748 | 3 |
| | | | | Q9NY65 C9J2C0 | |
| | | | | Q71U36 Q9NY65 | |
| | | | | P68366 | |
| LC*YVALDFENEMATAASSSSLEK | (SEQ ID NO: 28) | C219 | 1.74 | P68133 P68032 | 3 |
| YATSCYSCC*PR | (SEQ ID NO: 29) | C173 | 1.71 | Q13057 Q13057 | 2 |
| VIGSGC*NLDSAR | (SEQ ID NO: 30) | C192 | 1.71 | P00338 P07195 | 2 |
| | | | | P00338 | |
| VLPMNTGVEAGETAC*K | (SEQ ID NO: 31) | | 1.71 | P04181 | 2 |
| AVLLVGLC*DSGK | (SEQ ID NO: 32) | C73 | 1.70 | Q9Y5M8 | 3 |
| VTDDLVC*LVYK | (SEQ ID NO: 33) | | 1.70 | P49458 | 2 |
| VC*EEIAIIPSKK | (SEQ ID NO: 34) | C35 | 1.69 | H0YN88 | 2 |
| | | | | A0A075B716 | |
| | | | | P08708 | |
| VQENSAYIC*SR | (SEQ ID NO: 35) | C585 | 1.67 | Q9Y3T9 | 2 |
| NCAVSC*AGEK | (SEQ ID NO: 36) | C141 | 1.67 | Q15813 Q15813 | 2 |
| GC*STVLSPEGSAQFAAQIFGLSNHLVWSK | (SEQ ID NO: 37) | C374 | 1.66 | P22234 | 2 |
| TIQFVDWC*PTGFK | (SEQ ID NO: 38) | C347 | 1.66 | Q13748 Q9BQE3 | 2 |
| | | | | Q9NY65 C9J2C0 | |
| | | | | Q71U36 Q9NY65 | |
| AGQPHSSSDAAQAPAEQPHSSSDAAQAPC*PR | (SEQ ID NO: 39) | C51 | 1.66 | P29372 P29372 | 2 |
| | | | | A2IDA3 P29372 | |
| | | | | P29372 | |
| LALFNPDVC*WDRNNPEPWNK | (SEQ ID NO: 40) | C44 | 1.65 | O00483 | 2 |
| AVASQLDC*NFLK | (SEQ ID NO: 41) | | 1.65 | A0A087X2I1 | 2 |
| | | | | P62333 | |
| C*SGIGDNPGSETAAPR | (SEQ ID NO: 42) | C1317 | 1.64 | H0YAC6 P50851 | 2 |
| QPAIMPGQSYGLEDGSC*SYK | (SEQ ID NO: 43) | | 1.64 | M0QXS5 P14866 | 2 |
| GNVAGDSKNDPPMEAAGFTAQVIILNHPGQISAGYAPVLDC* | (SEQ ID NO: 44) | | 1.64 | P68104 | 2 |
| HTAHIACK | | | | A0A087WVQ9 | |
| YWLC*AATGPSIK | (SEQ ID NO: 45) | | 1.64 | P63244 | 2 |
| LQSGIC*HLFR | (SEQ ID NO: 46) | | 1.64 | P14868 | 2 |
| TIYAGNALC*TVK | (SEQ ID NO: 47) | C155 | 1.63 | P13804 | 3 |
| TSC*GSPNYAAPEVISGR | (SEQ ID NO: 48) | C200 | 1.63 | Q13131 Q13131 | 2 |
| TFC*GTPEYLAPEVLEDNDYGR | (SEQ ID NO: 49) | C310 | 1.62 | P31749 P31751 | 2 |
| | | | | Q9Y243 Q9Y243 | |
| | | | | M0R0P9 | |
| ISC*MSKPPAPNPTPPR | (SEQ ID NO: 50) | | 1.62 | P46734 P46734 | 2 |
| RPYGVGLLIAGYDDMGPHIFQTC*PSANYFDCR | (SEQ ID NO: 51) | C123 | 1.60 | P25786 P25786 | 3 |
| | | C154 | | F5GX11 | |
| LADQC*TGLQGFLVFHSFGGGTGSGFTSLLMER | (SEQ ID NO: 52) | C129 | 1.59 | Q9BQE3 P68363 | 2 |
| | | C199 | | F5H5D3 Q71U36 | |
| LNLSC*IHSPVVNELMR | (SEQ ID NO: 53) | | 1.59 | Q9Y2X3 | 2 |
| LVVPATQC*GSLIGK | (SEQ ID NO: 54) | C109 | 1.58 | Q15365 | 2 |
| NLSDLIDLVPSLC*EDLLSSVDQPLK | (SEQ ID NO: 55) | C65 | 1.58 | P47756 P47756 | 2 |
| | | C24 | | B1AK88 B1AK87 | |
| | | | | B1AK85 | |
| SVHYC*PATK | (SEQ ID NO: 56) | C193 | 1.57 | P25205 P25205 | 2 |
| NFYGGNGIVGAQVPLGAGIALAC*K | (SEQ ID NO: 57) | C188 | 1.57 | P08559 P08559 | 2 |
| | | C219 | | P08559 | |
| HISPTAPDTLGC*YPFYK | (SEQ ID NO: 58) | C384 | 1.56 | A0A087WWF6 | 2 |
| | | C419 | | P49005 | |
| QGEYGLASIC*NGGGGASAMLIQKL | (SEQ ID NO: 59) | | 1.56 | P24752 | 3 |
| HSMNPFC*EIAVEEAVR | (SEQ ID NO: 60) | C133 | 1.56 | P38117 P38117 | 2 |
| LGMLSPEGTC*K | (SEQ ID NO: 61) | C212 | 1.56 | P49327 | 2 |
| GLC*GAIHSSIAK | (SEQ ID NO: 62) | C103 | 1.56 | P36542 P36542 | 2 |
| LVAFC*PFASSQVALENANAVSEGVVHEDLR | (SEQ ID NO: 63) | | 1.55 | O00567 | 2 |
| GSDC*GIVNVNIPTSGAEIGGAFGGEK | (SEQ ID NO: 64) | C450 | 1.55 | P49419 P49419 | 2 |
| LNQQQHPDC*K | (SEQ ID NO: 65) | C239 | 1.55 | Q5T280 | 3 |
| C*LHNFLTDGVPAEGAFTEDFQGLR | (SEQ ID NO: 66) | C268 | 1.54 | G3V1A6 P57764 | 2 |
| | | C316 | | | |
| TPC*NAGTFSQPEK | (SEQ ID NO: 67) | C129 | 1.54 | O43684 J3QT28 | 2 |
| | | | | O43684 | |
| CPEALFQPSFLGMESC*GIHETTFNSIMK | (SEQ ID NO: 68) | | 1.54 | P63261 P60709 | 2 |
| SWC*PDCVQAEPVVR | (SEQ ID NO: 69) | | 1.54 | Q9BRA2 | 2 |
| KLDTNSDGQLDFSEFLNLIGGLAMAC*HDSFLK | (SEQ ID NO: 70) | | 1.53 | | 2 |
| SIQFVDWC*PTGFK | (SEQ ID NO: 71) | C347 | 1.53 | P68366 P68363 | 2 |
| | | | | P68366 | |
| SGGSGGC*SGAGGASNCGTGSGR | (SEQ ID NO: 72) | | 1.53 | E9PI68 | 3 |
| | | | | A0A087WUC6 | |
| | | | | Q15005 | |
| FTLDC*THPVEDGIMDAANFEQFLQER | (SEQ ID NO: 73) | | 1.53 | P35268 | 2 |
| DLNYC*FSGMSDHR | (SEQ ID NO: 74) | C267 | 1.53 | P31943 G8JLB6 | 3 |
| | | | | P55795 | |
| C*EFEEVQGFLDQVAHK | (SEQ ID NO: 75) | | 1.53 | E9PI14 Q9NX20 | 2 |
| NLPFPTYFPDGDEEELPEDLYDENVC*QPGAPSITFA | (SEQ ID NO: 76) | C365 | 1.53 | E7ETU7 H0Y9G6 | 2 |
| | | C338 | | P09001 | |
| GQVC*LPVISAENWKPATK | (SEQ ID NO: 77) | C144 | 1.52 | P68036 P68036 | 2 |
| | | | | P68036 | |
| C*FGTGAAGNR | (SEQ ID NO: 78) | | 1.52 | P78527 | 3 |
| ADPDGPEAQAEAC*SGER | (SEQ ID NO: 79) | C18 | 1.52 | Q9NX24 | 2 |
| VHPAMATAAGGC*R | (SEQ ID NO: 80) | | 1.52 | H7C0N4 H7C561 | 3 |
| EMFPYEASTPTGISASC*R | (SEQ ID NO: 81) | C323 | 1.51 | G5E972 P42167 | 2 |
| | | | | P42167 | |
| LNIISNLDC*VNEVIGIR | (SEQ ID NO: 82) | C357 | 1.51 | P30154 P30154 | 2 |
| | | C402 | | P30154 P30153 | |
| | | C390 | | | |
| YMACC*LLYR | (SEQ ID NO: 83) | C386 | 1.51 | P68366 Q9BQE3 | 2 |
| | | C316 | | P68363 F5H5D3 | |
| | | | | Q71U36 P68366 | |
| SC*SGVEFSTSGSSNTDTGK | (SEQ ID NO: 84) | C36 | 1.51 | A0A0A0MR02 | |
| | | | | P45880 | 3 |
| AGSDGESIGNC*PFSQR | (SEQ ID NO: 85) | C35 | 1.51 | Q9Y696 | 3 |
| GC*EVVVSGK | (SEQ ID NO: 86) | | 1.50 | P23396 | 2 |
| NILGGTVFREPIIC*K | (SEQ ID NO: 87) | C154 | 1.50 | P48735 P48735 | 3 |
| AGAIAPC*EVTVPAQNTGLGPEK | (SEQ ID NO: 88) | | 1.50 | P05388 | 3 |
| TIGGGDDSFTTFFC*ETGAGK | (SEQ ID NO: 89) | | 1.49 | P68366 P68366 | 2 |
| FNNWGGSLSLGHPFGATGC*R | (SEQ ID NO: 90) | C413 | 1.49 | P55084 P55084 | 2 |
| NWYVQPSC*ATSGDGLYEGLTWLTSNYKS | (SEQ ID NO: 91) | C155 | 1.49 | P62330 | 2 |
| APC*QAGDLR | (SEQ ID NO: 92) | | 1.49 | P48431 | 2 |
| QVLMGPYNPDTC*PEVGFFDVLGNDR | (SEQ ID NO: 93) | | 1.49 | Q9H3P7 | 3 |
| VTEDENDEPIEIPSEDDGTVLLSTVTAQFPGAC*GLR | (SEQ ID NO: 94) | C39 | 1.49 | A0A087X260 | 2 |
| | | | | A0A087WYY0 | |
| | | | | B1AKP7 Q13148 | |
| | | | | G3V162 | |
| IQCTLQDVGSALATPC*SSAR | (SEQ ID NO: 95) | C80 | 1.49 | Q96EY8 54R3P5 | 2 |
| NLSFFLTPPC*AR | (SEQ ID NO: 96) | C492 | 1.48 | P42224 P42224 | 2 |
| | | | | J3KPM9 | |
| LLACIASRPGQC*GR | (SEQ ID NO: 97) | | 1.48 | P62241 | 2 |
| C*SDNSSYEEPLSPISASSSTSR | (SEQ ID NO: 98) | C205 | 1.48 | Q8IXK0 | 2 |
| | | C711 | | A0A0A0MSI2 | |
| | | C346 | | Q8IXK0 Q8IXK0 | |
| | | | | Q8IXK0 B3KPJ4 | |
| | | | | Q8IXK0 | |
| C*LAQEVNIPDWIVDLR | (SEQ ID NO: 99) | | 1.48 | Q9Y4W2 Q9Y4W2 | 2 |
| HEEFEEGC*K | (SEQ ID NO: 100) | C41 | 1.48 | I3L3Q4 Q9HC38 | 2 |
| | | C245 | | Q9HC38 F6TLX2 | |
| GLDYEGGGC*R | (SEQ ID NO: 101) | C691 | 1.48 | O60568 | 2 |
| LLQC*DPSSASQF | (SEQ ID NO: 102) | | 1.47 | P37235 | 3 |
| TVPFLPLLGGC*IDDTILSR | (SEQ ID NO: 103) | C180 | 1.47 | Q7Z7H8 Q7Z7H8 | 3 |
| IINDNATYC*R | (SEQ ID NO: 104) | | 1.47 | O00567 | 2 |
| SLLINAVEASC*IR | (SEQ ID NO: 105) | C262 | 1.47 | E2QRB3 P32322 | 2 |
| | | C188 | | P32322 | |
| | | | | A0A087WTV6 | |
| | | | | P32322 J3KR12 | |
| | | | | Q96C36 | |
| PPMEAAGFTAQVIILNHPGQISAGYAPVLDCHTAHIAC*K | (SEQ ID NO: 106) | | 1.47 | P68104 | 2 |
| | | | | A0A087WVQ9 | |
| HPLTQELKEC*EGIVPVPLAEK | (SEQ ID NO: 107) | | 1.46 | P82932 | 2 |
| HTGPGILSMANAGPNTNGSQFFIC*TAK | (SEQ ID NO: 108) | C115 | 1.46 | P62937 | 2 |
| IISDNLTYC*K | (SEQ ID NO: 109) | | 1.46 | Q9Y2X3 | 3 |
| LIDFLEC*GK | (SEQ ID NO: 110) | C234 | 1.45 | P17844 J3KTA4 | 2 |
| KAVVVC*PK | (SEQ ID NO: 111) | C588 | 1.45 | Q00839 Q00839 | 2 |
| LTTPTYGDLNHLVSATMSGVTTC*LR | (SEQ ID NO: 112) | C239 | 1.45 | Q13885 P68371 | 3 |
| | | C221 | | Q9BVA1 P04350 | |
| | | | | Q5JP53 | |
| VCETDGC*SSEAK | (SEQ ID NO: 113) | C10 | 1.45 | D6RF24 H0Y9L0 | 3 |
| | | C14 | | P53582 | |
| VC*PPHMLPEDGANLSSAR | (SEQ ID NO: 114) | C29 | 1.44 | Q9GZY8 | 2 |
| | | | | A0A0A0MS29 | |
| | | | | Q9GZY8 H7C433 | |
| | | | | E9PQX8 | |
| TFVDFFSQC*LHEEYR | (SEQ ID NO: 115) | | 1.44 | Q53GQ0 | 2 |
| FALAC*NASDKIIEPIQSR | (SEQ ID NO: 116) | | 1.44 | P35250 P35250 | 2 |
| TIAEC*LADELINAAK | (SEQ ID NO: 117) | C172 | 1.44 | P46782 M0R0R2 | 3 |
| | | | | M0R0F0 | |
| FMTPVIQDNPSGWGPC*AVPEQFR | (SEQ ID NO: 118) | C19 | 1.44 | O15371 O15371 | 3 |
| | | | | O15371 | |
| SVLLCGIEAQAC*ILNTTLDLLDR | (SEQ ID NO: 119) | C114 | 1.44 | K7ENV7 K7EKW4 | 2 |
| | | | | Q96AB3 | |
| AVAILC*NHQR | (SEQ ID NO: 120) | | 1.44 | P11387 | 3 |
| GNFTLPEVAEC*FDEITYVELQKEEAQK | (SEQ ID NO: 121) | C629 | 1.44 | Q00839 Q00839 | 2 |
| TIIPLISQC*TPK | (SEQ ID NO: 122) | C212 | 1.44 | P40926 | 3 |
| DVQIGDIVTVGEC*RPLSK | (SEQ ID NO: 123) | C131 | 1.44 | P62280 | 2 |
| LLDLVQQSC*NYK | (SEQ ID NO: 124) | C30 | 1.43 | B1AHD1 P55769 | 3 |
| C*MTNTPVVVR | (SEQ ID NO: 125) | C120 | 1.43 | P32322 P32322 | 2 |
| | | | | P32322 | |
| VLGLGLGC*LR | (SEQ ID NO: 126) | | 1.43 | Q9BRJ7 K7EIN2 | 3 |
| VC*ISILHAPGDDPMGYESSAER | (SEQ ID NO: 127) | | 1.43 | P60604 P60604 | 2 |
| AYHEQLSVAEITNAC*FEPANQMVK | (SEQ ID NO: 128) | | 1.43 | P68366 Q13748 | 3 |
| | | | | Q71U36 P68366 | |
| AGQC*VIGLQMGTNK | (SEQ ID NO: 129) | C153 | 1.43 | Q99439 B4DDF4 | 3 |
| | | C155 | | B4DUT8 | |
| | | C164 | | A0A087X271 | |
| SGDAAIVDMVPGKPMC*VESFSDYPPLGR | (SEQ ID NO: 130) | | 1.42 | P68104 | 2 |
| | | | | A0A087WVQ9 | |
| VSDTVVEPYNATLSVHQLVENTDETYC*IDNEALYDICFR | (SEQ ID NO: 131) | C201 | 1.42 | P68371 Q9BVA1 | 2 |
| | | C183 | | P04350 Q5JP53 | |
| | | | | Q9BUF5 | |
| C*EFQDAYVLLSEKK | (SEQ ID NO: 132) | | 1.42 | P10809 | 2 |
| SSQC*HTGSSPR | (SEQ ID NO: 133) | C439 | 1.42 | O15226 O15226 | 2 |
| LNISFPATGC*QK | (SEQ ID NO: 134) | | 1.42 | P62753 | 3 |
| FHADSVC*K | (SEQ ID NO: 135) | | 1.42 | Q9BW61 | 2 |
| C*MQLTDFILK | (SEQ ID NO: 136) | C54 | 1.42 | E7EPB3 P50914 | 2 |
| ELEVLLMC*NK | (SEQ ID NO: 137) | C91 | 1.42 | P62910 F8W727 | 2 |
| | | C109 | | D3YTB1 | |
| NHLLPDIVTC*VQSSR | (SEQ ID NO: 138) | C184 | 1.42 | Q9BSD7 | 2 |
| VNGTTPC*AFPTHYEEALKDEEK | (SEQ ID NO: 139) | | 1.41 | O15479 | 2 |
| LTEGC*SFR | (SEQ ID NO: 140) | C93 | 1.41 | H0YMV8 Q71UM5 | 2 |
| | | C77 | | P42677 | |
| C*ALSSPSLAFTPPIK | (SEQ ID NO: 141) | C120 | | Q8NFH5 Q8NFH5 | 2 |
| | | | | Q8NFH5 | |
| SSVQEEC*VSTISSSKDEDPLAATR | (SEQ ID NO: 142) | | 1.41 | Q7L0Y3 | 3 |
| TQNLPNC*QLISR | (SEQ ID NO: 143) | | 1.41 | P37268 | 2 |
| C*SEGSFLLTTFPRPVTVEPMDQLDDEEGLPEK | (SEQ ID NO: 144) | C119 | 1.41 | Q15233 Q15233 | 3 |
| SMVSPVPSPTGTISVPNSC*PASPR | (SEQ ID NO: 145) | | 1.41 | P85037 | 2 |
| EITSLDTENIDEILNNADVALVNFYADWC*R | (SEQ ID NO: 146) | C58 | 1.40 | Q9B526 | 2 |
| GHSSDSNPAIC*R | (SEQ ID NO: 147) | C31 | 1.40 | Q5JTH9 | 2 |
| FC*IWTESAFR | (SEQ ID NO: 148) | C250 | 1.40 | P36578 | 2 |
| SLC*NLEESITSAGRDDLESFQLEISGFLK | (SEQ ID NO: 149) | | 1.40 | Q52LJ0 Q52LJ0 | 2 |
| IDC*FSEVPTSVFGEK | (SEQ ID NO: 150) | | 1.40 | O00567 | 2 |
| NMSVHLSPC*FR | (SEQ ID NO: 151) | C116 | 1.40 | P62280 | 3 |
| GLPC*TELFVAPVGVASKR | (SEQ ID NO: 152) | C79 | 1.40 | H0Y901 E9PDI4 | 3 |
| | | C428 | | O00515 | |
| VAC*ITEQVLTLVNKR | (SEQ ID NO: 153) | | 1.40 | P04843 | 3 |
| NSQWVPTLPNSSHHLDAVPC*STTINR | (SEQ ID NO: 154) | C138 | 1.40 | Q12824 Q12824 | 2 |
| | | | | G5E975 | |
| NVQLLSQFVSPFTGC*IYGR | (SEQ ID NO: 155) | | 1.40 | Q9Y3D5 | 2 |
| LC*SSSSSDTSSR | (SEQ ID NO: 156) | C385 | 1.40 | Q86WB0 Q86WB0 | 2 |
| | | | | C9J0I9 | |
| LVVPASQC*GSLIGK | (SEQ ID NO: 157) | C109 | 1.40 | Q15366 Q15366 | 3 |
| | | | | Q15366 Q15366 | |
| VNAAGTDPSSPVGFVLGVDLLHIFPLEGATFLC*PADVTDPR | (SEQ ID NO: 158) | | 1.39 | Q9UI43 | 2 |
| C*PFTGNVSIR | (SEQ ID NO: 159) | C60 | 1.39 | P62280 | 2 |
| LTALDYHNPAGFNC*KDETEFR | (SEQ ID NO: 160) | C19 | 1.39 | Q9Y224 | 3 |
| VPTANVSVVDLTC*R | (SEQ ID NO: 161) | C247 | 1.39 | P04406 | 2 |
| DSGYGDIWC*PERGEFLAPPR | (SEQ ID NO: 162) | C176 | 1.39 | J3KP06 F8WD26 | 2 |
| | | C228 | | Q8WWI1 Q8WWI1 | |
| | | C213 | | Q8WWI1 Q8WWI1 | |
| NFNYHILSPC*DLSNYTDLAMSTVK | (SEQ ID NO: 163) | C461 | 1.39 | G5E9W3 Q9UKF6 | 3 |
| AC*QSIYPLHDVFVR | (SEQ ID NO: 164) | C201 | 1.39 | D6RB09 D6RAT0 | |
| | | | | P61247 D6RG13 | |
| WC*EYGLTFTEK | (SEQ ID NO: 165) | C65 | 1.38 | A0A0A0MR02 | 2 |
| | | | | P45880 | |
| KTPC*GEGSK | (SEQ ID NO: 166) | C70 | 1.38 | P60866 P60866 | 2 |
| NC*LTNFHGMDLTR | (SEQ ID NO: 167) | C96 | 1.38 | D6RB09 D6RAT0 | 2 |
| | | | | P61247 D6RG13 | |
| YGIIC*MEDLIHEIYTVGKR | (SEQ ID NO: 168) | C186 | 1.38 | P18124 A8MUD9 | 3 |
| C*ASQAGMTAYGTR | (SEQ ID NO: 169) | C127 | 1.38 | E9PDU6 Q15417 | 2 |
| | | C132 | | Q15417 | |
| KAQC*PIVER | (SEQ ID NO: 170) | C66 | 1.38 | P46782 M0R0R2 | 2 |
| | | | | M0R0F0 | |
| NGDIC*ETSGKPK | (SEQ ID NO: 171) | C60 | 1.38 | Q16637 E7EQZ4 | 2 |
| | | | | Q16637 Q16637 | |
| | | | | Q16637 B4DP61 | |
| KGAGNPQASTLALQSNITQC*LLGQPWPLNEAQVQASVVK | (SEQ ID NO: 172) | C1260 | 1.38 | E7ETY2 Q13428 | 3 |
| | | C1261 | | Q13428 Q13428 | |
| | | C1299 | | Q13428 Q13428 | |
| | | | | Q13428 Q13428 | |
| | | | | J3KQ96 | |
| FCSFSPC*IEQVQR | (SEQ ID NO: 173) | C209 | 1.37 | Q96FX7 | 2 |
| IHMGSC*AENTAK | (SEQ ID NO: 174) | | 1.37 | P24752 | 3 |
| GC*IVDANLSVLNLVIVK | (SEQ ID NO: 175) | | 1.37 | P62753 | 3 |
| IAILTC*PFEPPKPK | (SEQ ID NO: 176) | C253 | 1.37 | E9PCA1 B7ZAR1 | 2 |
| | | | | P48643 | |
| TLTSC*FLSCVVCVEGIVTK | (SEQ ID NO: 177) | C164 | 1.37 | P25205 P25205 | 2 |
| LGYILTC*PSNLGTGLR | (SEQ ID NO: 178) | C347 | 1.37 | P12532 P12532 | 2 |
| LMWLFGC*PLLLDDVAR | (SEQ ID NO: 179) | | 1.37 | O15067 | 2 |
| INISEGNC*PER | (SEQ ID NO: 180) | C54 | 1.37 | Q15366 Q15366 | 3 |
| | | | | Q15366 Q15366 | |
| | | | | Q15365 | |
| SGQGAFGNMC*R | (SEQ ID NO: 181) | C96 | 1.37 | P36578 | 3 |
| GLC*AIAQAESLR | (SEQ ID NO: 182) | | 1.37 | P23396 | 3 |
| IIPTLEEGLQLPSPTATSQLPLESDAVEC*LNYQHYK | (SEQ ID NO: 183) | | 1.37 | P61978 P61978 | 3 |
| C*PEALFQPSFLGMESCGIHETTFNSIMK | (SEQ ID NO: 184) | | 1.37 | P60709 P63261 | 2 |
| LC*FSTAQHAS | (SEQ ID NO: 185) | | 1.37 | M0QXS5 P14866 | 2 |
| LGTLAPFC*CPWEQLTQDWESR | (SEQ ID NO: 186) | C705 | 1.37 | Q99575 | 2 |
| AAVEEGIVLGGGC*ALLR | (SEQ ID NO: 187) | | 1.37 | P10809 | 3 |
| LDSLGLCSVSC*ALEFIPNSK | (SEQ ID NO: 188) | C256 | 1.36 | Q9BSH4 | 2 |
| VC *NFLASQVPFPSR | (SEQ ID NO: 189) | C205 | 1.36 | Q99714 | 3 |
| VTDGALVVVDCVSGVC*VQTETVLR | (SEQ ID NO: 190) | C136 | 1.36 | P13639 | 2 |
| YAIC*SALAASALPALVMSK | (SEQ ID NO: 191) | C125 | 1.36 | P36578 | 3 |
| TATAVAHC*K | (SEQ ID NO: 192) | C25 | 1.36 | MOR210 P62249 | 3 |
| GC*TATLGNFAK | (SEQ ID NO: 193) | | 1.36 | P15880 | 2 |
| VC*NYGLTFTQK | (SEQ ID NO: 194) | C66 | 1.36 | Q9Y277 Q9Y277 | 2 |
| SLHDALC*VLAQTVK | (SEQ ID NO: 195) | C348 | 1.36 | P78371 | 2 |
| AWVWNTHADFADEC* | (SEQ ID NO: 196) | C209 | 1.36 | C9JXG8 P43487 | 2 |
| PKPELLAIR | | C132 | | C9JJ34 F6WQW2 | |
| | | | | C9JGV6 P43487 | |
| C*HTPPLYR | (SEQ ID NO: 197) | | 1.36 | M0R117 Q02543 | 2 |
| YIGENLQLLVDRPDGTYC*FR | (SEQ ID NO: 198) | | 1.36 | Q9Y221 | 2 |
| ALANVNIGSLIC*NVGAGGPAPAAGAAPAGGPAPSTAAAPAE | (SEQ ID NO: 199) | | 1.36 | P05386 | 2 |
| EK | | | | | |
| ANC*IDSTASAEAVFASEVKK | (SEQ ID NO: 200) | C268 | 1.36 | M0QXL5 M0R299 | 2 |
| | | C183 | | P22087 M0R2Q4 | |
| NC*IVLIDSTPYR | (SEQ ID NO: 201) | | 1.35 | P62241 | 2 |
| YSTGSDSASFPHTTPSMC*LNPDLEGPPLEAYTIQGQY | (SEQ ID NO: 202) | C217 | 1.35 | Q15366 Q15366 | 2 |
| LGEWVGLC*K | (SEQ ID NO: 203) | | 1.35 | P25398 | 2 |
| C*ASQVGMTAPGTR | (SEQ ID NO: 204) | C215 | 1.35 | Q99439 B4DDF4 | 2 |
| | | C204 | | B4DUT8 | |
| TTSSANNPNLMYQDEC*DR | (SEQ ID NO: 205) | C586 | 1.35 | Q92841 H3BLZ8 | 2 |
| | | C507 | | Q92841 Q92841 | |
| | | C505 | | Q92841 | |
| ISLGLPVGAVINC*ADNTGAK | (SEQ ID NO: 206) | | 1.35 | P62829 | 2 |
| AYGGSMC*AK | (SEQ ID NO: 207) | | 1.35 | P49207 | 2 |
| VVNSETPVVVDFHAQWC*GPCK | (SEQ ID NO: 208) | C90 | 1.35 | Q99757 F8WDN2 | 2 |
| GVPGAIVNVSSQC*SQR | (SEQ ID NO: 209) | C137 | 1.34 | Q7Z4W1 J3QS36 | 2 |
| | | | | J3KS22 | |
| GC*LWALNPAKIDK | (SEQ ID NO: 210) | | 1.34 | O15353 | 2 |
| DLC*FSPGLMEASHVVNDVNEAVQLVFR | (SEQ ID NO: 211) | C362 | 1.34 | Q9BXW7 Q9BXW7 | 2 |
| HGFC*GIPITDTGR | (SEQ ID NO: 212) | | 1.34 | P12268 | 3 |
| ASDHGWVC*DQR | (SEQ ID NO: 213) | C309 | 1.34 | Q9HC36 | 2 |
| AVC*MLSNTTAVAEAWAR | (SEQ ID NO: 214) | C376 | 1.33 | Q9BQE3 | 3 |
| HIGDGC*CLTR | (SEQ ID NO: 215) | C202 | 1.33 | A0A087WZT2 | 3 |
| | | | | Q6UX53 | |
| SDLGPC*EK | (SEQ ID NO: 216) | | 1.33 | D6RDI2 J3KPP4 | 2 |
| | | | | O95232 | |
| LPITVLNGAPGFINLC*DALNAWQLVK | (SEQ ID NO: 217) | C240 | 1.33 | P31939 P31939 | 2 |
| VDEFPLC*GHMVSDEYEQLSSEALEAAR | (SEQ ID NO: 218) | C49 | 1.33 | X1WI28 P27635 | 3 |
| IISNASC*TTNCLAPLAK | (SEQ ID NO: 219) | C152 | 1.33 | P04406 | 3 |
| DLTTAGAVTQC*YR | (SEQ ID NO: 220) | | 1.33 | Q02543 | 2 |
| SAC*SLESNLEGLAGVLEADLPNYK | (SEQ ID NO: 221) | C44 | 1.32 | Q09161 | 2 |
| KITAFVPNDGC*LNFIEENDEVLVAGFGR | (SEQ ID NO: 222) | | 1.32 | P62266 | 2 |
| TGC*TFPEKPDFH | (SEQ ID NO: 223) | C318 | 1.32 | P55263 P55263 | 3 |
| | | C336 | | P55263 | |
| NQSFC*PTVNLDKLWTLVSEQTR | (SEQ ID NO: 224) | C70 | 1.32 | P46776 E9PLL6 | 3 |
| | | | | E9PJD9 | |
| SGEEDFESLASQFSDC*SSAK | (SEQ ID NO: 225) | C113 | 1.32 | K7EN45 K7EMU7 | 2 |
| | | | | Q13526 | |
| SPWLAGNELTVADVVLWSVLQQIGGC*SVTVPANVQR | (SEQ ID NO: 226) | | 1.32 | Q13155 | 2 |
| VNQAIWLLC*TGAR | (SEQ ID NO: 227) | C155 | 1.32 | P46782 M0R0R2 | 3 |
| | | | | M0R0F0 | |
| AC*DLPAWVHFPDTER | (SEQ ID NO: 228) | | 1.32 | H7BXI1 | 2 |
| YYALCGFGGVLSC*GLTHTAVVPLDLVK | (SEQ ID NO: 229) | | 1.31 | Q00325 | 2 |
| EC*LPLIIFLR | (SEQ ID NO: 230) | | 1.31 | P62701 | 3 |
| VLVTTNVC*AR | (SEQ ID NO: 231) | C392 | 1.31 | Q9UMR2 Q9UMR2 | 2 |
| | | C393 | | Q9NUU7 Q9NUU7 | |
| | | C367 | | H3BQK0 F6QDS0 | |
| | | C310 | | I3L352 | |
| SYC*AEIAHNVSSK | (SEQ ID NO: 232) | C114 | 1.31 | P62910 F8W727 | 2 |
| | | C96 | | D3YTB1 | |
| TAFQEALDAAGDKLVVVDFSATWC*GPCK | (SEQ ID NO: 233) | | 1.31 | P10599 | 2 |
| LC*YVALDFEQEMATAASSSSLEK | (SEQ ID NO: 234) | | 1.30 | P60709 P63261 | 3 |
| | | | | Q658J3 | |
| VRPSTGNSASTPQSQC*LPSEIEVK | (SEQ ID NO: 235) | C131 | 1.30 | Q9UJX3 Q9UJX3 | 2 |
| AATGEEVSAEDLGGADLHC*R | (SEQ ID NO: 236) | | 1.30 | Q9HCCO | 2 |
| PGHLQEGFGC*VVTNRFDQLFDDESDPFEVLK | (SEQ ID NO: 237) | C11 | 1.30 | Q8NC51 Q8NC51 | 3 |
| | | | | Q8NC51 Q8NC51 | |
| C*ASQSGMTAYGTR | (SEQ ID NO: 238) | C166 | 1.30 | Q99439 B4DDF4 | 3 |
| | | C164 | | B4DUT8 | |
| | | C175 | | A0A087X271 | |
| LC*VQNSPQEAR | (SEQ ID NO: 239) | C150 | 1.30 | P33240 P33240 | 3 |
| | | | | E7EWR4 E9PID8 | |
| | | | | A0A0A0MT56 | |
| PC*SEETPAISPSKR | (SEQ ID NO: 240) | C3 | 1.30 | P33316 H0YNJ9 | 2 |
| LDINLLDNVVNC*LYHGEGAQQR | (SEQ ID NO: 241) | | 1.30 | O14980 | 2 |
| NMMAAC*DPR | (SEQ ID NO: 242) | C285 | 1.29 | Q13509 Q13885 | 3 |
| | | C650 | | P68371 A6NNZ2 | |
| | | C303 | | A0A0B4J269 | |
| | | C266 | | Q9BVA1 Q3ZCM7 | |
| | | | | P04350 K7ESM5 | |
| | | | | Q5JP53 Q9BUF5 | |
| IAVHC*TVR | (SEQ ID NO: 243) | C72 | 1.29 | P62913 Q5VVC8 | 2 |
| | | | | P62913 | |
| C*PQVEEAIVQSGQKK | (SEQ ID NO: 244) | C146 | 1.29 | Q9BVP2 Q9BVP2 | 2 |
| SQAPC*ANKDEADLSSK | (SEQ ID NO: 245) | C300 | 1.29 | Q96SK2 Q96SK2 | 2 |
| C*LGHPEEFYNLVR | (SEQ ID NO: 246) | | 1.29 | P37268 | 2 |
| NTVLC*NVVEQFLQADLAR | (SEQ ID NO: 247) | C70 | 1.29 | Q14258 | 2 |
| AFQYVETHGEVC*PANWTPDSPTIKPSPAASK | (SEQ ID NO: 248) | | 1.29 | P30048 | 3 |
| QAVLGAGLPISTPC*TTINK | (SEQ ID NO: 249) | C119 | 1.29 | P24752 | 3 |
| GTPEQPQC*GFSNAVVQILR | (SEQ ID NO: 250) | C67 | 1.29 | Q86SX6 | 3 |
| ADHQPLTEASYVNLPTIALC*NTDSPLR | (SEQ ID NO: 251) | C148 | 1.29 | A0A0C4DG17 | 3 |
| | | | | C9J9K3 P08865 | |
| GSDELFSTC*VTNGPFIIVISSNSASAANGNDSKK | (SEQ ID NO: 252) | C23 | 1.29 | A0A0U1RRM4 | 3 |
| | | | | P26599 P26599 | |
| | | | | P26599 | |
| AYHEQLTVAEITNAC*FEPANQMVK | (SEQ ID NO: 253) | C295 | 1.28 | Q9BQE3 F5H5D3 | 2 |
| | | C365 | | | |
| INPYMSSPC*HIEMILTEK | (SEQ ID NO: 254) | C106 | 1.28 | J3KRX5 J3QLC8 | 2 |
| | | C144 | | A0A087WXM6 | |
| | | | | P18621 | |
| | | | | A0A0A6YYL6 | |
| | | | | J3QQT2 P18621 | |
| YIYDQC*PAVAGYGPIEQLPDYNR | (SEQ ID NO: 255) | C453 | 1.28 | P31930 | 2 |
| C*ELSSSVQTDINLPYLTMDSSGPK | (SEQ ID NO: 256) | | 1.28 | P38646 | 3 |
| GC*LLYGPPGTGK | (SEQ ID NO: 257) | | 1.27 | A0A087X2I1 | 3 |
| | | | | P62333 | |
| ALNALC*DGLIDELNQALK | (SEQ ID NO: 258) | | 1.27 | P30084 | 3 |
| NTPSFLIAC*NK | (SEQ ID NO: 259) | C179 | 1.27 | Q9Y5M8 | 2 |
| C*MPTFQFFK | (SEQ ID NO: 260) | | 1.27 | P10599 | 3 |
| TGNGPMSVC*GR | (SEQ ID NO: 261) | C493 | 1.27 | O95793 O95793 | 2 |
| | | | | O95793 | |
| | | | | A0A087X1A5 | |
| | | | | Q5JW30 | |
| HELQANC*YEEVKDR | (SEQ ID NO: 262) | C139 | 1.27 | E9PK25 G3V1A4 | 3 |
| | | C177 | | P23528 | |
| AFAFVTFADDQIAQSLC*GEDLIIK | (SEQ ID NO: 263) | C244 | 1.27 | A0A087X260 | 2 |
| | | | | A0A087WYY0 | |
| | | | | B1AKP7 Q13148 | |
| | | | | G3V162 | |
| LTPGC*EAEAETEAICFFVQQFTDMEHNR | (SEQ ID NO: 264) | C2359 | 1.26 | P49327 | 2 |
| LECVEPNC*R | (SEQ ID NO: 265) | | 1.26 | Q969Q0 | 2 |
| SYC*NDQSTGDIK | (SEQ ID NO: 266) | C106 | 1.26 | P00492 | 2 |
| ATC*APQHGAPGPGPADASK | (SEQ ID NO: 267) | C2516 | 1.26 | P21333 | 2 |
| | | C2535 | | A0A087WWY3 | |
| | | | | Q60FE5 P21333 | |
| ATYDKLC*K | (SEQ ID NO: 268) | | 1.26 | P62851 | 2 |
| GSC*STEVEKETQEK | (SEQ ID NO: 269) | C69 | 1.26 | O75348 | 2 |
| TVDSQGPTPVC*TPTFLER | (SEQ ID NO: 270) | | 1.26 | Q9BYG3 | 2 |
| LC*YVALDFEQEMAMVASSSSLEK | (SEQ ID NO: 271) | C880 | 1.26 | P0CG39 | 3 |
| VFIMDSC*DELIPEYLNFIR | (SEQ ID NO: 272) | C366 | 1.25 | P08238 | 3 |
| HC*SQVDSVR | (SEQ ID NO: 273) | C112 | 1.25 | Q14247 Q14247 | 2 |
| | | | | Q14247 | |
| AAAPAPEEEMDEC*EQALAAEPK | (SEQ ID NO: 274) | C266 | 1.25 | P26641 P26641 | 2 |
| EKTAC*AINK | (SEQ ID NO: 275) | C293 | 1.25 | Q8NCA5 E9PH82 | 2 |
| | | | | Q52LJ0 Q8NCA5 | |
| | | | | Q52LJ0 | |
| SGTIC*SSELPGAFEAAGFHLNEHLYNMIIR | (SEQ ID NO: 276) | C200 | 1.24 | A0A0C4DGQ5 | 2 |
| | | | | P04632 K7ELJ7 | |
| TASISSSPSEGTPTVGS YGC*TPQSLPK | (SEQ ID NO: 277) | C787 | 1.24 | Q6PKG0 Q6PKG0 | 3 |
| EVIAVSCGPAQC*QETIR | (SEQ ID NO: 278) | C162 | 1.24 | P38117 P38117 | 2 |
| NC*PHVVVGTPGR | (SEQ ID NO: 279) | C164 | 1.24 | O00148 | 3 |
| SC*PSFSASSEGTR | (SEQ ID NO: 280) | C9 | 1.24 | D6RCP9 P27707 | 2 |
| | | | | D6RFG8 D6RG38 | |
| GLIAAIC*AGPTALLAHEIGFGSK | (SEQ ID NO: 281) | C86 | 1.23 | Q99497 K7ELW0 | 2 |
| | | | | K7EN27 | |
| LYYFQYPC*YQEGLR | (SEQ ID NO: 282) | | 1.23 | Q9NRW3 | 3 |
| GC*WDSIHVVEVQEK | (SEQ ID NO: 283) | C135 | 1.22 | P47756 P47756 | 2 |
| | | C176 | | B1AK88 B1AK87 | |
| | | | | B1AK85 | |
| SCYDLSC*HAR | (SEQ ID NO: 284) | | 1.22 | P41250 | 2 |
| VGSFC*LSEAGAGSDSFALK | (SEQ ID NO: 285) | C73 | 1.21 | P45954 P45954 | 2 |
| AINC*ATSGVVGLVNCLR | (SEQ ID NO: 286) | C1448 | 1.21 | P49327 | 2 |
| TSAVPSPC*GK | (SEQ ID NO: 287) | C260 | 1.21 | P49748 P49748 | 2 |
| | | | | P49748 | |
| INEIVYFLPFC*HSELIQLVNK | (SEQ ID NO: 288) | C513 | 1.21 | Q9H078 Q9H078 | 2 |
| | | C371 | | Q9H078 Q9H078 | |
| | | C572 | | Q9H078 H0YGM0 | |
| IGFPETTEEELEEIASENSDC*IFPSAPDVK | (SEQ ID NO: 289) | C353 | 1.21 | Q9Y3F4 Q9Y3F4 | 2 |
| IVGYFVSGC*DPSIMGIGPVPAISGALK | (SEQ ID NO: 290) | C287 | 1.21 | A0A0B4J2A4 | 2 |
| | | | | P42765 | |
| RGPC*IIYNEDNGIIK | (SEQ ID NO: 291) | C208 | 1.21 | P36578 | 2 |
| AALVTSFC*MFKYMALYSMIQR | (SEQ ID NO: 292) | C542 | 1.20 | H0Y4Z2 | 2 |
| ESLNASIVDAINQAADC*WGIR | (SEQ ID NO: 293) | C167 | 1.20 | Q9UJZ1 | 2 |
| EEHLC*TQR | (SEQ ID NO: 294) | | 1.20 | J3KN67 | 2 |
| GC*GVVKFESPEVAER | (SEQ ID NO: 295) | | 1.20 | P52272 P52272 | 3 |
| | | | | A0A087X0X3 | |
| KC*SASNR | (SEQ ID NO: 296) | C17 | 1.19 | Q8WVC2 Q9BYK1 | 2 |
| | | | | P63220 | |
| TPC*SSLLPLLNAHAATSGK | (SEQ ID NO: 297) | C307 | 1.18 | B8ZZZ7 Q9NUQ6 | 2 |
| | | C367 | | A0A0A0MSG5 | |
| | | C397 | | Q9NUQ6 Q9NUQ6 | |
| | | | | Q9NUQ6 | |
| VSLDPELEEALTSASDTELC*DLAAILGMHNLITNTK | (SEQ ID NO: 298) | C132 | 1.17 | Q9NYL9 | 3 |
| FDPTQFQDC*IIQGLTETGTDLEAVAK | (SEQ ID NO: 299) | C35 | 1.17 | Q7L1Q6 C9IZ80 | 2 |
| | | C39 | | Q7L1Q6 Q7L1Q6 | |
| | | | | Q7L1Q6 | |
| LGGSLIVAFEGC*PV | (SEQ ID NO: 300) | C146 | 1.16 | P60981 P60981 | 2 |
| TQYSCYC*CK | (SEQ ID NO: 301) | C229 | 1.15 | Q9UGI8 Q9UGI8 | 2 |
| IC*PVEFNPNFVAR | (SEQ ID NO: 302) | | 1.15 | Q9UI30 F5GX77 | 2 |
| TPSYSISSTLNPQAPEFILGC*TASK | (SEQ ID NO: 303) | C142 | 1.15 | Q14694 H3BQC6 | 2 |
| | | | | Q14694 Q14694 | |
| ASVGFGGSC*FQK | (SEQ ID NO: 304) | C209 | 1.14 | O60701 O60701 | 2 |
| NTGQTC*VCSNQFLVQR | (SEQ ID NO: 305) | | 1.13 | C9J8Q5 P01763 | 2 |
| | | | | P51649 P51649 | |
| FQSSAVMALQEASEAYLVGLFEDTNLC*AIHAK | (SEQ ID NO: 306) | | 1.12 | Q71DI3 | 2 |
| AGAVVAVPTDTLYGLACAASC*SAALR | (SEQ ID NO: 307) | C99 | 1.11 | Q86U90 | 3 |
| AVLLASDAQEC*TLEEVVER | (SEQ ID NO: 308) | C332 | 1.10 | Q27J81 Q27J81 | 2 |
| FQSSAVMALQEACEAYLVGLFEDTNLC*AIHAK | (SEQ ID NO: 309) | C111 | 1.08 | P68431 | 2 |
| NMITGTSQADC*AVLIVAAGVGEFEAGISK | (SEQ ID NO: 310) | | 1.08 | P68104 Q05639 | 2 |
| | | | | A0A087WVQ9 | |
| GNHEC*ASINR | (SEQ ID NO: 311) | C83 | 1.05 | P62136 P62140 | 3 |
| | | C126 | | P62136 | |
| LC*DFGVSGQLIDSMANSFVGTR | (SEQ ID NO: 312) | C207 | 1.04 | G5E9C7 Q02750 | 2 |
| | | C211 | | P36507 Q02750 | |
| | | C114 | | | |
| SC*GSSTPDEFPTDIPGTK | (SEQ ID NO: 313) | | 1.02 | P41091 | 2 |
| GTLTLC*PYHSDR | (SEQ ID NO: 314) | C620 | 1.00 | Q13200 Q13200 | 2 |
| | | | | Q13200 | |
| LSLDGQNIYNAC*CTLR | (SEQ ID NO: 315) | C250 | 0.95 | A0A0U1RRM4 | 2 |
| | | C281 | | P26599 P26599 | |
| | | | | P26599 | |
| ADASSTPSFQQAFASSC*TISSNGPGQR | (SEQ ID NO: 316) | C688 | 0.94 | Q68CZ2 Q68CZ2 | 2 |
| STLTDSLVC*K | (SEQ ID NO: 317) | C41 | 0.94 | P13639 | 3 |
| SDITKLEVDAIVNAA | (SEQ ID NO: 318) | C186 | 0.91 | Q9BQ69 | 2 |
| NSSLLGGGGVDGC*IHR | | | | | |
| LC*EPEVLNSLEETYSPFFR | (SEQ ID NO: 319) | C261 | 0.90 | H0YJA2 Q6PJT7 | 2 |
| | | C177 | | Q6PJT7 G3V5I6 | |
| | | C224 | | Q6PJT7 G3V256 | |
| | | | | Q6PJT7 Q6PJT7 | |
| | | | | Q6PJT7 Q6PJT7 | |
| | | | | Q6PJT7 | |
| C*PAPPRGPPAPAPEVEELAR | (SEQ ID NO: 320) | C161 | 0.81 | P48681 | 2 |
| AAC*LESAQEPAGAWGNK | (SEQ ID NO: 321) | C53 | 0.76 | A0A024R4E5 | 2 |
| LHTGPLPEQC*R | (SEQ ID NO: 322) | C163 | 0.68 | A0JLT2 J3KR33 | 2 |
| | | | | A0JLT2 | |
| FQSAAIGALQEASEAYLVGLFEDTNLC*A | (SEQ ID NO: 323) | | 0.66 | K7EK07 P84243 | 2 |
| FQSSAVMALQEAREAYLVGLFEDTNLC*AIRAK | (SEQ ID NO: 324) | C111 | 0.65 | Q5TEC6 | 2 |
| HLNEIDLFHC*IDPNDSK | (SEQ ID NO: 325) | C62 | 0.59 | A0A087WYT3 | 2 |
| | | C58 | | Q15185 Q15185 | |
| QC*PIMDPAWEAPEGVPIDAIIFGGR | (SEQ ID NO: 326) | C297 | 0.29 | B4DW73 Q16822 | 3 |
| SEC*DQDYIPETDQDC*SMSPCPQRTPDSGLAQHPFQNEDYR | (SEQ ID NO: 327) | C1290 | 0.12 | Q9P2N4 Q9P2N4 | 2 |
| | | C1250 | | H0Y859 Q9P2N4 | |
| | | C1278 | | | |
| | | C1262 | | | |

To determine the relevance of RTN4 in colorectal cancer, we performed isoTOP-ABPP analysis to quantitatively map proteome-wide reactivity of cysteines in pooled primary human colorectal tumors through comparative ratiometric analysis of IAyne labeling at 100 (heavy) versus 10 μM (light) concentrations. Previous studies by Weerapana et al. have shown that hyper-reactive cysteines, which show saturated IAyne labeling at lower concentrations and thus exhibit a lower (<3) heavy to light ratio, are highly enriched in functional cysteines, compared to those sites that are not hyper-reactive that show heavy to light ratios of ˜10¹⁰. We identify RTN4 labeling of C1101 in primary human colorectal tumors. RTN4 C1101 shows a ratio of 6.2 indicating that this cysteine is not hyper-reactive (FIG. 2B). Our data thus show that RTN4 is present and that C1101 within RTN4 is accessible in primary human colorectal tumors.

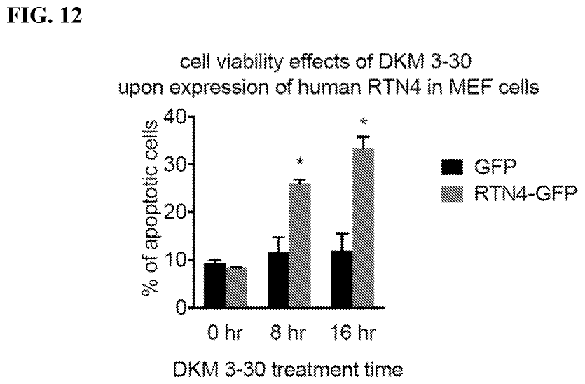

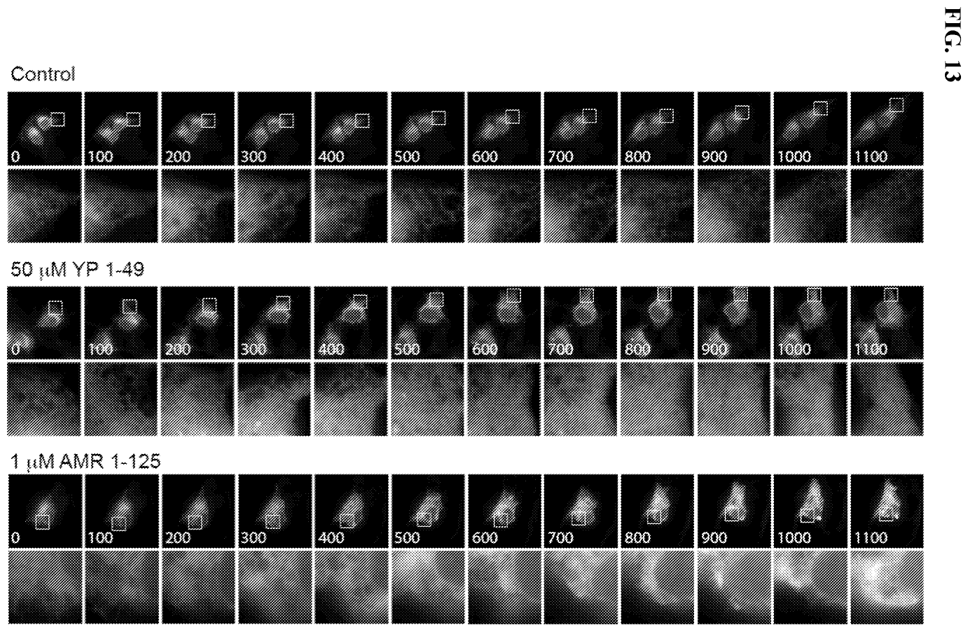

We further confirm the relevance of RTN4 in colorectal cancer by showing that transient or stable knockdown of RTN4 by RNA interference phenocopies the impaired survival, proliferation, and anti-tumorigenic effects observed with DKM 3-30 in SW620 colorectal cancer cells (FIGS. 2C-2D). To further confirm that the cell viability impairments conferred by DKM 3-30 are due to RTN4, we tested the effect of this compound in mouse embryonic fibroblasts (MEF) with or without the expression of human RTN4. Mouse Rtn4 possesses a serine instead of cysteine at the analogous site to human RTN4(C1101). We show that DKM 3-30 does not show viability impairments in GFP-expressing MEF cells but induces apoptosis in MEF cells expressing human RTN4-GFP (FIG. 12).

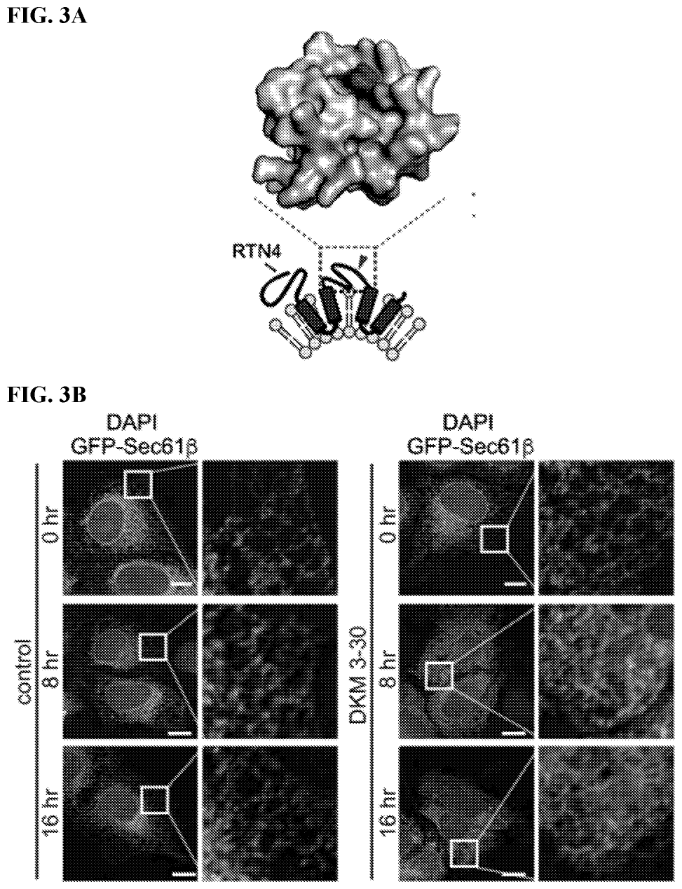

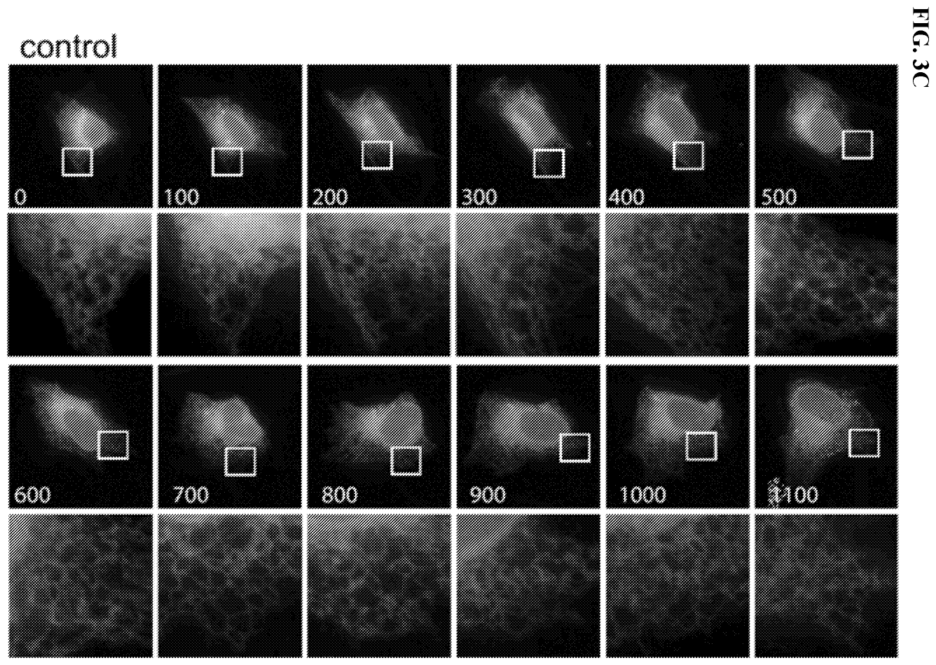

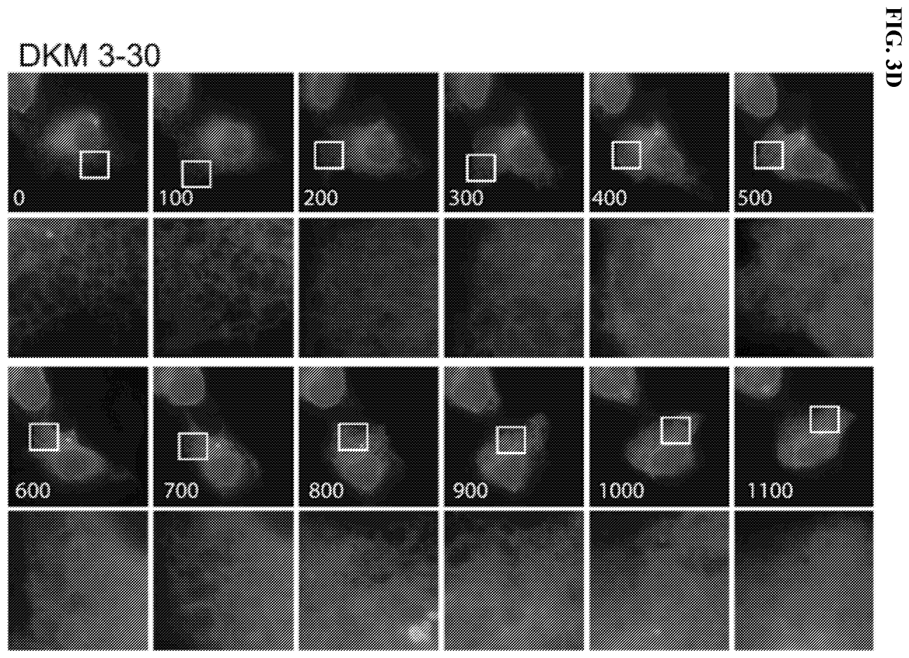

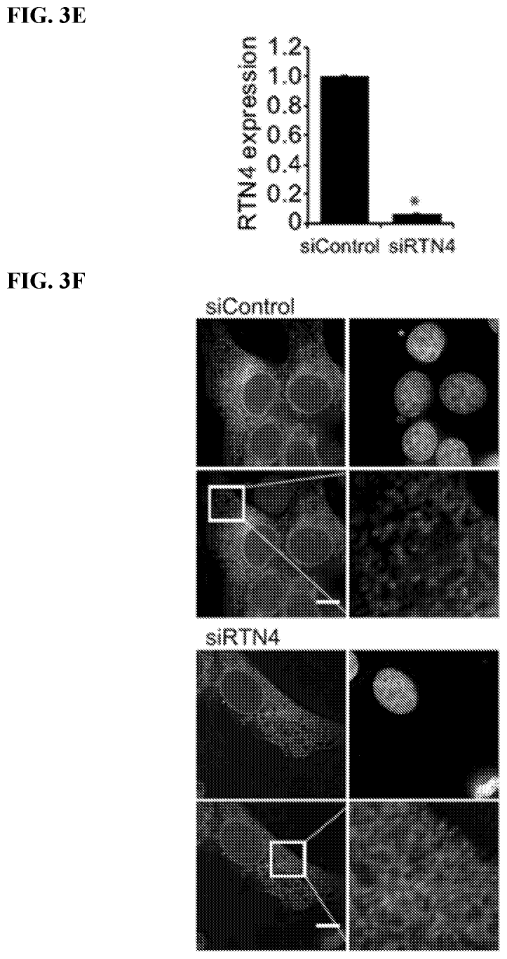

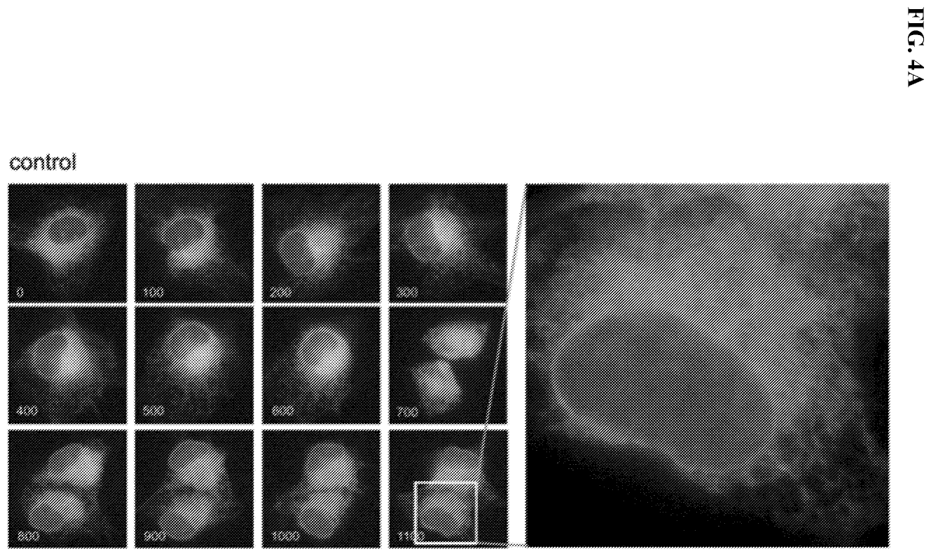

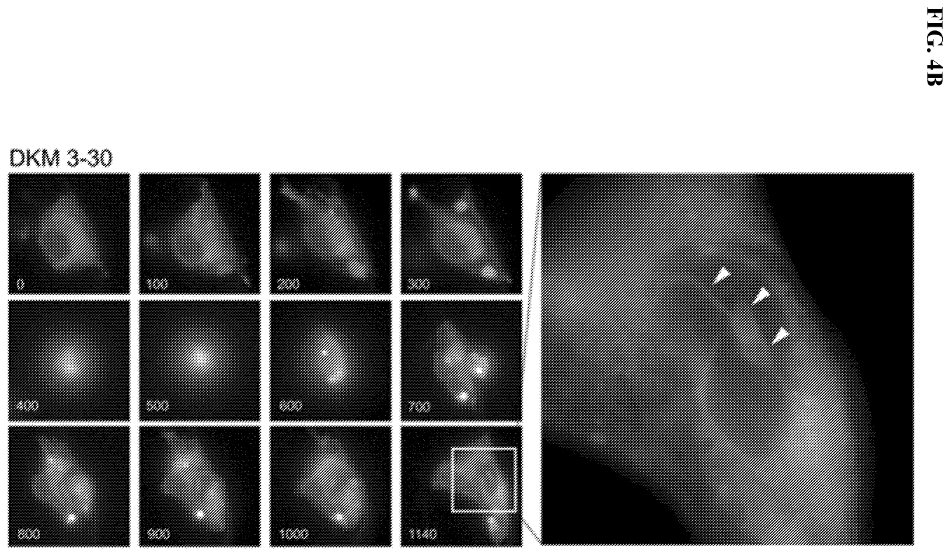

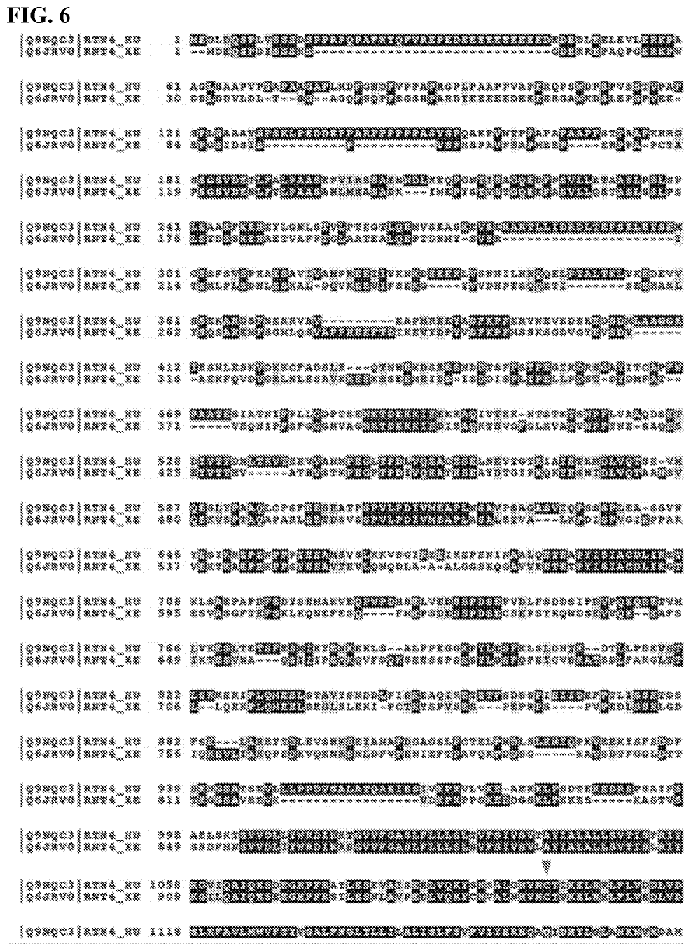

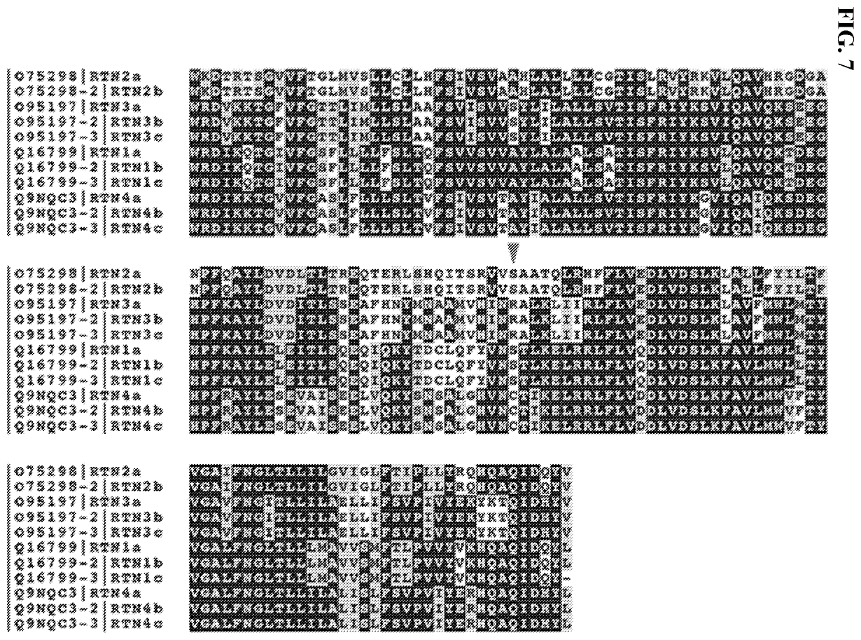

RTN4 is known to be a critical mediator of endoplasmic reticulum (ER) tubule formation¹¹⁻¹³. Interestingly, Voeltz et al. found that tubular ER network formation in a reconstituted in vitro system was disrupted by thiol modifying agents and discovered that Xenopus RTN4 was responsible for this action¹⁵. Intriguingly, one of these cysteines, C952 of Xenopus RTN4¹², corresponds to C1101 of human RTN4 identified in our study (FIG. 6). C1101 is present in all human RTN4 isoforms, but is absent in other reticulon family members (RTN1-3) (FIG. 7). This cysteine is positioned within a cytosolically exposed linker between two tandem hydrophobic regions (FIG. 3A), which allow RTN4 to adopt a characteristic wedge-shaped hairpin conformation required for generating highly curved membranes and tubular ER structures¹³. A solution NMR structure of a mouse RTN4 fragment revealed that this linker region forms a compact helical bundle with a portion associated with the membrane¹⁴ and a threaded homology model of the human RTN4 linker region indicates that C1101 is present in a cytosolically accessible helix (FIG. 3A).

We postulated that covalent modification of RTN4(C1101) by DKM 3-30 would impact the formation of ER tubular networks in cells. We attempted to analyze the effects of

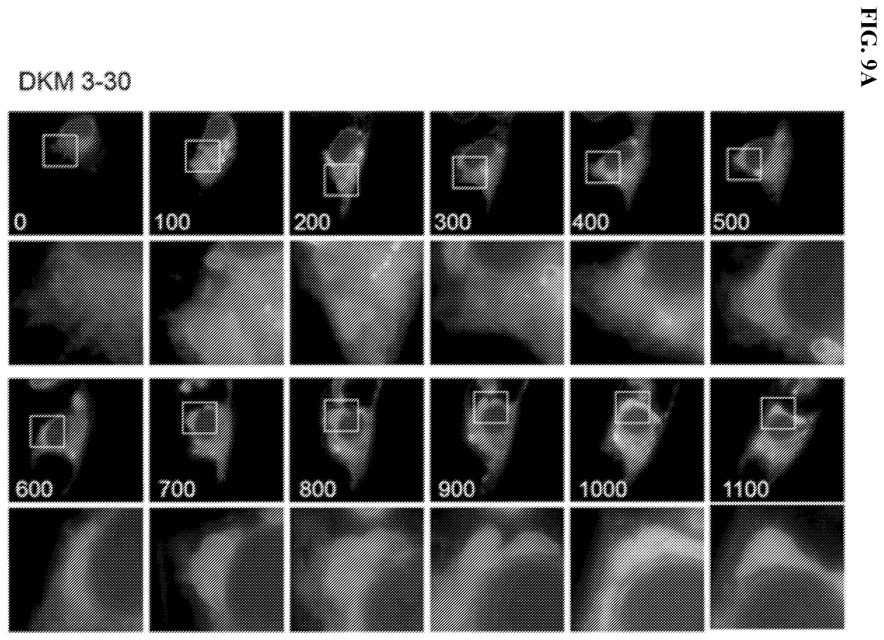

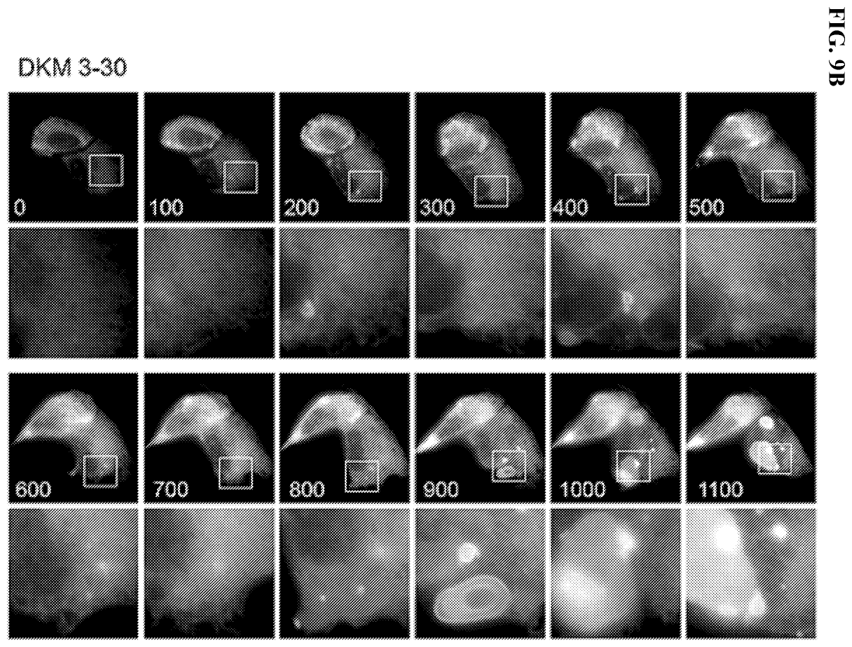

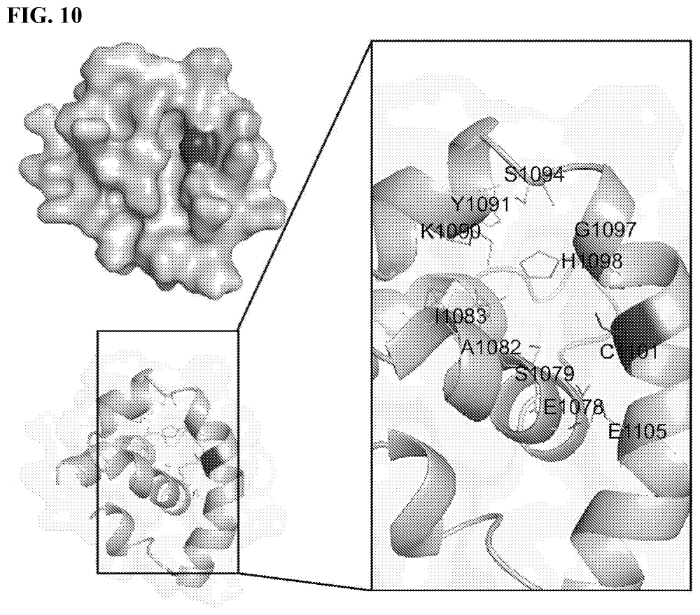

DKM 3-30 in SW620 colorectal cancer cells, and while the images suggest alterations in the ER morphology (FIG. 8), the reticular nature of the ER was difficult to visualize in this cell type. Therefore, we utilized U2OS osteosarcoma cells, which are a well-established cell line for the analysis of ER morphology. As expected, control U2OS cells expressing the ER marker GFP-Sec61β displayed a highly reticular ER with clearly visible tubular ER in the cell periphery (FIG. 3B). Treatment of U2OS cells with DKM 3-30 for 8 hr and 16 hr resulted in a striking loss of nearly all peripheral ER tubules and an increase in ER that exhibited sheet-like morphology (FIG. 3B). To more precisely define the temporal dynamics of DKM 3-30 on ER structure, we performed time-lapse imaging of GFP-Sec61β expressing cells (FIG. 3C). In contrast to vehicle-treated control cells (FIG. 3C), treatment with DKM 3-30 resulted in the loss of peripheral ER tubules and the accumulation of sheet-like ER structures (FIG. 3D). The alterations in the ER morphology were evident as early as 0.5-1 hr and the ER architecture became progressively more distorted, with some cells exhibiting extremely aberrant, circular ER structures (FIG. 9A-9B). Consistent with the importance of RTN4 in ER structure, siRNA-mediated depletion of RTN4 resulted in the appearance of similarly altered ER morphologies (FIGS. 3E,F). Together, these results suggest that DKM 3-30 acutely impairs RTN4 function in ER tubules formation or maintenance.

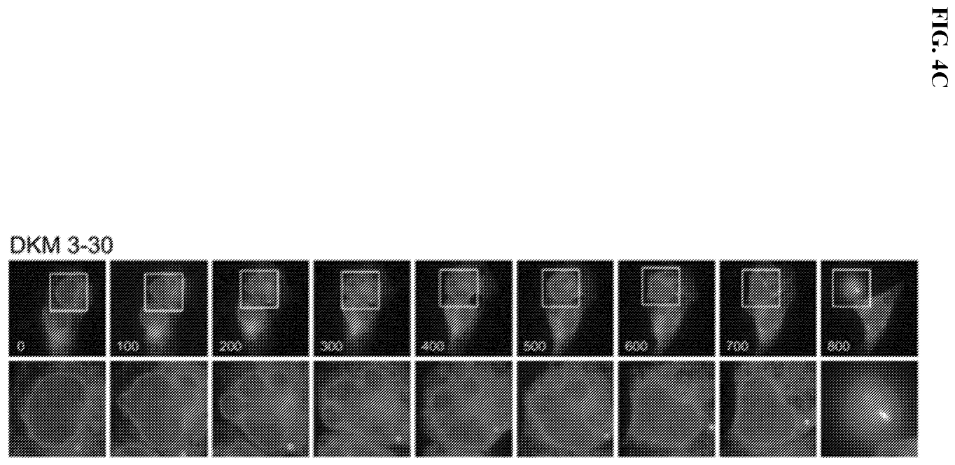

Cell division requires elaborate rearrangements in the ER and the nuclear envelope to ensure correct inheritance of DNA and segregation of DNA within a single nucleus¹⁵. During prophase the nuclear envelope retracts into the ER and then reforms during telophase. The reticulon family of proteins, and the transition between ER tubules and sheets, have been implicated in nuclear envelope assembly and disassembly during mitosis¹⁶⁻¹⁸ Time-lapse imaging of mitotic cells revealed that control cells divided rapidly (˜50-60 min) (FIG. 4A). In contrast, DKM 3-30-treated cells exhibited prolonged mitosis (˜3-4 hr) (FIG. 4B), possibly reflecting complications in the division process. Indeed, following mitosis, DKM 3-30-treated cells contained aberrant nuclei that were bisected by GFP-Sec61β positive structures (FIG. 4B). Distortions in the nuclear envelope were also frequently observed during interphase in DKM 3-30-treated cells, including multi-lobed, cloverleaf-like nuclear envelope morphologies that often preceded cell death (FIG. 4C). Thus, disrupting RTN4-mediated ER remodeling may impair colorectal cancer pathogenicity by altering ER homeostasis and nuclear envelope assembly and disassembly during mitosis.

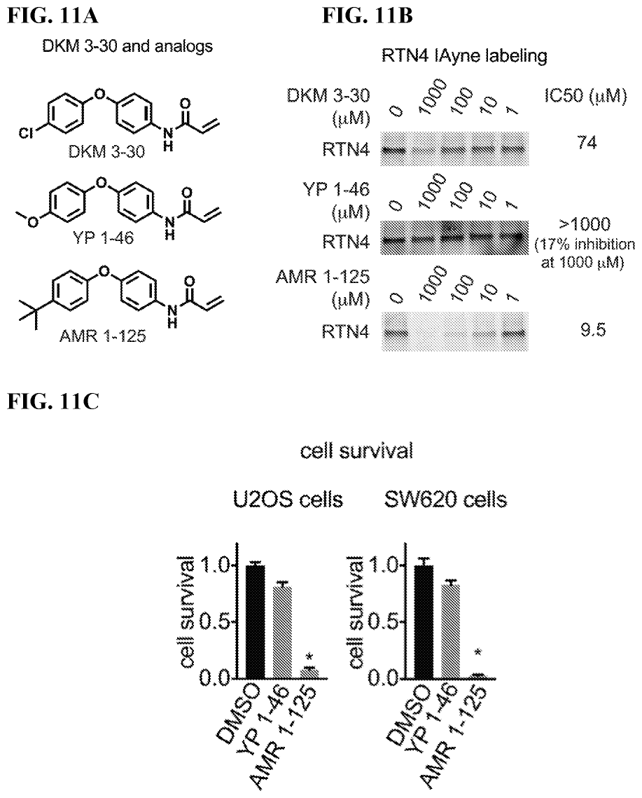

We also synthesized analogs of DKM 3-30 and showed that YP 1-46 demonstrated less displacement of IAyne labelling of RTN4, whereas AMR 1-125 exhibited ˜7-fold improved potency compared to DKM 3-30. We further showed that AMR 1-125, but not YP 1-46, impaired cell survival in U2OS and SW620 cells and ER morphology in U2OS cells (FIGS. 11A-11C).

In summary, we identify RTN4 as a novel colorectal cancer therapeutic target, and reveal a unique druggable hotspot within this classically undruggable protein, which can be targeted by cysteine-reactive ligands such as DKM 3-30 to impair ER and nuclear envelope morphology and colorectal cancer pathogenicity. We also show that DKM 3-30 impairs osteosarcoma cell survival as well, suggesting that RTN4 may have broader impacts upon other types of cancers. We recognize that DKM 3-30 may have additional off-targets that may contribute its anti-cancer activity, but nonetheless show compelling evidence that DKM 3-30 and its analogs phenocopy what is observed with RTN4 knockdown and that DKM 3-30 confers sensitivity in MEF cells only when expressing human RTN4. DKM 3-30 and AMR 1-125 may serve as initial starting points for subsequent medicinal chemistry to develop a more potent and selective RTN4 inhibitors for cancer therapy. Overall, we highlight the utility of coupling the screening of covalent ligand libraries with isoTOP-ABPP for mining the proteome for novel druggable nodes that can be targeted for cancer therapy.

Methods.

Materials. IAyne was obtained from CHESS Gmbh. Heavy and light TEV-biotin tags were synthesized per previously described methods 5,21

Synthesis of Cysteine Fragment Library. The synthesis of the cysteine-reactive ligand library is described below. All compounds in the library were confirmed to be >95% pure.

Cell Culture. SW620 cells were purchased from ATCC. SW620 cells were grown in L-15 media with 10% fetal bovine serum (FBS) in ambient CO₂. U2OS cells were grown in DMEM media supplemented with 10% FBS at 37° C. with 5% CO₂.

Survival and Proliferation Assays. Cells were plated the evening before the experiment, and allowed to adhere overnight. For serum-free cell survival assays, cells were plated in media not containing FBS. For cell proliferation assays, cells were plated in regular media. For the chemical genetics screen, cells were treated with either DMSO or the cysteine-reactive fragment for 48 h and cell viability was assessed by Hoescht stain using our previously described methods

Tumor Xenograft Growth Studies. C.B17 SCID male mice (6-8 weeks old) were injected subcutaneously into the flank with 2,000,000 cells in serum-free media. For pharmacological treatments, mice were exposed by intraperitoneal (ip) injection with either vehicle (18:1:1 PBS/ethanol/PEG40) or 50 mg/kg DKM 3-30 once per day starting ten days after the initiation of the xenograft experiment and until the completion of the study. Tumors were measured every 7 days by caliper measurements. Animal experiments were conducted in accordance with the guidelines of the Institutional Animal Care and Use Committee of the University of California, Berkeley.

IsoTOP-ABPP Analysis. IsoTOP-ABPP analyses were performed as previously described⁵⁻⁷. For competitive IsoTOP-ABPP, SW620 cell lysates were pre-incubated with DMSO vehicle or DKM 3-30 (50 μM) for 30 min at 37° C. in phosphate-buffered saline (PBS), and then labeled with IAyne (100 μM) for 1 h at room temperature. They were subsequently treated with isotopically light (control) or heavy (treated) TEV-biotin (100 μM) and CuAAC was performed as previously described^{5,6}. For analysis of cysteine reactivity in primary colorectal tumor tissue, tumors were pooled and incubated with either 100 μM IAyne and isotopically heavy TEV-biotin or 10 μM IAyne and isotopically light TEV-biotin followed by CuAAC. Proteins were precipitated over one hour and pelleted by centrifugation at 6500×g. Proteins were washed 3 times with cold methanol then denatured and resolubilized by heating in 1.2% SDS/PBS to 85° C. for 5 min. Insoluble components were precipitated by centrifugation at 6500×g and soluble proteome was diluted in 5 ml PBS, for a final concentration of 0.2% SDS. Labeled proteins were bound to avidin-agarose beads (170 μL resuspended beads/sample, Thermo Pierce) while rotating overnight at 4° C. Bead-linked proteins were enriched by washing three times each in PBS and water, then resuspended in 6 M urea/PBS (Sigma-Aldrich) and reduced in dithiothreitol (1 mM, Sigma-Aldrich), alkylated with iodoacetamide (18 mM, Sigma-Aldrich), then washed and resuspended in 2 M urea/PBS with 1 mM calcium chloride and trypsinized overnight with 0.5 μg/μ1 sequencing grade trypsin (Promega). Tryptic peptides were discarded and beads were washed three times each in PBS and water, then washed with one wash of TEV buffer containing 1 μM DTT. TEV-biotin tag was digested overnight in TEV buffer containing 1 μM DTT and 5 μL Ac-TEV protease at 29° C. Peptides were diluted in water and acidified with final concentration of 5% formic acid (1.2 M, Spectrum).

Peptides from all proteomic experiments were pressure-loaded onto a 250 mm inner diameter fused silica capillary tubing packed with 4 cm of Aqua C18 reverse-phase resin (Phenomenex #04A-4299) which was previously equilibrated on an Agilent 600 series HPLC using gradient from 100% buffer A to 100% buffer B over 10 min, followed by a 5 min wash with 100% buffer B and a 5 min wash with 100% buffer A. The samples were then attached using a MicroTee PEEK 360 μm fitting (Thermo Fisher Scientific # p-888) to a 13 cm laser pulled column packed with 10 cm Aqua C18 reverse-phase resin and 3 cm of strong-cation exchange resin for isoTOP-ABPP studies. Samples were analyzed using an Q Exactive Plus mass spectrometer (Thermo Fisher Scientific) using a 5-step Multidimensional Protein Identification Technology (MudPIT) program, using 0%, 25%, 50%, 80%, and 100% salt bumps of 500 mM aqueous ammonium acetate and using a gradient of 5-55% buffer B in buffer A (buffer A: 95:5 water:acetonitrile, 0.1% formic acid; buffer B 80:20 acetonitrile:water, 0.1% formic acid). Data was collected in data-dependent acquisition mode with dynamic exclusion enabled (60 s). One full MS (MS1) scan (400-1800 m/z) was followed by 15 MS2 scans (ITMS) of the nth most abundant ions. Heated capillary temperature was set to 200° C. and the nanospray voltage was set to 2.75 kV.

Data were extracted in the form of MS1 and MS2 files using Raw Extractor 1.9.9.2 (Scripps Research Institute) and searched against the Uniprot mouse database using ProLuCID search methodology in IP2 v.3 (Integrated Proteomics Applications, Inc)²³. Cysteine residues were searched with a static modification for carboxyaminomethylation (+57.02146) and up to two differential modifications for methionine oxidation and either the light or heavy TEV tags (+464.28596 or +470.29977, respectively). Peptides were required to have at least one tryptic end and to contain the TEV modification. ProLUCID data was filtered through DTASelect to achieve a peptide false-positive rate below 1%.

Gel-Based ABPP. Gel-based ABPP methods were performed as previously described²⁴. Recombinant RTN4 (0.06 μg) protein (RTN4-Fisher Scientific) were pre-treated with DMSO or DKM 3-30, respectively, for 1 h at 37° C. in an incubation volume of 50 μL PBS, and were subsequently treated with IAyne (10 μM final concentration) for 30 min at 37° C. CuAAC was performed to append rhodamine-azide onto IAyne probe-labeled proteins. The samples were separated by SDS/PAGE and scanned using a ChemiDoc MP (Bio-Rad Laboratories, Inc). Inhibition of target labeling was assessed by densitometry using ImageStudio Light software.

RTN4 Knockdown. Targets were knocked down transiently with siRNA or stably with shRNA as previously described^{22,25}. For siRNA studies, SW620 cells (200,000 cells/well) were seeded overnight after which siControl (non-targeting siRNA) or siRTN4 oligonucleotides (5 pooled siRNAs targeting each target purchased from Dharmacon) were transfected into cells using Dharmafect 1. Cells were harvested after 48 h for qPCR and for seeding for cell viability assays.

For shRNA studies, shControl (targeting GFP) or shRTN4 constructs (purchased from Sigma) were transfected into HEK293T cells alongside lentiviral vectors using FuGENE. Lentivirus was collected from filtered cultured medium 48 h post-transfection and used to infect the target cancer cell line with Polybrene (0.01 mg/ml) Target cells were selected over 3 days with 1 mg/ml puromycin. The short hairpin sequences for the generation of RTN4 knockdown lines were: CCGGGCAGTGTTGATGTGGGTATTTCTCGAGAAATACCCACATCAACACTGCTTTTT TG (SEQ ID NO:328) and CCGGGCTATATCTGAGGAGTTGGTTCTCGAGAACCAACTCCTCAGATATAGCTTTTT TG (SEQ ID NO:329). The control shRNA was targeted against GFP with the target sequence GCAAGCTGACCCTGAAGTTCAT (SEQ ID NO:330). Knockdown was confirmed by qPCR.

qPCR. qPCR was performed using the manufacturer's protocol for Fisher Maxima SYBR Green with 10 mM primer concentrations or for Bio-Rad SsoAdvanced Universal Probes Supermix. Primer sequences for Fisher Maxima SYBR Green were derived from Harvard Primer Bank. Primer sequences for Bio-Rad SsoAdvanced Universal Probes Supermix were designed with Primer 3 Plus.

Fluorescence microscopy. SW620 and U2OS cells were transiently transfected with a plasmid encoding GFP-tagged Sec61β using fuGENE6 (Roche) according to the manufacturer's instructions. Transfected cells plated on poly-L-lysine treated coverslips were treated, washed in PBS, and fixed by incubation in 4% paraformaldehyde in PBS for 10 min. Fixed cells were washed extensively in PBS and nuclei stained by addition of 4′,6-diamidino-2-phenylindole (DAPI) (Thermo Fisher Scientific) for 10 min. Coverslips were mounted using Fluoromount-G (SouthernBiotech) and visualized using a DeltaVision Elite microscope outfitted with a 60× oil immersion objective. Acquired stacks of images of fixed cells were deconvolved and analyzed using SoftWoRx and ImageJ. For time-lapse imaging of live cells, transfected cells were plated on poly-L-lysine treated glass-bottom 4-well imaging chambers (Lab-Tek II; Thermo Fisher Scientific). Imaging was performed using a DeltaVision Elite microscope encased in a chamber that was maintained at 37° C. and was continuously perfused with humidified 5% CO2. Acquired images were analyzed using SoftWoRx and ImageJ.

Homology modeling and multiple sequence alignments. The threaded homology model of the human Rtn4 (amino acids 1054-1120 of Rtn4a) on the NMR solution structure of the corresponding region of mouse Rtn4 (PDB 2KO2) was generated using Protein Homology/analogY Recognition Engine V 2.0 (Phyre2). Figures were made using PyMOL. Multiple sequence alignments were generated using Clustal Omega and figures were made using BoxShade.

Primary Human Colorectal Tumors. Eligible patients completed written consent for our tissue banking protocol that is approved by the University of Alabama at Birmingham Institutional Review Board. During the colorectal tumor resection, a 1 cm³ portion of the tumor was dissected free of the fresh resection specimen, divided into 4-5 aliquots, placed into 1.5 mL cryovials, flash frozen, and stored at −80° C. Adjacent non-tumor bearing colorectal tissue was also collected and banked in a similar manner.

General Synthetic Methods

Chemicals and reagents were purchased from major commercial suppliers and used without further purification. Reactions were performed under a nitrogen atmosphere unless otherwise noted. Silica gel flash column chromatography was performed using EMD or Sigma Aldrich silica gel 60 (230-400 mesh). Proton and carbon nuclear magnetic resonance (¹H NMR and ¹³C NMR) data was acquired on a Bruker AVB 400, AVQ 400, or AV 600 spectrometer at the University of California, Berkeley. High resolution mass spectrum were obtained from the QB3 mass spectrometry facility at the University of California, Berkeley using positive or negative electrospray ionization (+ESI or −ESI). Yields are reported as a single run.

General Procedure A. The amine (1 eq.) was dissolved in DCM (5 mL/mmol) and cooled to 0° C. To the solution was added acryloyl chloride (1.2 eq.) followed by triethylamine (1.2 eq.). The solution was warmed to room temperature and stirred overnight. The solution was then washed with brine and the crude product was purified by silica gel chromatography (and recrystallization if necessary) to afford the corresponding acrylamide.

General Procedure B. The amine (1 eq.) was dissolved in DCM (5 mL/mmol) and cooled to 0° C. To the solution was added chloroacetyl chloride (1.2 eq.) followed by triethylamine (1.2 eq.). The solution was warmed to room temperature and stirred overnight. The solution was then washed with brine and the crude product was purified by silica gel chromatography (and recrystallization if necessary) to afford the corresponding chloroacetamide.

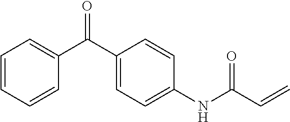 [SMILES: C=CC(=O)Nc1ccc(C(=O)c2ccccc2)cc1]

N-(4-benzoylphenyl)acrylamide (DKM 2-117). Following General Procedure A starting from 4-aminobenzophenone (587 mg, 3.0 mmol), product was obtained after silica gel chromatography (10% to 30% ethyl acetate in hexanes) in 37% yield as a yellow solid (275 mg). ¹H NMR (400 MHz, CDCl₃): δ 8.77 (s, 1H), 7.80-7.73 (m, 6H), 7.57 (tt, J=1.5, 7.4 Hz, 1H), 7.46 (t, J=7.6 Hz, 2H), 6.46 (dd, J=1.6 16.9 Hz, 1H), 6.37 (dd, J=9.9, 16.9 Hz, 1H), 5.75 (dd, J=1.6, 9.9 Hz, 1H). ¹³C NMR (100 MHz, CDCl₃): δ 196.3, 164.4, 142.3, 137.8, 133.0, 132.5, 131.7, 131.0, 130.0, 128.8, 128.4, 119.3. HRMS (+ESI): Calculated: 252.1019 (C₁₆H₁₄NO₂). Observed: 252.1014.

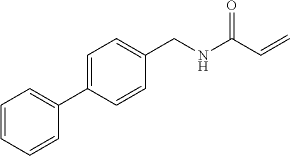 [SMILES: C=CC(=O)NCc1ccc(-c2ccccc2)cc1]

N-([1,1′-biphenyl]-4-ylmethyl)acrylamide (DKM 2-37). Following General Procedure A starting from 4-phenylbenzylamine (552 mg, 3.0 mmol), product was obtained after silica gel chromatography (0% to 80% ethyl acetate in hexanes) in 10% yield as an off-white solid (73 mg). ¹H NMR (400 MHz, CDCl₃): δ 7.58-7.55 (m, 4H), 7.44 (t, J=7.5 Hz, 2H), 7.38-7.33 (m, 3H), 6.35 (dd, J=1.3, 17.0 Hz, 1H), 6.13 (dd, J=10.3, 17.0 Hz, 1H), 6.01 (s, 1H), 5.68 (dd, J=1.3, 10.3 Hz, 1H), 4.56 (d, J=5.8 Hz, 2H). ¹³C NMR (100 MHz, CDCl₃): δ 165.5, 140.77, 140.73, 137.2, 130.7, 128.9, 128.5, 127.6, 127.5, 127.2, 127.1, 43.5. HRMS (+ESI): Calculated: 238.1226 (C₁₆H₁₆NO). Observed: 238.1224.

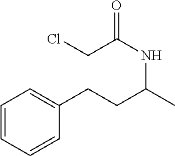

2-Chloro-N-(4-phenylbutan-2-yl)acetamide (DKM 2-76). Following General Procedure B starting from 1-methyl-3-phenylpropylamine (614 mg, 4.1 mmol) product was obtained after silica gel chromatography (0% to 30% ethyl acetate in hexanes) in 81% yield as a white solid (662 mg). ¹H NMR (400 MHz, CDCl₃): δ 7.34-7.31 (m, 2H), 7.24-7.21 (m, 3H), 6.55 (d, J=7.4 Hz, 1H), 4.15-4.07 (m, 1H), 4.04 (s, 2H), 2.70 (t, J=8.2 Hz, 2H), 1.89-1.83 (m, 2H), 1.26 (d, J=6.4 Hz, 3H). ¹³C NMR (100 MHz, CDCl₃): δ 165.1, 141.3, 128.4, 128.2, 125.9, 45.7, 42.7, 381, 32.3, 20.7. HRMS (+ESI): Calculated: 226.0993 (C₁₂H₁₇ClNO). Observed: 226.0992.

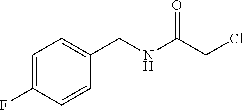 [SMILES: O=C(CCl)NCc1ccc(F)cc1]

2-chloro-N-(4-fluorobenzyl)acetamide (DKM 2-80). Following General Procedure B starting from 4-fluorobenzylamine (369 mg, 2.9 mmol) product was obtained after silica gel chromatography (0% to 30% ethyl acetate in hexanes) in 77% yield as a white solid (452 mg). ¹H NMR (400 MHz, CDCl₃): δ 7.28-7.24 (m, 2H), 7.05-7.01 (m, 2H), 6.97 (s, 1H), 4.45 (d, J=5.6 Hz, 2H), 4.09 (s, 2H). ¹³C NMR (100 MHz, CDCl₃): δ 166.1, 163.6, 161.2, 133.20, 133.17, 129.64, 129.56, 115.9, 115.7, 43.2, 42.7. HRMS (−ESI): Calculated: 200.0284 (C₉H₈NOClF). Observed: 200.0284.

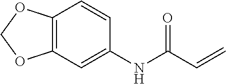

N-(benzo[d][1,3]dioxol-5-yl)acrylamide (DKM 2-86). Following General Procedure A starting from 3,4-(methylenedioxy)aniline (486 mg, 2.9 mmol), product was obtained after silica gel chromatography (0% to 30% ethyl acetate in hexanes) in 68% yield as a white solid (438 mg). ¹H NMR (400 MHz, (CD₃)₂SO): δ 10.05 (s, 1H), 7.39 (d, J=2.0 Hz, 1H), 7.02 (dd, J=2.0, 8.4 Hz, 1H), 6.87 (d, J=8.4 Hz, 1H), 6.38 (dd, J=10.1, 17.0 Hz, 1H), 6.22 (dd, J=2.1, 17.0 Hz, 1H), 5.99 (s, 2H), 5.72 (dd, J=2.1, 10.1 Hz, 1H). ¹³C NMR (100 MHz, (CD₃)₂SO): δ 162.8, 147.0, 143.1, 133.4, 131.8, 126.5, 112.1, 108.1, 101.4, 101.0. HRMS (+ESI): Calculated: 192.0655 (C₁₀H₁₀NO₃). Observed: 192.0651.

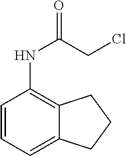 [SMILES: O=C(CCl)Nc1cccc2c1CCC2]

2-chloro-N-(2,3-dihydro-1H-inden-4-yl)acetamide (DKM 2-91). Following General Procedure B starting from 4-aminoindan (372 mg, 2.8 mmol) product was obtained after silica gel chromatography (0% to 40% ethyl acetate in hexanes) in 49% yield as an off-white solid (289 mg). ¹H NMR (400 MHz, CDCl₃): δ 8.19 (s, 1H), 7.74 (d, J=8.4 Hz, 1H), 7.15 (t, J=7.8 Hz, 1H), 7.05 (d, J=7.6 Hz, 1H), 4.16 (s, 2H), 2.94 (t, J=7.6 Hz, 2H), 2.82 (t, J=7.4 Hz, 2H), 2.10 (quint, J=7.5 Hz, 2H). ¹³C NMR (100 MHz, CDCl₃): δ 163.8, 145.5, 134.5, 132.8, 127.3, 121.6, 118.5, 43.1, 33.2, 29.8, 24.8. HRMS (+ESI): Calculated: 210.0680 (C₁₁H₁₃ClNO). Observed: 210.0680.

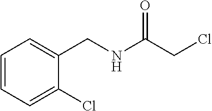 [SMILES: O=C(CCl)NCc1ccccc1Cl]

2-Chloro-N-(2-chlorobenzyl)acetamide (DKM 2-94). Following General Procedure B starting from 2-chlorobenzylamine (432 mg, 3.1 mmol) product was obtained after silica gel chromatography (0% to 30% ethyl acetate in hexanes) in 67% yield as a white solid (443 mg). ¹H NMR (400 MHz, CDCl₃): δ 7.36-7.18 (m, 5H), 4.51 (d, J=6.4 Hz, 2H), 4.01 (s, 2H). ¹³C NMR (100 MHz, CDCl₃): δ 166.1, 134.7, 133.5 129.8, 129.5, 129.1, 127.1, 42.5, 41.6. HRMS (−ESI): Calculated: 215.9988 (C₉H₈NOCl₂). Observed: 215.9988.

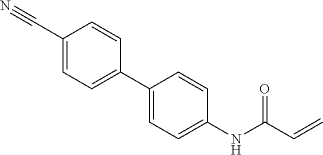 [SMILES: C=CC(=O)Nc1ccc(-c2ccc(C#N)cc2)cc1]

N-(4′-cyano-[1,1′-biphenyl]-4-yl)acrylamide (DKM 2-98). Following General Procedure A starting from 4-(4-aminophenyl)benzonitrile (387 mg, 2.0 mmol), product was obtained after silica gel chromatography (1% to 2% ethyl methanol in DCM) in 70% yield as a yellow solid (348 mg). ¹H NMR (600 MHz, (D₃C)₂CO): 9.52 (s, 1H), 7.90-7.89 (m, 2H), 7.87-7.86 (m, 2H), 7.84-7.82 (m, 2H), 7.73-7.71 (m, 2H), 6.49 (dd, J=10.0, 16.9 Hz, 1H), 6.39 (dd, J=2.0, 16.9 Hz, 1H), 5.76 (dd, J=2.0, 10.0 Hz, 1H). ¹³C NMR (150 MHz, (D₃C)₂CO): δ 164.3, 145.7, 140.9, 134.8, 133.6, 132.7, 128.5, 128.2, 127.6, 120.8, 119.5, 111.3. HRMS (−ESI): Calculated: 247.0877 (C₁₆H₁₁N₂O). Observed: 247.0875.

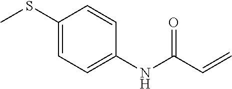

N-(4-(methylthio)phenyl)acrylamide (DKM 3-10). Following General Procedure A starting from 4-(methylthio)aniline (405 mg, 2.9 mmol), product was obtained after silica gel chromatography (10% to 40% ethyl acetate in hexanes) in 64% yield as a clear oil (362 mg). ¹H NMR (400 MHz, MeOD): δ 7.59-7.56 (m, 2H), 7.26-7.22 (m, 2H), 6.42 (dd, J=9.6, 17.0 Hz, 1H), 6.34 (dd, J=2.3, 17.0 Hz, 1H), 5.75 (dd, J=2.3, 9.6 Hz, 1H), 2.45 (s, 3H). ¹³C NMR (100 MHz, MeOD): δ 166.0, 137.2, 135.4, 132.4, 128.6, 127.7, 121.9, 16.4. HRMS (+ESI): Calculated: 194.0634 (C₁₀H₁₂NOS). Observed: 194.0631.

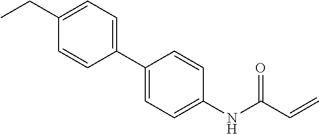 [SMILES: C=CC(=O)Nc1ccc(-c2ccc(CC)cc2)cc1]

N-(4′-ethyl-[1,1′-biphenyl]-4-yl)acrylamide (DKM 3-16). Following General Procedure A starting from 4-amino-4-ethylbiphenyl (386 mg, 2.0 mmol), product was obtained after silica gel chromatography (10% to 70% ethyl acetate in hexanes) in 65% yield as a white solid (164 mg). 41 NMR (400 MHz, (CD₃)₂CO): δ 7.82 (d, J=8.2 Hz, 2H), 7.62-7.59 (m, 2H), 7.58-7.54 (m, 2H), 7.29 (d, J=8.2 Hz, 2H), 6.47 (dd, J=9.9, 16.9 Hz, 1H), 6.36 (dd, J=2.2, 16.9 Hz, 1H), 5.72 (dd, J=2.2, 9.9 Hz, 1H), 2.67 (q, J=7.6 Hz, 2H), 1.24 (t, J=7.6 Hz, 3H). ¹³C NMR (100 MHz, (CD₃)₂CO): δ 164.1, 144.0, 139.5, 13.9, 137.1, 132.9, 129.3, 127.9, 127.4, 127.2, 120.7, 29.2, 16.2. HRMS (+ESI): Calculated: 252.1383 (C₁₇H₁₈NO). Observed: 252.1379.

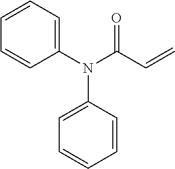 [SMILES: C=CC(=O)N(c1ccccc1)c1ccccc1]

N,N-diphenylacrylamide (DKM 3-70). A solution of diphenylamine (347 mg, 2.1 mmol) in DCM (10 mL) was cooled to 0° C. To the solution was added acryloyl chloride (222 mg, 2.5 mmol) followed by triethylamine (279 mg, 2.8 mmol). The solution was allowed to warm to room temperature and stirred overnight. The solution was washed with brine and citric acid and the crude product was purified via silica gel chromatography (20% to 60% ethyl acetate in hexanes) to afford the product in 24% yield as a dark yellow oil (112 mg). ¹H NMR (400 MHz, CDCl₃): δ 7.43-7.28 (m, 10H), 6.52 (dd, J=2.0, 16.8 Hz, 1H), 6.25 (dd, J=10.2, 16.8 Hz, 1H), 5.67 (dd, J=1.8, 10.2 Hz, 1H). ¹³C NMR (100 MHz, CDCl₃): δ 165.8, 142.6, 129.7, 129.3, 128.5, 127.0. HRMS (+ESI): Calculated: 246.0889 (C₁₅H₁₃NONa). Observed: 246.0887.

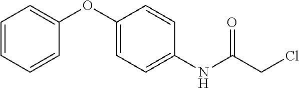 [SMILES: O=C(CCl)Nc1ccc(Oc2ccccc2)cc1]

2-Chloro-N-(4-phenoxyphenyl)acetamide (TRH 1-23). Following General Procedure B starting from 4-phenoxyaniline (370 mg, 2.0 mmol) product was obtained after silica gel chromatography (10% to 30% ethyl acetate in hexanes) in 46% yield as a white solid (315 mg). ¹H NMR (400 MHz, CDCl₃): δ 8.42 (s, 1H), 7.52-7.48 (m, 2H), 7.35-7.31 (m, 2H), 7.10 (t, J=7.3 Hz, 1H), 7.01-6.98 (m, 4H), 4.17 (s, 2H). ¹³C NMR (100 MHz, CDCl₃): δ 164.2, 157.2, 154.4, 132.1, 129.8, 123.4, 122.2, 119.4, 118.7, 42.9. HRMS (−ESI): Calculated: 260.0484 (C₁₄H₁₁NO₂Cl). Observed: 260.0482.

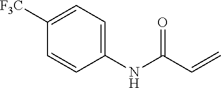

N-(4-(trifluoromethyl)phenyl)acrylamide (TRH 1-50). Following General Procedure A starting from 4-(trifluoromethyl)aniline (328 mg, 2.0 mmol), product was obtained after silica gel chromatography (10% to 30% ethyl acetate in hexanes) in 55% yield as a white solid (239 mg). ¹H NMR (400 MHz, MeOD): δ 7.78 (d, J=8.3 Hz, 2H), 7.55 (d, J=8.6 Hz, 2H), 6.44-6.32 (m, 2H), 5.75 (dd, J=8.4, 2.8 Hz, 1H). ¹³C NMR (100 MHz, MeOD): δ 166.3, 143.3, 132.1, 128.6, 127.04, 127.00, 126.97, 126.93, 126.6, 124.3, 120.9. HRMS (−ESI): Calculated: 214.0485 (C₁₀H₇NOF₃). Observed: 214.0484.

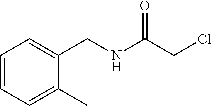

2-Chloro-N-(2-methylbenzyl)acetamide (TRH 1-55). Following General Procedure B starting from 2-methylbenzylamine (239 mg, 2.0 mmol) product was obtained after silica gel chromatography (30% ethyl acetate in hexanes) and recrystallization from 5% ethyl acetate in hexanes in 64% yield as a white solid (191 mg). ¹H NMR (400 MHz, CDCl₃): δ 7.25-7.19 (m, 4H), 6.85 (s, 1H), 4.46 (d, J=5.6 Hz, 2H), 4.04 (s, 2H), 2.33 (s, 3H). ¹³C NMR (100 MHz, CDCl₃): δ 165.8, 136.4, 135.0, 130.6, 128.4, 128.0, 126.3, 42.6, 42.0, 19.0. HRMS (−ESI): Calculated: 196.0535 (C₁₀H₁₁NOCl). Observed: 196.0534.

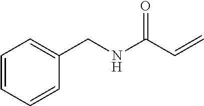

N-benzylacrylamide (DKM 2-31). Following General Procedure A starting from benzylamine (334 mg, 3.1 mmol), product was obtained after silica gel chromatography (0% to 50% ethyl acetate in hexanes) in 75% yield as a white solid (376 mg). ¹H NMR (400 MHz, CDCl₃): δ 7.28-7.18 (m, 6H), 6.19-6.16 (m, 2H), 5.53 (dd, J=4.6, 7.3 Hz, 1H), 4.36 (d, J=5.9 Hz, 2H). ¹³C NMR (100 MHz, CDCl₃): δ 165.8, 138.1, 130.8, 128.6, 127.7, 127.3, 126.5, 43.5. HRMS (+ESI): Calculated: 162.0913 (C₁₀H₁₂NO). Observed: 162.0912.

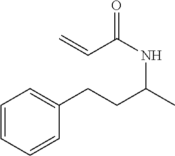

N-(4-phenylbutan-2-yl)acrylamide (DKM 2-32). Following General Procedure A starting from 1-methyl-3-phenylpropylamine (606 mg, 4.0 mmol), product was obtained after silica gel chromatography (0% to 50% ethyl acetate in hexanes) in 89% yield as a clear oil (735 mg). ¹H NMR (400 MHz, CDCl₃): δ 7.32-7.29 (m, 2H), 7.23-7.20 (m, 3H), 6.84 (d, J=8.4 Hz, 1H), 6.36-6.24 (m, 2H), 5.64 (dd, J=2.8, 9.2 Hz, 1H), 4.21-4.14 (m, 1H), 2.70 (t, J=7.8 Hz, 2H), 1.93-1.77 (m, 2H), 1.24 (d, J=6.4 Hz, 3H). ¹³C NMR (100 MHz, CDCl₃): δ 165.1, 141.7, 131.3, 128.3, 128.2, 125.80, 125.77, 45.1, 38.4, 32.5, 20.8. HRMS (+ESI): Calculated: 204.1383 (C₁₃H₁₈NO). Observed: 204.1380.

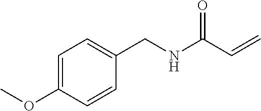

N-(4-methoxybenzyl)acrylamide (DKM 2-33). Following General Procedure A starting from 4-methoxybenzylamine (424 mg, 3.1 mmol), product was obtained after silica gel chromatography (0% to 50% ethyl acetate in hexanes) in 60% yield as a clear oil (343 mg). ¹H NMR (400 MHz, CDCl₃): δ 7.14 (d, J=8.8 Hz, 2H), 6.85 (s, 1H), 6.79 (d, J=8.4 Hz, 2H), 6.24-6.14 (m, 2H), 5.56 (dd, J=2.0, 9.6 Hz, 1H), 4.33 (d, J=5.6 Hz, 2H), 3.73 (s, 3H). ¹³C NMR (100 MHz, CDCl₃): δ 165.6, 158.9, 130.9, 130.3, 129.1, 126.4, 113.9, 55.2, 42.9. HRMS (+ESI): Calculated: 192.1019 (C₁₁H₁₄NO₂). Observed: 192.1017.

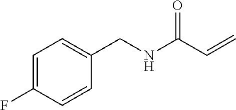

N-(4-fluorobenzyl)acrylamide (DKM 2-34). Following General Procedure A starting from 4-fluorobenzylamine (368 mg, 2.9 mmol), product was obtained after silica gel chromatography (0% to 60% ethyl acetate in hexanes) in 52% yield as an off-white solid (276 mg). ¹H NMR (400 MHz, CDCl₃): δ 7.24-7.19 (m, 2H), 6.97 (t, J=8.5 Hz, 2H), 6.42 (s, 1H), 6.27 (d, J=17.0 Hz, 1H), 6.12 (dd, J=17.0, 10.2 Hz, 1H), 5.63 (d, J=10.2 Hz, 1H), 4.42 (d, J=5.8 Hz, 2H). ¹³C NMR (100 MHz, CDCl₃): δ 165.7, 163.5, 134.0, 130.6, 129.6, 129.5, 127.0, 115.7, 115.5, 43.0. HRMS (+ESI): Calculated: 180.0819 (C₁₀H₁₁NOF). Observed: 180.0818.

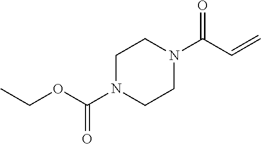

Ethyl 4-acryloylpiperazine-1-carboxylate (DKM 2-39). Following General Procedure A starting from ethyl 1-piperazinecarboxylate (477 mg, 3.0 mmol), product was obtained after silica gel chromatography (0% to 70% ethyl acetate in hexanes) in 58% yield as a yellow oil (372 mg). ¹H NMR (400 MHz, CDCl₃): δ 6.46 (dd, J=10.5, 16.8 Hz, 1H), 6.18 (dd, J=1.9, 16.8 Hz), 5.60 (dd, J=1.9, 10.5 Hz), 4.03 (q, J=7.1 Hz, 2H), 3.54 (s, 2H), 3.44 (s, 2H), 3.39-3.36 (m, 4H), 1.15 (t, J=7.1 Hz, 3H). ¹³C NMR (100 MHz, CDCl₃): δ 165.3, 155.1, 128.2, 127.1, 61.5, 45.4, 43.6, 43.3, 41.5, 14.5. HRMS (+ESI): Calculated: 213.1234 (C₁₀H₁₇N₂O₃). Observed: 213.1232.

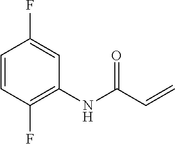

N-(2,5-difluorophenyl)acrylamide (DKM 2-40). Following General Procedure A starting from 2,5-difluoroaniline (369 mg, 2.9 mmol), product was obtained after silica gel chromatography (0% to 15% ethyl acetate in hexanes) in 27% yield as a white solid (141 mg). ¹H NMR (400 MHz, (CD₃)₂CO): δ 9.26 (s, 1H), 8.29-8.24 (m, 1H), 7.24-7.18 (m, 1H), 6.90-6.84 (m, 1H), 6.67 (dd, J=10.2, 16.9 Hz, 1H), 6.41 (dd, J=1.9, 16.9 Hz, 1H), 5.79 (dd, J=1.9, 10.2 Hz, 1H). ¹³C NMR (100 MHz, (CD₃)₂CO): δ 164.6, 160.4, 151.0, 148.7, 132.0, 128.9, 128.8, 128.5, 116.7, 116.6, 116.5, 116.4, 111.1, 111.0, 110.8, 110.7, 110.0, 109.7. HRMS (+ESI): Calculated: 184.0568 (C₉H₈F₂NO). Observed: 184.0567.

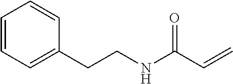

N-phenethylacrylamide (DKM 2-42). Following General Procedure A starting from phenylethylamine (367 mg, 3.0 mmol), product was obtained after silica gel chromatography (0% to 50% ethyl acetate in hexanes) in 85% yield as a yellow oil (450 mg). ¹H NMR (400 MHz, CDCl₃): δ 7.30-7.18 (m, 5H), 6.63 (s, 1H), 6.25 (dd, J=1.8, 17.0 Hz, 1H), 6.13 (dd, J=10.0, 17.0 Hz 1H), 5.59 (dd, J=1.6, 10.0 Hz, 1H), 3.56 (q, J=6.8 Hz, 2H), 2.85 (t, J=7.3 Hz, 2H). ¹³C NMR (100 MHz, CDCl₃): δ 165.8, 138.8, 131.0, 128.7, 128.6, 126.4, 126.1, 40.8, 35.6. HRMS (+ESI): Calculated: 176.1070 (C₁₁H₁₄NO). Observed: 176.1068.

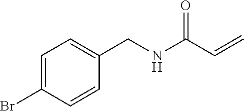

N-(4-bromobenzyl)acrylamide (DKM 2-43). Following General Procedure A starting from 4-bromobenzylamine (535 mg, 2.9 mmol), product was obtained after silica gel chromatography (0% to 50% ethyl acetate in hexanes) in 59% yield as a white solid (407 mg). ¹H NMR (400 MHz, CDCl₃): δ 7.37 (d, J=8.4 Hz, 2H), 7.07 (d, J=8.4 Hz, 2H), 7.00 (s, 1H), 6.24-6.10 (m, 2H), 5.59 (dd, J=2.0, 9.7 Hz, 1H), 4.32 (d, J=6.0 Hz, 2H). ¹³C NMR (100 MHz, CDCl₃): δ 165.9, 137.2, 131.7, 130.6, 129.4, 126.9, 121.2, 42.8. HRMS (+ESI): Calculated: 240.0019 (C₁₀H₁₁BrNO). Observed: 240.0016.

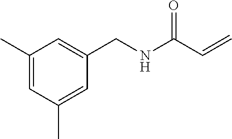

N-(3,5-dimethylbenzyl)acrylamide (DKM 2-47). Following General Procedure A starting from 3,5-dimethylbenzylamine (257 mg, 1.9 mmol), product was obtained after silica gel chromatography (0% to 40% ethyl acetate in hexanes) in 77% yield as a white solid (276 mg). ¹H NMR (400 MHz, CDCl₃): δ 6.89-6.87 (m, 4H), 6.26 (dd, J=2.1, 17.0 Hz, 1H), 6.18 (dd, J=9.7, 17.0 Hz, 1H) 5.59 (dd, J=2.1, 9.7 Hz, 1H), 4.35 (d, J=6.0 Hz, 2H), 2.28 (s, 6H). ¹³C NMR (100 MHz, CDCl₃): δ 165.6, 138.1, 138.0, 130.9, 129.0, 126.3, 125.6, 43.4, 12.2. HRMS (+ESI): Calculated: 190.1226 (C₁₂H₁₆NO). Observed: 190.1225.

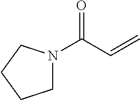

1-(pyrrolidin-1-yl)prop-2-en-1-one (DKM 2-48). Following General Procedure A starting from pyrrolidine (223 mg, 3.1 mmol), product was obtained after silica gel chromatography (0% to 80% ethyl acetate in hexanes) in 38% yield as a pale yellow oil (148 mg). ¹H NMR (400 MHz, CDCl₃): δ 6.40 (dd, J=10.0, 16.8 Hz, 1H), 6.29 (dd, J=2.4, 16.8 Hz, 1H), 5.60 (dd, J=2.4, 10.0 Hz, 1H), 3.48 (t, J=6.8 Hz, 4H), 1.91 (quint, J=6.7 Hz, 2H), 1.82 (quint, J=6.7 Hz, 2H). ¹³C NMR (100 MHz, CDCl₃): δ 164.4, 128.8, 127.2, 46.6, 45.9, 26.1, 24.3. HRMS (+ESI): Calculated: 126.0913 (C₇H₁₂NO). Observed: 126.0912.

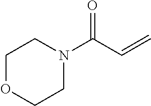

1-morpholinoprop-2-en-1-one (DKM 2-49). Following General Procedure A starting from morpholine (273 mg, 3.1 mmol), product was obtained after silica gel chromatography (0% to 80% ethyl acetate in hexanes) in 46% yield as a yellow oil (205 mg). ¹H NMR (400 MHz, CDCl₃): δ 6.45 (dd, J=10.5, 16.8 Hz, 1H), 6.20 (dd, J=1.9, 16.8 Hz, 1H), 5.61 (dd, J=1.9, 10.5 Hz, 1H), 5.38 (s, 6H), 3.46 (s, 2H). ¹³C NMR (100 MHz, CDCl₃): δ 165.3, 128.1, 126.9, 66.6, 46.0, 42.1. HRMS (+ESI): Calculated: 142.0863 (C₇H₁₂NO₂). Observed: 142.0861.

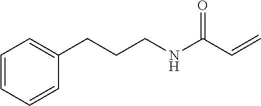

N-(3-phenylpropyl)acrylamide (DKM 2-50). Following General Procedure A starting from 3-phenyl-1-propylamine (275 mg, 2.0 mmol), product was obtained after silica gel chromatography (0% to 60% ethyl acetate in hexanes) in 58% yield as a yellow oil (223 mg). ¹H NMR (400 MHz, CDCl₃): δ 7.29-7.25 (m, 2H), 7.20-7.16 (m, 3H), 6.99 (s, 1H), 6.29-6.17 (m, 2H), 5.59 (dd, J=2.6, 9.0 Hz, 1H), 3.34 (q, J=6.7 Hz, 2H), 2.65 (t, J=7.6 Hz, 2H), 1.87 (quint, J=7.4 Hz, 2H). ¹³C NMR (100 MHz, CDCl₃): δ 166.0, 141.4, 131.1, 128.33, 128.26, 125.9, 39.2, 33.2, 31.0. HRMS (+ESI): Calculated: 190.1226 (C₁₂H₁₆NO). Observed: 190.1225.

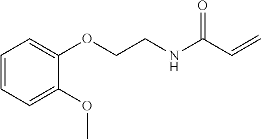 [SMILES: C=CC(=O)NCCOc1ccccc1OC]

N-(2-(2-methoxyphenoxy)ethyl)acrylamide (DKM 2-58). Following General Procedure A starting from 2-(2-methoxyphenoxy)ethanamine (509 mg, 3.0 mmol), product was obtained after silica gel chromatography (0% to 30% ethyl acetate in hexanes) in 70% yield as a yellow oil (470 mg). ¹H NMR (400 MHz, CDCl₃): δ 6.95-6.84 (m, 4H), 6.77 (s, 1H), 6.26 (d, J=17.1 Hz, 1H), 6.11 (dd, J=10.2, 17.1 Hz, 1H), 5.59 (d, J=10.2 Hz, 1H), 4.07 (t, J=5.2 Hz, 2H), 3.79 (s, 3H), 3.69 (q, J=5.4 Hz, 2H). ¹³C NMR (100 MHz, CDCl₃): δ 165.7, 149.6, 147.7, 130.8, 126.4, 122.1, 121.0, 114.8, 111.8, 68.5, 55.7, 38.9. HRMS (+ESI): Calculated: 244.0944 (C₁₂H₁₅NO₃Na). Observed: 244.0940.

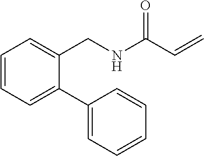 [SMILES: C=CC(=O)NCc1ccccc1-c1ccccc1]

N-([1,1′-biphenyl]-2-ylmethyl)acrylamide (DKM 2-59). Following General Procedure A starting from 2-phenylbenzylamine (202 mg, 1.1 mmol), product was obtained after silica gel chromatography (0% to 40% ethyl acetate in hexanes) in 70% yield as a yellow oil (184 mg). ¹H NMR (400 MHz, CDCl₃): δ 7.41-7.22 (m, 9H), 6.16 (dd, J=1.2, 17.2 Hz, 1H), 6.03-5.97 (m, 2H), 5.55 (dd, J=1.2, 10.0 Hz, 1H), 4.44 (d, J=5.6 Hz, 2H). ¹³C NMR (100 MHz, CDCl₃): δ 165.3, 141.6, 140.6, 135.2, 1306, 130.2, 129.0, 128.7, 128.4, 127.8, 127.4, 127.3, 126.4, 41.4. HRMS (+ESI): Calculated: 238.1226 (C₁₆H₁₆NO). Observed: 238.1223.

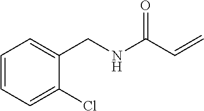

N-(2-chlorobenzyl)acrylamide (DKM 2-60). Following General Procedure A starting from 2-chlorobenzylamine (406 mg, 2.9 mmol), product was obtained after silica gel chromatography (0% to 30% ethyl acetate in hexanes) in 34% yield as a white solid (162 mg). ¹H NMR (400 MHz, CDCl₃): δ 7.34-30 (m, 2H), 7.20-7.16 (m, 2H), 6.84 (s, 1H), 6.25 (dd, J=2.0, 17.0 Hz, 1H), 6.16 (dd, J=9.7, 17.0 Hz, 2H), 5.60 (dd, J=2.0, 9.7 Hz, 1H), 4.52 (d, J=6.1 Hz, 2H). ¹³C NMR (100 MHz, CDCl₃): δ 165.9, 135.5, 133.5, 130.6, 129.8, 129.5, 128.8, 127.1, 126.8, 41.4. HRMS (+ESI): Calculated: 196.0524 (C₁₀H₁₁ClNO). Observed: 196.0521

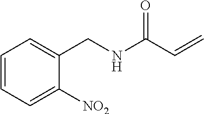

N-(2-nitrobenzyl)acrylamide (DKM 2-62). Following General Procedure A starting from 2-nitrobenzylamine hydrochloride (406 mg, 2.9 mmol) with an extra equivalent of triethylamine, product was obtained after silica gel chromatography (50% ethyl acetate in hexanes) in 42% yield as a yellow solid (255 mg). ¹H NMR (400 MHz, CDCl₃): δ 7.98 (dd, J=1.1, 8.2 Hz, 1H), 7.58-7.52 (m, 2H), 7.41-7.37 (m, 1H), 7.03 (s, 1H), 6.22 (dd, J=2.0, 17.0 Hz, 1H), 6.14 (dd, J=9.7, 17.0 Hz, 1H), 5.59 (dd, J=2.0, 9.7 Hz, 1H), 4.68 (d, J=6.4 Hz, 2H). ¹³C NMR (100 MHz, CDCl₃): δ 165.8, 148.2, 134.1, 133.6, 131.9, 130.4, 128.7, 127.1, 125.1, 41.2. HRMS (+ESI): Calculated: 207.0764 (C₁₀H₁₁N₂O₃). Observed: 207.0760.

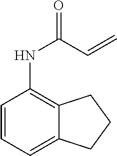

N-(2,3-dihydro-1H-inden-4-yl)acrylamide (DKM 2-84). Following General Procedure A starting from 4-aminoindan (402 mg, 3.0 mmol), product was obtained after silica gel chromatography (30% ethyl acetate in hexanes) in 59% yield as a white solid (332 mg). ¹H NMR (400 MHz, CDCl₃): δ 7.72 (d, J=7.5 Hz, 1H), 7.54 (s, 1H), 7.10 (t, J=7.7 Hz, 1H), 7.01 (d, J=7.2 Hz, 1H), 6.40-6.26 (m, 2H), 5.69 (dd, J=1.9, 9.7 Hz, 1H), 2.91 (t, J=7.4 Hz, 2H), 2.78 (t, J=7.4 Hz, 2H), 2.05 (quint, J=7.4 Hz, 2H). ¹³C NMR (100 MHz, CDCl₃): 163.5, 145.3, 134.4, 133.6, 131.2, 127.5, 127.2, 12.0, 19.2, 33.2, 30.1, 24.8. HRMS (+ESI): Calculated: 188.1070 (C₁₂H₁₄NO). Observed: 188.1069.

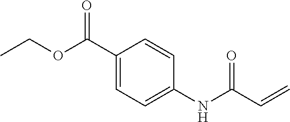 [SMILES: C=CC(=O)Nc1ccc(C(=O)OCC)cc1]

Ethyl 4-acrylamidobenzoate (DKM 2-85). Following General Procedure A starting from benzocaine (486 mg, 2.9 mmol), product was obtained after silica gel chromatography (0% to 30% ethyl acetate in hexanes) in 68% yield as a white solid (438 mg). ¹H NMR (400 MHz, CDCl₃): δ 9.39 (s, 1H), 7.95 (d, J=8.7 Hz, 2H), 7.74 (d, J=8.7 Hz, 2H), 6.43-6.41 (m, 2H), 5.71 (dd, J=4.7, 6.9 Hz, 2H), 4.31 (q, J=7.1 Hz, 2H), 1.33 (s, J=7.1 Hz, 3H). ¹³C NMR (100 MHz, CDCl₃): δ 166.5, 164.6, 142.5, 131.0, 130.6, 128.4, 125.7, 119.4, 61.0, 14.2. HRMS (−ESI): Calculated: 218.0823 (C₁₂H₁₂NO₃). Observed: 218.0822.

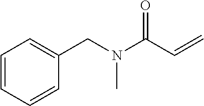

N-benzyl-N-methylacrylamide (DKM 2-95). Following General Procedure A starting from N-methylbenzylamine (350 mg, 2.9 mmol), product was obtained after silica gel chromatography (20% ethyl acetate in hexanes) in 60% yield as a clear oil (304 mg). ¹H NMR (˜48:52 rotamer ratio, asterisks denote minor peaks, 400 MHz, CDCl₃): δ 7.34-7.23 (m, 4H), 7.16 (s, 1H), 7.14* (s, 1H), 6.61 (dd, J=10.4, 16.8 Hz, 1H), 6.57* (dd, J=10.4, 16.8 Hz, 1H), 6.38 (dd, J=1.9, 16.8 Hz, 1H), 6.36* (dd, J=1.9, 16.8 Hz, 1H), 5.71 (dd, J=1.9, 10.4 Hz, 1H), 5.64* (dd, J=1.9, 10.4 Hz), 4.63 (s, 2H), 4.56* (s, 2H), 2.98* (s, 3H), 2.96 (s, 3H). ¹³C NMR (100 MHz, CDCl₃): δ 167.0, 166.4, 137.1, 136.5, 128.8, 128.5, 128.2, 128.0, 17.62, 127.59, 127.3, 126.3, 53.3, 51.0, 34.8, 34.0. HRMS (+ESI): Calculated: 176.1070 (C₁₁H₁₄NO). Observed: 176.1070.

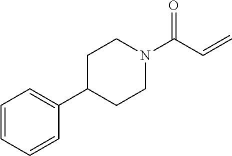 [SMILES: C=CC(=O)N1CCC(c2ccccc2)CC1]

1-(4-phenylpiperidin-1-yl)prop-2-en-1-one (DKM 2-97). Following General Procedure A starting from 4-phenylpiperidine (331 mg, 2.1 mmol), product was obtained after silica gel chromatography (0% to 50% ethyl acetate in hexanes) in 86% yield as a yellow oil (379 mg). ¹H NMR (400 MHz, CDCl₃): δ 7.32-7.28 (m, 2H), 7.22-7.17 (m, 3H), 6.62 (dd, J=10.6, 16.8 Hz, 1H), 6.30 (dd, J=1.9, 16.8 Hz, 1H), 5.68 (dd, J=1.9, 10.6, Hz, 1H), 4.82 (d, J=12.9 Hz, 1H), 4.11 (d, J=13.2 Hz, 1H), 3.15 (t, J=8.5 Hz, 1H), 2.78-2.67 (m, 2H), 1.90 (d, J=12.9 Hz, 2H), 1.64 (quint, J=12.3 Hz, 2H). ¹³C NMR (100 MHz, CDCl₃): 165.3, 145.0, 128.5, 127.8, 127.4, 126.6, 126.4, 46.4, 42.7, 33.9, 32.7. HRMS (+ESI): Calculated: 216.1383 (C₁₄H₁₈NO). Observed: 216.1383.

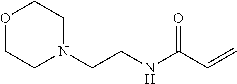

N-(2-morpholinoethyl)acrylamide (DKM 2-100). Following General Procedure A starting from 2-morpholinoethylamine (580 mg, 3.0 mmol), product was obtained after silica gel chromatography (2% to 6% methanol in dichloromethane) in 33% yield as a white solid (184 mg). ¹H NMR (400 MHz, CDCl₃): δ 6.39 (s, 1H), 6.21 (dd, J=1.7, 17.0 Hz, 1H), 6.08 (dd, J=10.1, 17.0 Hz, 1H), 5.56 (dd, J=1.7, 10.1 Hz, 1H), 3.63 (t, J=4.6 Hz, 4H), 3.36 (q, J=6.2 Hz, 2H), 2.45 (t, J=6.2 Hz, 2H), 2.40-2.38 (m, 4H). ¹³C NMR (100 MHz, CDCl₃): δ 165.5, 130.9, 126.2, 66.9, 57.0, 53.3, 35.7. HRMS (+ESI): Calculated: 185.1285 (C₉H₁₇N₂O₂). Observed: 185.1280.

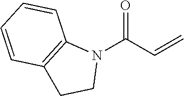

1-(indolin-1-yl)prop-2-en-1-one (DKM 2-101). Following General Procedure A starting from indoline (580 mg, 3.0 mmol), product was obtained after silica gel chromatography (0% to 20% ethyl acetate in hexanes) in 56% yield as a green solid (285 mg). ¹H NMR (400 MHz, CDCl₃): δ 8.30 (d, J=7.7 Hz, 1H), 7.22-7.17 (m, 2H), 7.03 (t, J=7.9 Hz, 1H), 6.60-6.48 (m, 2H), 5.79 (dd, J=2.6, 9.5 Hz, 1H), 4.15 (t, J=8.5 Hz, 2H), 3.20 (t, J=8.1, 2H). ¹³C NMR (100 MHz, CDCl₃): δ 163.6, 142.6, 131.5, 129.0, 128.6, 127.2, 124.4, 123.8, 117.2, 47.8, 27.7. HRMS (+ESI): Calculated: 174.0913 (C₁₁H₁₂NO). Observed: 174.0911.

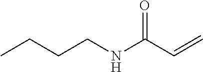

N-butylacrylamide (DKM 2-102). Following General Procedure A starting from butylamine (223 mg, 3.0 mmol), product was obtained after silica gel chromatography (20% ethyl acetate in hexanes) in 61% yield as a clear oil (237 mg). ¹H NMR (400 MHz, (CDCl₃): δ 6.81 (s, 1H), 6.21-6.10 (m, 2H), 5.52 (dd, J=3.6, 8.3 Hz, 1H), 3.26-3.21 (m, 2H), 1.48-1.41 (m, 2H), 1.33-1.23 (m, 2H), 0.84 (t, J=7.3 Hz, 3H). ¹³C NMR (100 MHz, CDCl₃): δ 166.0, 131.2, 125.6, 39.3, 31.5, 20.1, 13.7. HRMS (+ESI): Calculated: 128.1070 (C₇H₁₄NO). Observed: 128.1068.

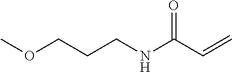

N-(3-methoxypropyl)acrylamide (DKM 2-103). Following General Procedure A starting from 3-methoxypropylamine (274 mg, 3.1 mmol), product was obtained after silica gel chromatography (35% to 60% ethyl acetate in hexanes) in 54% yield as a clear oil (236 mg). ¹H NMR (400 MHz, CDCl₃): δ 6.84 (s, 1H), 6.15 (dd, J=2.0, 17.0 Hz. 1H), 6.07 (dd, J=9.8, 17.0 Hz, 1H), 5.51 (dd, J=2.0, 9.8 Hz, 1H), 3.39 (t, J=5.9 Hz, 2H), 3.33 (q, J=6.3 Hz, 2H), 3.25 (s, 3H), 1.72 (quint, J=6.3 Hz, 2H). ¹³C NMR (100 MHz, CDCl₃): δ 165.8, 131.2, 125.7, 71.3, 58.7, 37.7, 29.0. HRMS (+ESI): Calculated: 144.1019 (C₇H₁₄NO₂). Observed: 144.1017.

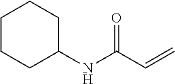

N-cyclohexylacrylamide (DKM 2-106). Following General Procedure A starting from cyclohexylamine (292 mg, 2.9 mmol), product was obtained after silica gel chromatography (20% to 30% ethyl acetate in hexanes) in 86% yield as a white solid (313 mg). ¹H NMR (400 MHz, (CDCl₃): δ 6.55 (d, J=6.7 Hz, 1H), 6.21-6.09 (m, 2H), 5.51 (dd, J=2.5, 9.1 Hz, 1H), 3.79-3.70 (m, 1H), 1.86-1.82 (m, 2H), 1.67-1.63 (m, 2H), 1.56-1.52 (m, 1H), 1.28-1.21 (m, 2H), 1.16-1.05 (m, 3H). ¹³C NMR (100 MHz, CDCl₃): δ 164.8, 131.5, 125.7, 48.3, 32.9, 25.5, 24.9. HRMS (+ESI): Calculated: 154.1226 (C₉H₁₆NO). Observed: 154.1224.

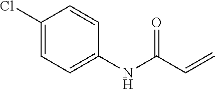

N-(4-chlorophenyl)acrylamide (DKM 2-107). Following General Procedure A starting from 4-chloroaniline (386 mg, 3.0 mmol), product was obtained after silica gel chromatography (0% to 40% ethyl acetate in hexanes) followed by recrystallization from toluene in 31% yield as a white solid (168 mg). ¹H NMR (400 MHz, (CD₃)₂CO): δ 9.47 (s, 1H), 7.77-7.74 (m, 2H), 7.35-7.31 (m, 2H), 6.43 (dd, J=9.6, 16.9 Hz, 1H), 6.35 (dd, J=2.5, 16.9 Hz, 1H), 5.73 (dd, J=2.5, 9.6 Hz, 1H). ¹³C NMR (100 MHz, (CD₃)₂CO): δ 164.1, 139.0, 132.5, 129.5, 128, 127.5, 121.7. HRMS (−ESI): Calculated: 180.0222 (C₉H₇NOCl). Observed: 180.0221.

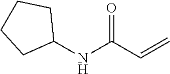

N-cyclopentylacrylamide (DKM 2-108). Following General Procedure A starting from cyclopentylamine (257 mg, 3.0 mmol), product was obtained after silica gel chromatography (20% to 30% ethyl acetate in hexanes) in 55% yield as a colorless oil (229 mg). ¹H NMR (400 MHz, (CDCl₃): δ 6.70 (s, 1H), 6.21-6.10 (m, 2H), 5.51 (dd, J=3.5, 8.5 Hz, 1H), 5.53-5.50 (sex, J=7.1 Hz, 1H), 1.94-1.86 (m, 2H), 1.65-1.46 (m, 4H), 1.41-1.32 (m, 2H). ¹³C NMR (100 MHz, CDCl₃): δ 165.4, 131.3, 125.7, 51.1, 32.9, 23.8. HRMS (+ESI): Calculated: 140.1070 (C₈₁H₁₄NO). Observed: 140.1067.

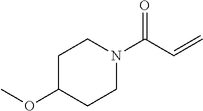

1-(4-methoxypiperidin-1-yl)prop-2-en-1-one (DKM 2-109). Following General Procedure A starting from 4-methoxypiperidine (461 mg, 3.0 mmol), product was obtained after silica gel chromatography (40% to 60% ethyl acetate in hexanes) in 75% yield as a pale yellow oil (386 mg). ¹H NMR (400 MHz, (CDCl₃): δ 6.45 (dd, J=10.6, 16.8 Hz, 1H), 6.09 (dd, J=2.0, 16.8 Hz, 1H), 5.51 (dd, J=2.0, 10.6 Hz, 1H), 3.80-3.74 (m, 1H), 3.65-3.58 (m, 1H), 3.33-3.17 (m, 6H), 1.74-1.67 (m, 2H), 1.47-1.39 (m, 2H). ¹³C NMR (100 MHz, CDCl₃): δ 165.1, 127.6, 127.2, 75.0, 55.5, 42.7, 38.9, 31.1, 29.9. HRMS (+ESI): Calculated: 170.1176 (C₉H₁₆NO₂). Observed: 170.1176.

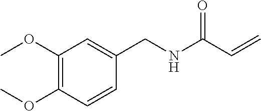 [SMILES: C=CC(=O)NCc1ccc(OC)c(OC)c1]

N-(3,4-dimethoxybenzyl)acrylamide (DKM 2-110). Following General Procedure A starting from 3,4-dimethoxybenzylamine (497 mg, 3.0 mmol), product was obtained after silica gel chromatography (30% to 40% ethyl acetate in hexanes) in 65% yield as a white solid (425 mg). ¹H NMR (400 MHz, CDCl₃): δ 7.07 (s, 1H), 6.70-6.64 (m, 3H), 6.18-6.08 (m, 2H), 5.50 (dd, J=3.1, 8.8 Hz, 1H), 4.26 (d, J=5.8 Hz, 2H), 3.70 (d, J=7.8 Hz, 6H). ¹³C NMR (400 MHz, CDCl₃): δ 165.5, 148.7, 148.0, 130.73, 130.67, 126.2, 119.9, 110.98, 110.96, 55.64, 55.55, 43.12. HRMS (+ESI): Calculated: 222.1125 (C₁₂H₁₆NO₃). Observed: 222.1121.

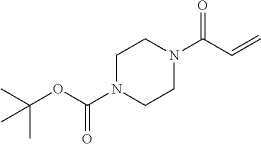 [SMILES: C=CC(=O)N1CCN(C(=O)OC(C)(C)C)CC1]

tert-butyl 4-acryloylpiperazine-1-carboxylate (DKM 2-111). Following General Procedure A starting from 1-boc-piperazine (552 mg, 3.0 mmol), product was obtained after silica gel chromatography (50% to 70% ethyl acetate in hexanes) in 75% yield as a pale yellow oil (534 mg). ¹H NMR (400 MHz, CDCl₃): δ 6.48 (dd, J=10.5, 16.8 Hz, 1H), 6.20 (dd, J=1.8, 16.8 Hz, 1H), 5.60 (dd, J=1.8, 10.5 Hz, 1H), 3.55 (s, 2H), 3.44 (s, 2H), 3.36-3.34 (m, 4H), 1.37 (s, 9H). ¹³C NMR (100 MHz, CDCl₃): δ 165.4, 154.4, 128.2, 127.2, 80.2, 45.5, 41.7, 28.3. HRMS (+ESI): Calculated: 241.1547 (C₁₂H₂₁N₂O₃). Observed: 241.1543.

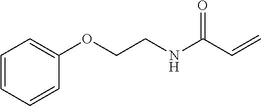

N-(2-phenoxyethyl)acrylamide (DKM 2-113). Following General Procedure A starting from 2-phenoxyethylamine (279 mg, 2.0 mmol), product was obtained after silica gel chromatography (30% to 70% ethyl acetate in hexanes) in 61% yield as a white solid (239 mg). ¹H NMR (400 MHz, CDCl₃): δ 7.31-7.25 (m, 2H), 6.98-6.94 (m, 1H), 6.90-6.87 (m, 2H), 6.58 (s, 1H), 6.31 (dd, J=1.6, 17.0 Hz, 1H), 6.17 (dd, J=10.2, 17.0 Hz, 1H), 5.64 (dd, J=1.6, 10.2 Hz, 1H), 4.05 (t, J=5.2 Hz, 2H), 3.73 (q, J=5.4 Hz, 2H). ¹³C NMR (100 MHz, CDCl₃): δ 165.9, 158.4, 130.7, 129.6, 126.7, 121.2, 114.4, 66.5, 39.1. HRMS (+ESI): Calculated: 192.1019 (C₁₁H₁₄NO₂). Observed: 192.1016.

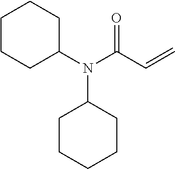 [SMILES: C=CC(=O)N(C1CCCCC1)C1CCCCC1]

N,N-dicyclohexylacrylamide (DKM 2-114). Following General Procedure A starting from dicyclohexylamine (537 mg, 3.0 mmol), product was obtained after silica gel chromatography (20% to 40% ethyl acetate in hexanes) in 55% yield as a white solid (382 mg). ¹H NMR (400 MHz, CDCl₃): δ 6.49 (dd, J=10.6, 16.8 Hz, 1H), 6.11 (dd, J=1.9, 16.8 Hz, 1H), 5.49 (dd, J=2.0, 10.6 Hz, 1H), 3.45 (s, 1H), 3.22 (s, 1H), 2.22 (s, 2H), 1.74-1.49 (m, 12H), 1.22-1.07 (m, 6H). ¹³C NMR (100 MHz, CDCl₃): δ 166.2, 130.9, 125.5, 57.5, 55.6, 31.6, 30.1, 26.4, 26.0, 25.3. HRMS (+ESI): Calculated: 236.2009 (C₁₅H₂₆NO). Observed: 236.2004.

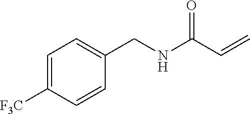 [SMILES: C=CC(=O)NCc1ccc(C(F)(F)F)cc1]

N-(4-(trifluoromethyl)benzyl)acrylamide (DKM 2-116). Following General Procedure A starting from 4-(trifluoromethyl)benzylamine (516 mg, 2.9 mmol), product was obtained after silica gel chromatography (20% to 30% ethyl acetate in hexanes) in 24% yield as a white solid (165 mg). ¹H NMR (600 MHz, CDCl₃): δ 7.53 (d, J=8.0 Hz, 2H), 7.35 (d, J=8.0 Hz, 2H), 6.58 (s, 1H), 6.28 (dd, J=1.5, 17.0 Hz, 1H), 6.14 (dd, J=10.1, 17.0 Hz, 1H), 5.64 (dd, J=1.5, 10.1 Hz, 1H), 4.50 (d, J=6.0 Hz, 2H). ¹³C NMR (150 MHz, CDCl₃): δ 165.9, 142.3, 130.5, 130.0, 129.7, 128.0, 127.3, 125.73, 125.69, 12566, 125.62, 43.1. HRMS (−ESI): Calculated: 228.0642 (C₁₁H₉NOF₃). Observed: 228.0641.

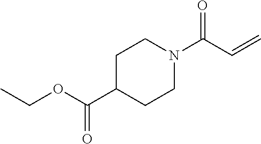

Ethyl 1-acryloylpiperidine-4-carboxylate (DKM 2-120). Following General Procedure A starting from ethyl isonipecotate (459 mg, 2.9 mmol), product was obtained after silica gel chromatography (20% to 45% ethyl acetate in hexanes) in 71% yield as a pale yellow liquid (440 mg). ¹H NMR (400 MHz, CDCl₃): δ 6.40 (dd, J=10.6, 16.8 Hz, 1H), 6.04 (dd, J=2.0, 16.8 Hz, 1H), 5.47 (dd, J=2.0, 10.6 Hz, 1H), 4.23 (d, J=13.2 1H), 3.93 (q, J=7.1 Hz, 2H), 3.76 (d, J=14.0 Hz, 1H), 2.99 (t, J=11.8 Hz, 1H), 2.70 (t, J=11.5 Hz, 1H), 2.37 (tt, J=4.1, 10.7 Hz, 1H), 1.77-1.73 (m, 2H), 1.51-1.42 (m, 2H), 1.05 (t, J=7.1 Hz, 3H). ¹³C NMR (100 MHz, CDCl₃): δ 173.7, 165.0, 127.5, 127.2, 60, 44.7, 41.0, 40.5, 28.2, 27.4, 13.8. HRMS (+ESI): Calculated: 212.1281 (C₁₁H₁₈NO₃). Observed: 212.1276.

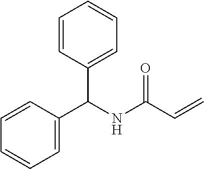 [SMILES: C=CC(=O)NC(c1ccccc1)c1ccccc1]

N-benzhydrylacrylamide (DKM 3-4). Following General Procedure A starting from benzhydrylamine (459 mg, 3.0 mmol), product was obtained after silica gel chromatography (0% to 20% ethyl acetate in hexanes) and recrystallization from toluene in 15% yield as a white solid (110 mg). ¹H NMR (400 MHz, (CD₃)₂CO): δ 7.35-7.23 (m, 10H), 6.45 (dd, J=10.2, 17.0 Hz, 1H), 6.36-6.34 (m, 1H), 6.25 (dd, J=2.2, 17.0 Hz, 1H), 5.61 (dd, J=2.2, 10.2 Hz, 1H). ¹³C NMR (100 MHz, (CD₃)₂CO): δ 164.84, 164.76, 143.51, 143.48, 132.51, 132.47, 129.4, 128.5, 1280, 126.3, 57.5, 57.4. HRMS (+ESI): Calculated: 238.1226 (C₁₆H₁₆NO). Observed: 238.1222.

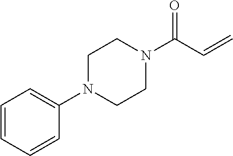 [SMILES: C=CC(=O)N1CCN(c2ccccc2)CC1]

1-(4-phenylpiperazin-1-yl)prop-2-en-1-one (DKM 3-5). Following General Procedure A starting from 1-phenylpiperazine (479 mg, 3.0 mmol), product was obtained after silica gel chromatography (30% to 70% ethyl acetate in hexanes) in 87% yield as a yellow oil (555 mg). ¹H NMR (400 MHz, CDCl₃): 7.30-7.25 (m, 2H), 6.92-6.87 (m, 3H), 6.60 (dd, J=10.5, 16.8 Hz 1H), 6.33 (dd, J=2.0, 16.8 Hz, 1H), 5.72 (dd, J=2.0, 10.5 Hz, 1H), 3.81 (s, 2H), 3.66 (s, 2H), 3.14 (t, J=5.2 Hz, 4H). ¹³C NMR (100 MHz, CDCl₃): δ 165.0, 150.6, 18.9, 127.8, 127.1, 120.2, 116.3, 49.4, 48.9, 45.3, 41.5. HRMS (+ESI): Calculated: 217.1335 (C₁₃H₁₇N₂O). Observed: 217.1332.

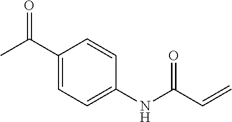

N-(4-acetylphenyl)acrylamide (DKM 3-7). Following General Procedure A starting from 4-aminoacetophenone (398 mg, 2.9 mmol), product was obtained after silica gel chromatography (20% to 50% ethyl acetate in hexanes) in 45% yield as a white solid (253 mg). ¹H NMR (400 MHz, CDCl₃): δ 8.40 (s, 1H), 7.92 (d, J=8.7 Hz, 2H), 7.73 (d, J=8.7 Hz, 2H), 6.46 (dd, J=1.3, 16.9 Hz, 1H), 6.34 (dd, J=10.1, 16.9 Hz, 1H), 5.79 (dd, J=1.3, 10.1 Hz, 1H), 2.57 (s, 3H). ¹³C NMR (100 MHz, CDCl₃): δ 197.5, 164.1, 142.5, 133.0, 130.9, 129.9, 128.9, 119.4, 26.6. HRMS (+ESI): Calculated: 190.0863 (C₁₁H₁₂NO₂). Observed: 190.0858.

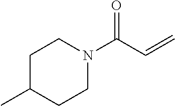

1-(4-methylpiperidin-1-yl)prop-2-en-1-one (DKM 3-8). Following General Procedure A starting from 4-methylpiperidine (295 mg, 3.0 mmol), product was obtained after silica gel chromatography (10% to 30% ethyl acetate in hexanes) in 84% yield as a yellow oil (385 mg). ¹H NMR (400 MHz, CDCl₃): δ 6.51 (dd, J=10.6, 16.5 Hz, 1H), 6.16 (dd, J=2.0, 16.5 Hz, 1H), 5.57 (dd, J=2.0, 10.6 Hz, 1H), 4.53 (d, J=13.1 Hz, 1H), 3.88 (d, J=13.3 Hz, 1H), 2.99-2.92 (m, 1H), 2.55 (td, J=2.1, 12.8 Hz, 1H), 1.62 (d, J=13.1 Hz, 2H), 1.57-1.49 (m, 1H), 1.10-0.98 (m, 2H), 0.87 (d, J=6.5 Hz, 3H). ¹³C NMR (100 MHz, CDCl₃): δ 165.2, 128.0, 127.0, 46.2, 42.4, 34.7, 33.7, 31.1, 21.7. HRMS (+ESI): Calculated: 154.1226 (C₉H₁₆NO). Observed: 154.1224.

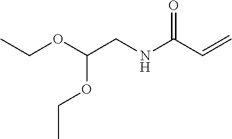

N-(2,2-diethoxyethyl)acrylamide (DKM 3-9). Following General Procedure A starting from aminoacetaldehyde diethyl acetal (402 mg, 3.0 mmol), product was obtained after silica gel chromatography (10% to 40% ethyl acetate in hexanes) in 75% yield as a clear oil (313 mg). ¹H NMR (400 MHz, CDCl₃): 6.25-6.19 (m, 2H), 6.09 (dd, J=10.1, 17.0 Hz, 1H), 5.56 (dd, J=1.7, 10.1 Hz, 1H), 4.48 (t, J=5.3 Hz, 1H), 3.64 (dq, J=7.1, 9.4 Hz, 2H), 3.47 (dq, J=7.1, 9.4 Hz, 2H), 3.38 (t, J=5.6 Hz, 2H), 1.13 (t, J=7.1 Hz, 6H). ¹³C NMR (100 MHz, CDCl₃): δ 165.7, 130.6, 126.4, 100.6, 62.8, 42.0, 15.2. HRMS (+ESI): Calculated: 188.1281 (C₉H₁₈NO₃). Observed: 188.1278.

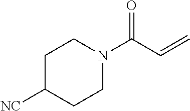

1-acryloylpiperidine-4-carbonitrile (DKM 3-11). Following General Procedure A starting from piperidine-4-carbonitrile (329 mg, 3.0 mmol), product was obtained after silica gel chromatography (30% to 70% ethyl acetate in hexanes) in 48% yield as a colorless oil (234 mg). ¹H NMR (400 MHz, CDCl₃): 6.49 (dd, J=10.6, 16.8 Hz, 1H), 6.19 (d, J=1.9, 16.8 Hz, 1H), 5.64 (dd, J=1.9, 10.6 Hz, 1H), 3.77-3.46 (m, 4H), 2.88-2.82 (sept, J=3.9 Hz, 1H), 1.90-1.73 (m, 4H). ¹³C NMR (100 MHz, CDCl₃): δ 165.4, 128.3, 127.3, 120.8, 43.8, 39.9, 29.1, 28.1, 26.3. HRMS (+ESI): Calculated: 165.1022 (C₉H₁₃N₂O). Observed: 165.1020.

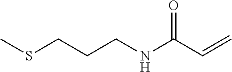

N-(3-(methylthio)propyl)acrylamide (DKM 3-12). Following General Procedure A starting from 3-(methylthio)propylamine (313 mg, 3.0 mmol), product was obtained after silica gel chromatography (20% to 60% ethyl acetate in hexanes) in 69% yield as a colorless oil (328 mg). ¹H NMR (400 MHz, CDCl₃): δ 6.79 (s, 1H), 6.19 (dd, J=2.2, 17.0 Hz, 1H), 6.11 (dd, J=9.6, 17.0 Hz, 1H), 5.55 (dd, J=2.2, 9.6 Hz, 1H), 3.35 (q, J=6.5 Hz, 2H), 2.47 (t, J=7.2 Hz, 2H), 2.02 (s, 3H), 1.78 (quint, J=7.0 Hz, 2H). ¹³C NMR (100 MHz, CDCl₃): δ 165.9, 131.0, 126.1, 38.6, 31.6, 28.6, 15.4. HRMS (+ESI): Calculated: 160.0791 (C₇H₁₄NOS). Observed: 160.0788.

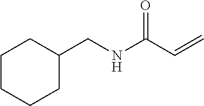

N-(cyclohexylmethyl)acrylamide (DKM 3-13). Following General Procedure A starting from cyclohexanemethylamine (331 mg, 2.9 mmol), product was obtained after silica gel chromatography (10% to 50% ethyl acetate in hexanes) in 67% yield as a pale yellow solid (330 mg). ¹H NMR (400 MHz, CDCl₃): 6.51 (s, 1H), 6.22 (dd, J=2.5, 17.0 Hz, 1H) 6.15 (dd, J=9.3, 17.0 Hz, 1H), 5.55 (dd, J=2.5, 9.3 Hz, 1H), 3.11 (t, J=6.5 Hz, 2H), 1.70-1.58 (m, 5H), 1.51-1.40 (m, 1H), 1.22-1.04 (m, 3H), 0.93-0.83 (m, 2H). ¹³C NMR (100 MHz, CDCl₃): δ 165.9, 131.2, 125.9, 45.9, 38.0, 30.9, 26.4, 25.8. HRMS (+ESI): Calculated: 168.1383 (C₁₀H₁₈NO). Observed: 168.1380.

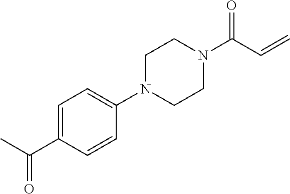 [SMILES: C=CC(=O)N1CCN(c2ccc(C(C)=O)cc2)CC1]

1-(4-(4-acetylphenyl)piperazin-1-yl)prop-2-en-1-one (DKM 3-29). Following General Procedure A starting from 4′-piperazinoacetophenone (607 mg, 3.0 mmol), product was obtained after silica gel chromatography (50% to 85% ethyl acetate in hexanes) in 65% yield as a yellow solid (496 mg). ¹H NMR (400 MHz, CDCl₃): δ 7.79 (d, J=9.0 Hz, 2H), 6.78 (d, J=9.0 Hz, 2H), 6.54 (dd, J=10.5, 16.8 Hz, 1H), 6.25 (dd, J=1.9, 16.8 Hz, 1H), 5.66 (dd, J=1.9, 10.5 Hz, 1H), 3.75 (s, 2H), 3.66 (s, 2H), 3.31-3.29 (m, 4H), 2.42 (s, 3H). ¹³C NMR (100 MHz, CDCl₃): δ 196.3, 165.2, 153.4, 130.2, 128.3, 127.9, 127.0, 113.5, 47.3, 47.0, 45.0, 41.2, 26.0. HRMS (+ESI): Calculated: 259.1441 (C₁₅H₁₉N₂O₂). Observed: 259.1436.

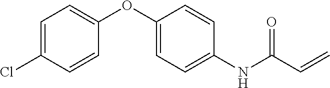 [SMILES: C=CC(=O)Nc1ccc(Oc2ccc(Cl)cc2)cc1]

N-(4-(4-chlorophenoxy)phenyl)acrylamide (DKM 3-30). Following General Procedure A starting from 4-(4-chlorophenoxy)aniline (440 mg, 2.0 mmol), product was obtained after silica gel chromatography (10% to 30% ethyl acetate in hexanes) in 33% yield as a white solid (180 mg). ¹H NMR (400 MHz, CDCl₃): δ 8.00 (s, 1H), 7.56 (d, J=8.9 Hz, 2H), 7.29-7.25 (m, 2H), 6.96-6.88 (m, 4H), 6.43 (dd, J=1.4, 16.9 Hz, 1H), 6.30 (dd, J=10.1, 16.9 Hz, 1H), 5.75 (dd, J=1.4, 10.1 Hz, 1H). ¹³C NMR (100 MHz, CDCl₃): δ 163.9, 156.2, 153.4, 133.7, 131.2, 129.8, 128.2, 128.0, 122.1, 119.8, 119.7. HRMS (+ESI): Calculated: 272.0484 (C₁₅H₁₁NO₂Cl). Observed: 272.0479.

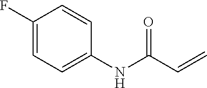

N-(4-fluorophenyl)acrylamide (DKM 3-31). Following General Procedure A starting from 4-fluoroaniline (239 mg, 2.2 mmol), product was obtained after silica gel chromatography (20% to 30% ethyl acetate in hexanes) in 16% yield as a white solid (56 mg). ¹H NMR (600 MHz, MeOD): δ 7.64-7.60 (m, 2H), 7.07-7.03 (m, 2H), 6.41 (dd, J=9.8, 17.0 Hz, 1H), 6.35 (dd, J=2.1, 17.0 Hz, 1H), 5.76 (dd, J=2.1, 9.8 Hz, 1H). ¹³C NMR (150 MHz, MeOD): δ 166.0, 161.56, 160.0, 135.93, 135.91, 132.3, 127.8, 123.2, 123.1, 116.4, 116.2. HRMS (−ESI): Calculated: 164.0517 (C₉H₇NOC). Observed: 164.0517.

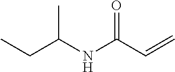

N-(sec-butyl)acrylamide (DKM 3-32). Following General Procedure A starting from sec-butylamine (222 mg, 3.0 mmol), product was obtained after silica gel chromatography (10% to 40% ethyl acetate in hexanes) in 74% yield as a yellow oil (287 mg). ¹H NMR (400 MHz, CDCl₃): δ 6.56 (d, J=5.6 Hz, 1H), 6.17 (s, 1H), 6.16 (d, J=3.5 Hz, 1H), 5.51 (dd, J=4.3, 7.6 Hz, 1H), 3.93-3.83 (m, 1H), 1.47-1.36 (m, 2H), 1.06 (d, J=6.6 Hz, 3H), 0.82 (t, J=7.5 Hz, 3H). ¹³C NMR (100 MHz, CDCl₃): δ 165.2, 131.4, 125.6, 46.6, 29.5, 20.2, 10.4. HRMS (+ESI): Calculated: 128.1070 (C₇H₁₄NO). Observed: 128.1069.

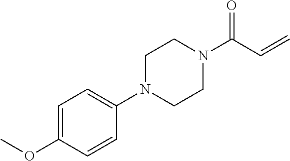 [SMILES: C=CC(=O)N1CCN(c2ccc(OC)cc2)CC1]

1-(4-(4-methoxyphenyl)piperazin-1-yl)prop-2-en-1-one (DKM 3-36). Following General Procedure A starting from 1-(4-methoxyphenyl)piperazine (388 mg, 2.0 mmol), product was obtained after silica gel chromatography (20% to 80% ethyl acetate in hexanes) in 29% yield as a white solid (143 mg). ¹H NMR (400 MHz, CDCl₃): δ 6.87-6.79 (m, 4H), 6.57 (dd, J=10.5, 16.8 Hz, 1H), 6.28 (dd, J=1.9, 16.8 Hz, 1H), 5.68 (dd, J=1.9, 10.5 Hz, 1H), 3.79 (s, 2H), 3.72 (s, 3H), 3.66 (s, 2H), 3.01 (t, J=5.1 Hz, 4H). ¹³C NMR (100 MHz, CDCl₃): δ 165.2, 154.3, 145.1, 128.0, 127.3, 118.8, 114.4, 55.4, 51.3, 50.7, 45.8, 41.9. HRMS (+ESI): Calculated: 247.1441 (C₁₄H₁₉N₂O₂). Observed: 247.1443.

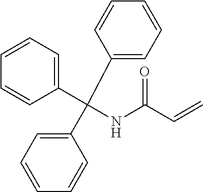 [SMILES: C=CC(=O)NC(c1ccccc1)(c1ccccc1)c1ccccc1]

N-tritylacrylamide (DKM 3-41). Following General Procedure A starting from triphenylmethylamine (386 mg, 1.5 mmol), product was obtained after silica gel chromatography (5% to 30% ethyl acetate in hexanes) in 74% yield as a white solid (346 mg). ¹H NMR (400 MHz, CDCl₃): δ 7.38-7.27 (m, 15H), 6.83 (s, 1H), 6.28-6.26 (m, 2H), 5.66 (dd, J=3.9, 7.2 Hz, 1H). ¹³C NMR (100 MHz, CDCl₃): δ 164.6, 144.6, 131.5, 128.8, 128.1, 127.2, 127.1, 70.7. HRMS (+ESI): Calculated: 314.1539 (C₂₂H₂₀NO). Observed: 314.1542.

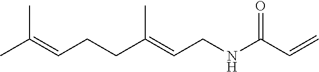

(E)-N-(3,7-dimethylocta-2,6-dien-1-yl)acrylamide (DKM 3-42). Following General Procedure A starting from geranylamine (462 mg, 3.0 mmol), product was obtained after silica gel chromatography (10% to 40% ethyl acetate in hexanes) in 23% yield as a colorless oil (141 mg). ¹H NMR (400 MHz, CDCl₃): δ 6.25 (dd, J=1.5, 17.0 Hz, 1H), 6.09 (dd, J=10.2, 17.0 Hz, 1H), 5.83 (s, 1H), 5.59 (dd, J=1.5, 10.2 Hz), 5.22-5.18 (m, 1H), 5.07-5.03 (m, 1H), 3.90 (t, J=6.2 Hz, 2H), 2.09-2.03 (m, 2H), 2.00-1.97 (m, 2H), 1.65 (s, 6H), 1.57 (s, 3H). ¹³C NMR (100 MHz, CDCl₃): δ 165.5, 140.2, 131.8, 131.0, 126.2, 123.9, 119.7, 39.6, 37.6, 265, 25.8, 17.8, 16.4. HRMS (+ESI): Calculated: 208.1696 (C₁₃H₂₂NO). Observed: 208.1697.

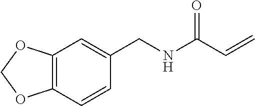

N-(benzo[d][1,3]dioxol-5-ylmethyl)acrylamide (DKM 3-43). Following General Procedure A starting from piperonylamine (312 mg, 2.1 mmol), product was obtained after silica gel chromatography (20% to 50% ethyl acetate in hexanes) in 74% yield as a white solid (315 mg). ¹H NMR (400 MHz, CDCl₃): δ 6.78 (s, 1H), 6.71 (s, 1H), 6.68 (s, 2H), 6.22 (dd, J=1.9, 17.0 Hz, 1H), 6.13 (dd, J=9.9, 17.0 Hz, 1H), 5.87 (s, 2H), 5.58 (dd, J=1.9, 9.9 Hz, 1H), 4.30 (d, J=5.8 Hz, 2H). ¹³C NMR (100 MHz, CDCl₃): δ 165.7, 147.8, 146.9, 132.0, 130.8, 126.6, 121.1, 108.4, 108.2, 101.0, 43.4. HRMS (+ESI): Calculated: 206.0812 (C₁₁H₁₂NO₃). Observed: 206.0808.

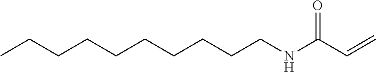

N-decylacrylamide (TRH 1-12). Following General Procedure A starting from decylamine (479 mg, 3.0 mmol), product was obtained after silica gel chromatography (20% to 40% ethyl acetate in hexanes) in 26% yield as a white solid (163 mg). ¹H NMR (400 MHz, CDCl₃): δ 6.54 (s, 1H), 6.21 (dd, J=2.0, 16.9 Hz, 1H) 6.13 (dd, J=9.7, 16.9 Hz, 1H), 5.55 (dd, J=2.0, 9.7 Hz, 1H), 3.25 (q, J=6.7 Hz, 2H), 1.50-1.45 (m, 2H), 1.29-1.20 (m, 14H), 0.83 (t, J=6.7 Hz, 3H). ¹³C NMR (100 MHz, CDCl₃): δ 165.8, 131.2, 125.9, 71.9, 39.7, 31.9, 29.6, 29.6, 29.38, 29.35, 27.0, 22.7, 14.1. HRMS (+ESI): Calculated: 212.2009 (C₁₃H₂₆NO). Observed: 212.2009.

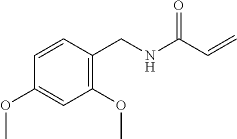 [SMILES: C=CC(=O)NCc1ccc(OC)cc1OC]

N-(2,4-dimethoxybenzyl)acrylamide (TRH 1-13). Following General Procedure A starting from 2,4-dimethoxybenzylamine (514 mg, 3.0 mmol), product was obtained after silica gel chromatography (20% to 60% ethyl acetate in hexanes) in 11% yield as a white solid (73 mg). ¹H NMR (400 MHz, CDCl₃): δ 7.17 (d, J=8.1 Hz, 1H), 6.43-6.39 (m, 2H), 6.26-6.22 (m, 2H), 6.07 (dd, J=10.7, 17.0 Hz, 1H), 5.57 (dd, J=1.4, 10.7 Hz, 1H), 4.41 (d, J=5.8 Hz, 2H), 3.79 (s, 3H), 3.77 (s, 3H). ¹³C NMR (100 MHz, CDCl₃): δ 165.2, 160.6, 158.6, 131.1, 130.7, 126.2, 118.7, 104.0, 98.6, 55.5, 55.4, 39.0. HRMS (+ESI): Calculated: 222.1125 (C₁₂H₁₆NO₃). Observed: 222.1124.

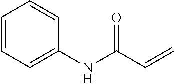

N-Phenylacrylamide (TRH 1-19). Following General Procedure A starting from aniline (277 mg, 3.0 mmol), product was obtained after recrystallization from a 1:20 ethyl acetate:hexanes mixture in 46% yield as a white solid (200 mg). ¹H NMR (400 MHz, CDCl₃): δ 8.59 (s, 1H), 7.63 (d, J=7.9 Hz, 2H), 7.30 (t, J=7.9 Hz, 2H), 7.11 (t, J=7.4 Hz, 1H), 6.44-6.33 (m, 2H), 5.70 (dd, J=2.8, 8.9 Hz, 1H). ¹³C NMR (100 MHz, CDCl₃): δ 164.3, 138.0, 131.4, 129.0, 127.7, 124.6, 120.5. HRMS (+ESI): Calculated: 148.0757 (C₉H₁₀NO). Observed: 148.0754.

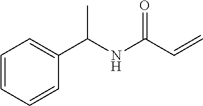

N-(1-phenylethyl)acrylamide (TRH 1-20). Following General Procedure A starting from 1-phenylethan-1-amine (387 mg, 3.0 mmol), product was obtained after silica gel chromatography (5% to 20% ethyl acetate in hexanes) in 46% yield as a white solid (315 mg). ¹H NMR (400 MHz, CDCl₃): δ 7.61 (d, J=7.8 Hz, 1H) 7.37-7.24 (m, 5H), 6.33-6.24 (m, 2H), 5.57 (dd, J=4.8, 7.9 Hz, 1H), 5.20 (quint, J=7.2 Hz, 1H), 1.49 (d, J=7.0 Hz, 3H). ¹³C NMR (100 MHz, CDCl₃): δ 165.0, 143.4, 131.1, 128.4, 126.9, 126.0, 126.0, 48.7, 21.8. HRMS (+ESI): Calculated: 176.1070 (C₁₁H₁₄NO). Observed: 176.1067.

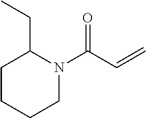

1-(2-ethylpiperidin-1-yl)prop-2-en-1-one (TRH 1-27). Following General Procedure A starting from 2-ethylpiperidine (238 mg, 2.0 mmol), product was obtained after silica gel chromatography (5% to 30% ethyl acetate in hexanes) in 72% yield as a white solid (253 mg). ¹H NMR (400 MHz, CDCl₃): δ 6.41 (dd, J=10.6, 16.7 Hz, 1H), 6.03 (d, J=16.4 Hz, 1H), 5.43 (dd, J=2.0, 10.6 Hz, 1H), 4.54-4.34 (m, 1H), 3.77-3.58 (m, 1H), 2.93-2.42 (m, 1H), 1.61-1.06 (m, 8H), 0.66 (t, J=7.5 Hz, 3H). ¹³C NMR (100 MHz, CDCl₃): δ 165.9, 130.0, 129.1, 128.4, 126.6, 54.4, 49.6, 41.1, 36.5, 28.8, 27.5, 26.2, 25.2, 23.0, 22.1, 18.8, 10.4. HRMS (+ESI): Calculated: 168.1383 (C₁₀H₁₈NO). Observed: 168.1380.

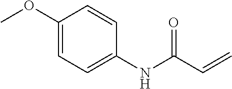

N-(4-methoxyphenyl)acrylamide (TRH 1-32). Following General Procedure A starting from p-anisidine (258 mg, 2.0 mmol), product was obtained after silica gel chromatography (10% to 50% ethyl acetate in hexanes) in 58% yield as a white solid (216 mg). ¹H NMR (400 MHz, CDCl₃): δ 8.94 (s, 1H), 7.48 (d, J=9.1 Hz, 2H), 6.78 (d, J=9.1 Hz, 2H), 6.34 (d, J=5.6 Hz, 2H), 5.61 (t, J=5.9 Hz, 1H), 3.73 (s, 3H). ¹³C NMR (100 MHz, CDCl₃): δ 164.3, 156.4, 131.4, 131.1, 127, 122.3, 114.0, 55.4. HRMS (+ESI): Calculated: 178.0863 (C₁₀H₁₂O₂N). Observed: 178.0859.

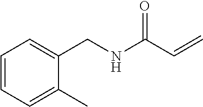

N-(2-methylbenzyl)acrylamide (TRH 1-54). Following General Procedure A starting from 2-methylbenzylamine (240 mg, 2.0 mmol), product was obtained after silica gel chromatography (30% to 40% ethyl acetate in hexanes) in 73% yield as a white solid (257 mg). ¹H NMR (400 MHz, CDCl₃): δ 7.26-7.12 (m, 4H), 6.66 (s, 1H), 6.24-6.12 (m, 2H), 5.57 (dd, J=9.5, 2.2 Hz, 1H), 4.39 (d, J=5.4 Hz, 2H), 2.27 (s, 3H). ¹³C NMR (100 MHz, CDCl₃): δ 165.6, 136.3, 135.7, 130.7, 130.4, 128.4, 127.6, 126.4, 126.1, 41.6, 19.0. HRMS (+ESI): Calculated: 176.1070 (C₁₁H₁₄NO). Observed: 176.1067.

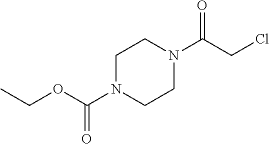

Ethyl 4-(2-chloroacetyl)piperazine-1-carboxylate (DKM 2-52). Following General Procedure B starting from ethyl 1-piperazinecarboxylate (477 mg, 3.0 mmol) product was obtained after silica gel chromatography (0% to 80% ethyl acetate in hexanes) in 80% yield as a pale yellow oil (569 mg). ¹H NMR (400 MHz, CDCl₃): δ 4.04-3.99 (m, 4H), 3.48-3.34 (m, 8H), 1.14 (t, J=7.1 Hz, 3H). ¹³C NMR (100 MHz, CDCl₃): δ 165.1, 155.0, 61.5, 45.8, 43.3, 43.0, 41.7, 40.7, 14.4. HRMS (+SI): Calculated: 235.0844 (C₉H₁₆ClN₂O₃). Observed: 235.0842.

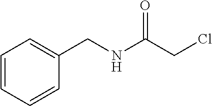 [SMILES: O=C(CCl)NCc1ccccc1]

N-benzyl-2-chloroacetamide (DKM 2-67). Following General Procedure B starting from benzylamine (430 mg, 3.1 mmol) product was obtained after silica gel chromatography (0% to 30% ethyl acetate in hexanes) in 70% yield as a white solid (416 mg). ¹H NMR (400 MHz, CDCl₃): δ 7.40-7.31 (m, 5H), 7.08 (s, 1s), 4.50 (d, J=5.8 Hz, 2H), 4.09 (s, 2H). ¹³C NMR (100 MHz, CDCl₃): δ 166.0, 137.4, 128.8, 127.8, 43.8, 42.6. HRMS (−ESI): Calculated: 182.0378 (C₉H₉NOCl). Observed: 182.0378.

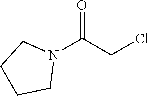

2-Chloro-1-(pyrrolidin-1-yl)ethan-1-one (DKM 2-71). Following General Procedure B starting from pyrrolidine (511 mg, 3.0 mmol) product was obtained after silica gel chromatography (0% to 30% ethyl acetate in hexanes) in 83% yield as a clear oil (368 mg). ¹H NMR (400 MHz, CDCl₃): δ 3.94 (s, 2H), 3.41 (quint, J=7.2 Hz, 4H), 1.91 (quint, J=6.3 Hz, 2H), 1.80 (quint, J=6.6 Hz, 2H). ¹³C NMR (100 MHz, CDCl₃): δ 164.7, 46.5, 46.3, 42.1, 26.1, 24.1. HRMS (+ESI): Calculated: 170.0343 (C₆H₁₀ClNNaO). Observed: 170.0343.

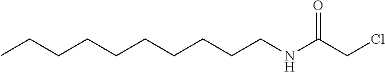

2-Chloro-N-decylacetamide (DKM 2-72). Following General Procedure B starting from decylamine (472 mg, 3.0 mmol) product was obtained after silica gel chromatography (0% to 40% ethyl acetate in hexanes) in 81% yield as a white solid (555 mg). ¹H NMR (400 MHz, CDCl₃): δ 6.71 (s, 1H), 3.97 (s, 2H), 3.22 (q, J=6.8 Hz, 2H), 1.51-1.44 (m, 2H), 1.24-1.19 (m, 14H), 0.81 (t, J=6.8 Hz, 3H). ¹³C NMR (100 MHz, CDCl₃): δ 165.8, 42.7, 39.9, 31.9, 29.5, 29.29, 29.27, 29.22, 26.8, 22.6, 14.1. HRMS (+ESI): Calculated: 234.1619 (C₁₂H₂₅ClNO). Observed: 234.1618.

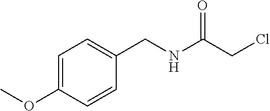

2-chloro-N-(4-methoxybenzyl)acetamide (DKM 2-83). Following General Procedure B starting from 4-methoxybenzylamine (430 mg, 3.1 mmol) product was obtained after silica gel chromatography (0% to 40% ethyl acetate in hexanes) in 55% yield as an off-white solid (369 mg). ¹H NMR (400 MHz, CDCl₃): δ 7.20 (d, J=8.6 Hz, 2H), 6.91 (s, 1H), 6.86 (d, J=8.6 Hz, 2H), 4.40 (d, J=5.7 Hz, 2H), 4.05 (s, 2H), 3.78 (s, 3H). ¹³C NMR (100 MHz, CDCl₃): δ 165.9, 159.2, 129.4, 129.2, 114.2, 55.3, 43.4, 42.7. HRMS (+ESI): Calculated: 214.0629 (C₁₀H₁₃ClNO₂). Observed: 214.0627.

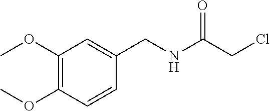 [SMILES: COc1ccc(CNC(=O)CCl)cc1OC]

2-chloro-N-(3,4-dimethoxybenzyl)acetamide (DKM 2-93). Following General Procedure B starting from 3,4-dimethoxybenzylamine (517 mg, 3.1 mmol) product was obtained after silica gel chromatography (0% to 50% ethyl acetate in hexanes) in 55% yield as an off-white solid (416 mg). ¹H NMR (400 MHz, CDCl₃): δ 6.97 (s, 1H), 6.77 (m, 3H), 4.35 (d, J=5.8 Hz, 2H), 4.01 (s, 2H), 3.81 (s, 3H), 3.80 (s, 3H). ¹³C NMR (100 MHz, CDCl₃): δ 165.8, 149.0, 148.5, 129.8, 120.1, 111.13, 111.07, 55.83, 55.79, 43.6, 42.5. HRMS (+ESI): Calculated: 266.0554 (C₁₁H₁₄NO₃ClNa). Observed: 266.0553.

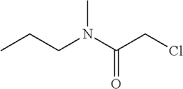

2-Chloro-N-methyl-N-propylacetamide (TRH 1-53). Following General Procedure B starting from N-methylpropylamine (147 mg, 2.0 mmol) product was obtained after silica gel chromatography (30% to 40% ethyl acetate in hexanes) in 64% yield as a white solid (191 mg). ¹H NMR (46:54 rotamer ratio, asterisks denote minor peaks, 400 MHz, CDCl₃): δ 4.03* (s, 2H), 4.02 (s, 2H), 3.28* (t, J=7.4 Hz, 2H), 3.23 (t, J=7.5 Hz, 2H), 3.00 (s, 3H), 2.88* (s, 3H), 1.64-1.56* (m, 2H), 1.53-1.46 (m, 2H), 0.87* (t, J=7.5 Hz, 3H), 0.83 (t, J=7.5 Hz, 3H). ¹³C NMR (asterisks denote minor rotamer peaks, 100 MHz, CDCl₃): δ 166.4, 166.3*, 51.9*, 49.8, 41.5, 40.9*, 35.6, 33.6*, 21.6*, 20.1, 11.1, 11.0*. HRMS (+ESI): Calculated: 150.0680 (C₆H₁₃NOCl). Observed: 150.0678.

Synthesis and Characterization of YP 1-46

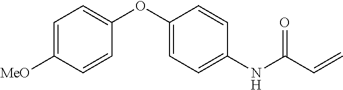 [SMILES: C=CC(=O)Nc1ccc(Oc2ccc(OC)cc2)cc1]

N-(4-(4-methoxyphenoxy)phenyl)acrylamide (YP-1-46). To a solution of 4-methoxyphenol (622 mg, 5 mmol) in DMF (2 mL) was added potassium carbonate (1.38 g, 10 mmol). After 10 minutes of stirring, 1-fluoro-4-nitrobenzene (0.43 mL, 4 mmol) was added and the reaction was stirred overnight. As the reaction was not complete by TLC after 21 hours, the reaction was heated to 90 degrees for 1 hour at which point the reaction was found to be complete. The reaction was then diluted with water and extracted three times with ethyl acetate. The combined organics were dried with magnesium sulfate and concentrated to give 1.07 g of crude 1-methox-4-(nitrophenoxy)benzene as a yellow solid that was used without further purification.

To a stirring solution of the resulting crude (490 mg, ˜2 mmol) and 10% palladium on activated charcoal (49 mg) in methanol (4 mL) was added triethylsilane (2.33 g, 20 mmol) dropwise through an addition funnel under a nitrogen-filled balloon. After 30 min, the mixture was filtered through celite and the solvent was removed in vacuo. Without further purification, the obtained crude product was dissolved in DCM (10 mL) and the resultant solution was cooled to 0° C. To the solution was added acryloyl chloride (217 mg, 2.4 mmol) followed by triethylamine (243 mg, 2.4 mmol). The solution was allowed to warm to room temperature after 20 min and stirred overnight. The solution was washed two times with brine and the crude product was purified via silica gel chromatography (30% to 70% ethyl acetate in hexanes) to afford 161 mg of the product as a white solid (33% yield over 3 steps). ¹H NMR (400 MHz, CDCl₃): δ 7.94 (s, 1H), 7.50-7.48 (m, 2H), 6.96-6.92 (m, 2H), 6.90-6.84 (m, 4H), 6.40 (dd, J=1.6, 16.8 Hz, 1H), 6.27 (dd, J=10.1, 16.8 Hz, 1H), (dd, J=1.6, 10.1 Hz, 1H), 3.79 (s, 3H). ¹³C NMR (100 MHz, CDCl₃): δ 163.8, 155.8, 155.1, 150.4, 132.6, 131.1, 127.6, 121.9, 120.5, 118.2, 114.9, 55.7. HRMS (+ESI): Calculated: 270.1125 (C₁₆H₁₆NO₃). Observed: 270.1125.

Synthesis and Characterization of AMR 1-125

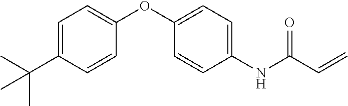 [SMILES: C=CC(=O)Nc1ccc(Oc2ccc(C(C)(C)C)cc2)cc1]

### N-(4-(4-(tert-butyl)phenoxy)phenyl)acrylamide (AMR 1-125)

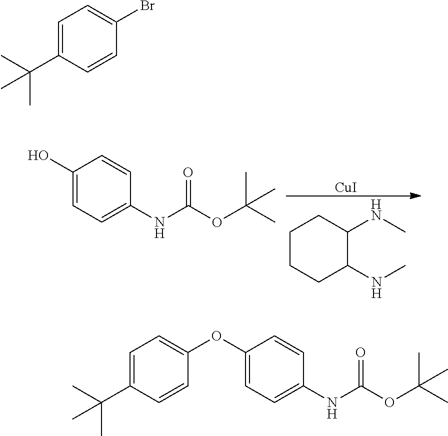

An oven dried round bottom flask was charged with a magnetic stir bar, copper (I) iodide (38 mg, 0.2 mmol), N-Boc-4-hydroxyaniline (502 mg, 2.4 mmol), potassium carbonate (552.8 mg, 2 mmol), and crushed 4 angstrom sieves (˜200 mg). The flask was evacuated and filled with nitrogen twice. Under nitrogen, 1-Bromo-4-tert-butylbenzene (346 uL, 2 mmol) and N,N′-Dimethyl-1,2-cyclohexanediamine (62 uL, 0.2 mmol) were added along with 2 mL of butyronitrile. The flask was allowed to react at 70 C for 24 hours. At the end of the reaction, the mixture was diluted with CH₂Cl₂ and rinsed through Celite to remove inorganic salts and other solids. The crude reaction mixture was purified using column chromatograph (10% ethyl acetate in hexanes). The product was a yellow oil (yield 31%).

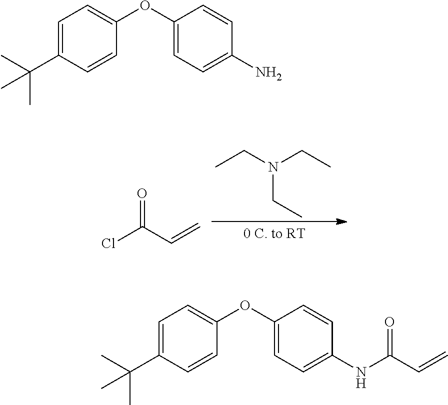

An oven dried round bottom flask was charged with the amine starting material (154.2 mg, 0.6 mmol) along with dry CH₂C₂ and allowed to cool to OC. Acryloyl chloride (69.4 mg, 0.8 mmol) was then added to the flask, followed by triethylamine (196 uL, 1.4 mmol), and the reaction was allowed to come to room temperature overnight. At the end of the reaction, the mixture was washed with brine and then purified using column chromatography (10% ethyl acetate in hexanes). The product was a waxy, white solid (yield <10%). ¹H NMR (900 MHz, CDCl₃): δ 7.51 (d, 2H, J=8.5 Hz), δ 7.31 (d, 2H, J=8.5 Hz), δ 6.97 (m, 2H), δ 6.90 (m, 2H), δ 6.42 (d, 1H, J=16.8 Hz), δ 6.22 (dd, 1H, J=16.8, 10.3 Hz), δ 5.75 (m, 1H), δ 1.3 (s, 9H). ¹³C NMR (900 MHz, CDCl₃): δ 163.4, 155.1, 154.2, 146.3, 133.0, 131.2, 127.9, 126.7, 121.7, 119.5, 118.3, 34.5, 31.7, 29.9. HRMS (+ESI): Calculated: 296.1645 (C₁₉H₂₁NO₂) Observed: 296.1643.

General Procedure Synthetic Scheme for Derivatives

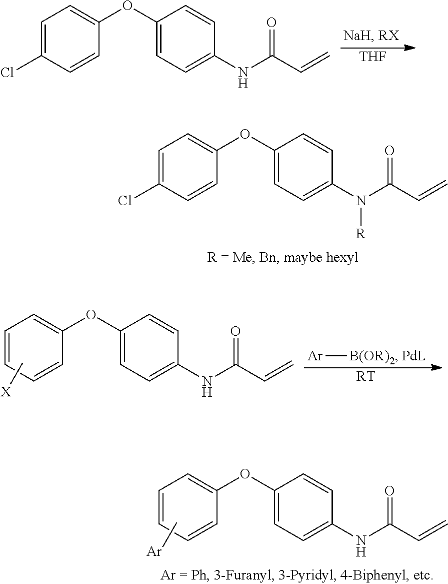

Derivatives being Synthesized

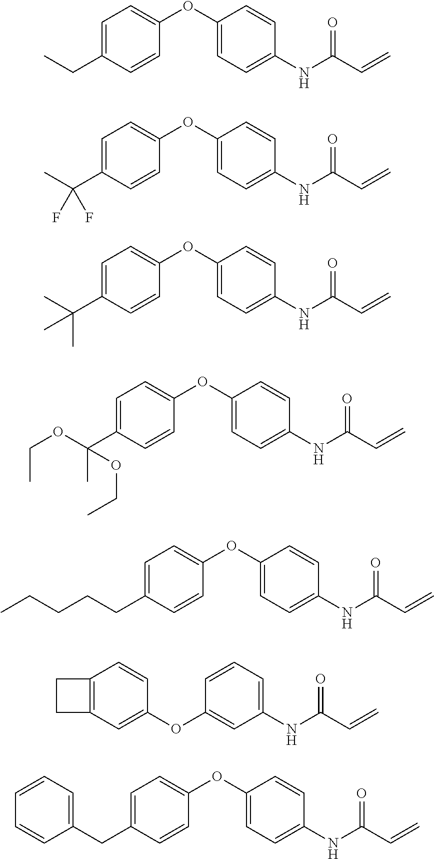

### REFERENCES

(1) NIH-SEER database; (2) Damin, D. C., and Lazzaron, A. R. (2014) Evolving treatment strategies for colorectal cancer: A critical review of current therapeutic options. World J. Gastroenterol. WJG 20, 877-887; (3) Gangadhar, N. M., and Stockwell, B. R. (2007) Chemical genetic approaches to probing cell death. Curr. Opin. Chem. Biol. 11, 83-87; (4) Smukste, I., and Stockwell, B. R. (2005) Advances in chemical genetics. Annu. Rev. Genomics Hum. Genet. 6, 261-286; (5) Weerapana, E., Wang, C., Simon, G. M., Richter, F., Khare, S., Dillon, M. B. D., Bachovchin, D. A., Mowen, K., Baker, D., and Cravatt, B. F. (2010) Quantitative reactivity profiling predicts functional cysteines in proteomes. Nature 468, 790-795; (6) Backus, K. M., Correia, B. E., Lum, K. M., Forli, S., Horning, B. D., Gonzalez-Paez, G. E., Chatterjee, S., Lanning, B. R., Teij aro, J. R., Olson, A. J., Wolan, D. W., and Cravatt, B. F. (2016) Proteome-wide covalent ligand discovery in native biological systems. Nature 534, 570-574; (7) Wang, C., Weerapana, E., Blewett, M. M., and Cravatt, B. F. (2014) A chemoproteomic platform to quantitatively map targets of lipid-derived electrophiles. Nat. Methods 11, 79-85; (8) Edfeldt, F. N. B., Folmer, R. H. A., and Breeze, A. L. (2011) Fragment screening to predict druggability (ligandability) and lead discovery success. Drug Discov. Today 16, 284-287; (9) Rostovtsev, V. V., Green, L. G., Fokin, V. V., and Sharpless, K. B. (2002) A stepwise huisgen cycloaddition process: copper(I)-catalyzed regioselective “ligation” of azides and terminal alkynes. Angew. Chem. Int. Ed Engl. 41, 2596-2599; (10) Weerapana, E., Simon, G. M., and Cravatt, B. F. (2008) Disparate proteome reactivity profiles of carbon electrophiles. Nat. Chem. Biol. 4, 405-407; (11) Jozsef, L., Tashiro, K., Kuo, A., Park, E. J., Skoura, A., Albinsson, S., Rivera-Molina, F., Harrison, K. D., Iwakiri, Y., Toomre, D., and Sessa, W. C. (2014) Reticulon 4 is necessary for endoplasmic reticulum tubulation, STIM1-Orail coupling, and store-operated calcium entry. J. Biol. Chem. 289, 9380-9395; (12) Voeltz, G. K., Prinz, W. A., Shibata, Y., Rist, J. M., and Rapoport, T. A. (2006) A class of membrane proteins shaping the tubular endoplasmic reticulum. Cell 124, 573-586; (13) Shibata, Y., Hu, J., Kozlov, M. M., and Rapoport, T. A. (2009) Mechanisms shaping the membranes of cellular organelles. Annu. Rev. Cell Dev. Biol. 25, 329-354; (14) Vasudevan, S. V., Schulz, J., Zhou, C., and Cocco, M. J. (2010) Protein folding at the membrane interface, the structure of Nogo-66 requires interactions with a phosphocholine surface. Proc. Natl. Acad. Sci. U.S.A 107, 6847-6851; (15) Ettinger, S., Laurell, E., and Kutay, U. (2009) Orchestrating nuclear envelope disassembly and reassembly during mitosis. Nat. Rev. Mol. Cell Biol. 10, 178-191; (16) Anderson, D. J., and Hetzer, M. W. (2008) Reshaping of the endoplasmic reticulum limits the rate for nuclear envelope formation. J. Cell Biol. 182, 911-924; (17) Kiseleva, E., Morozova, K. N., Voeltz, G. K., Allen, T. D., and Goldberg, M. W. (2007) Reticulon 4a/NogoA locates to regions of high membrane curvature and may have a role in nuclear envelope growth. J. Struct. Biol. 160, 224-235; (18) Audhya, A., Desai, A., and Oegema, K. (2007) A role for Rab5 in structuring the endoplasmic reticulum. J. Cell Biol. 178, 43-56; (19) Yan, R., Shi, Q., Hu, X., and Zhou, X. (2006) Reticulon proteins: emerging players in neurodegenerative diseases. Cell. Mol. Life Sci. CMLS 63, 877-889; (20) Karnezis, T., Mandemakers, W., McQualter, J. L., Zheng, B., Ho, P. P., Jordan, K. A., Murray, B. M., Barres, B., Tessier-Lavigne, M., and Bernard, C. C. A. (2004) The neurite outgrowth inhibitor Nogo A is involved in autoimmune-mediated demyelination. Nat. Neurosci. 7, 736-744; (21) Weerapana, E., Speers, A. E., and Cravatt, B. F. (2007) Tandem orthogonal proteolysis-activity-based protein profiling (TOP-ABPP)—a general method for mapping sites of probe modification in proteomes. Nat. Protoc. 2, 1414-1425; (22) Louie, S. M., Grossman, E. A., Crawford, L. A., Ding, L., Camarda, R., Huffman, T. R., Miyamoto, D. K., Goga, A., Weerapana, E., and Nomura, D. K. (2016) GSTP1 Is a Driver of Triple-Negative Breast Cancer Cell Metabolism and Pathogenicity. Cell Chem. Biol. 23, 567-578; (23) Xu, T., Park, S. K., Venable, J. D., Wohlschlegel, J. A., Diedrich, J. K., Cociorva, D., Lu, B., Liao, L., Hewel, J., Han, X., Wong, C. C. L., Fonslow, B., Delahunty, C., Gao, Y., Shah, H., and Yates, J. R. (2015) ProLuCID: An improved SEQUEST-like algorithm with enhanced sensitivity and specificity. J. Proteomics 129, 16-24; (24) Medina-Cleghorn, D., Bateman, L. A., Ford, B., Heslin, A., Fisher, K. J., Dalvie, E. D., and Nomura, D. K. (2015) Mapping Proteome-Wide Targets of Environmental Chemicals Using Reactivity-Based Chemoproteomic Platforms. Chem. Biol. 22, 1394-1405; (25) Benjamin, D. I., Louie, S. M., Mulvihill, M. M., Kohnz, R. A., Li, D. S., Chan, L. G., Sorrentino, A., Bandyopadhyay, S., Cozzo, A., Ohiri, A., Goga, A., Ng, S.-W., and Nomura, D. K. (2014) Inositol phosphate recycling regulates glycolytic and lipid metabolism that drives cancer aggressiveness. ACS Chem. Biol. 9, 1340-1350.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating cancer, said method comprising administering to a subject in need thereof an effective amount of a compound having the formula: 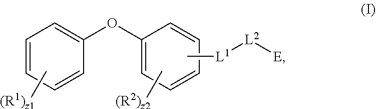
wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OC^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 5;
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;
z2 is an integer from 0 to 4;
L¹ is a bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
Each R^{1A}, R^{1B}, R^{1C}, R^{1D}, R^{2A}, R^{2B}, R^{2C}, R^{2D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
each X, X¹, X², X⁴, and X⁵ is independently —F, —Cl, —Br, or —I;
n1, n2, n4, and n5 are independently an integer from 0 to 4; and
m1, m2, m4, m5, v1, v2, v4, and v5 are independently an integer from 1 to 2.

2. The method of claim 1, wherein the compound has the formula:

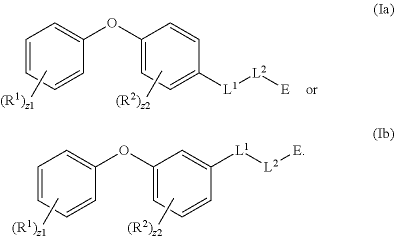

3. (canceled)

4. (canceled)

5. The method of claim 1, wherein the compound has the formula:

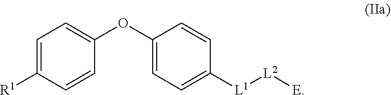

6. (canceled)

7. The method of claim 1, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ aryl, or substituted or unsubstituted 5 to 12 membered heteroaryl.

8. (canceled)

9. The method of claim 1, wherein R¹ is independently —Cl.

10. The method of claim 1, wherein two adjacent R¹ substituents are joined to form a substituted or unsubstituted cycloalkyl, substituted or unsubstituted heterocycloalkyl, substituted or unsubstituted aryl, or substituted or unsubstituted heteroaryl.

11. (canceled)

12. The method of claim 1, wherein two adjacent R¹ substituents are joined to form an unsubstituted C₃-C₆ cycloalkyl.

13. (canceled)

14. The method of claim 1, wherein L¹ is a bond.

15. The method of claim 1, wherein L² is —NR⁵— or substituted or unsubstituted heterocycloalkylene comprising a ring nitrogen bonded directly to E.

16. The method of claim 1, wherein L² is —NR⁵—.

17. The method of claim 16, wherein R⁵ is hydrogen, substituted or unsubstituted C₁-C₆ alkyl, or substituted or unsubstituted 2 to 6 membered heteroalkyl.

18. (canceled)

19. The method of claim 16, wherein R⁵ is hydrogen, unsubstituted methyl, unsubstituted ethyl, unsubstituted hexyl, or unsubstituted benzyl.

20. The method of claim 16, wherein R⁵ is hydrogen.

21. (canceled)

22. The method of claim 1, wherein E is: 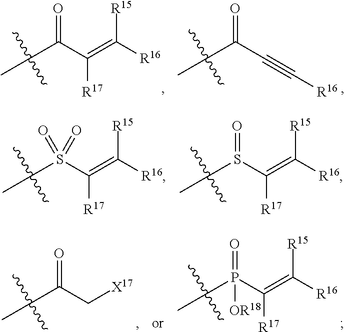
R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, and R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;
each X, X¹⁵, X¹⁶, X¹⁷ and X¹⁸ is independently —F, —Cl, —Br, or —I;
n15, n16, n17, v15, v16, and v17, are independently an integer from 0 to 4; and
m15, m16, and m17 are independently and integer from 1 to 2.

23. (canceled)

24. The method of claim 22, wherein E is:

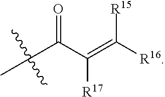

25. The method of claim 1, having the formula:

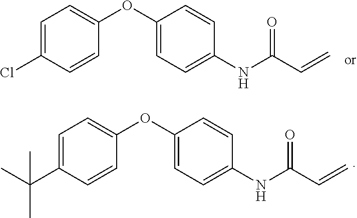

26. The method of claim 1, wherein the cancer is colorectal cancer.

27. (canceled)

28. (canceled)

29. A pharmaceutical composition comprising a Reticulon 4 inhibitor and a pharmaceutically acceptable excipient, wherein the Reticulon 4 inhibitor is a compound having the formula: 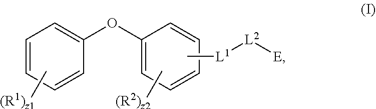
wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 5;
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;
z2 is an integer from 0 to 4;
L¹ is a bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
Each R^{1A}, R^{1B}, R^{1C}, R^{1D}, R^{2A}, R^{2B}, R^{2C}, R^{2D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
each X, X¹, X², X⁴, and X⁵ is independently —F, —Cl, —Br, or —I;
n1, n2, n4, and n5 are independently an integer from 0 to 4; and
m1, m2, m4, m5, v1, v2, v4, and v5 are independently an integer from 1 to 2.

30. (canceled)

31. (canceled)

32. A method inhibiting Reticulon 4 protein activity, said method comprising contacting a Reticulon 4 protein with an effective amount of a Reticulon 4 inhibitor, wherein said Reticulon 4 inhibitor is a compound having the formula: 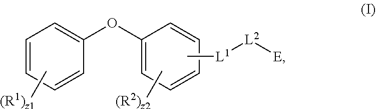
wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 5;
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;
z2 is an integer from 0 to 4;
L¹ is a bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
Each R^{1A}, R^{1B}, R^{1C}, R^{1D}, R^{2A}, R^{2B}, R^{2C}, R^{2D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
each X, X¹, X², X⁴, and X⁵ is independently —F, —Cl, —Br, or —I;
n1, n2, n4, and n5 are independently an integer from 0 to 4; and
m1, m2, m4, m5, v1, v2, v4, and v5 are independently an integer from 1 to 2.

33. (canceled)

34. A reticulon 4 protein covalently bonded to a compound having the formula: 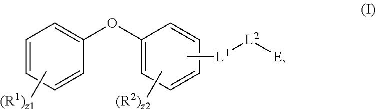
wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OC^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 5;
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;
z2 is an integer from 0 to 4;
L¹ is a bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
Each R^{1A}, R^{1B}, R^{1C}, R^{1D}, R^{2A}, R^{2B}, R^{2C}, R^{2D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
each X, X¹, X², X⁴, and X⁵ is independently —F, —Cl, —Br, or —I;
n1, n2, n4, and n5 are independently an integer from 0 to 4; and
m1, m2, m4, m5, v1, v2, v4, and v5 are independently an integer from 1 to 2;
wherein the reticulon 4 protein is covalently bonded to said compound through said reacted electrophilic moiety.

35. (canceled)

36. (canceled)


SEQUENCE LISTING (343 sequences)

SEQ ID NO: 1 - protein, 14 aa, Artificial Sequence
  Synthetic Polypeptide
YSNSALGHVNCTIK

SEQ ID NO: 2 - protein, 10 aa, Artificial Sequence
  Synthetic Polypeptide
AYEYVECPIR

SEQ ID NO: 3 - protein, 23 aa, Artificial Sequence
  Synthetic Polypeptide
APPWVPAMGFTLAPSLGCFVGSR

SEQ ID NO: 4 - protein, 17 aa, Artificial Sequence
  Synthetic Polypeptide
YYGGAEVVDEIELLCQR

SEQ ID NO: 5 - protein, 16 aa, Artificial Sequence
  Synthetic Polypeptide
KVIGIECSSISDYAVK

SEQ ID NO: 6 - protein, 15 aa, Artificial Sequence
  Synthetic Polypeptide
TYDPSGDSTLPTCSK

SEQ ID NO: 7 - protein, 38 aa, Artificial Sequence
  Synthetic Polypeptide
FTTSCMTGYSPQLQGLSSGGSGSYSPGVTYSPVSGYNK

SEQ ID NO: 8 - protein, 22 aa, Artificial Sequence
  Synthetic Polypeptide
SLVQNNCLSRPNIFLCPEIEPK

SEQ ID NO: 9 - protein, 9 aa, Artificial Sequence
  Synthetic Polypeptide
LWNTLGVCK

SEQ ID NO: 10 - protein, 12 aa, Artificial Sequence
  Synthetic Polypeptide
SLPSAVYCIEDK

SEQ ID NO: 11 - protein, 27 aa, Artificial Sequence
  Synthetic Polypeptide
EGGQYGLVAACAAGGQGHAMIVEAYPK

SEQ ID NO: 12 - protein, 16 aa, Artificial Sequence
  Synthetic Polypeptide
AAIGCGIVESILNWVK

SEQ ID NO: 13 - protein, 20 aa, Artificial Sequence
  Synthetic Polypeptide
LCPLKDEPWPIHPWEPGSFR

SEQ ID NO: 14 - protein, 21 aa, Artificial Sequence
  Synthetic Polypeptide
VIEASDVVLEVLDARDPLGCR

SEQ ID NO: 15 - protein, 12 aa, Artificial Sequence
  Synthetic Polypeptide
YVAAAFPSACGK

SEQ ID NO: 16 - protein, 16 aa, Artificial Sequence
  Synthetic Polypeptide
VCTLAIIDPGDSDIIR

SEQ ID NO: 17 - protein, 15 aa, Artificial Sequence
  Synthetic Polypeptide
STPYECGFDPMSPAR

SEQ ID NO: 18 - protein, 20 aa, Artificial Sequence
  Synthetic Polypeptide
VALEGLRPTIPPGISPHVCK

SEQ ID NO: 19 - protein, 18 aa, Artificial Sequence
  Synthetic Polypeptide
LLEETGICVVPGSGFGQR

SEQ ID NO: 20 - protein, 18 aa, Artificial Sequence
  Synthetic Polypeptide
FGVICLEDLIHEIAFPGK

SEQ ID NO: 21 - protein, 10 aa, Artificial Sequence
  Synthetic Polypeptide
ASCLYGQLPK

SEQ ID NO: 22 - protein, 14 aa, Artificial Sequence
  Synthetic Polypeptide
ATGHSGGGCISQGR

SEQ ID NO: 23 - protein, 12 aa, Artificial Sequence
  Synthetic Polypeptide
HVVCAAETGSGK

SEQ ID NO: 24 - protein, 35 aa, Artificial Sequence
  Synthetic Polypeptide
STFFNVLTNSQASAENFPFCTIDPNESRVPVPDER

SEQ ID NO: 25 - protein, 36 aa, Artificial Sequence
  Synthetic Polypeptide
VDDEILGFISEATPLGGIQAASTESCNQQLDLALCR

SEQ ID NO: 26 - protein, 18 aa, Artificial Sequence
  Synthetic Polypeptide
IDRYTQQGFGNLPICMAK

SEQ ID NO: 27 - protein, 17 aa, Artificial Sequence
  Synthetic Polypeptide
AVCMLSNTTAIAEAWAR

SEQ ID NO: 28 - protein, 23 aa, Artificial Sequence
  Synthetic Polypeptide
LCYVALDFENEMATAASSSSLEK

SEQ ID NO: 29 - protein, 11 aa, Artificial Sequence
  Synthetic Polypeptide
YATSCYSCCPR

SEQ ID NO: 30 - protein, 12 aa, Artificial Sequence
  Synthetic Polypeptide
VIGSGCNLDSAR

SEQ ID NO: 31 - protein, 16 aa, Artificial Sequence
  Synthetic Polypeptide
VLPMNTGVEAGETACK

SEQ ID NO: 32 - protein, 12 aa, Artificial Sequence
  Synthetic Polypeptide
AVLLVGLCDSGK

SEQ ID NO: 33 - protein, 11 aa, Artificial Sequence
  Synthetic Polypeptide
VTDDLVCLVYK

SEQ ID NO: 34 - protein, 12 aa, Artificial Sequence
  Synthetic Polypeptide
VCEEIAIIPSKK

SEQ ID NO: 35 - protein, 11 aa, Artificial Sequence
  Synthetic Polypeptide
VQENSAYICSR

SEQ ID NO: 36 - protein, 10 aa, Artificial Sequence
  Synthetic Polypeptide
NCAVSCAGEK

SEQ ID NO: 37 - protein, 29 aa, Artificial Sequence
  Synthetic Polypeptide
GCSTVLSPEGSAQFAAQIFGLSNHLVWSK

SEQ ID NO: 38 - protein, 13 aa, Artificial Sequence
  Synthetic Polypeptide
TIQFVDWCPTGFK

SEQ ID NO: 39 - protein, 31 aa, Artificial Sequence
  Synthetic Polypeptide
AGQPHSSSDAAQAPAEQPHSSSDAAQAPCPR

SEQ ID NO: 40 - protein, 20 aa, Artificial Sequence
  Synthetic Polypeptide
LALFNPDVCWDRNNPEPWNK

SEQ ID NO: 41 - protein, 12 aa, Artificial Sequence
  Synthetic Polypeptide
AVASQLDCNFLK

SEQ ID NO: 42 - protein, 16 aa, Artificial Sequence
  Synthetic Polypeptide
CSGIGDNPGSETAAPR

SEQ ID NO: 43 - protein, 20 aa, Artificial Sequence
  Synthetic Polypeptide
QPAIMPGQSYGLEDGSCSYK

SEQ ID NO: 44 - protein, 49 aa, Artificial Sequence
  Synthetic Polypeptide
GNVAGDSKNDPPMEAAGFTAQVIILNHPGQISAGYAPVLDCHTAHIACK

SEQ ID NO: 45 - protein, 12 aa, Artificial Sequence
  Synthetic Polypeptide
YWLCAATGPSIK

SEQ ID NO: 46 - protein, 10 aa, Artificial Sequence
  Synthetic Polypeptide
LQSGICHLFR

SEQ ID NO: 47 - protein, 12 aa, Artificial Sequence
  Synthetic Polypeptide
TIYAGNALCTVK

SEQ ID NO: 48 - protein, 17 aa, Artificial Sequence
  Synthetic Polypeptide
TSCGSPNYAAPEVISGR

SEQ ID NO: 49 - protein, 21 aa, Artificial Sequence
  Synthetic Polypeptide
TFCGTPEYLAPEVLEDNDYGR

SEQ ID NO: 50 - protein, 16 aa, Artificial Sequence
  Synthetic Polypeptide
ISCMSKPPAPNPTPPR

SEQ ID NO: 51 - protein, 32 aa, Artificial Sequence
  Synthetic Polypeptide
RPYGVGLLIAGYDDMGPHIFQTCPSANYFDCR

SEQ ID NO: 52 - protein, 32 aa, Artificial Sequence
  Synthetic Polypeptide
LADQCTGLQGFLVFHSFGGGTGSGFTSLLMER

SEQ ID NO: 53 - protein, 16 aa, Artificial Sequence
  Synthetic Polypeptide
LNLSCIHSPVVNELMR

SEQ ID NO: 54 - protein, 14 aa, Artificial Sequence
  Synthetic Polypeptide
LVVPATQCGSLIGK

SEQ ID NO: 55 - protein, 25 aa, Artificial Sequence
  Synthetic Polypeptide
NLSDLIDLVPSLCEDLLSSVDQPLK

SEQ ID NO: 56 - protein, 9 aa, Artificial Sequence
  Synthetic Polypeptide
SVHYCPATK

SEQ ID NO: 57 - protein, 24 aa, Artificial Sequence
  Synthetic Polypeptide
NFYGGNGIVGAQVPLGAGIALACK

SEQ ID NO: 58 - protein, 17 aa, Artificial Sequence
  Synthetic Polypeptide
HISPTAPDTLGCYPFYK

SEQ ID NO: 59 - protein, 24 aa, Artificial Sequence
  Synthetic Polypeptide
QGEYGLASICNGGGGASAMLIQKL

SEQ ID NO: 60 - protein, 16 aa, Artificial Sequence
  Synthetic Polypeptide
HSMNPFCEIAVEEAVR

SEQ ID NO: 61 - protein, 11 aa, Artificial Sequence
  Synthetic Polypeptide
LGMLSPEGTCK

SEQ ID NO: 62 - protein, 12 aa, Artificial Sequence
  Synthetic Polypeptide
GLCGAIHSSIAK

SEQ ID NO: 63 - protein, 30 aa, Artificial Sequence
  Synthetic Polypeptide
LVAFCPFASSQVALENANAVSEGVVHEDLR

SEQ ID NO: 64 - protein, 26 aa, Artificial Sequence
  Synthetic Polypeptide
GSDCGIVNVNIPTSGAEIGGAFGGEK

SEQ ID NO: 65 - protein, 10 aa, Artificial Sequence
  Synthetic Polypeptide
LNQQQHPDCK

SEQ ID NO: 66 - protein, 24 aa, Artificial Sequence
  Synthetic Polypeptide
CLHNFLTDGVPAEGAFTEDFQGLR

SEQ ID NO: 67 - protein, 13 aa, Artificial Sequence
  Synthetic Polypeptide
TPCNAGTFSQPEK

SEQ ID NO: 68 - protein, 28 aa, Artificial Sequence
  Synthetic Polypeptide
CPEALFQPSFLGMESCGIHETTFNSIMK

SEQ ID NO: 69 - protein, 14 aa, Artificial Sequence
  Synthetic Polypeptide
SWCPDCVQAEPVVR

SEQ ID NO: 70 - protein, 32 aa, Artificial Sequence
  Synthetic Polypeptide
KLDTNSDGQLDFSEFLNLIGGLAMACHDSFLK

SEQ ID NO: 71 - protein, 13 aa, Artificial Sequence
  Synthetic Polypeptide
SIQFVDWCPTGFK

SEQ ID NO: 72 - protein, 22 aa, Artificial Sequence
  Synthetic Polypeptide
SGGSGGCSGAGGASNCGTGSGR

SEQ ID NO: 73 - protein, 26 aa, Artificial Sequence
  Synthetic Polypeptide
FTLDCTHPVEDGIMDAANFEQFLQER

SEQ ID NO: 74 - protein, 13 aa, Artificial Sequence
  Synthetic Polypeptide
DLNYCFSGMSDHR

SEQ ID NO: 75 - protein, 16 aa, Artificial Sequence
  Synthetic Polypeptide
CEFEEVQGFLDQVAHK

SEQ ID NO: 76 - protein, 36 aa, Artificial Sequence
  Synthetic Polypeptide
NLPFPTYFPDGDEEELPEDLYDENVCQPGAPSITFA

SEQ ID NO: 77 - protein, 18 aa, Artificial Sequence
  Synthetic Polypeptide
GQVCLPVISAENWKPATK

SEQ ID NO: 78 - protein, 10 aa, Artificial Sequence
  Synthetic Polypeptide
CFGTGAAGNR

SEQ ID NO: 79 - protein, 17 aa, Artificial Sequence
  Synthetic Polypeptide
ADPDGPEAQAEACSGER

SEQ ID NO: 80 - protein, 13 aa, Artificial Sequence
  Synthetic Polypeptide
VHPAMATAAGGCR

SEQ ID NO: 81 - protein, 18 aa, Artificial Sequence
  Synthetic Polypeptide
EMFPYEASTPTGISASCR

SEQ ID NO: 82 - protein, 17 aa, Artificial Sequence
  Synthetic Polypeptide
LNIISNLDCVNEVIGIR

SEQ ID NO: 83 - protein, 9 aa, Artificial Sequence
  Synthetic Polypeptide
YMACCLLYR

SEQ ID NO: 84 - protein, 19 aa, Artificial Sequence
  Synthetic Polypeptide
SCSGVEFSTSGSSNTDTGK

SEQ ID NO: 85 - protein, 16 aa, Artificial Sequence
  Synthetic Polypeptide
AGSDGESIGNCPFSQR

SEQ ID NO: 86 - protein, 9 aa, Artificial Sequence
  Synthetic Polypeptide
GCEVVVSGK

SEQ ID NO: 87 - protein, 15 aa, Artificial Sequence
  Synthetic Polypeptide
NILGGTVFREPIICK

SEQ ID NO: 88 - protein, 22 aa, Artificial Sequence
  Synthetic Polypeptide
AGAIAPCEVTVPAQNTGLGPEK

SEQ ID NO: 89 - protein, 20 aa, Artificial Sequence
  Synthetic Polypeptide
TIGGGDDSFTTFFCETGAGK

SEQ ID NO: 90 - protein, 20 aa, Artificial Sequence
  Synthetic Polypeptide
FNNWGGSLSLGHPFGATGCR

SEQ ID NO: 91 - protein, 28 aa, Artificial Sequence
  Synthetic Polypeptide
NWYVQPSCATSGDGLYEGLTWLTSNYKS

SEQ ID NO: 92 - protein, 9 aa, Artificial Sequence
  Synthetic Polypeptide
APCQAGDLR

SEQ ID NO: 93 - protein, 25 aa, Artificial Sequence
  Synthetic Polypeptide
QVLMGPYNPDTCPEVGFFDVLGNDR

SEQ ID NO: 94 - protein, 36 aa, Artificial Sequence
  Synthetic Polypeptide
VTEDENDEPIEIPSEDDGTVLLSTVTAQFPGACGLR

SEQ ID NO: 95 - protein, 20 aa, Artificial Sequence
  Synthetic Polypeptide
IQCTLQDVGSALATPCSSAR

SEQ ID NO: 96 - protein, 12 aa, Artificial Sequence
  Synthetic Polypeptide
NLSFFLTPPCAR

SEQ ID NO: 97 - protein, 14 aa, Artificial Sequence
  Synthetic Polypeptide
LLACIASRPGQCGR

SEQ ID NO: 98 - protein, 22 aa, Artificial Sequence
  Synthetic Polypeptide
CSDNSSYEEPLSPISASSSTSR

SEQ ID NO: 99 - protein, 16 aa, Artificial Sequence
  Synthetic Polypeptide
CLAQEVNIPDWIVDLR

SEQ ID NO: 100 - protein, 9 aa, Artificial Sequence
  Synthetic Polypeptide
HEEFEEGCK

SEQ ID NO: 101 - protein, 10 aa, Artificial Sequence
  Synthetic Polypeptide
GLDYEGGGCR

SEQ ID NO: 102 - protein, 12 aa, Artificial Sequence
  Synthetic Polypeptide
LLQCDPSSASQF

SEQ ID NO: 103 - protein, 19 aa, Artificial Sequence
  Synthetic Polypeptide
TVPFLPLLGGCIDDTILSR

SEQ ID NO: 104 - protein, 10 aa, Artificial Sequence
  Synthetic Polypeptide
IINDNATYCR

SEQ ID NO: 105 - protein, 13 aa, Artificial Sequence
  Synthetic Polypeptide
SLLINAVEASCIR

SEQ ID NO: 106 - protein, 39 aa, Artificial Sequence
  Synthetic Polypeptide
PPMEAAGFTAQVIILNHPGQISAGYAPVLDCHTAHIACK

SEQ ID NO: 107 - protein, 21 aa, Artificial Sequence
  Synthetic Polypeptide
HPLTQELKECEGIVPVPLAEK

SEQ ID NO: 108 - protein, 27 aa, Artificial Sequence
  Synthetic Polypeptide
HTGPGILSMANAGPNTNGSQFFICTAK

SEQ ID NO: 109 - protein, 10 aa, Artificial Sequence
  Synthetic Polypeptide
IISDNLTYCK

SEQ ID NO: 110 - protein, 9 aa, Artificial Sequence
  Synthetic Polypeptide
LIDFLECGK

SEQ ID NO: 111 - protein, 8 aa, Artificial Sequence
  Synthetic Polypeptide
KAVVVCPK

SEQ ID NO: 112 - protein, 25 aa, Artificial Sequence
  Synthetic Polypeptide
LTTPTYGDLNHLVSATMSGVTTCLR

SEQ ID NO: 113 - protein, 12 aa, Artificial Sequence
  Synthetic Polypeptide
VCETDGCSSEAK

SEQ ID NO: 114 - protein, 18 aa, Artificial Sequence
  Synthetic Polypeptide
VCPPHMLPEDGANLSSAR

SEQ ID NO: 115 - protein, 15 aa, Artificial Sequence
  Synthetic Polypeptide
TFVDFFSQCLHEEYR

SEQ ID NO: 116 - protein, 18 aa, Artificial Sequence
  Synthetic Polypeptide
FALACNASDKIIEPIQSR

SEQ ID NO: 117 - protein, 15 aa, Artificial Sequence
  Synthetic Polypeptide
TIAECLADELINAAK

SEQ ID NO: 118 - protein, 23 aa, Artificial Sequence
  Synthetic Polypeptide
FMTPVIQDNPSGWGPCAVPEQFR

SEQ ID NO: 119 - protein, 23 aa, Artificial Sequence
  Synthetic Polypeptide
SVLLCGIEAQACILNTTLDLLDR

SEQ ID NO: 120 - protein, 10 aa, Artificial Sequence
  Synthetic Polypeptide
AVAILCNHQR

SEQ ID NO: 121 - protein, 27 aa, Artificial Sequence
  Synthetic Polypeptide
GNFTLPEVAECFDEITYVELQKEEAQK

SEQ ID NO: 122 - protein, 12 aa, Artificial Sequence
  Synthetic Polypeptide
TIIPLISQCTPK

SEQ ID NO: 123 - protein, 18 aa, Artificial Sequence
  Synthetic Polypeptide
DVQIGDIVTVGECRPLSK

SEQ ID NO: 124 - protein, 12 aa, Artificial Sequence
  Synthetic Polypeptide
LLDLVQQSCNYK

SEQ ID NO: 125 - protein, 10 aa, Artificial Sequence
  Synthetic Polypeptide
CMTNTPVVVR

SEQ ID NO: 126 - protein, 10 aa, Artificial Sequence
  Synthetic Polypeptide
VLGLGLGCLR

SEQ ID NO: 127 - protein, 22 aa, Artificial Sequence
  Synthetic Polypeptide
VCISILHAPGDDPMGYESSAER

SEQ ID NO: 128 - protein, 24 aa, Artificial Sequence
  Synthetic Polypeptide
AYHEQLSVAEITNACFEPANQMVK

SEQ ID NO: 129 - protein, 14 aa, Artificial Sequence
  Synthetic Polypeptide
AGQCVIGLQMGTNK

SEQ ID NO: 130 - protein, 28 aa, Artificial Sequence
  Synthetic Polypeptide
SGDAAIVDMVPGKPMCVESFSDYPPLGR

SEQ ID NO: 131 - protein, 39 aa, Artificial Sequence
  Synthetic Polypeptide
VSDTVVEPYNATLSVHQLVENTDETYCIDNEALYDICFR

SEQ ID NO: 132 - protein, 14 aa, Artificial Sequence
  Synthetic Polypeptide
CEFQDAYVLLSEKK

SEQ ID NO: 133 - protein, 11 aa, Artificial Sequence
  Synthetic Polypeptide
SSQCHTGSSPR

SEQ ID NO: 134 - protein, 12 aa, Artificial Sequence
  Synthetic Polypeptide
LNISFPATGCQK

SEQ ID NO: 135 - protein, 8 aa, Artificial Sequence
  Synthetic Polypeptide
FHADSVCK

SEQ ID NO: 136 - protein, 10 aa, Artificial Sequence
  Synthetic Polypeptide
CMQLTDFILK

SEQ ID NO: 137 - protein, 10 aa, Artificial Sequence
  Synthetic Polypeptide
ELEVLLMCNK

SEQ ID NO: 138 - protein, 15 aa, Artificial Sequence
  Synthetic Polypeptide
NHLLPDIVTCVQSSR

SEQ ID NO: 139 - protein, 22 aa, Artificial Sequence
  Synthetic Polypeptide
VNGTTPCAFPTHYEEALKDEEK

SEQ ID NO: 140 - protein, 8 aa, Artificial Sequence
  Synthetic Polypeptide
LTEGCSFR

SEQ ID NO: 141 - protein, 15 aa, Artificial Sequence
  Synthetic Polypeptide
CALSSPSLAFTPPIK

SEQ ID NO: 142 - protein, 24 aa, Artificial Sequence
  Synthetic Polypeptide
SSVQEECVSTISSSKDEDPLAATR

SEQ ID NO: 143 - protein, 12 aa, Artificial Sequence
  Synthetic Polypeptide
TQNLPNCQLISR

SEQ ID NO: 144 - protein, 32 aa, Artificial Sequence
  Synthetic Polypeptide
CSEGSFLLTTFPRPVTVEPMDQLDDEEGLPEK

SEQ ID NO: 145 - protein, 24 aa, Artificial Sequence
  Synthetic Polypeptide
SMVSPVPSPTGTISVPNSCPASPR

SEQ ID NO: 146 - protein, 30 aa, Artificial Sequence
  Synthetic Polypeptide
EITSLDTENIDEILNNADVALVNFYADWCR

SEQ ID NO: 147 - protein, 12 aa, Artificial Sequence
  Synthetic Polypeptide
GHSSDSNPAICR

SEQ ID NO: 148 - protein, 10 aa, Artificial Sequence
  Synthetic Polypeptide
FCIWTESAFR

SEQ ID NO: 149 - protein, 29 aa, Artificial Sequence
  Synthetic Polypeptide
SLCNLEESITSAGRDDLESFQLEISGFLK

SEQ ID NO: 150 - protein, 15 aa, Artificial Sequence
  Synthetic Polypeptide
IDCFSEVPTSVFGEK

SEQ ID NO: 151 - protein, 11 aa, Artificial Sequence
  Synthetic Polypeptide
NMSVHLSPCFR

SEQ ID NO: 152 - protein, 18 aa, Artificial Sequence
  Synthetic Polypeptide
GLPCTELFVAPVGVASKR

SEQ ID NO: 153 - protein, 15 aa, Artificial Sequence
  Synthetic Polypeptide
VACITEQVLTLVNKR

SEQ ID NO: 154 - protein, 26 aa, Artificial Sequence
  Synthetic Polypeptide
NSQWVPTLPNSSHHLDAVPCSTTINR

SEQ ID NO: 155 - protein, 19 aa, Artificial Sequence
  Synthetic Polypeptide
NVQLLSQFVSPFTGCIYGR

SEQ ID NO: 156 - protein, 12 aa, Artificial Sequence
  Synthetic Polypeptide
LCSSSSSDTSSR

SEQ ID NO: 157 - protein, 14 aa, Artificial Sequence
  Synthetic Polypeptide
LVVPASQCGSLIGK

SEQ ID NO: 158 - protein, 41 aa, Artificial Sequence
  Synthetic Polypeptide
VNAAGTDPSSPVGFVLGVDLLHIFPLEGATFLCPADVTDPR

SEQ ID NO: 159 - protein, 10 aa, Artificial Sequence
  Synthetic Polypeptide
CPFTGNVSIR

SEQ ID NO: 160 - protein, 21 aa, Artificial Sequence
  Synthetic Polypeptide
LTALDYHNPAGFNCKDETEFR

SEQ ID NO: 161 - protein, 14 aa, Artificial Sequence
  Synthetic Polypeptide
VPTANVSVVDLTCR

SEQ ID NO: 162 - protein, 20 aa, Artificial Sequence
  Synthetic Polypeptide
DSGYGDIWCPERGEFLAPPR

SEQ ID NO: 163 - protein, 24 aa, Artificial Sequence
  Synthetic Polypeptide
NFNYHILSPCDLSNYTDLAMSTVK

SEQ ID NO: 164 - protein, 14 aa, Artificial Sequence
  Synthetic Polypeptide
ACQSIYPLHDVFVR

SEQ ID NO: 165 - protein, 11 aa, Artificial Sequence
  Synthetic Polypeptide
WCEYGLTFTEK

SEQ ID NO: 166 - protein, 9 aa, Artificial Sequence
  Synthetic Polypeptide
KTPCGEGSK

SEQ ID NO: 167 - protein, 13 aa, Artificial Sequence
  Synthetic Polypeptide
NCLTNFHGMDLTR

SEQ ID NO: 168 - protein, 19 aa, Artificial Sequence
  Synthetic Polypeptide
YGIICMEDLIHEIYTVGKR

SEQ ID NO: 169 - protein, 13 aa, Artificial Sequence
  Synthetic Polypeptide
CASQAGMTAYGTR

SEQ ID NO: 170 - protein, 9 aa, Artificial Sequence
  Synthetic Polypeptide
KAQCPIVER

SEQ ID NO: 171 - protein, 12 aa, Artificial Sequence
  Synthetic Polypeptide
NGDICETSGKPK

SEQ ID NO: 172 - protein, 39 aa, Artificial Sequence
  Synthetic Polypeptide
KGAGNPQASTLALQSNITQCLLGQPWPLNEAQVQASVVK

SEQ ID NO: 173 - protein, 13 aa, Artificial Sequence
  Synthetic Polypeptide
FCSFSPCIEQVQR

SEQ ID NO: 174 - protein, 12 aa, Artificial Sequence
  Synthetic Polypeptide
IHMGSCAENTAK

SEQ ID NO: 175 - protein, 17 aa, Artificial Sequence
  Synthetic Polypeptide
GCIVDANLSVLNLVIVK

SEQ ID NO: 176 - protein, 14 aa, Artificial Sequence
  Synthetic Polypeptide
IAILTCPFEPPKPK

SEQ ID NO: 177 - protein, 19 aa, Artificial Sequence
  Synthetic Polypeptide
TLTSCFLSCVVCVEGIVTK

SEQ ID NO: 178 - protein, 16 aa, Artificial Sequence
  Synthetic Polypeptide
LGYILTCPSNLGTGLR

SEQ ID NO: 179 - protein, 16 aa, Artificial Sequence
  Synthetic Polypeptide
LMWLFGCPLLLDDVAR

SEQ ID NO: 180 - protein, 11 aa, Artificial Sequence
  Synthetic Polypeptide
INISEGNCPER

SEQ ID NO: 181 - protein, 11 aa, Artificial Sequence
  Synthetic Polypeptide
SGQGAFGNMCR

SEQ ID NO: 182 - protein, 12 aa, Artificial Sequence
  Synthetic Polypeptide
GLCAIAQAESLR

SEQ ID NO: 183 - protein, 36 aa, Artificial Sequence
  Synthetic Polypeptide
IIPTLEEGLQLPSPTATSQLPLESDAVECLNYQHYK

SEQ ID NO: 184 - protein, 28 aa, Artificial Sequence
  Synthetic Polypeptide
CPEALFQPSFLGMESCGIHETTFNSIMK

SEQ ID NO: 185 - protein, 10 aa, Artificial Sequence
  Synthetic Polypeptide
LCFSTAQHAS

SEQ ID NO: 186 - protein, 21 aa, Artificial Sequence
  Synthetic Polypeptide
LGTLAPFCCPWEQLTQDWESR

SEQ ID NO: 187 - protein, 17 aa, Artificial Sequence
  Synthetic Polypeptide
AAVEEGIVLGGGCALLR

SEQ ID NO: 188 - protein, 20 aa, Artificial Sequence
  Synthetic Polypeptide
LDSLGLCSVSCALEFIPNSK

SEQ ID NO: 189 - protein, 14 aa, Artificial Sequence
  Synthetic Polypeptide
VCNFLASQVPFPSR

SEQ ID NO: 190 - protein, 24 aa, Artificial Sequence
  Synthetic Polypeptide
VTDGALVVVDCVSGVCVQTETVLR

SEQ ID NO: 191 - protein, 19 aa, Artificial Sequence
  Synthetic Polypeptide
YAICSALAASALPALVMSK

SEQ ID NO: 192 - protein, 9 aa, Artificial Sequence
  Synthetic Polypeptide
TATAVAHCK

SEQ ID NO: 193 - protein, 11 aa, Artificial Sequence
  Synthetic Polypeptide
GCTATLGNFAK

SEQ ID NO: 194 - protein, 11 aa, Artificial Sequence
  Synthetic Polypeptide
VCNYGLTFTQK

SEQ ID NO: 195 - protein, 14 aa, Artificial Sequence
  Synthetic Polypeptide
SLHDALCVLAQTVK

SEQ ID NO: 196 - protein, 23 aa, Artificial Sequence
  Synthetic Polypeptide
AWVWNTHADFADECPKPELLAIR

SEQ ID NO: 197 - protein, 8 aa, Artificial Sequence
  Synthetic Polypeptide
CHTPPLYR

SEQ ID NO: 198 - protein, 20 aa, Artificial Sequence
  Synthetic Polypeptide
YIGENLQLLVDRPDGTYCFR

SEQ ID NO: 199 - protein, 43 aa, Artificial Sequence
  Synthetic Polypeptide
ALANVNIGSLICNVGAGGPAPAAGAAPAGGPAPSTAAAPAEEK

SEQ ID NO: 200 - protein, 20 aa, Artificial Sequence
  Synthetic Polypeptide
ANCIDSTASAEAVFASEVKK

SEQ ID NO: 201 - protein, 12 aa, Artificial Sequence
  Synthetic Polypeptide
NCIVLIDSTPYR

SEQ ID NO: 202 - protein, 37 aa, Artificial Sequence
  Synthetic Polypeptide
YSTGSDSASFPHTTPSMCLNPDLEGPPLEAYTIQGQY

SEQ ID NO: 203 - protein, 9 aa, Artificial Sequence
  Synthetic Polypeptide
LGEWVGLCK

SEQ ID NO: 204 - protein, 13 aa, Artificial Sequence
  Synthetic Polypeptide
CASQVGMTAPGTR

SEQ ID NO: 205 - protein, 18 aa, Artificial Sequence
  Synthetic Polypeptide
TTSSANNPNLMYQDECDR

SEQ ID NO: 206 - protein, 20 aa, Artificial Sequence
  Synthetic Polypeptide
ISLGLPVGAVINCADNTGAK

SEQ ID NO: 207 - protein, 9 aa, Artificial Sequence
  Synthetic Polypeptide
AYGGSMCAK

SEQ ID NO: 208 - protein, 21 aa, Artificial Sequence
  Synthetic Polypeptide
VVNSETPVVVDFHAQWCGPCK

SEQ ID NO: 209 - protein, 16 aa, Artificial Sequence
  Synthetic Polypeptide
GVPGAIVNVSSQCSQR

SEQ ID NO: 210 - protein, 13 aa, Artificial Sequence
  Synthetic Polypeptide
GCLWALNPAKIDK

SEQ ID NO: 211 - protein, 27 aa, Artificial Sequence
  Synthetic Polypeptide
DLCFSPGLMEASHVVNDVNEAVQLVFR

SEQ ID NO: 212 - protein, 13 aa, Artificial Sequence
  Synthetic Polypeptide
HGFCGIPITDTGR

SEQ ID NO: 213 - protein, 11 aa, Artificial Sequence
  Synthetic Polypeptide
ASDHGWVCDQR

SEQ ID NO: 214 - protein, 17 aa, Artificial Sequence
  Synthetic Polypeptide
AVCMLSNTTAVAEAWAR

SEQ ID NO: 215 - protein, 10 aa, Artificial Sequence
  Synthetic Polypeptide
HIGDGCCLTR

SEQ ID NO: 216 - protein, 8 aa, Artificial Sequence
  Synthetic Polypeptide
SDLGPCEK

SEQ ID NO: 217 - protein, 26 aa, Artificial Sequence
  Synthetic Polypeptide
LPITVLNGAPGFINLCDALNAWQLVK

SEQ ID NO: 218 - protein, 27 aa, Artificial Sequence
  Synthetic Polypeptide
VDEFPLCGHMVSDEYEQLSSEALEAAR

SEQ ID NO: 219 - protein, 17 aa, Artificial Sequence
  Synthetic Polypeptide
IISNASCTTNCLAPLAK

SEQ ID NO: 220 - protein, 13 aa, Artificial Sequence
  Synthetic Polypeptide
DLTTAGAVTQCYR

SEQ ID NO: 221 - protein, 24 aa, Artificial Sequence
  Synthetic Polypeptide
SACSLESNLEGLAGVLEADLPNYK

SEQ ID NO: 222 - protein, 28 aa, Artificial Sequence
  Synthetic Polypeptide
KITAFVPNDGCLNFIEENDEVLVAGFGR

SEQ ID NO: 223 - protein, 12 aa, Artificial Sequence
  Synthetic Polypeptide
TGCTFPEKPDFH

SEQ ID NO: 224 - protein, 22 aa, Artificial Sequence
  Synthetic Polypeptide
NQSFCPTVNLDKLWTLVSEQTR

SEQ ID NO: 225 - protein, 20 aa, Artificial Sequence
  Synthetic Polypeptide
SGEEDFESLASQFSDCSSAK

SEQ ID NO: 226 - protein, 36 aa, Artificial Sequence
  Synthetic Polypeptide
SPWLAGNELTVADVVLWSVLQQIGGCSVTVPANVQR

SEQ ID NO: 227 - protein, 13 aa, Artificial Sequence
  Synthetic Polypeptide
VNQAIWLLCTGAR

SEQ ID NO: 228 - protein, 15 aa, Artificial Sequence
  Synthetic Polypeptide
ACDLPAWVHFPDTER

SEQ ID NO: 229 - protein, 27 aa, Artificial Sequence
  Synthetic Polypeptide
YYALCGFGGVLSCGLTHTAVVPLDLVK

SEQ ID NO: 230 - protein, 10 aa, Artificial Sequence
  Synthetic Polypeptide
ECLPLIIFLR

SEQ ID NO: 231 - protein, 10 aa, Artificial Sequence
  Synthetic Polypeptide
VLVTTNVCAR

SEQ ID NO: 232 - protein, 13 aa, Artificial Sequence
  Synthetic Polypeptide
SYCAEIAHNVSSK

SEQ ID NO: 233 - protein, 28 aa, Artificial Sequence
  Synthetic Polypeptide
TAFQEALDAAGDKLVVVDFSATWCGPCK

SEQ ID NO: 234 - protein, 23 aa, Artificial Sequence
  Synthetic Polypeptide
LCYVALDFEQEMATAASSSSLEK

SEQ ID NO: 235 - protein, 24 aa, Artificial Sequence
  Synthetic Polypeptide
VRPSTGNSASTPQSQCLPSEIEVK

SEQ ID NO: 236 - protein, 20 aa, Artificial Sequence
  Synthetic Polypeptide
AATGEEVSAEDLGGADLHCR

SEQ ID NO: 237 - protein, 31 aa, Artificial Sequence
  Synthetic Polypeptide
PGHLQEGFGCVVTNRFDQLFDDESDPFEVLK

SEQ ID NO: 238 - protein, 13 aa, Artificial Sequence
  Synthetic Polypeptide
CASQSGMTAYGTR

SEQ ID NO: 239 - protein, 11 aa, Artificial Sequence
  Synthetic Polypeptide
LCVQNSPQEAR

SEQ ID NO: 240 - protein, 14 aa, Artificial Sequence
  Synthetic Polypeptide
PCSEETPAISPSKR

SEQ ID NO: 241 - protein, 22 aa, Artificial Sequence
  Synthetic Polypeptide
LDINLLDNVVNCLYHGEGAQQR

SEQ ID NO: 242 - protein, 9 aa, Artificial Sequence
  Synthetic Polypeptide
NMMAACDPR

SEQ ID NO: 243 - protein, 8 aa, Artificial Sequence
  Synthetic Polypeptide
IAVHCTVR

SEQ ID NO: 244 - protein, 15 aa, Artificial Sequence
  Synthetic Polypeptide
CPQVEEAIVQSGQKK

SEQ ID NO: 245 - protein, 16 aa, Artificial Sequence
  Synthetic Polypeptide
SQAPCANKDEADLSSK

SEQ ID NO: 246 - protein, 13 aa, Artificial Sequence
  Synthetic Polypeptide
CLGHPEEFYNLVR

SEQ ID NO: 247 - protein, 18 aa, Artificial Sequence
  Synthetic Polypeptide
NTVLCNVVEQFLQADLAR

SEQ ID NO: 248 - protein, 31 aa, Artificial Sequence
  Synthetic Polypeptide
AFQYVETHGEVCPANWTPDSPTIKPSPAASK

SEQ ID NO: 249 - protein, 19 aa, Artificial Sequence
  Synthetic Polypeptide
QAVLGAGLPISTPCTTINK

SEQ ID NO: 250 - protein, 19 aa, Artificial Sequence
  Synthetic Polypeptide
GTPEQPQCGFSNAVVQILR

SEQ ID NO: 251 - protein, 27 aa, Artificial Sequence
  Synthetic Polypeptide
ADHQPLTEASYVNLPTIALCNTDSPLR

SEQ ID NO: 252 - protein, 32 aa, Artificial Sequence
  Synthetic Polypeptide
GSDELFSTCVTNGPFIMSSNSASAANGNDSKK

SEQ ID NO: 253 - protein, 24 aa, Artificial Sequence
  Synthetic Polypeptide
AYHEQLTVAEITNACFEPANQMVK

SEQ ID NO: 254 - protein, 18 aa, Artificial Sequence
  Synthetic Polypeptide
INPYMSSPCHIEMILTEK

SEQ ID NO: 255 - protein, 23 aa, Artificial Sequence
  Synthetic Polypeptide
YIYDQCPAVAGYGPIEQLPDYNR

SEQ ID NO: 256 - protein, 24 aa, Artificial Sequence
  Synthetic Polypeptide
CELSSSVQTDINLPYLTMDSSGPK

SEQ ID NO: 257 - protein, 12 aa, Artificial Sequence
  Synthetic Polypeptide
GCLLYGPPGTGK

SEQ ID NO: 258 - protein, 18 aa, Artificial Sequence
  Synthetic Polypeptide
ALNALCDGLIDELNQALK

SEQ ID NO: 259 - protein, 11 aa, Artificial Sequence
  Synthetic Polypeptide
NTPSFLIACNK

SEQ ID NO: 260 - protein, 9 aa, Artificial Sequence
  Synthetic Polypeptide
CMPTFQFFK

SEQ ID NO: 261 - protein, 11 aa, Artificial Sequence
  Synthetic Polypeptide
TGNGPMSVCGR

SEQ ID NO: 262 - protein, 14 aa, Artificial Sequence
  Synthetic Polypeptide
HELQANCYEEVKDR

SEQ ID NO: 263 - protein, 24 aa, Artificial Sequence
  Synthetic Polypeptide
AFAFVTFADDQIAQSLCGEDLIIK

SEQ ID NO: 264 - protein, 28 aa, Artificial Sequence
  Synthetic Polypeptide
LTPGCEAEAETEAICFFVQQFTDMEHNR

SEQ ID NO: 265 - protein, 9 aa, Artificial Sequence
  Synthetic Polypeptide
LECVEPNCR

SEQ ID NO: 266 - protein, 12 aa, Artificial Sequence
  Synthetic Polypeptide
SYCNDQSTGDIK

SEQ ID NO: 267 - protein, 19 aa, Artificial Sequence
  Synthetic Polypeptide
ATCAPQHGAPGPGPADASK

SEQ ID NO: 268 - protein, 8 aa, Artificial Sequence
  Synthetic Polypeptide
ATYDKLCK

SEQ ID NO: 269 - protein, 14 aa, Artificial Sequence
  Synthetic Polypeptide
GSCSTEVEKETQEK

SEQ ID NO: 270 - protein, 18 aa, Artificial Sequence
  Synthetic Polypeptide
TVDSQGPTPVCTPTFLER

SEQ ID NO: 271 - protein, 23 aa, Artificial Sequence
  Synthetic Polypeptide
LCYVALDFEQEMAMVASSSSLEK

SEQ ID NO: 272 - protein, 19 aa, Artificial Sequence
  Synthetic Polypeptide
VFIMDSCDELIPEYLNFIR

SEQ ID NO: 273 - protein, 9 aa, Artificial Sequence
  Synthetic Polypeptide
HCSQVDSVR

SEQ ID NO: 274 - protein, 22 aa, Artificial Sequence
  Synthetic Polypeptide
AAAPAPEEEMDECEQALAAEPK

SEQ ID NO: 275 - protein, 9 aa, Artificial Sequence
  Synthetic Polypeptide
EKTACAINK

SEQ ID NO: 276 - protein, 30 aa, Artificial Sequence
  Synthetic Polypeptide
SGTICSSELPGAFEAAGFHLNEHLYNMIIR

SEQ ID NO: 277 - protein, 27 aa, Artificial Sequence
  Synthetic Polypeptide
TASISSSPSEGTPTVGSYGCTPQSLPK

SEQ ID NO: 278 - protein, 17 aa, Artificial Sequence
  Synthetic Polypeptide
EVIAVSCGPAQCQETIR

SEQ ID NO: 279 - protein, 12 aa, Artificial Sequence
  Synthetic Polypeptide
NCPHVVVGTPGR

SEQ ID NO: 280 - protein, 13 aa, Artificial Sequence
  Synthetic Polypeptide
SCPSFSASSEGTR

SEQ ID NO: 281 - protein, 23 aa, Artificial Sequence
  Synthetic Polypeptide
GLIAAICAGPTALLAHEIGFGSK

SEQ ID NO: 282 - protein, 14 aa, Artificial Sequence
  Synthetic Polypeptide
LYYFQYPCYQEGLR

SEQ ID NO: 283 - protein, 14 aa, Artificial Sequence
  Synthetic Polypeptide
GCWDSIHVVEVQEK

SEQ ID NO: 284 - protein, 10 aa, Artificial Sequence
  Synthetic Polypeptide
SCYDLSCHAR

SEQ ID NO: 285 - protein, 19 aa, Artificial Sequence
  Synthetic Polypeptide
VGSFCLSEAGAGSDSFALK

SEQ ID NO: 286 - protein, 17 aa, Artificial Sequence
  Synthetic Polypeptide
AINCATSGVVGLVNCLR

SEQ ID NO: 287 - protein, 10 aa, Artificial Sequence
  Synthetic Polypeptide
TSAVPSPCGK

SEQ ID NO: 288 - protein, 21 aa, Artificial Sequence
  Synthetic Polypeptide
INEIVYFLPFCHSELIQLVNK

SEQ ID NO: 289 - protein, 30 aa, Artificial Sequence
  Synthetic Polypeptide
IGFPETTEEELEEIASENSDCIFPSAPDVK

SEQ ID NO: 290 - protein, 27 aa, Artificial Sequence
  Synthetic Polypeptide
IVGYFVSGCDPSIMGIGPVPAISGALK

SEQ ID NO: 291 - protein, 15 aa, Artificial Sequence
  Synthetic Polypeptide
RGPCIIYNEDNGIIK

SEQ ID NO: 292 - protein, 21 aa, Artificial Sequence
  Synthetic Polypeptide
AALVTSFCMFKYMALYSMIQR

SEQ ID NO: 293 - protein, 21 aa, Artificial Sequence
  Synthetic Polypeptide
ESLNASIVDAINQAADCWGIR

SEQ ID NO: 294 - protein, 8 aa, Artificial Sequence
  Synthetic Polypeptide
EEHLCTQR

SEQ ID NO: 295 - protein, 15 aa, Artificial Sequence
  Synthetic Polypeptide
GCGVVKFESPEVAER

SEQ ID NO: 296 - protein, 7 aa, Artificial Sequence
  Synthetic Polypeptide
KCSASNR

SEQ ID NO: 297 - protein, 19 aa, Artificial Sequence
  Synthetic Polypeptide
TPCSSLLPLLNAHAATSGK

SEQ ID NO: 298 - protein, 36 aa, Artificial Sequence
  Synthetic Polypeptide
VSLDPELEEALTSASDTELCDLAAILGMHNLITNTK

SEQ ID NO: 299 - protein, 26 aa, Artificial Sequence
  Synthetic Polypeptide
FDPTQFQDCIIQGLTETGTDLEAVAK

SEQ ID NO: 300 - protein, 14 aa, Artificial Sequence
  Synthetic Polypeptide
LGGSLIVAFEGCPV

SEQ ID NO: 301 - protein, 9 aa, Artificial Sequence
  Synthetic Polypeptide
TQYSCYCCK

SEQ ID NO: 302 - protein, 13 aa, Artificial Sequence
  Synthetic Polypeptide
ICPVEFNPNFVAR

SEQ ID NO: 303 - protein, 25 aa, Artificial Sequence
  Synthetic Polypeptide
TPSYSISSTLNPQAPEFILGCTASK

SEQ ID NO: 304 - protein, 12 aa, Artificial Sequence
  Synthetic Polypeptide
ASVGFGGSCFQK

SEQ ID NO: 305 - protein, 16 aa, Artificial Sequence
  Synthetic Polypeptide
NTGQTCVCSNQFLVQR

SEQ ID NO: 306 - protein, 32 aa, Artificial Sequence
  Synthetic Polypeptide
FQSSAVMALQEASEAYLVGLFEDTNLCAIHAK

SEQ ID NO: 307 - protein, 26 aa, Artificial Sequence
  Synthetic Polypeptide
AGAVVAVPTDTLYGLACAASCSAALR

SEQ ID NO: 308 - protein, 19 aa, Artificial Sequence
  Synthetic Polypeptide
AVLLASDAQECTLEEVVER

SEQ ID NO: 309 - protein, 32 aa, Artificial Sequence
  Synthetic Polypeptide
FQSSAVMALQEACEAYLVGLFEDTNLCAIHAK

SEQ ID NO: 310 - protein, 29 aa, Artificial Sequence
  Synthetic Polypeptide
NMITGTSQADCAVLIVAAGVGEFEAGISK

SEQ ID NO: 311 - protein, 10 aa, Artificial Sequence
  Synthetic Polypeptide
GNHECASINR

SEQ ID NO: 312 - protein, 22 aa, Artificial Sequence
  Synthetic Polypeptide
LCDFGVSGQLIDSMANSFVGTR

SEQ ID NO: 313 - protein, 18 aa, Artificial Sequence
  Synthetic Polypeptide
SCGSSTPDEFPTDIPGTK

SEQ ID NO: 314 - protein, 12 aa, Artificial Sequence
  Synthetic Polypeptide
GTLTLCPYHSDR

SEQ ID NO: 315 - protein, 16 aa, Artificial Sequence
  Synthetic Polypeptide
LSLDGQNIYNACCTLR

SEQ ID NO: 316 - protein, 27 aa, Artificial Sequence
  Synthetic Polypeptide
ADASSTPSFQQAFASSCTISSNGPGQR

SEQ ID NO: 317 - protein, 10 aa, Artificial Sequence
  Synthetic Polypeptide
STLTDSLVCK

SEQ ID NO: 318 - protein, 31 aa, Artificial Sequence
  Synthetic Polypeptide
SDITKLEVDAIVNAANSSLLGGGGVDGCIHR

SEQ ID NO: 319 - protein, 19 aa, Artificial Sequence
  Synthetic Polypeptide
LCEPEVLNSLEETYSPFFR

SEQ ID NO: 320 - protein, 20 aa, Artificial Sequence
  Synthetic Polypeptide
CPAPPRGPPAPAPEVEELAR

SEQ ID NO: 321 - protein, 17 aa, Artificial Sequence
  Synthetic Polypeptide
AACLESAQEPAGAWGNK

SEQ ID NO: 322 - protein, 11 aa, Artificial Sequence
  Synthetic Polypeptide
LHTGPLPEQCR

SEQ ID NO: 323 - protein, 28 aa, Artificial Sequence
  Synthetic Polypeptide
FQSAAIGALQEASEAYLVGLFEDTNLCA

SEQ ID NO: 324 - protein, 32 aa, Artificial Sequence
  Synthetic Polypeptide
FQSSAVMALQEAREAYLVGLFEDTNLCAIHAK

SEQ ID NO: 325 - protein, 17 aa, Artificial Sequence
  Synthetic Polypeptide
HLNEIDLFHCIDPNDSK

SEQ ID NO: 326 - protein, 25 aa, Artificial Sequence
  Synthetic Polypeptide
QCPIMDPAWEAPEGVPIDAIIFGGR

SEQ ID NO: 327 - protein, 40 aa, Artificial Sequence
  Synthetic Polypeptide
SECDQDYIPETDQDCSMSPCPQRTPDSGLAQHPFQNEDYR

SEQ ID NO: 328 - DNA, 59 bp, Artificial Sequence
  Synthetic Polynucleotide
ccgggcagtgttgatgtgggtatttctcgagaaatacccacatcaacactgcttttttg

SEQ ID NO: 329 - DNA, 59 bp, Artificial Sequence
  Synthetic Polynucleotide
ccgggctatatctgaggagttggttctcgagaaccaactcctcagatatagcttttttg

SEQ ID NO: 330 - DNA, 22 bp, Artificial Sequence
  Synthetic Polynucleotide
gcaagctgaccctgaagttcat

SEQ ID NO: 331 - protein, 1192 aa, Homo sapiens
MEDLDQSPLVSSSDSPPRPQPAFKYQFVREPEDEEEEEEEEEEDEDEDLEELEVLERKPAAGLSAAPVPTAPAAGAPLMDFGNDFVPPAPRGPLPAAPPVAPERQPSWDPSPVSSTVPAPSPLSAAAVSPSKLPEDDEPPARPPPPPPASVSPQAEPVWTPPAPAPAAPPSTPAAPKRRGSSGSVDETLFALPAASEPVIRSSAENMDLKEQPGNTISAGQEDFPSVLLETAASLPSLSPLSAASFKEHEYLGNLSTVLPTEGTLQENVSEASKEVSEKAKTLLIDRDLTEFSELEYSEMGSSFSVSPKAESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEKDSESSNDDTSFPSTPEGIKDRSGAYITCAPFNPAATESIATNIFPLLGDPTSENKTDEKKIEEKKAQIVTEKNTSTKTSNPFLVAAQDSETDYVTTDNLTKVTEEVVANMPEGLTPDLVQEACESELNEVTGTKIAYETKMDLVQTSEVMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLFSDDSIPDVPQKQDETVMLVKESLTETSFESMIEYENKEKLSALPPEGGKPYLESFKLSLDNTKDTLLPDEVSTLSKKEKIPLQMEELSTAVYSNDDLFISKEAQIRETETFSDSSPIEIIDEFPTLISSKTDSFSKLAREYTDLEVSHKSEIANAPDGAGSLPCTELPHDLSLKNIQPKVEEKISFSDDFSKNGSATSKVLLLPPDVSALATQAEIESIVKPKVLVKEAEKKLPSDTEKEDRSPSAIFSAELSKTSVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 332 - protein, 1043 aa, Xenopus laevis
MDEQSPDISSSHSGDERREPAQPGERKPWDDLDDVLDLTGGAGQFSQPFSGSHPARDIEEEEEDEEEERGAWKDSLEPSPVEEEPGSIDSISPVSPHSPAVPSAPMEEPERPPAPCTAPSGSVDENLFTLPAASAHLMHASADKIMEPYSTVSTGQEEFASVLLQSTASLSSLPSLSTDSSKEHAETVAFPTGLAATEALQEPTDNMYSVSRITSHLPLSDNLESKALDQVKEEVIFSEKGYVVDHPTSQQETISEEHAKLYSQSAKEMFSGMLQSVAPPHEEFTDIKEVYDPYVDFKPFMSSKSGDVGYEVSDVAEKFQVDVGRLNLESAVKHEEKSSEEMEIDSISDDISPLTPELLPDSTDYDMFATVEQNIPFSFGGGHVAGNKTDEKKIEDIEAQKTSVGFGLKVATVNPFYNESAQESEYVTTHVATHVSTKPEGPTPDIVQEAYESEAYDTGIPKQKYESNIDLVQTAANSVQEKVSPTAQAPARLEETDSVSSPVLPDIVMEAPLASALETVALKPDISPVGIKPPARVEKTKAEPEKPPSYEEAVTEVLQNQDLAAALGGSKQGAVVEETETPYISIACDLIKGTESVASGFTEFSKLKQNEFESQFMEPSDESSPDSECSEPSYKQWDSEVVQKEAFSIKTESVNAQSIIIPEQKQVFDQKSEESSPSKSYLDSFQPEICVSKATSDLFAKGLTTLLQEKPLQMEELDEGLSLEKIPCTKYSPVSESPEPRPSPVPEDLSSKLGDIQKEVLIAKQPEDKVQKNRSNLDFVPENIEFTPAVQKPDDSGKAVSDTFGGLDTTTKGGSAVHEVKVDKPKPPSKEDDGSKLPKKESKASTVSSSDFMNSVVDLIYWRDIKRSGVVFGASLFLLLSLSVFSIVSVLAYIALALLSVTISLRIYKGILQAIQKSEEGHPFRSILESNLAVPEDLVQKYCNVALNHVNCTVKELRHLFLVEDLVDSLKFAVLMWVFTYIGALFNGLTLLIVALISLFSIPVIYERHQTQVDHYLALVNKNLKSTSDLILSKVPGLKRKAE

SEQ ID NO: 333 - protein, 545 aa, Homo sapiens
MGQVLPVFAHCKEAPSTASSTPDSTEGGNDDSDFRELHTAREFSEEDEEETTSQDWGTPRELTFSYIAFDGVVGSGGRRDSTARRPRPQGRSVSEPRDQHPQPSLGDSLESIPSLSQSPEPGRRGDPDTAPPSERPLEDLRLRLDHLGWVARGTGSGEDSSTSSSTPLEDEEPQEPNRLETGEAGEELDLRLRLAQPSSPEVLTPQLSPGSGTPQAGTPSPSRSRDSNSGPEEPLLEEEEKQWGPLEREPVRGQCLDSTDQLEFTVEPRLLGTAMEWLKTSLLLAVYKTVPILELSPPLWTAIGWVQRGPTPPTPVLRVLLKWAKSPRSSGVPSLSLGADMGSKVADLLYWKDTRTSGVVFTGLMVSLLCLLHFSIVSVAAHLALLLLCGTISLRVYRKVLQAVHRGDGANPFQAYLDVDLTLTREQTERLSHQITSRVVSAATQLRHFFLVEDLVDSLKLALLFYILTFVGAIFNGLTLLILGVIGLFTIPLLYRQHQAQIDQYVGLVTNQLSHIKAKIRAKIPGTGALASAAAAVSGSKAKAE

SEQ ID NO: 334 - protein, 472 aa, Artificial Sequence
  Synthetic Polypeptide
MGQVLPVFAHCKEAPSTASSTPDSTEGGNDDSDFRELHTAREFSEEDEEETTSQDWGTPRELTFSYIAFDGVVGSGGRRDSTARRPRPQGRSVSEPRDQHPQPSLGDSLESIPSLSQSPEPGRRGDPDTAPPSERPLEDLRLRLDHLGWVARGTGSGEDSSTSSSTPLEDEEPQEPNRLETGEAGEELDLRLRLAQPSSPEVLTPQLSPGSGTPQAGTPSPSRSRDSNSGPEEPLLEEEEKQWGPLEREPVRGQCLDSTDQLEFTVEPRLLVADLLYWKDTRTSGVVFTGLMVSLLCLLHFSIVSVAAHLALLLLCGTISLRVYRKVLQAVHRGDGANPFQAYLDVDLTLTREQTERLSHQITSRVVSAATQLRHFFLVEDLVDSLKLALLFYILTFVGAIFNGLTLLILGVIGLFTIPLLYRQHQAQIDQYVGLVTNQLSHIKAKIRAKIPGTGALASAAAAVSGSKAKAE

SEQ ID NO: 335 - protein, 1032 aa, Homo sapiens
MAEPSAATQSHSISSSSFGAEPSAPGGGGSPGACPALGTKSCSSSCADSFVSSSSSQPVSLFSTSQEGLSSLCSDEPSSEIMTSSFLSSSEIHNTGLTILHGEKSHVLGSQPILAKEGKDHLDLLDMKKMEKPQGTSNNVSDSSVSLAAGVHCDRPSIPASFPEHPAFLSKKIGQVEEQIDKETKNPNGVSSREAKTALDADDRFTLLTAQKPPTEYSKVEGIYTYSLSPSKVSGDDVIEKDSPESPFEVIIDKAAFDKEFKDSYKESTDDFGSWSVHTDKESSEDISETNDKLFPLRNKEAGRYPMSALLSRQFSHTNAALEEVSRCVNDMHNFTNEILTWDLVPQVKQQTDKSSDCITKTTGLDMSEYNSEIPVVNLKTSTHQKTPVCSIDGSTPITKSTGDWAEASLQQENAITGKPVPDSLNSTKEFSIKGVQGNMQKQDDTLAELPGSPPEKCDSLGSGVATVKVVLPDDHLKDEMDWQSSALGEITEADSSGESDDTVIEDITADTSFENNKIQAEKPVSIPSAVVKTGEREIKEIPSCEREEKTSKNFEELVSDSELHQDQPDILGRSPASEAACSKVPDTNVSLEDVSEVAPEKPITTENPKLPSTVSPNVFNETEFSLNVTTSAYLESLHGKNVKHIDDSSPEDLIAAFTETRDKGIVDSERNAFKAISEKMTDFKTTPPVEVLHENESGGSEIKDIGSKYSEQSKETNGSEPLGVFPTQGTPVASLDLEQEQLTIKALKELGERQVEKSTSAQRDAELPSEEVLKQTFTFAPESWPQRSYDILERNVKNGSDLGISQKPITIRETTRVDAVSSLSKTELVKKHVLARLLTDFSVHDLIFWRDVKKTGFVFGTTLIMLLSLAAFSVISVVSYLILALLSVTISFRIYKSVIQAVQKSEEGHPFKAYLDVDITLSSEAFHNYMNAAMVHINRALKLIIRLFLVEDLVDSLKLAVFMWLMTYVGAVFNGITLLILAELLIFSVPIVYEKYKTQIDHYVGIARDQTKSIVEKIQAKLPGIAKKKAE

SEQ ID NO: 336 - protein, 1013 aa, Artificial Sequence
  Synthetic Polypeptide
MAEPSAATQSHSISSSSFGAEPSAPGGGGSPGACPALGTKSCSSSCAEGLSSLCSDEPSSEIMTSSFLSSSEIHNTGLTILHGEKSHVLGSQPILAKEGKDHLDLLDMKKMEKPQGTSNNVSDSSVSLAAGVHCDRPSIPASFPEHPAFLSKKIGQVEEQIDKETKNPNGVSSREAKTALDADDRFTLLTAQKPPTEYSKVEGIYTYSLSPSKVSGDDVIEKDSPESPFEVIIDKAAFDKEFKDSYKESTDDFGSWSVHTDKESSEDISETNDKLFPLRNKEAGRYPMSALLSRQFSHTNAALEEVSRCVNDMHNFTNEILTWDLVPQVKQQTDKSSDCITKTTGLDMSEYNSEIPVVNLKTSTHQKTPVCSIDGSTPITKSTGDWAEASLQQENAITGKPVPDSLNSTKEFSIKGVQGNMQKQDDTLAELPGSPPEKCDSLGSGVATVKVVLPDDHLKDEMDWQSSALGEITEADSSGESDDTVIEDITADTSFENNKIQAEKPVSIPSAVVKTGEREIKEIPSCEREEKTSKNFEELVSDSELHQDQPDILGRSPASEAACSKVPDTNVSLEDVSEVAPEKPITTENPKLPSTVSPNVFNETEFSLNVTTSAYLESLHGKNVKHIDDSSPEDLIAAFTETRDKGIVDSERNAFKAISEKMTDFKTTPPVEVLHENESGGSEIKDIGSKYSEQSKETNGSEPLGVFPTQGTPVASLDLEQEQLTIKALKELGERQVEKSTSAQRDAELPSEEVLKQTFTFAPESWPQRSYDILERNVKNGSDLGISQKPITIRETTRVDAVSSLSKTELVKKHVLARLLTDFSVHDLIFWRDVKKTGFVFGTTLIMLLSLAAFSVISVVSYLILALLSVTISFRIYKSVIQAVQKSEEGHPFKAYLDVDITLSSEAFHNYMNAAMVHINRALKLIIRLFLVEDLVDSLKLAVFMWLMTYVGAVFNGITLLILAELLIFSVPIVYEKYKTQIDHYVGIARDQTKSIVEKIQAKLPGIAKKKAE

SEQ ID NO: 337 - protein, 236 aa, Artificial Sequence
  Synthetic Polypeptide
MAEPSAATQSHSISSSSFGAEPSAPGGGGSPGACPALGTKSCSSSCAVHDLIFWRDVKKTGFVFGTTLIMLLSLAAFSVISVVSYLILALLSVTISFRIYKSVIQAVQKSEEGHPFKAYLDVDITLSSEAFHNYMNAAMVHINRALKLIIRLFLVEDLVDSLKLAVFMWLMTYVGAVFNGITLLILAELLIFSVPIVYEKYKTQIDHYVGIARDQTKSIVEKIQAKLPGIAKKKAE

SEQ ID NO: 338 - protein, 776 aa, Homo sapiens
MAAPGDPQDELLPLAGPGSQWLRHRGEGENEAVTPKGATPAPQAGEPSPGLGARAREAASREAGSGPARQSPVAMETASTGVAGVSSAMDHTFSTTSKDGEGSCYTSLISDICYPPQEDSTYFTGILQKENGHVTISESPEELGTPGPSLPDVPGIESRGLFSSDSGIEMTPAESTEVNKILADPLDQMKAEAYKYIDITRPEEVKHQEQHHPELEDKDLDFKNKDTDISIKPEGVREPDKPAPVEGKIIKDHLLEESTFAPYIDDLSEEQRRAPQITTPVKITLTEIEPSVETTTQEKTPEKQDICLKPSPDTVPTVTVSEPEDDSPGSITPPSSGTEPSAAESQGKGSISEDELITAIKEAKGLSYETAENPRPVGQLADRPEVKARSGPPTIPSPLDHEASSAESGDSEIELVSEDPMAAEDALPSGYVSFGHVGGPPPSPASPSIQYSILREEREAELDSELIIESCDASSASEESPKREQDSPPMKPSALDAIREETGVRAEERAPSRRGLAEPGSFLDYPSTEPQPGPELPPGDGALEPETPMLPRKPEEDSSSNQSPAATKGPGPLGPGAPPPLLFLNKQKAIDLLYWRDIKQTGIVFGSFLLLLFSLTQFSVVSVVAYLALAALSATISFRIYKSVLQAVQKTDEGHPFKAYLELEITLSQEQIQKYTDCLQFYVNSTLKELRRLFLVQDLVDSLKFAVLMWLLTYVGALFNGLTLLLMAVVSMFTLPVVYVKHQAQIDQYLGLVRTHINAVVAKIQAKIPGAKRHAE

SEQ ID NO: 339 - protein, 356 aa, Artificial Sequence
  Synthetic polypeptide
MAAEDALPSGYVSFGHVGGPPPSPASPSIQYSILREEREAELDSELIIESCDASSASEESPKREQDSPPMKPSALDAIREETGVRAEERAPSRRGLAEPGSFLDYPSTEPQPGPELPPGDGALEPETPMLPRKPEEDSSSNQSPAATKGPGPLGPGAPPPLLFLNKQKAIDLLYWRDIKQTGIVFGSFLLLLFSLTQFSVVSVVAYLALAALSATISFRIYKSVLQAVQKTDEGHPFKAYLELEITLSQEQIQKYTDCLQFYVNSTLKELRRLFLVQDLVDSLKFAVLMWLLTYVGALFNGLTLLLMAVVSMFTLPVVYVKHQAQIDQYLGLVRTHINAVVAKIQAKIPGAKRHAE

SEQ ID NO: 340 - protein, 208 aa, Artificial Sequence
  Synthetic polypeptide
MQATADSTKMDCVWSNWKSQAIDLLYWRDIKQTGIVFGSFLLLLFSLTQFSVVSVVAYLALAALSATISFRIYKSVLQAVQKTDEGHPFKAYLELEITLSQEQIQKYTDCLQFYVNSTLKELRRLFLVQDLVDSLKFAVLMWLLTYVGALFNGLTLLLMAVVSMFTLPVVYVKHQAQIDQYLGLVRTHINAVVAKIQAKIPGAKRHAE

SEQ ID NO: 341 - protein, 1192 aa, Homo sapiens; identical to SEQ ID NO: 331

SEQ ID NO: 342 - protein, 373 aa, Artificial Sequence
  Synthetic polypeptide
MEDLDQSPLVSSSDSPPRPQPAFKYQFVREPEDEEEEEEEEEEDEDEDLEELEVLERKPAAGLSAAPVPTAPAAGAPLMDFGNDFVPPAPRGPLPAAPPVAPERQPSWDPSPVSSTVPAPSPLSAAAVSPSKLPEDDEPPARPPPPPPASVSPQAEPVWTPPAPAPAAPPSTPAAPKRRGSSGSVVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 343 - protein, 199 aa, Artificial Sequence
  Synthetic Polypeptide
MDGQKKNWKDKVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE